(19) United States
(12) Patent Application Publication
(10) Pub. No.: US 2025/0295642 A1
(43) Pub. Date: Sep. 25, 2025

(54) FLUOROQUINOXALINONE DERIVATIVE FOR SELECTIVELY INHIBITING PARP1

(71) Applicant: MEDSHINE DISCOVERY INC., Jiangsu (CN)

(72) Inventors: Zhengxia CHEN, Shanghai (CN); Meibi DAI, Shanghai (CN); Yang ZHANG, Shanghai (CN); Shuhui CHEN, Shanghai (CN)

(21) Appl. No.: 18/862,180

(22) PCT Filed: May 6, 2023
(86) PCT No.: PCT/CN2023/092572

§ 371 Date: Nov. 1, 2024

(30) Foreign Application Priority Data
May 7, 2022 (CN) 202210495459.8
Jun. 17, 2022 (CN) 202210693545.X
Sep. 29, 2022 (CN) 202211204558.2
Oct. 31, 2022 (CN) 202211351783.9
Nov. 10, 2022 (CN) 202211408423.8
Dec. 9, 2022 (CN) 202211593592.3
Feb. 2, 2023 (CN) 202310088997.X
Feb. 15, 2023 (CN) 202310123790.1
Mar. 10, 2023 (CN) 202310232367.5
Mar. 23, 2023 (CN) 202310303425.9
Mar. 31, 2023 (CN) 202310340861.3
Apr. 12, 2023 (CN) 202310388951.X

(51) Int. Cl. A61K 31/4375; A61K 31/4985; C07D 487/04; C07D 498/14; C07D 519/00

(52) U.S. Cl. CPC A61K 31/4375; A61K 31/4985; C07D 487/04; C07D 498/14; C07D 519/00

(57) ABSTRACT

Disclosed is a series of fluorine-substituted heterocyclic compounds, and specifically, disclosed are a compound represented by formula (XII) and a pharmaceutically acceptable salt thereof.

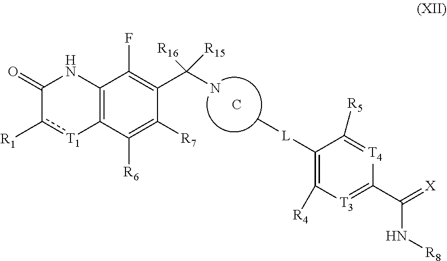

### SPECIFICATION

The present application claims the right of the following priorities:
CN202210495459.8, application date: May 7, 2022;
CN202210693545.X, application date: Jun. 17, 2022;
CN202211204558.2, application date: Sep. 29, 2022;
CN202211351783.9, application date: Oct. 31, 2022;
CN202211408423.8, application date: Nov. 10, 2022;
CN202211593592.3, application date: Dec. 9, 2022;
CN202310088997X, application date: Feb. 2, 2023;
CN2023101237901, application date: Feb. 15, 2023;
CN2023102323675, application date: Mar. 10, 2023;
CN2023103034259, application date: Mar. 23, 2023;
CN2023103408613, application date: Mar. 31, 2023;
CN202310388951X, application date: Apr. 12, 2023.

### TECHNICAL FIELD

The present disclosure relates to a series of fluorine-substituted heterocyclic compounds, specifically to a compound of formula (XII) and a pharmaceutically acceptable salt thereof.

### BACKGROUND

Poly-ADP-ribose polymerase (PARP) is a superfamily of proteases, currently consisting of 18 members. It plays a significant role in various cell cycle processes, such as replication, recombination, chromatin remodeling, and DNA damage repair.

Among them, PARP1 and PARP2 have been extensively studied due to their roles in DNA damage repair. PARP1 is activated by DNA damage breaks and catalyzes the addition of poly(ADP-ribose) (PAR) chains to target proteins. This post-translational modification, known as PARylation, mediates the recruitment of additional DNA repair factors to the site of DNA damage. After completing the recruitment of DNA repair factors, PARP undergoes auto-PARylation, which triggers the release of PARP bound to DNA, thereby allowing other DNA repair proteins to access and complete the repair.

Inhibiting PARP family enzymes has been developed as an anti-tumor strategy, which selectively kills cancer cells by inactivating complementary DNA repair pathways. Numerous preclinical and clinical studies have shown that tumor cells with BRCA1 or BRCA2 mutations, where BRCA is a key tumor suppressor protein involved in double-strand DNA break (DSB) repair through homologous recombination (HR), possess defective homologous recombination repair (HRD) pathways and rely on the function of PARP enzyme for survival. The growth of tumors with BRCA mutations becomes more dependent on the PARP pathway, making the tumors particularly sensitive to PARP1 inhibitors. In addition to being effective against BRCA-mutated cancers, clinical studies have also demonstrated certain efficacy of PARP inhibitors in non-BRCA-mutated tumors that exhibit homologous recombination defects.

Compared to other clinical PARP1/2 inhibitors, highly selective PARP1 inhibitors enhance selectivity for PARP1 over PARP2, which can reduce hematological toxicity caused by PARP2 inhibition, thereby achieving better clinical efficacy with lower toxicity. Therefore, there remains an unmet medical need for effective and safe PARP inhibitors, particularly for PARP inhibitors that selectively target PARP1.

PARP1-selective inhibitors can be used alone for solid tumors such as ovarian cancer, breast cancer, triple-negative breast cancer, and prostate cancer with BRCA1/2 mutations (synthetic lethality mechanism). They can also be used as combination therapies to enhance the effects of DNA-damage-based anti-cancer drugs (e.g., DNA alkylating agents, topoisomerase inhibitors, and platinum-based chemotherapeutics) and radiotherapy. Additionally, they can be used in combination with other targeted therapies, such as PD-1 inhibitors, androgen receptor (AR) inhibitors, cell cycle checkpoint kinase (Chk) inhibitors, and c-Met inhibitors, for the treatment of various BRCA-negative or BRCA-positive solid tumors.

### CONTENT OF THE PRESENT INVENTION

The present disclosure provides a compound of formula (XII) or a pharmaceutically acceptable salt thereof,

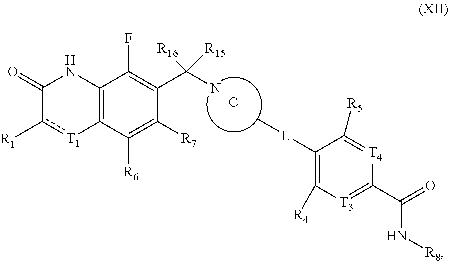

wherein
X is selected from O and S;
the structural moiety

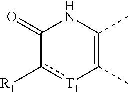

is selected from

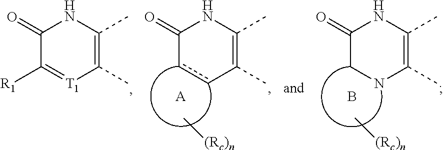

ring A is selected from phenyl and 6-membered heteroaryl;
ring B is selected from 6-membered heteroaryl;
L is selected from a single bond, and ring C is selected from

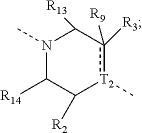

[symbol] is selected from a single bond or a double bond;
T₂ is selected from C, N, and CH;
T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₃ is selected from H and halogen;
R₉ is absent, or selected from H and halogen;
R₄ is selected from H, halogen, CN, C₁₋₃ alkyl, and C₁₋₃ alkoxy;
R₅ is selected from H, halogen, CN, C₁₋₃ alkyl, and C₁₋₃ alkoxy;
R₆ and R₇ are each independently selected from H, halogen, C₁₋₃ alkyl, and C₁₋₃ alkoxy, and the C₁₋₃ alkyl and C₁₋₃ alkoxy are each independently and optionally substituted by 1, 2, or 3 halogens;

R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R;
R₁₀ is selected from H and halogen;
R₁₃ and R₁₄ are each independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are each independently and optionally substituted by 1, 2, or 3 halogens;
R₁₅ and R₁₆ are each independently selected from H, D, C₁₋₃ alkyl, and C₁₋₃ alkoxy, and the C₁₋₃ alkyl and C₁₋₃ alkoxy are each independently and optionally substituted by 1, 2, or 3 halogens;
or, R₂ and R₄ form a ring, so that the structural moiety

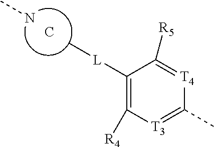

is selected from

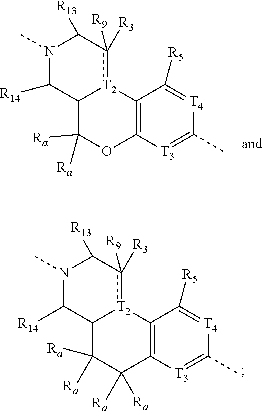

or, R₃ and R₅ form a ring, so that the structural moiety

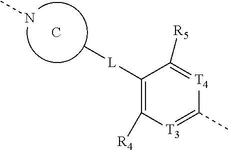

is selected from

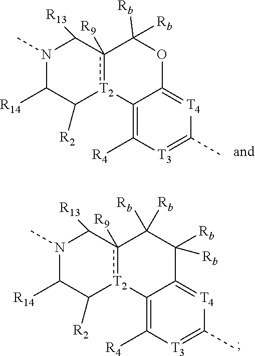

each Rₐ is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
or, two Rₐ on adjacent atoms, together with the atom to which they are attached, form a double bond or a cyclopropyl group;
each R_{b} is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
each R_{c} is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
each R is independently selected from halogen and D;
n is selected from 0, 1, 2, 3, and 4;
given that when the structural moiety

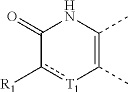

is selected from

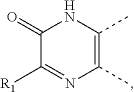

L is selected from a single bond, and ring C is selected from

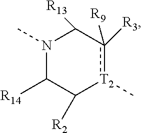

then R₂ and R₄ form a ring, or R₃ and R₅ form a ring;
the halogen represents F, Cl, Br, and I atoms;
“hetero” in the 5- to 6-membered heteroaryl represents 1, 2, 3, or 4 heteroatoms or heteroatom groups, each independently selected from —O—, —S—, and —N—.

The present disclosure further provides a compound of formula (XII) or a pharmaceutically acceptable salt thereof,

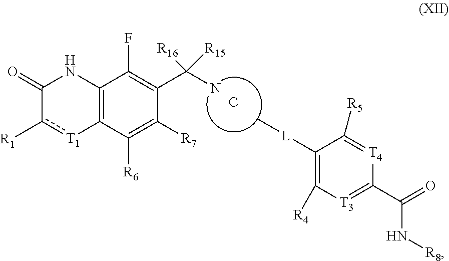

wherein
X is selected from O, S, and N(R_{d});
the structural moiety

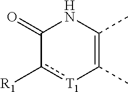

is selected from

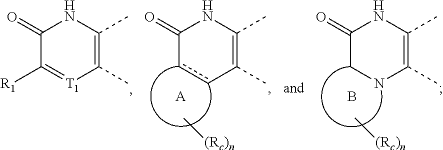

ring A is selected from phenyl and 5- to 6-membered heteroaryl;
ring B is selected from 5- to 6-membered heteroaryl;
L is selected from a single bond, and ring C is selected from

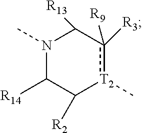

or, L is selected from N(R₁₂), and ring C is selected from

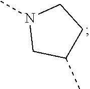

[symbol] is selected from a single bond or a double bond;
T₂ is selected from C, N, and CH;
T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₃ is selected from H and halogen;
R₉ is absent, or selected from H and halogen;
R₄ is selected from H, halogen, CN, C₁₋₃ alkyl, and C₁₋₃ alkoxy;
R₅ is selected from H, halogen, CN, C₁₋₃ alkyl, and C₁₋₃ alkoxy;
R₆ and R₇ are each independently selected from H, halogen, C₁₋₃ alkyl, and C₁₋₃ alkoxy, and the C₁₋₃ alkyl and C₁₋₃ alkoxy are each independently and optionally substituted by 1, 2, or 3 halogens;
R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R;
R₁₀ is selected from H and halogen;
R₁₂ is selected from H, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are each independently and optionally substituted by 1, 2, or 3 halogens;
R₁₃ and R₁₄ are each independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are each independently and optionally substituted by 1, 2, or 3 halogens;
R₁₅ and R₁₆ are each independently selected from H, D, C₁₋₃ alkyl, and C₁₋₃ alkoxy, and the C₁₋₃ alkyl and C₁₋₃ alkoxy are each independently and optionally substituted by 1, 2, or 3 halogens;
or, R₂ and R₄ form a ring, so that the structural moiety

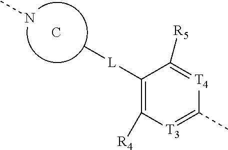

is selected from

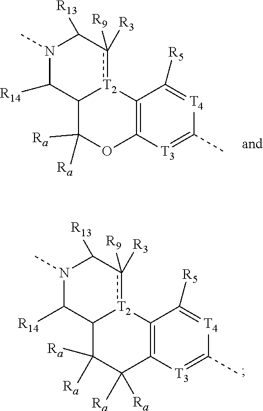

or, R₃ and R₅ form a ring, so that the structural moiety

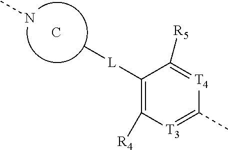

is selected from

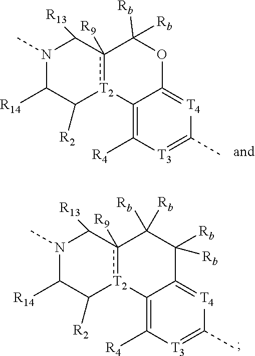

each Rₐ is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
or, two Rₐ on adjacent atoms, together with the atom to which they are attached, form a double bond or a cyclopropyl group;
each R_{b} is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
each R_{c} is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
R_{d} is selected from C₁₋₃ alkoxy, and the C₁₋₃ alkoxy is optionally substituted by 1, 2, or 3 halogens;
each R is independently selected from halogen and D;
n is selected from 0, 1, 2, 3, and 4;
given that
1) when the structural moiety

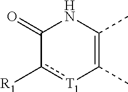

is selected from

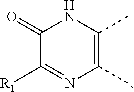

L is selected from a single bond, ring C is selected from

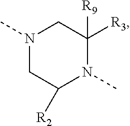

one of T₃ or T₄ is selected from N, and the other is selected from CR₁₀, then R₂, R₃, and R₉ are not simultaneously H; or
2) when the structural moiety

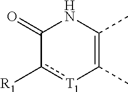

is selected from

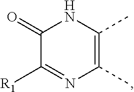

L is selected from a single bond, ring C is selected from

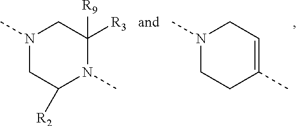

one of T₃ or T₄ is selected from N, and the other is selected from CR₁₀, then R₆ and Ry are not simultaneously H; or
3) when the structural moiety

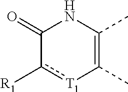

is selected from

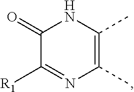

L is selected from a single bond, ring C is selected from

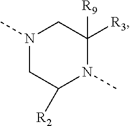

T₃ is selected from N, and T₄ is selected from CR₁₀, then R₅ is not H; or
4) when the structural moiety

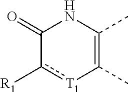

is selected from

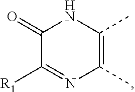

L is selected from a single bond, ring C is selected from

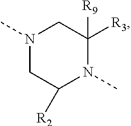

T₄ is selected from N, and T₃ is selected from CR₁₀, then R₄ is not H; or
5) when the structural moiety

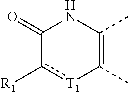

is selected from

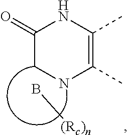

R₁₅ is selected from H, R₁₆ is selected from H, L is selected from a single bond, ring C is selected from

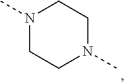

one of T₃ or T₄ is selected from N, and the other is selected from CR₁₀, then ring B is not pyrrolyl or pyrazolyl; or
6) when the structural moiety

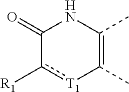

is selected from

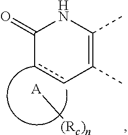

R₁₅ is selected from H, R₁₆ is selected from H, L is selected from a single bond, ring C is selected from

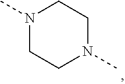

one of T₃ or T₄ is selected from N, and the other is selected from CR₁₀, then ring A is not furanyl.

The present disclosure further provides a compound of formula (XI) or a pharmaceutically acceptable salt thereof,

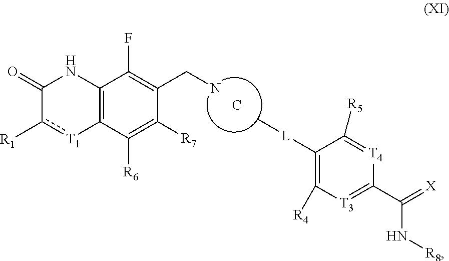

wherein
X is selected from O, S, and N(R_{d});
the structural moiety

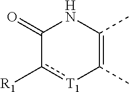

is selected from

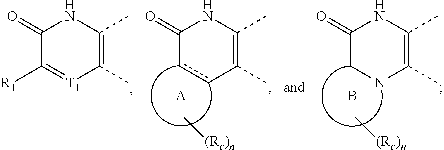

ring A is selected from phenyl and 5- to 6-membered heteroaryl;
ring B is selected from 5- to 6-membered heteroaryl;
L is selected from a single bond, and ring C is selected from

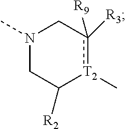

or, Lis selected from N(R₁₂), and ring C is selected from

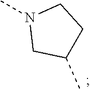

[symbol] is selected from a single bond or a double bond;
T₂ is selected from C, N, and CH;
T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₃ is selected from H and halogen;
R₉ is absent, or selected from H and halogen;
R₄ is selected from H and halogen;
R₅ is selected from H and halogen;
R₆ and R₇ are each independently selected from H and halogen;
R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R;
R₁₀ is selected from H and halogen;
R₁₂ is selected from H, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are each independently and optionally substituted by 1, 2, or 3 halogens;

or, R₂ and R₄ form a ring, so that the structural moiety

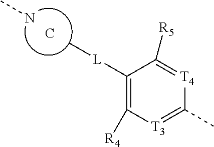

is selected from

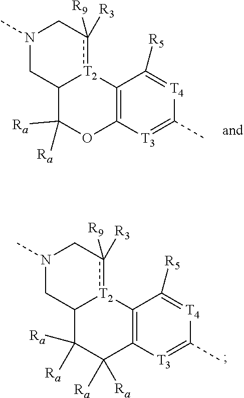

or, R₃ and R₅ form a ring, so that the structural moiety

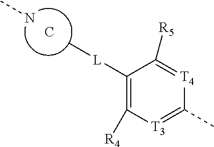

is selected from

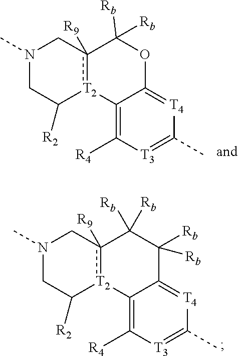

each Rₐ is independently selected from H, halogen, and C₁₋₃ alkyl, and the C₁₋₃ alkyl is optionally substituted by 1, 2, or 3 halogens;
or, two Rₐ on adjacent atoms, together with the atom to which they are attached, form a double bond or a cyclopropyl group;
each R_{b} is independently selected from H, halogen, and C₁₋₃ alkyl, and the C₁₋₃ alkyl is optionally substituted by 1, 2, or 3 halogens;
each R_{c} is independently selected from H, halogen, and C₁₋₃ alkyl, and the C₁₋₃ alkyl is optionally substituted by 1, 2, or 3 halogens;
R_{d} is selected from C₁₋₃ alkoxy, and the C₁₋₃ alkoxy is optionally substituted by 1, 2, or 3 halogens;
each R is independently selected from halogen and D;
n is selected from 0, 1, 2, 3, and 4;
given that
1) when the structural moiety

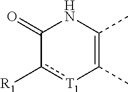

is selected from

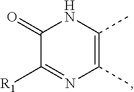

L is selected from a single bond, ring C is selected from

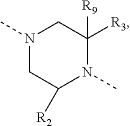

and one of T₃ or T₄ is selected from N, then R₂, R₃, and Ry are not simultaneously H; or
2) when the structural moiety

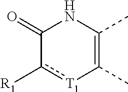

is selected from

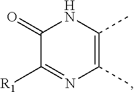

L is selected from a single bond, ring C is selected from

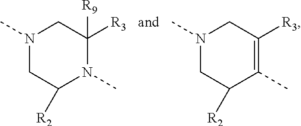

and one of T₃ or T₄ is selected from N, then R₆ and R₇ are not simultaneously H; or
3) when the structural moiety

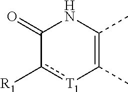

is selected from

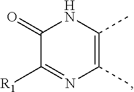

L is selected from a single bond, ring C is selected from

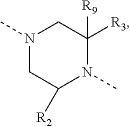

T₃ is selected from N, and T₄ is selected from CR₁₀, then R₅ is selected from halogen; or
4) when the structural moiety

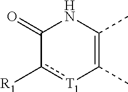

is selected from

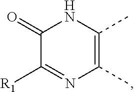

L is selected from a single bond, ring C is selected from

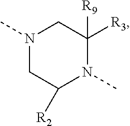

T₄ is selected from N, and T₃ is selected from CR₁₀, then R₄ is selected from halogen.

The present disclosure further provides a compound of formula (VIII) or a pharmaceutically acceptable salt thereof,

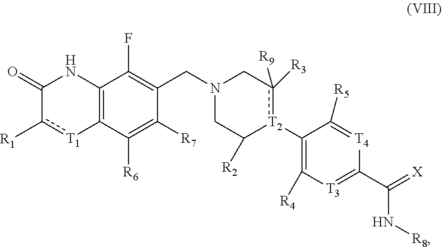

wherein
X is selected from O, S, and N(R_{d});
the structural moiety

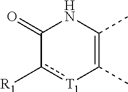

is selected from

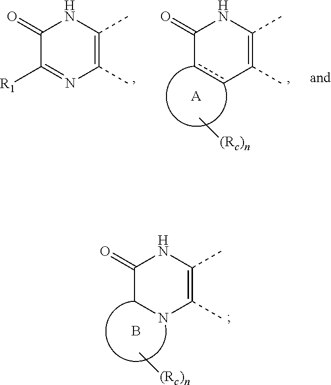

ring A is selected from phenyl and 5- to 6-membered heteroaryl;
ring B is selected from 5- to 6-membered heteroaryl;
[symbol] is selected from a single bond or a double bond;
T₂ is selected from C, N, and CH;
T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₃ is selected from H and halogen;
R₉ is absent, or selected from H and halogen;
R₄ is selected from H and halogen;
R₅ is selected from H and halogen;
R₆ and R₇ are each independently selected from H and halogen;
R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R;
R₁₀ is selected from H and halogen;
or, R₂ and R₄ form a ring, so that the structural moiety

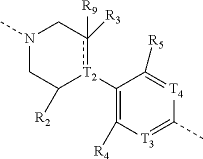

is selected from

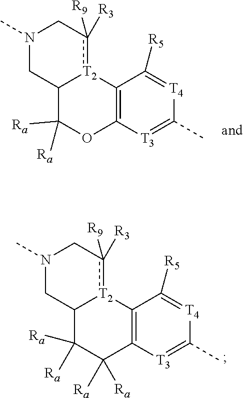

or, R₃ and R₅ form a ring, so that the structural moiety

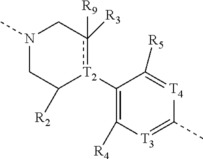

is selected from

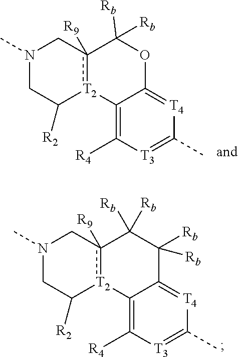

each Rₐ is independently selected from H, halogen, and C₁₋₃ alkyl, and the C₁₋₃ alkyl is optionally substituted by 1, 2, or 3 halogens;
or, two Rₐ on adjacent atoms, together with the atom to which they are attached, form a double bond or a cyclopropyl group;
each R_{b} is independently selected from H, halogen, and C₁₋₃ alkyl, and the C₁₋₃ alkyl is optionally substituted by 1, 2, or 3 halogens;
each R_{c} is independently selected from H, halogen, and C₁₋₃ alkyl, and the C₁₋₃ alkyl is optionally substituted by 1, 2, or 3 halogens;
R_{d} is selected from OCH₃;
each R is independently selected from halogen and D;
n is selected from 0, 1, 2, 3, and 4;
given that
1) when the structural moiety

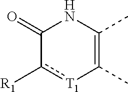

is selected from

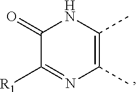

the structural moiety

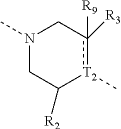

is selected from

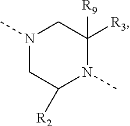

and one of T₃ or T₄ is selected from N, then R₂, R₃, and R₉ are not simultaneously H; or
2) when the structural moiety

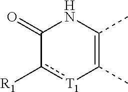

is selected from

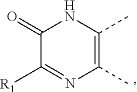

the structural moiety

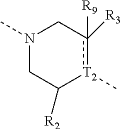

is selected from

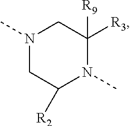

and one of T₃ or T₄ is selected from N, then R₆ and R₇ are not simultaneously H; or
3) when the structural moiety

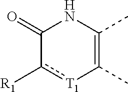

is selected from

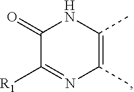

the structural moiety

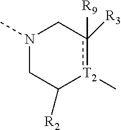

is selected from

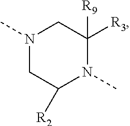

T₃ is selected from N, and T₄ is selected from CR₁₀, then R₅ is selected from halogen; or
4) when the structural moiety

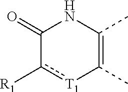

is selected from

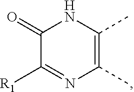

the structural moiety

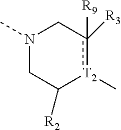

is selected from

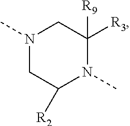

T₄ is selected from N, and T₃ is selected from CR₁₀, then R₄ is selected from halogen; or
5) when the structural moiety

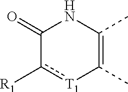

is selected from

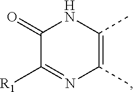

the structural moiety

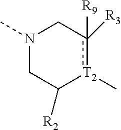

is selected from

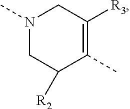

and one of T₃ or T₄ is selected from N, then R₆ and R₇ are not simultaneously H.

The present disclosure further provides a compound of formula (VIII) or a pharmaceutically acceptable salt thereof,

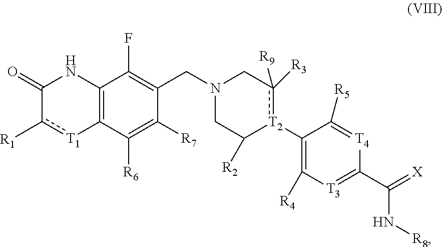

wherein
X is selected from O, S, and N(R_{d});
the structural moiety

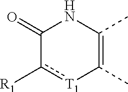

is selected from

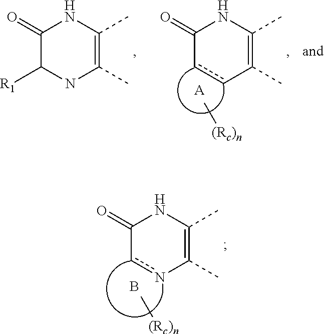

ring A is selected from phenyl and 5- to 6-membered heteroaryl;
ring B is selected from 5- to 6-membered heteroaryl;
[symbol] is selected from a single bond or a double bond;
the structural moiety

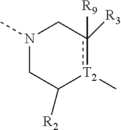

is selected from

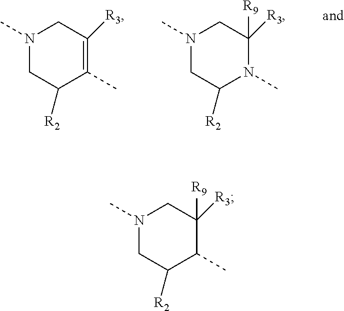

T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₃ and R₉ are each independently selected from H and halogen;
R₄ is selected from H and halogen;
R₅ is selected from H and halogen;
R₆ and R₇ are each independently selected from H and halogen;
R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R;
R₁₀ is selected from H and halogen;
or, R₂ and R₄ form a ring, so that the structural moiety

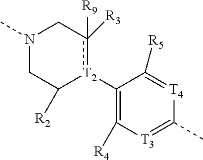

is selected from

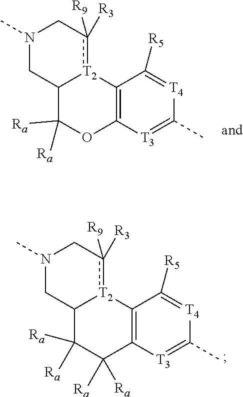

or, R₃ and R₅ form a ring, so that the structural moiety

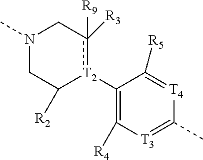

is selected from

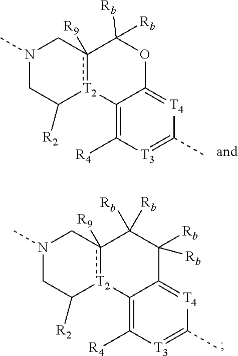

T₂ is selected from C, N, and CH;
each Rₐ is independently selected from H, halogen, and C₁₋₃ alkyl, and the C₁₋₃ alkyl is optionally substituted by 1, 2, or 3 halogens;
or, two Rₐ on adjacent atoms, together with the atom to which they are attached, form a double bond or a cyclopropyl group;
each R_{b} is independently selected from H, halogen, and C₁₋₃ alkyl, and the C₁₋₃ alkyl is optionally substituted by 1, 2, or 3 halogens;
each R_{c} is independently selected from H, halogen, and C₁₋₃ alkyl, and the C₁₋₃ alkyl is optionally substituted by 1, 2, or 3 halogens;
R_{d} is selected from OCH₃;
each R is independently selected from halogen and D;
n is selected from 0, 1, 2, 3, and 4;
given that
1) when the structural moiety

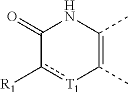

is selected from

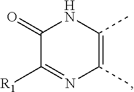

the structural moiety

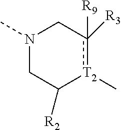

is selected from

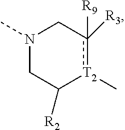

and one of T₃ or T₄ is selected from N, then R₂, R₃, and R₉ are not simultaneously H; or
2) when the structural moiety

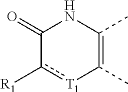

is selected from

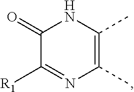

the structural moiety

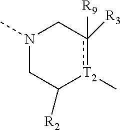

is selected from

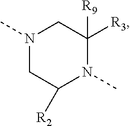

and one of T₃ or T₄ is selected from N, then R₆ and R₇ are not simultaneously H; or
3) when the structural moiety

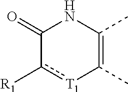

is selected from

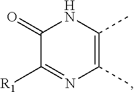

the structural moiety

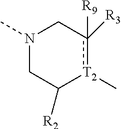

is selected from

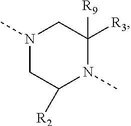

T₃ is selected from N, and T₄ is selected from CR₁₀, then R₅ is selected from halogen; or
4) when the structural moiety

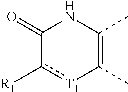

is selected from

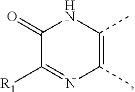

the structural moiety

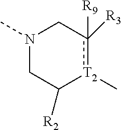

is selected from

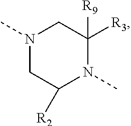

T₄ is selected from N, and T₃ is selected from CR₁₀, then R₄ is selected from halogen; or
5) when the structural moiety

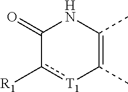

is selected from

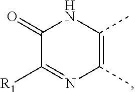

the structural moiety

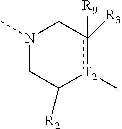

is selected from

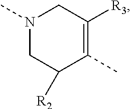

and one of T₃ or T₄ is selected from N, then R₆ and R₇ are not simultaneously H.

The present disclosure further provides a compound of formula (VII-1) or a pharmaceutically acceptable salt thereof,

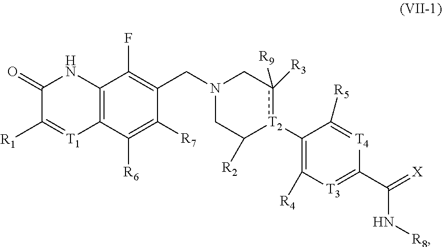

wherein
when [symbol] is selected from a double bond, then T₂ is selected from C, R₉ is absent, and R₃ is selected from H and halogen;
when [symbol] is selected from a single bond, then T₂ is selected from N and CH, R₉ and R₃ are each independently selected from H and halogen;
X is selected from O, S, and N(R_{d});
T₁ is selected from N;
or, T₁ and R₁ form a ring, so that the structural moiety

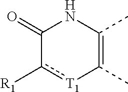

is selected from

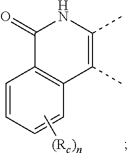

T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₄ is selected from H and halogen;
R₅ is selected from H and halogen;
R₆ and R₇ are each independently selected from H and halogen;
R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R; R₁₀ is selected from H and halogen;
or, R₂ and R₄ form a ring, so that the structural moiety

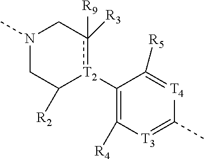

is selected from

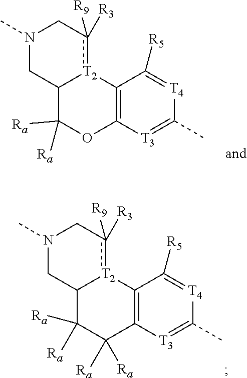

or, R₃ and R₅ form a ring, so that the structural moiety

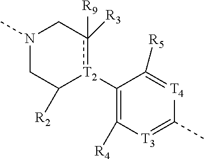

is selected from

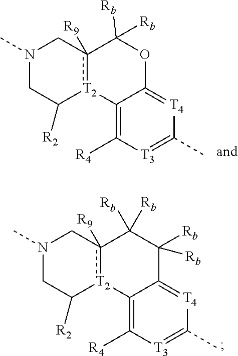

each Rₐ is independently selected from H, halogen, and C₁₋₃ alkyl, and the C₁₋₃ alkyl is optionally substituted by 1, 2, or 3 halogens;
or, Rₐ on two adjacent atoms, together with the atom to which they are attached, form a double bond;
each R_{b} is independently selected from H, halogen, and C₁₋₃ alkyl, and the C₁₋₃ alkyl is optionally substituted by 1, 2, or 3 halogens;
each R_{c} is independently selected from H, halogen, and C₁₋₃ alkyl, and the C₁₋₃ alkyl is optionally substituted by 1, 2, or 3 halogens;
R_{d} is selected from OCH₃;
each R is independently selected from halogen and D;
n is selected from 0, 1, 2, 3, and 4;
given that
1) when T₁ is selected from N, T₂ is selected from N, and one of T₃ or T₄ is selected from N, then R₂, R₃, and R₉ are not simultaneously H; or
2) when T₁ is selected from N, T₂ is selected from N, and one of T₃ or T₄ is selected from N, then R₆ and R₇ are not simultaneously H; or
3) when T₁ is selected from N, T₂ is selected from N, T₃ is selected from N, and T₄ is selected from CR₁₀, then R₅ is selected from halogen; or
4) when T₁ is selected from N, T₂ is selected from N, T₄ is selected from N, and T₃ is selected from CR₁₀, then R₄ is selected from halogen; or
5) when T₁ is selected from N, the structural moiety

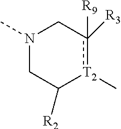

is selected from

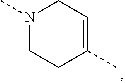

and one of T₃ or T₄ is selected from N, then R₆ and R₇ are not simultaneously H.

The present disclosure further provides a compound of formula (VI) or a pharmaceutically acceptable salt thereof,

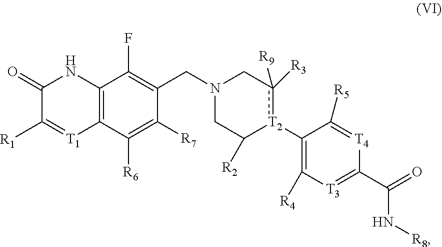

wherein
when [symbol] is selected from a double bond, then T₂ is selected from C, R₉ is absent, and R₃ is selected from H and halogen;
when [symbol] is selected from a single bond, then T₂ is selected from N and CH, R₉ and R₃ are each independently selected from H and halogen;
T₁ is selected from N and CRI;
T₃ and T₄ are selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₄ is selected from H and halogen;
R₅ is selected from H and halogen;
or, R₂ and R₄ form a ring, so that the structural moiety

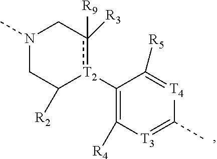

is selected from

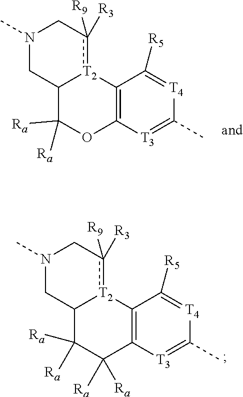

or, R₃ and R₅ form a ring, so that the structural moiety

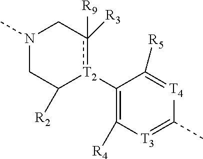

is selected from

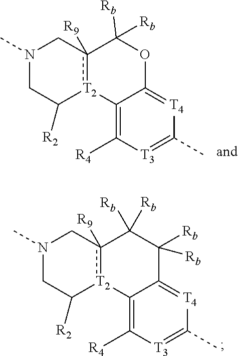

R₆ and R₇ are each independently selected from H and halogen;
R₈ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₁₁ and R₁ form a benzene ring, so that the structural moiety

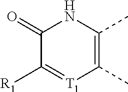

is selected from

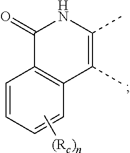

R₁₀ is selected from H and halogen;
each Rₐ is independently selected from H, halogen, and C₁₋₃ alkyl, and the C₁₋₃ alkyl is optionally substituted by 1, 2, or 3 halogens;
each R_{b} is independently selected from H, halogen, and C₁₋₃ alkyl, and the C₁₋₃ alkyl is optionally substituted by 1, 2, or 3 halogens;
each R_{c} is independently selected from H, halogen, and C₁₋₃ alkyl, and the C₁₋₃ alkyl is optionally substituted by 1, 2, or 3 halogens;
n is selected from 0, 1, 2, 3, and 4;
given that when T₁ is selected from N, T₂ is selected from N, and T₃ is selected from N, then
1) R₂, R₃, and Ry are not simultaneously H; or
2) R₆ and R₇ are not simultaneously H; or
3) R₅ is selected from halogen.

The present disclosure further provides a compound of formula (I) or a pharmaceutically acceptable salt thereof,

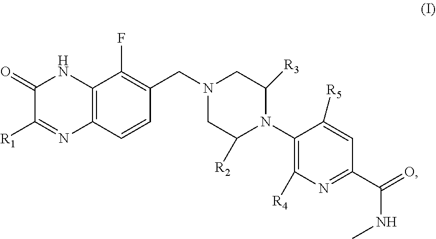

wherein
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from C₁₋₃ alkyl, and the C₁₋₃ alkyl is optionally substituted by 1, 2, or 3 halogens;
R₃ is selected from H;
R₄ is selected from halogen;
R₅ is selected from H;
or, R₂ and R₄ form a ring, so that the structural moiety

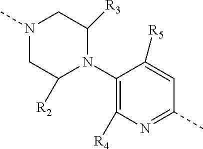

is selected from

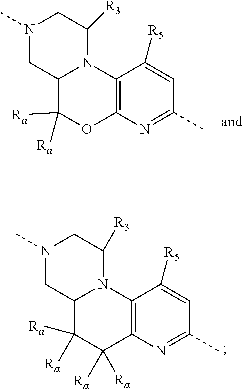

or, R₃ and R₅ form a ring, so that the structural moiety

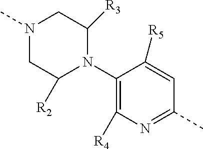

is selected from

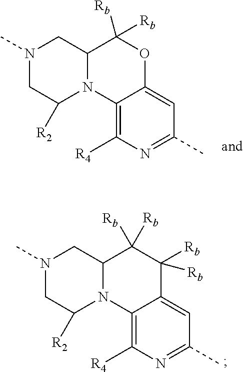

each Rₐ is independently selected from H, halogen, and C₁₋₃ alkyl, and the C₁₋₃ alkyl is optionally substituted by 1, 2, or 3 halogens;
each R_{b} is independently selected from H, halogen, and C₁₋₃ alkyl, and the C₁₋₃ alkyl is optionally substituted by 1, 2, or 3 halogens.

The present disclosure further provides a compound of formula (VII-2) or a pharmaceutically acceptable salt thereof,

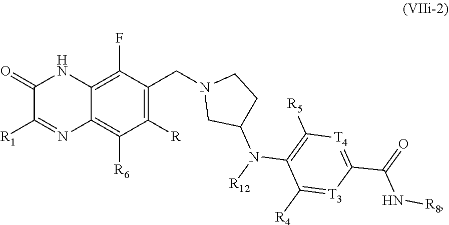

wherein
T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₄ is selected from H and halogen;
R₅ is selected from H and halogen;
R₆ and R₇ are each independently selected from H and halogen;

R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R;
R₁₀ is selected from H and halogen;
R₁₂ is selected from H and C₁₋₃ alkyl;
each R is independently selected from halogen and D.

The present disclosure further provides a compound of formula (I) or a pharmaceutically acceptable salt

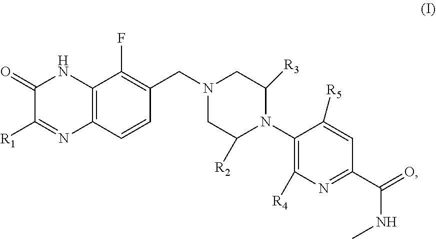

wherein
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from C₁₋₃ alkyl, and the C₁₋₃ alkyl is optionally substituted by 1, 2, or 3 halogens;
R₃ is selected from H;
R₄ is selected from halogen;
R₅ is selected from H;
or, R₂ and R₄ form a ring, so that the structural moiety

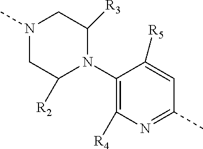

is selected from

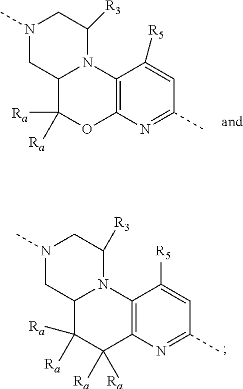

or, R₃ and R₅ form a ring, so that the structural moiety

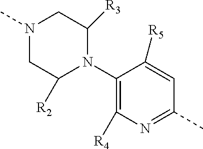

is selected from

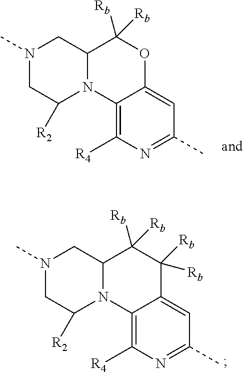

each Rₐ is independently selected from H, halogen, and C₁₋₃ alkyl, and the C₁₋₃ alkyl is optionally substituted by 1, 2, or 3 halogens;
each R_{b} is independently selected from H, halogen, and C₁₋₃ alkyl, and the C₁₋₃ alkyl is optionally substituted by 1, 2, or 3 halogens.

The present disclosure further provides a compound of formula (I) or a pharmaceutically acceptable salt thereof,

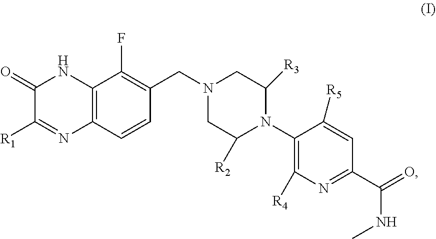

wherein
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from C₁₋₃ alkyl, and the C₁₋₃ alkyl is optionally substituted by 1, 2, or 3 halogens;
R₃ is selected from H;
R₄ is selected from halogen;
R₅ is selected from H;
or, R₂ and R₄ form a ring, so that the structural moiety

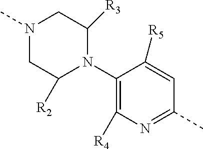

is selected from

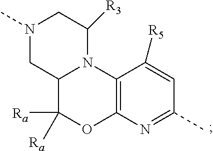

or, R₃ and R₅ form a ring, so that the structural moiety

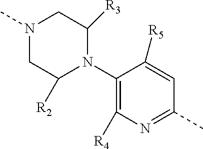

is selected from

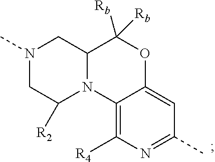

each Rₐ is independently selected from H, halogen, and C₁₋₃ alkyl, and the C₁₋₃ alkyl is optionally substituted by 1, 2, or 3 halogens;
each R_{b} is independently selected from H, halogen, and C₁₋₃ alkyl, and the C₁₋₃ alkyl is optionally substituted by 1, 2, or 3 halogens.

In some embodiments of the present disclosure, the X is selected from O, S, and N(OCH₃), and other variables are as defined in the present disclosure.

In some embodiments of the present disclosure, the X is selected from O and S, and other variables are as defined in the present disclosure.

In some embodiments of the present disclosure, the X is selected from O, and other variables are as defined in the present disclosure.

In some embodiments of the present disclosure, each Rₐ is independently selected from H, F, Cl, Br, I, CH₃, CH₂CH₃, and CH₂CH₂CH₃, and the CH₃, CH₂CH₃, and CH₂CH₂CH₃ are optionally substituted by 1, 2, or 3 halogens, and other variables are as defined in the present disclosure.

In some embodiments of the present disclosure, each Rₐ is independently selected from H, F and CH₃, and other variables are as defined in the present disclosure.

In some embodiments of the present disclosure, each Rₐ is independently selected from H and CH₃, and other variables are as defined in the present disclosure.

In some embodiments of the present disclosure, two Rₐ on adjacent atoms, together with the atom to which they are attached, form a double bond or a cyclopropyl group, so that the structural moiety

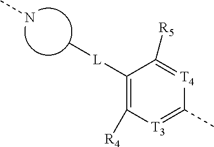

is selected from

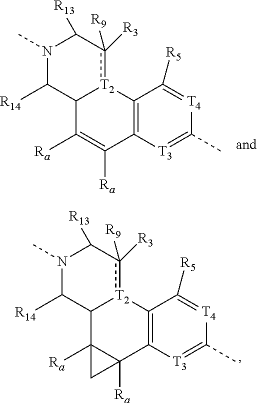

and other variables are as defined in the present disclosure.

In some embodiments of the present disclosure, two Rₐ on adjacent atoms, together with the atom to which they are attached, form a double bond or a cyclopropyl group, so that the structural moiety

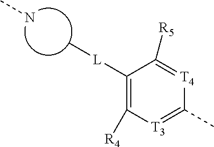

is selected from

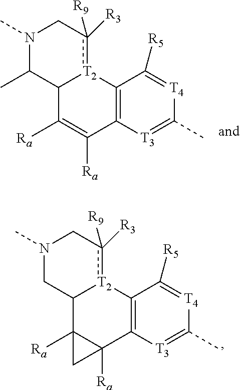

and other variables are as defined in the present disclosure.

In some embodiments of the present disclosure, two Rₐ on adjacent atoms, together with the atom to which they are attached, form a double bond or a cyclopropyl group, so that the structural moiety

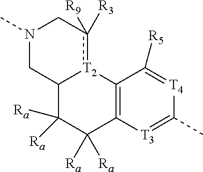

is selected from

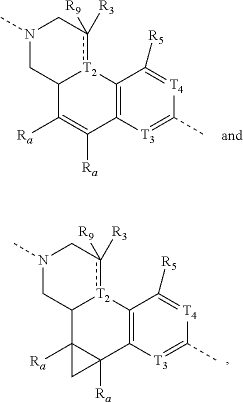

and other variables are as defined in the present disclosure.

In some embodiments of the present disclosure, Rₐ on the two adjacent atoms, together with the neighboring atom, form a double bond, so that the structural moiety

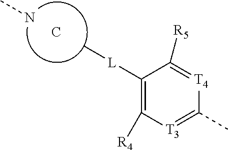

is selected from

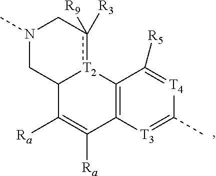

and other variables are as defined in the present disclosure.

In some embodiments of the present disclosure, Rₐ on the two adjacent atoms, together with the neighboring atom, form a double bond, so that the structural moiety

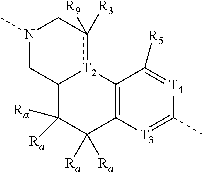

is selected from

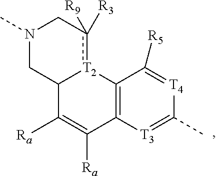

and other variables are as defined in the present disclosure.

In some embodiments of the present disclosure, each R_{b} is independently selected from H, F, Cl, Br, I, CH₃, CH₂CH₃, and CH₂CH₂CH₃, and the CH₃, CH₂CH₃, and CH₂CH₂CH₃ are optionally substituted by 1, 2, or 3 halogens, and other variables are as defined in the present disclosure.

In some embodiments of the present disclosure, each R_{b} is independently selected from H, F, and CH₃, and other variables are as defined in the present disclosure.

In some embodiments of the present disclosure, each R_{b} is independently selected from H and CH₃, and other variables are as defined in the present disclosure.

In some embodiments of the present disclosure, each R_{c} is independently selected from H, F, Cl, Br, I, CH₃, CH₂CH₃, and CH₂CH₂CH₃, and the CH₃, CH₂CH₃, and CH₂CH₂CH₃ are optionally substituted by 1, 2, or 3 halogens, and other variables are as defined in the present disclosure.

In some embodiments of the present disclosure, each R_{c} is independently selected from H, F, and CH₃, and other variables are as defined in the present disclosure.

In some embodiments of the present disclosure, each R_{c} is independently selected from H and F, and other variables are as defined in the present disclosure.

In some embodiments of the present disclosure, the Rₐ is selected from OCH₃, and other variables are as defined in the present disclosure.

In some embodiments of the present disclosure, the T₃ is selected from N, CH, and CF, and other variables are as defined in the present disclosure.

In some embodiments of the present disclosure, the T₃ is selected from N and CF, and other variables are as defined in the present disclosure.

In some embodiments of the present disclosure, the T₃ is selected from N, and other variables are as defined in the present disclosure.

In some embodiments of the present disclosure, the T₄ is selected from N and CH, and other variables are as defined in the present disclosure.

In some embodiments of the present disclosure, the R₁ is selected from CH₃ and CH₂CH₃, and other variables are as defined in the present disclosure.

In some embodiments of the present disclosure, the R₁ is selected from CH₃, and other variables are as defined in the present disclosure.

In some embodiments of the present disclosure, the R₂ is selected from H and CH₃, and other variables are as defined in the present disclosure.

In some embodiments of the present disclosure, the R₂ is selected from CH₃, and other variables are as defined in the present disclosure.

In some embodiments of the present disclosure, the R₄, R₅, R₆, and R₇ are each independently selected from H, F, and CH₃, and other variables are as defined in the present disclosure.

In some embodiments of the present disclosure, the R₃ is selected from H and F, and other variables are as defined in the present disclosure.

In some embodiments of the present disclosure, the R₄ is selected from H, F, Cl, CN, and CH₃, and other variables are as defined in the present disclosure.

In some embodiments of the present disclosure, the R₄ is selected from H, F, Cl, and CN, and other variables are as defined in the present disclosure.

In some embodiments of the present disclosure, the R₄ is selected from H, F, and CH₃, and other variables are as defined in the present disclosure.

In some embodiments of the present disclosure, the R₄ is selected from H and F, and other variables are as defined in the present disclosure.

In some embodiments of the present disclosure, the R₄ is selected from F, and other variables are as defined in the present disclosure.

In some embodiments of the present disclosure, the R₅ is selected from H, F, Cl, and CN, and other variables are as defined in the present disclosure.

In some embodiments of the present disclosure, the R₅ is selected from H, F, CN, and CH₃, and other variables are as defined in the present disclosure.

In some embodiments of the present disclosure, the R₅ is selected from H, F, and CH₃, and other variables are as defined in the present disclosure.

In some embodiments of the present disclosure, the R₅ is selected from H and F, and other variables are as defined in the present disclosure.

In some embodiments of the present disclosure, the R₅ is selected from H, and other variables are as defined in the present disclosure.

In some embodiments of the present disclosure, the R₆ and R₇ are each independently selected from H, F, and CH₃, and other variables are as defined in the present disclosure.

In some embodiments of the present disclosure, the R₆ is selected from H, F, and CH₃, and other variables are as defined in the present disclosure.

In some embodiments of the present disclosure, the R₇ is selected from H, F, and CH₃, and other variables are as defined in the present disclosure

In some embodiments of the present disclosure, the R₆ is selected from H and F, and other variables are as defined in the present disclosure.

In some embodiments of the present disclosure, the R₇ is selected from H and F, and other variables are as defined in the present disclosure.

In some embodiments of the present disclosure, the R₈ is selected from CH₃, CH₂CH₃, CH₂CF₃, cyclopropyl, and CD₃, and other variables are as defined in the present disclosure.

In some embodiments of the present disclosure, the R₈ is selected from CH₃, CH₂CF₃, cyclopropyl, and CD₃, and other variables are as defined in the present disclosure.

In some embodiments of the present disclosure, the R₈ is selected from CH₃, cyclopropyl, and CD₃, and other variables are as defined in the present disclosure.

In some embodiments of the present disclosure, the R₈ is selected from CH₃ and cyclopropyl, and other variables are as defined in the present disclosure.

In some embodiments of the present disclosure, the R₉ is selected from H and F, and other variables are as defined in the present disclosure.

In some embodiments of the present disclosure, the R₁₀ is selected from H and F, and other variables are as defined in the present disclosure.

In some embodiments of the present disclosure, the R₁₂ is selected from H and CH₃, and other variables are as defined in the present disclosure.

In some embodiments of the present disclosure, the R₁₃ is selected from H and CH₃, and other variables are as defined in the present disclosure.

In some embodiments of the present disclosure, the R₁₄ is selected from H and CH₃, and other variables are as defined in the present disclosure.

In some embodiments of the present disclosure, the R₁₅ is selected from H, D, and CH₃, and other variables are as defined in the present disclosure

In some embodiments of the present disclosure, the R₁₆ is selected from H, D, and CH₃, and other variables are as defined in the present disclosure

In some embodiments of the present disclosure, ring A is selected from phenyl and 6-membered heteroaryl ring, and other variables are as defined in the present disclosure.

In some embodiments of the present disclosure, ring A is selected from phenyl, pyrrolyl, pyrazolyl, imidazolyl, triazolyl, furanyl, thienyl, pyridyl, pyrazinyl, and pyrimidinyl, and other variables are as defined in the present disclosure.

In some embodiments of the present disclosure, ring A is selected from phenyl, pyridyl, pyrazinyl, and pyrimidinyl, and other variables are as defined in the present disclosure.

In some embodiments of the present disclosure, ring B is selected from 6-membered heteroaryl ring, and other variables are as defined in the present disclosure.

In some embodiments of the present disclosure, ring B is selected from pyrrolyl, pyrazolyl, imidazolyl, triazolyl, furanyl, thienyl, pyridyl, pyrazinyl, and pyrimidinyl, and other variables are as defined in the present disclosure.

In some embodiments of the present disclosure, ring B is selected from pyridyl, pyrazinyl, and pyrimidinyl, and other variables are as defined in the present disclosure.

In some embodiments of the present disclosure, the structural moiety

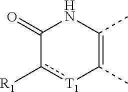

is selected from

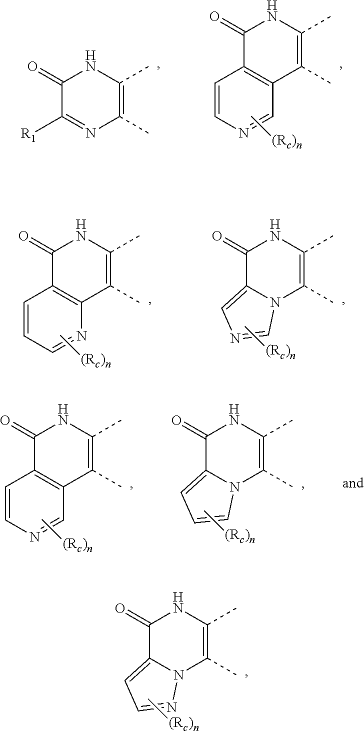

and other variables are as defined in the present disclosure.

In some embodiments of the present disclosure, the structural moiety

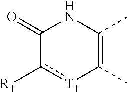

is selected from

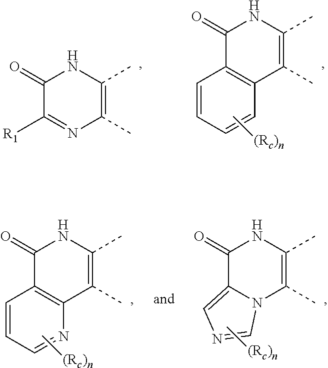

and other variables are as defined in the present disclosure.

In some embodiments of the present disclosure, the structural moiety

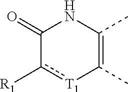

is selected from

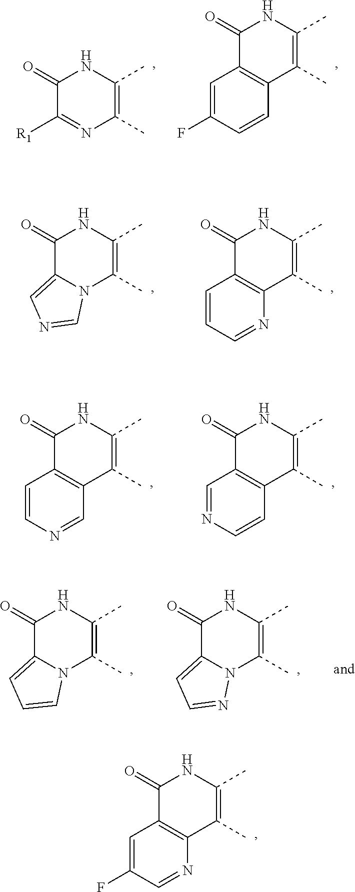

and other variables are as defined in the present disclosure.

In some embodiments of the present disclosure, the structural moiety

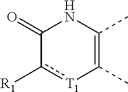

is selected from

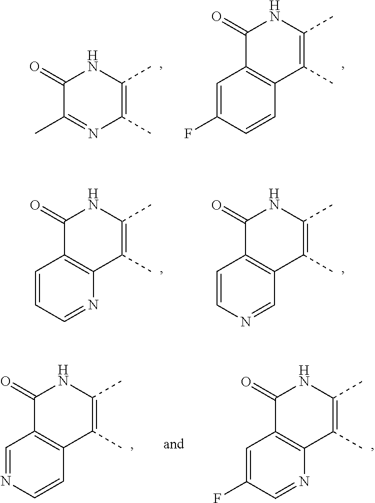

and other variables are as defined in the present disclosure.

In some embodiments of the present disclosure, the structural moiety

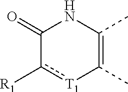

is selected from

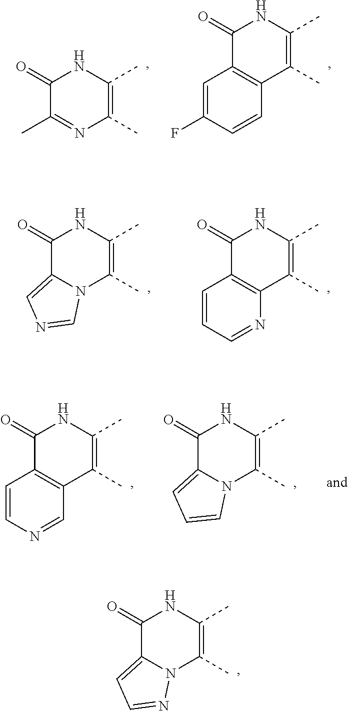

and other variables are as defined in the present disclosure.

In some embodiments of the present disclosure, the structural moiety

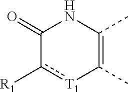

is selected from

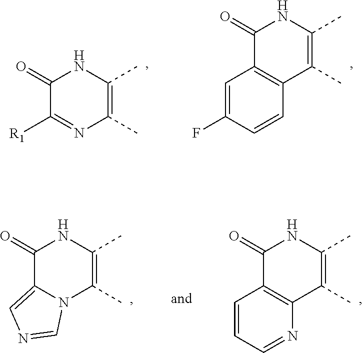

and other variables are as defined in the present disclosure.

In some embodiments of the present disclosure, R₁₁ and R₁ form a benzene ring, so that the structural moiety

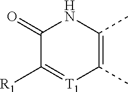

is selected from

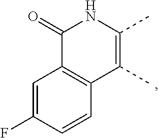

and other variables are as defined in the present disclosure. In some embodiments of the present disclosure, the structural moiety

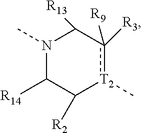

is selected from

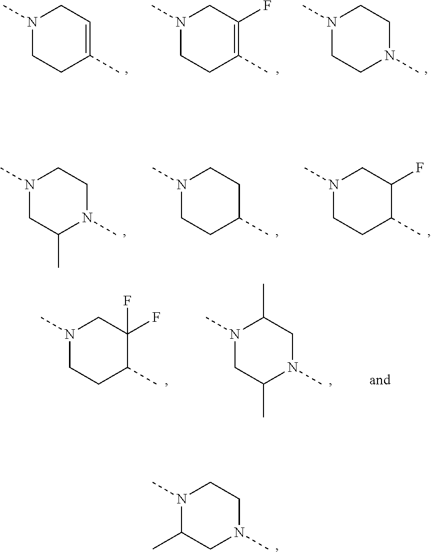

and other variables are as defined in the present disclosure.

In some embodiments of the present disclosure, the structural moiety

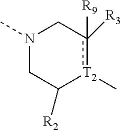

is selected from

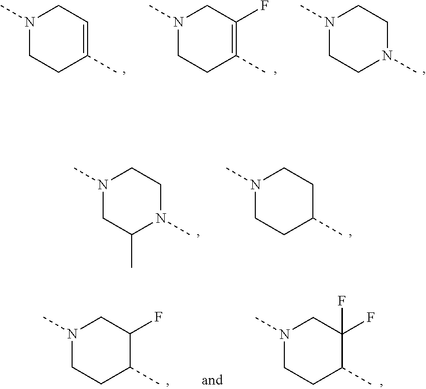

and other variables are as defined in the present disclosure.

In some embodiments of the present disclosure, the structural moiety

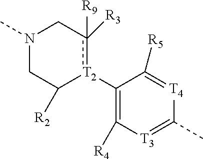

is selected from

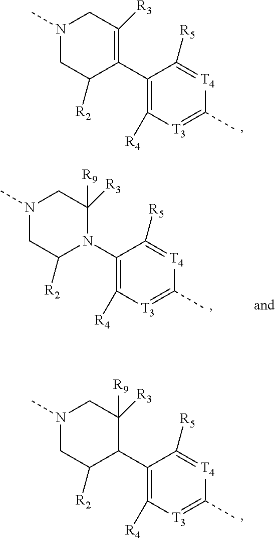

and other variables are as defined in the present disclosure.

In some embodiments of the present disclosure, the R₂ and R₄ form a ring, so that the structural moiety

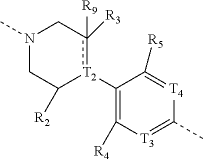

is selected from

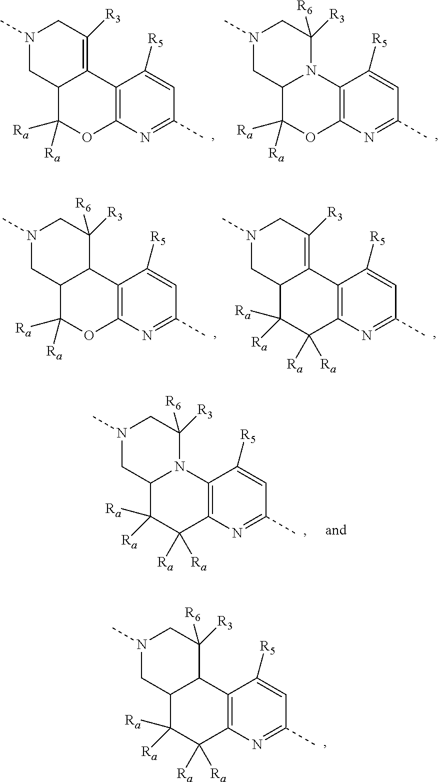

and other variables are as defined in the present disclosure.

In some embodiments of the present disclosure, the R₃ and R₅ form a ring, so that the structural moiety

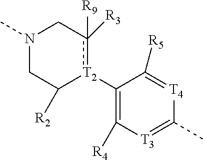

is selected from

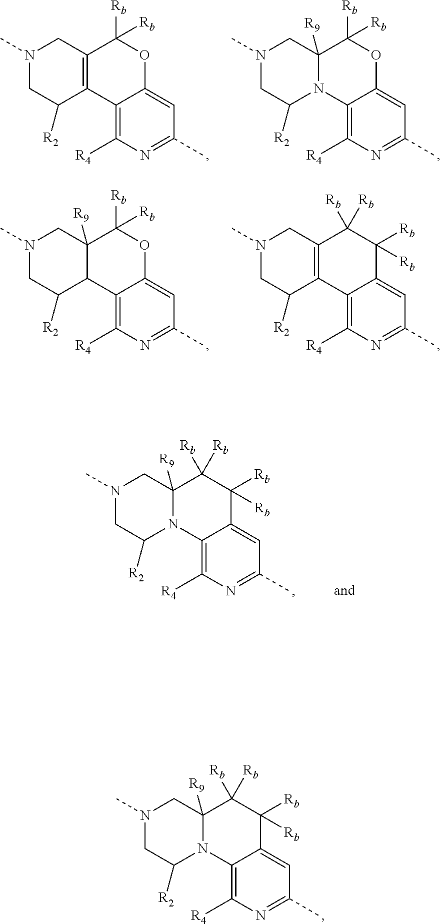

and other variables are as defined in the present disclosure.

In some embodiments of the present disclosure, the R₃ and R₅ form a ring, so that the structural moiety

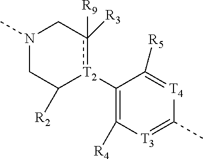

is selected from

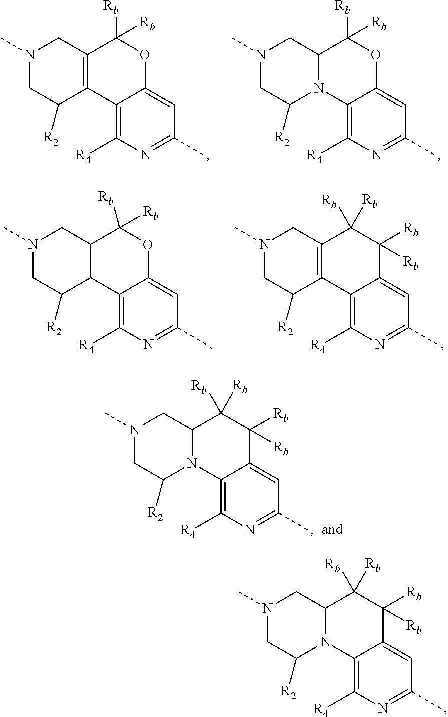

and other variables are as defined in the present disclosure.

In some embodiments of the present disclosure, the R₂ and R₄ form a ring, so that the structural moiety

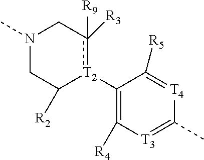

is selected from

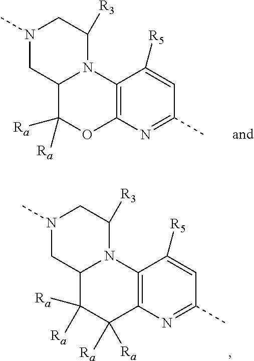

and other variables are as defined in the present disclosure.

In some embodiments of the present disclosure, the R₃ and R₅ form a ring, so that the structural moiety

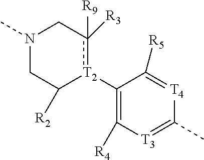

is selected from

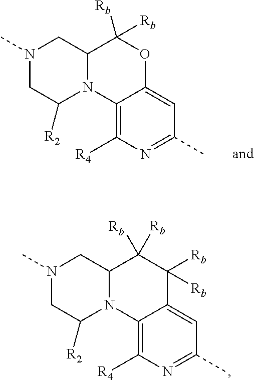

and other variables are as defined in the present disclosure.

In some embodiments of the present disclosure, the structural moiety

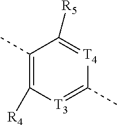

is selected from

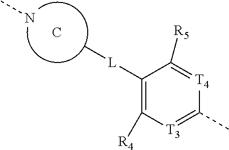

and other variables are as defined in the present disclosure.

In some embodiments of the present disclosure, the R₂ and R₄ form a ring, so that the structural moiety

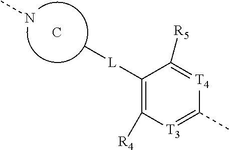

is selected from

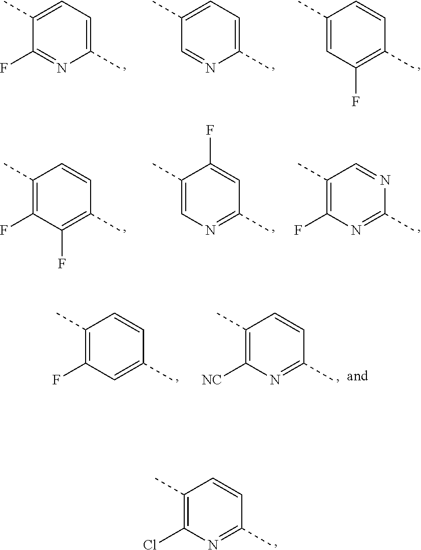

and other variables are as defined in the present disclosure.

In some embodiments of the present disclosure, the R₃ and R₅ form a ring, so that the structural moiety

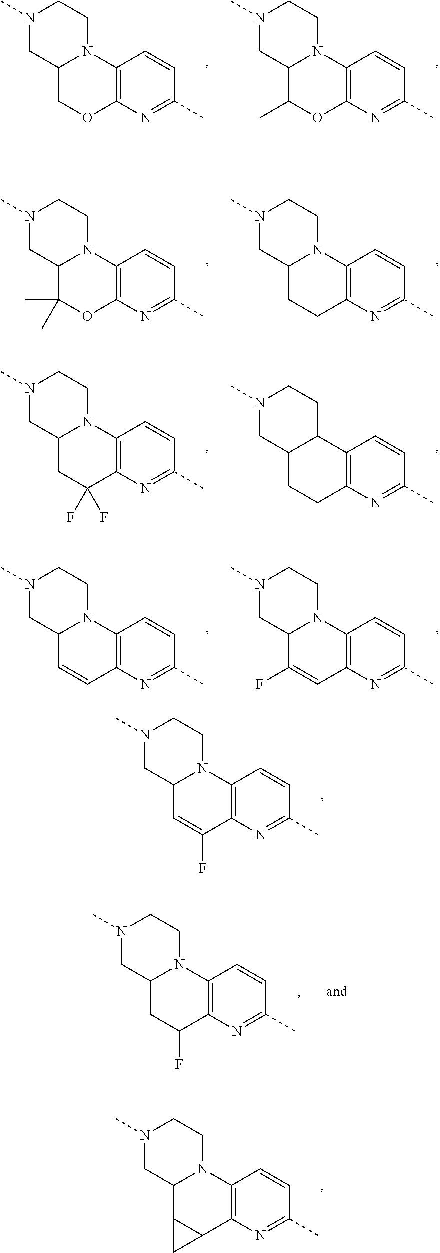

is selected from

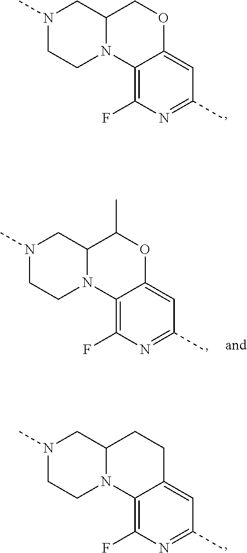

and other variables are as defined in the present disclosure.

In some embodiments of the present disclosure, the L is selected from a single bond, the structural moiety

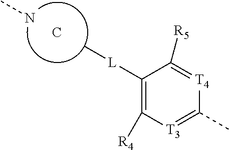

is selected from

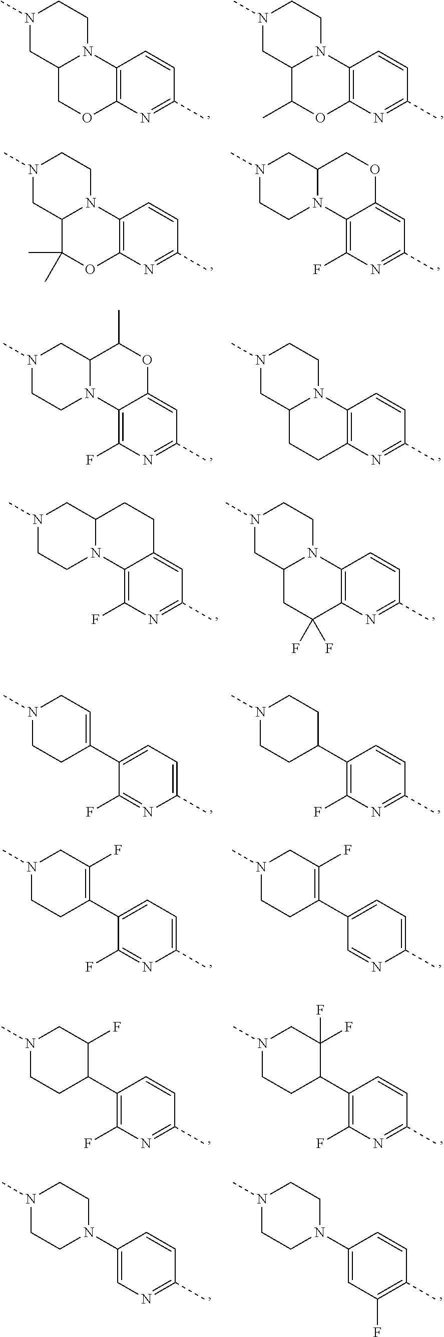

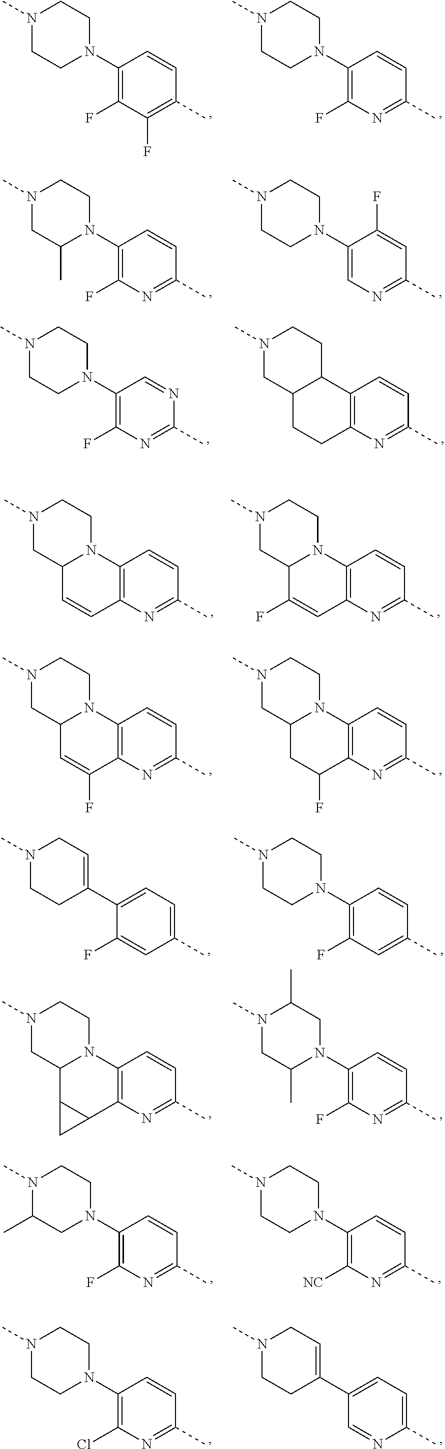

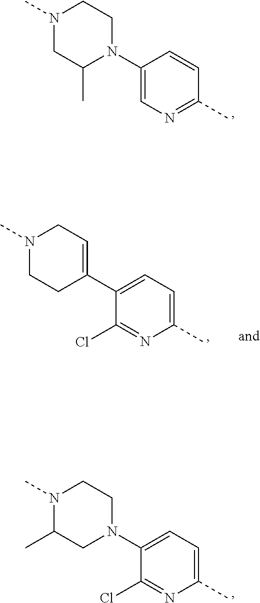

and other variables are as defined in the present disclosure.

In some embodiments of the present disclosure, the L is selected from a single bond, the structural moiety

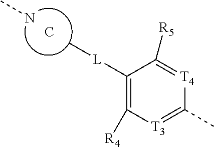

is selected from

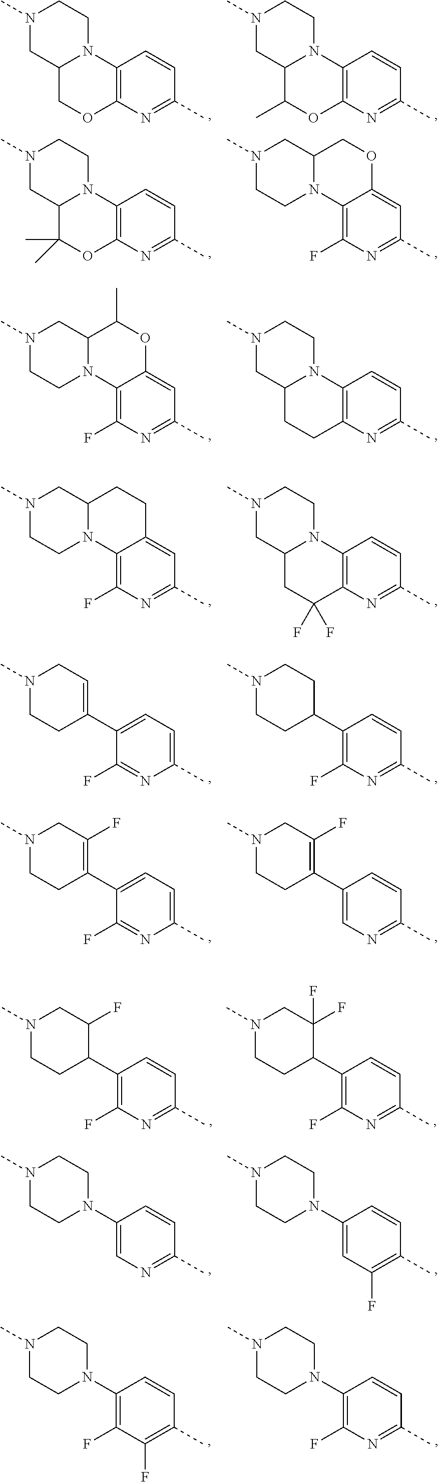

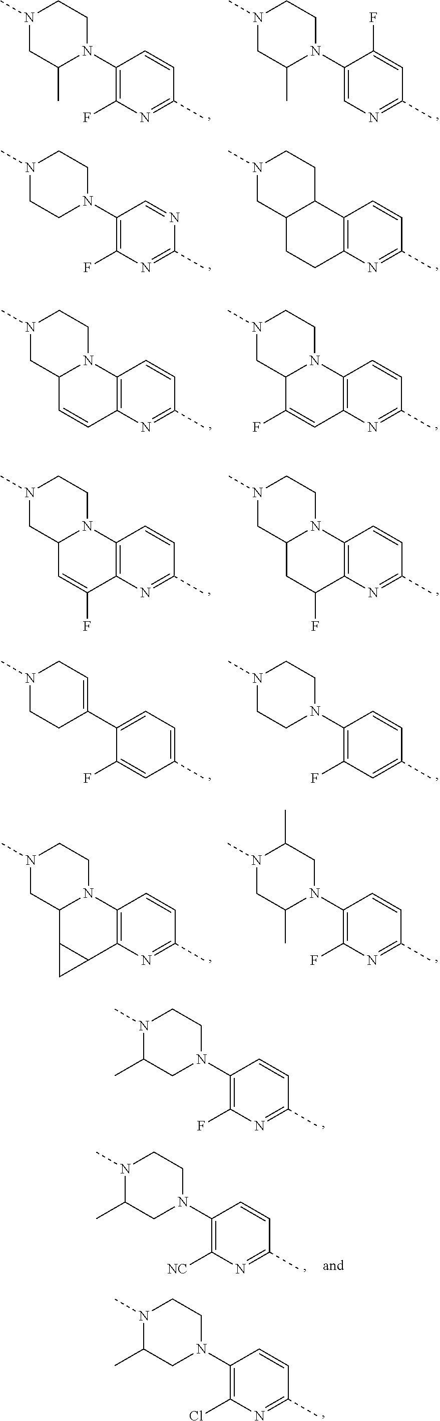

and other variables are as defined in the present disclosure.

In some embodiments of the present disclosure, the L is selected from a single bond, the structural moiety

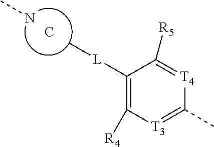

is selected from

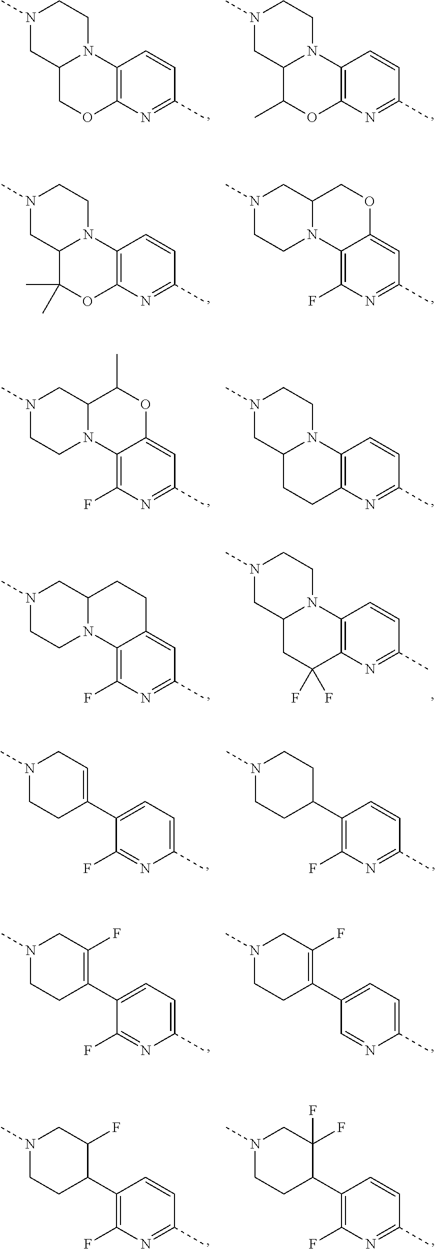

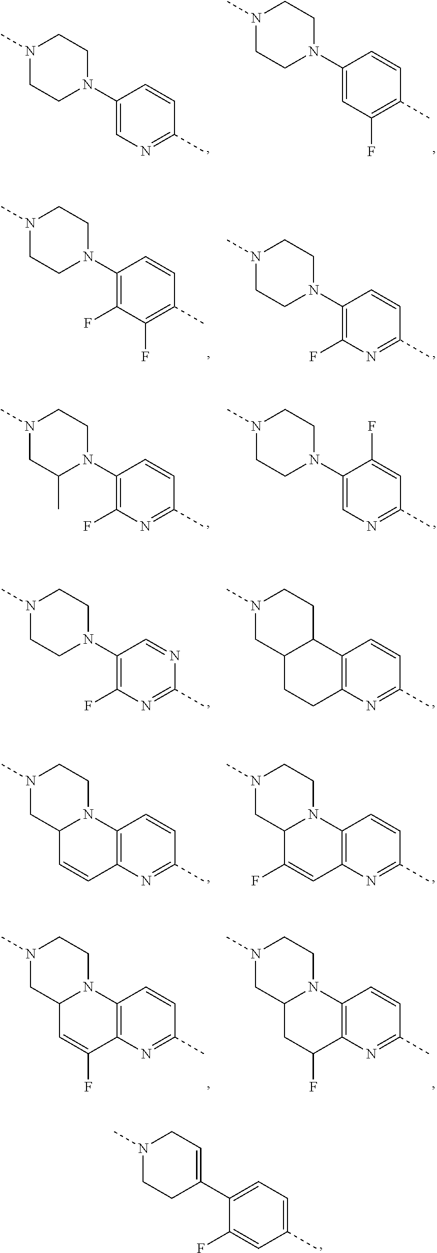

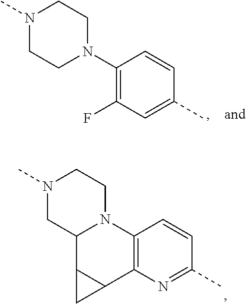

and other variables are as defined in the present disclosure.

In some embodiments of the present disclosure, the structural moiety

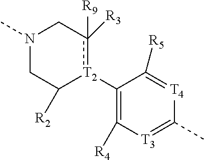

is selected from

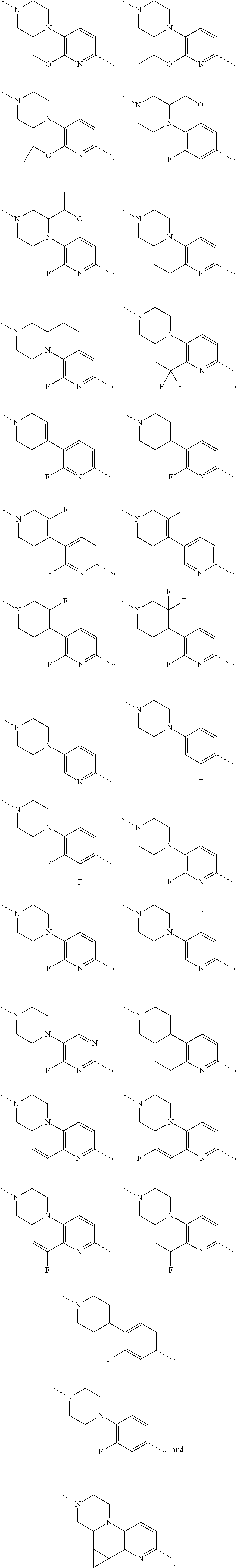

and other variables are as defined in the present disclosure.

In some embodiments of the present disclosure, the structural moiety

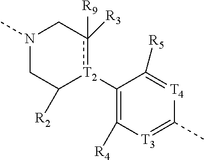

is selected from

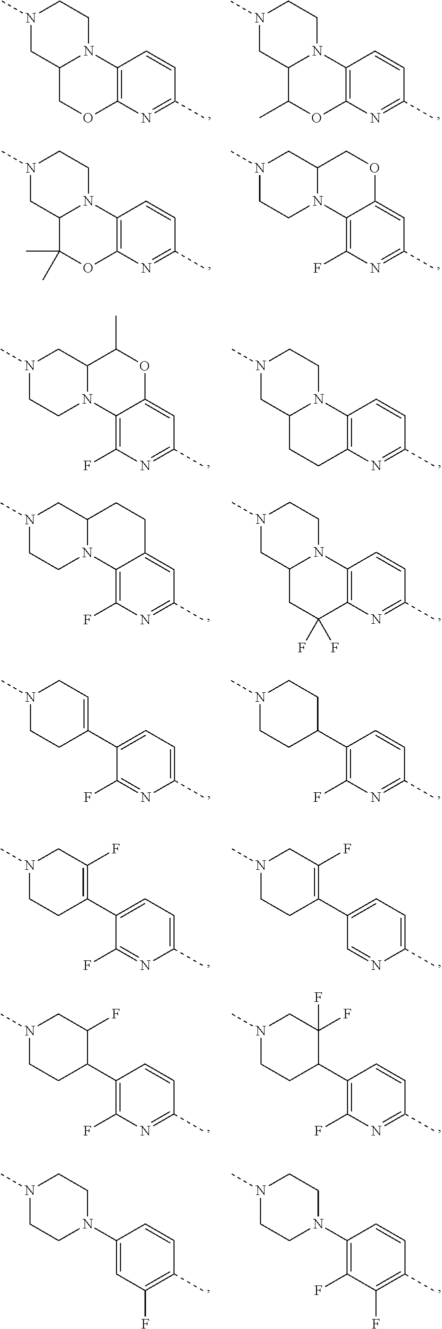

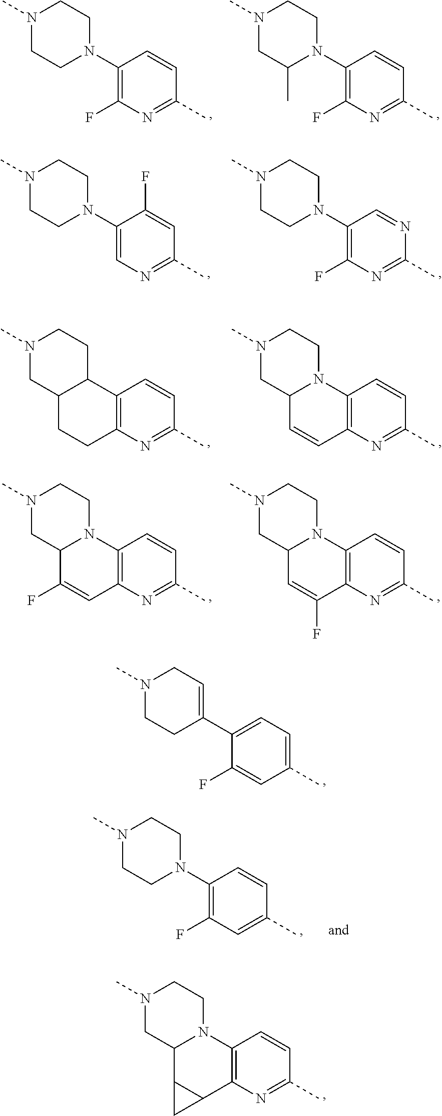

and other variables are as defined in the present disclosure.

In some embodiments of the present disclosure, the structural moiety

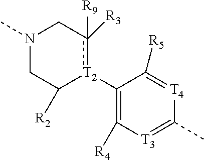

is selected from

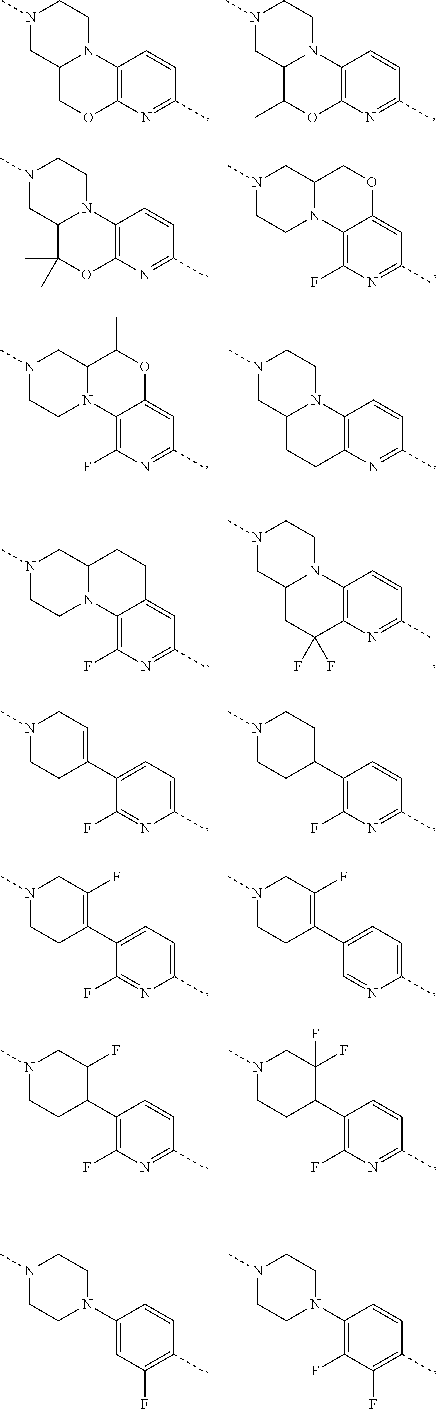

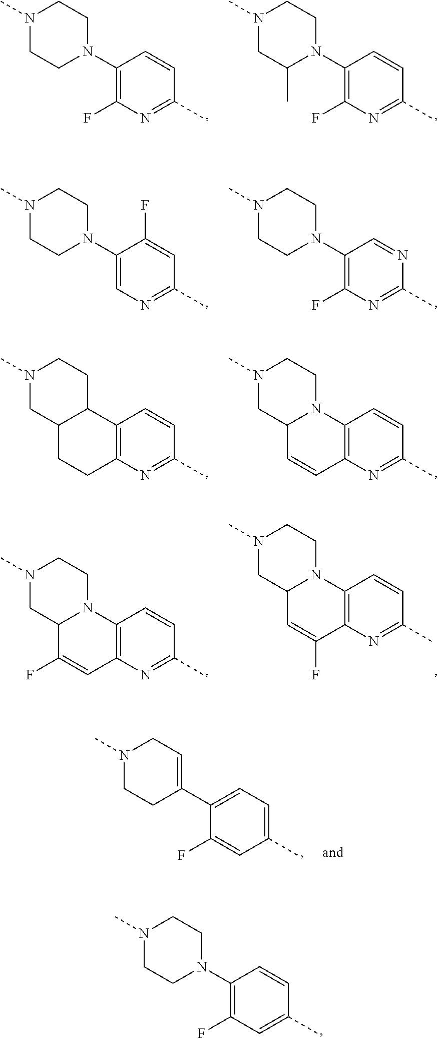

and other variables are as defined in the present disclosure.

In some embodiments of the present disclosure, the structural moiety

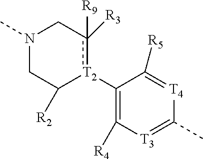

is selected from

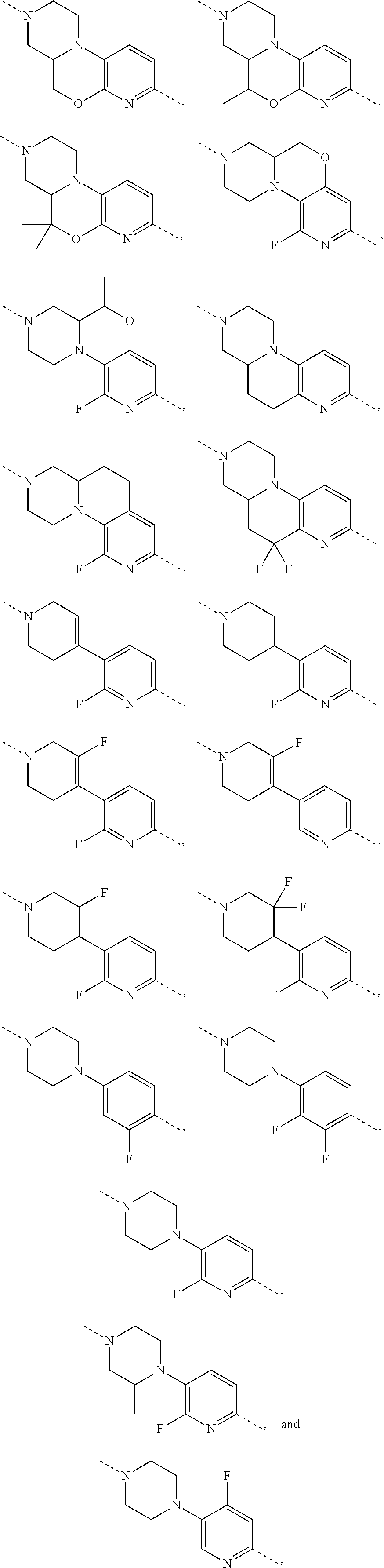

and other variables are as defined in the present disclosure.

In some embodiments of the present disclosure, the structural moiety

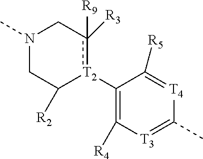

is selected from

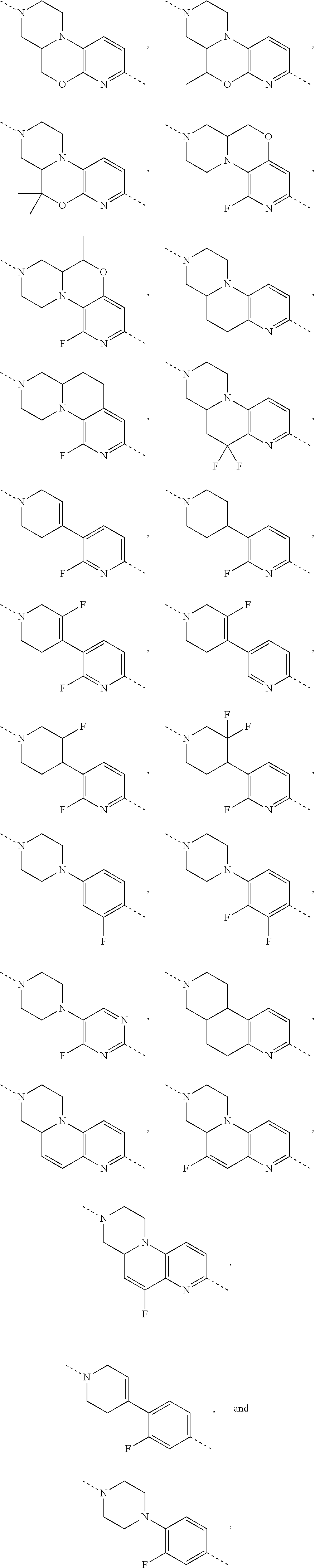

and other variables are as defined in the present disclosure.

In some embodiments of the present disclosure, the structural moiety

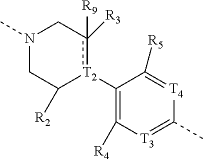

is selected from

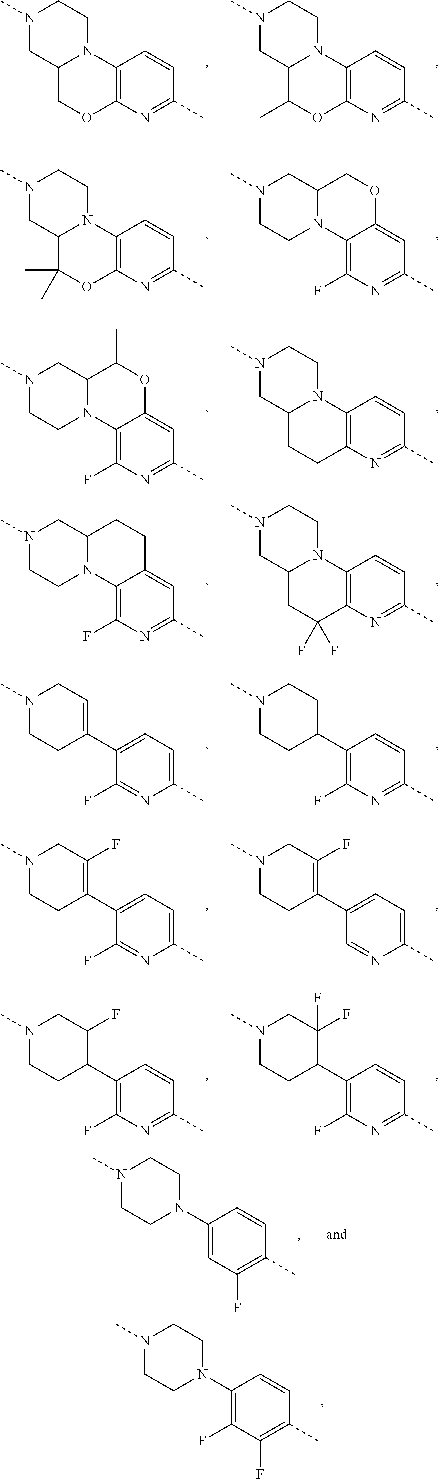

and other variables are as defined in the present disclosure.

In some embodiments of the present disclosure, the structural moiety

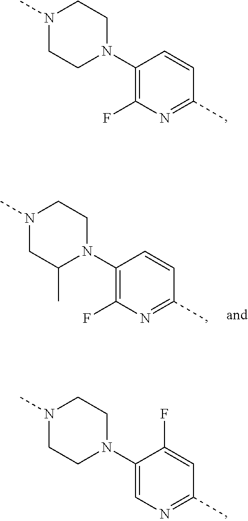

is selected from

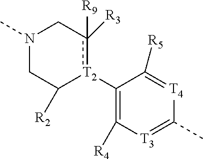

and other variables are as defined in the present disclosure.

In some embodiments of the present disclosure, the structural moiety

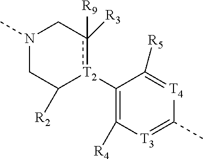

is selected from and other

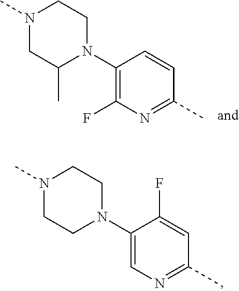

variables are as defined in the present disclosure.

In some embodiments of the present disclosure, the structural moiety

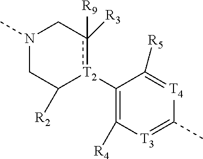

is selected from

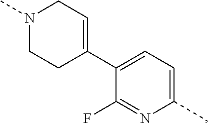

and other variables are as defined in the present disclosure.

In some embodiments of the present disclosure, when the structural moiety

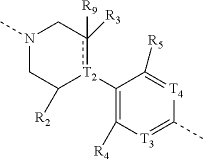

is selected from

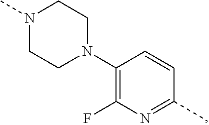

then R₆ and R₇ are not simultaneously H, and other variables are as defined in the present disclosure.

In some embodiments of the present disclosure, when the structural moiety

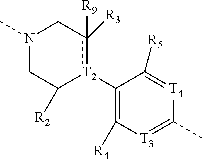

is selected from

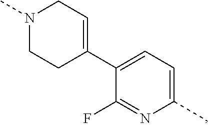

then R₆ and R₇ are not simultaneously H, and other variables are as defined in the present disclosure.

In some embodiments of the present disclosure, the structural moiety

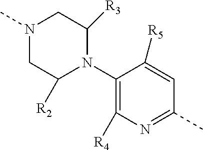

is selected from

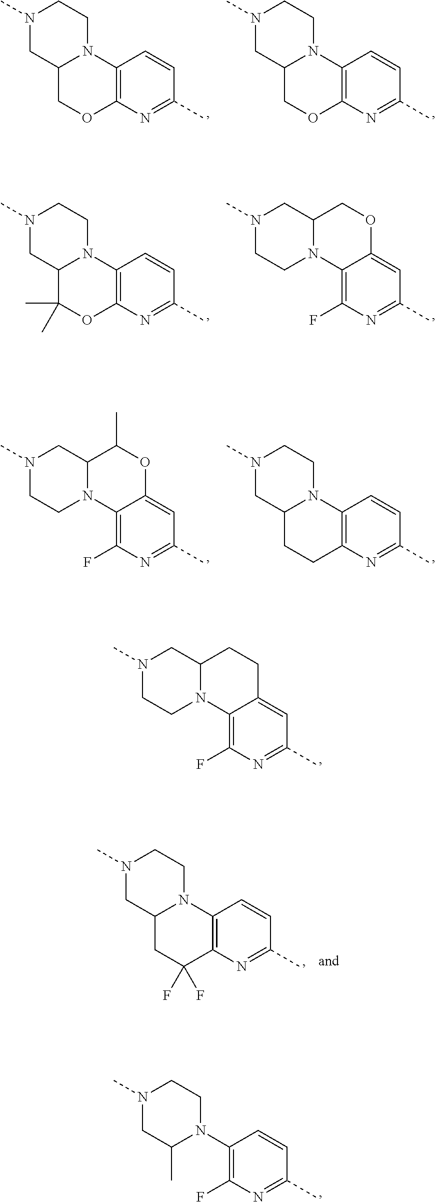

and other variables are as defined in the present disclosure.

In some embodiments of the present disclosure, the structural moiety

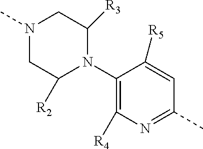

is selected from

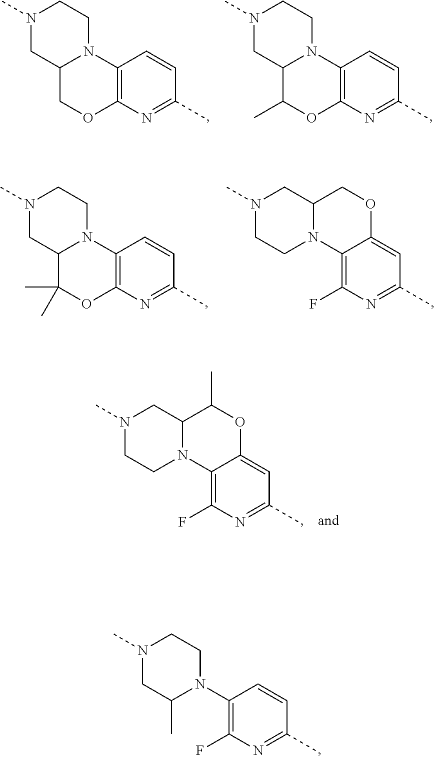

and other variables are as defined in the present disclosure.

In some embodiments of the present disclosure, the compound or the pharmaceutically acceptable salt thereof is selected from:

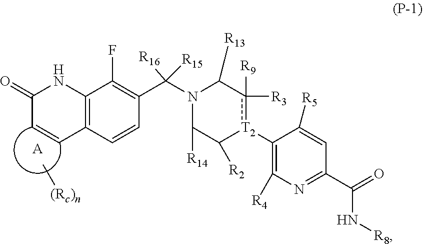

wherein ring A is selected from pyridyl;
R₂, R₃, R₄, R₅, R₈, R₉, R₁₃, R₁₄, R₁₅, R₁₆, R_{c}, T₂, n, and [symbol] are as defined in the present disclosure.

In some embodiments of the present disclosure, the compound or the pharmaceutically acceptable salt thereof is selected from:

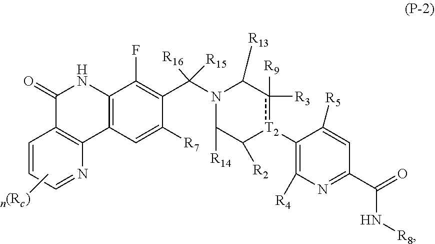

wherein [symbol] is selected from a single bond or a double bond;
T₂ is selected from C and N;
R₂ is selected from H and C₁₋₃ alkyl, and the C₁₋₃ alkyl is optionally substituted by 1, 2, or 3 halogens;
R₃ is selected from H and halogen;
R₄ is selected from H, halogen, CN, C₁₋₃ alkyl, and C₁₋₃ alkoxy;
R₅ is selected from H, halogen, CN, C₁₋₃ alkyl, and C₁₋₃ alkoxy;
R₉ is absent, or selected from H and halogen;
R₇, R₈, R₁₃, R₁₄, R₁₅, R₁₆, R_{c}, and n are as defined in the present disclosure.

In some embodiments of the present disclosure, the compound or the pharmaceutically acceptable salt thereof is selected from:

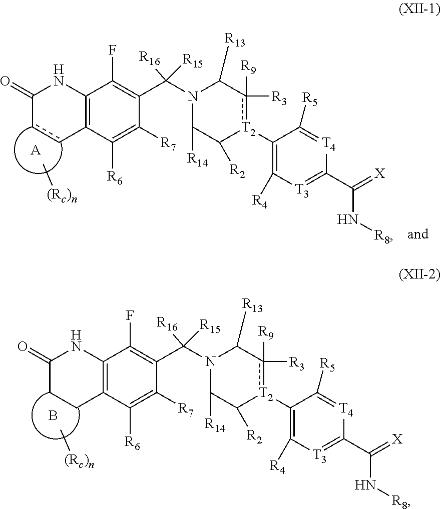

wherein
ring A is selected from phenyl and 6-membered heteroaryl;
ring B is selected from 6-membered heteroaryl;
R₂, R₃, R₄, R₅, R₆, R₇, R₈, R₉, R₁₃, R₁₄, R₁₅, R₁₆, R_{c}, X, T₂, T₃, T₄, n, and [symbol] are as defined in the present disclosure.

In some embodiments of the present disclosure, the ring A is selected from a pyridine ring, and other variables are as defined in the present disclosure.

In some embodiments of the present disclosure, the compound or the pharmaceutically acceptable salt thereof is selected from:

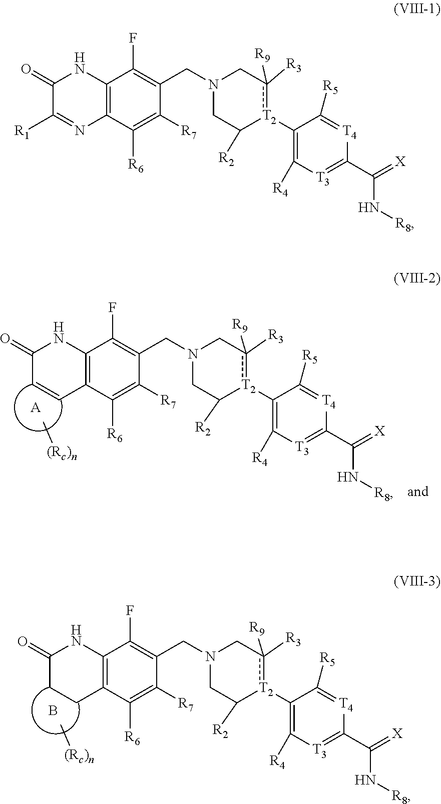

wherein ring A, ring B, R₁, R₂, R₃, R₄, R₅, R₆, R₇, R₈, R₉, X, T₃, T₄, and the structural moiety

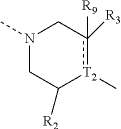

are as defined in the present disclosure.

In some embodiments of the present disclosure, the compound or the pharmaceutically acceptable salt thereof is selected from:

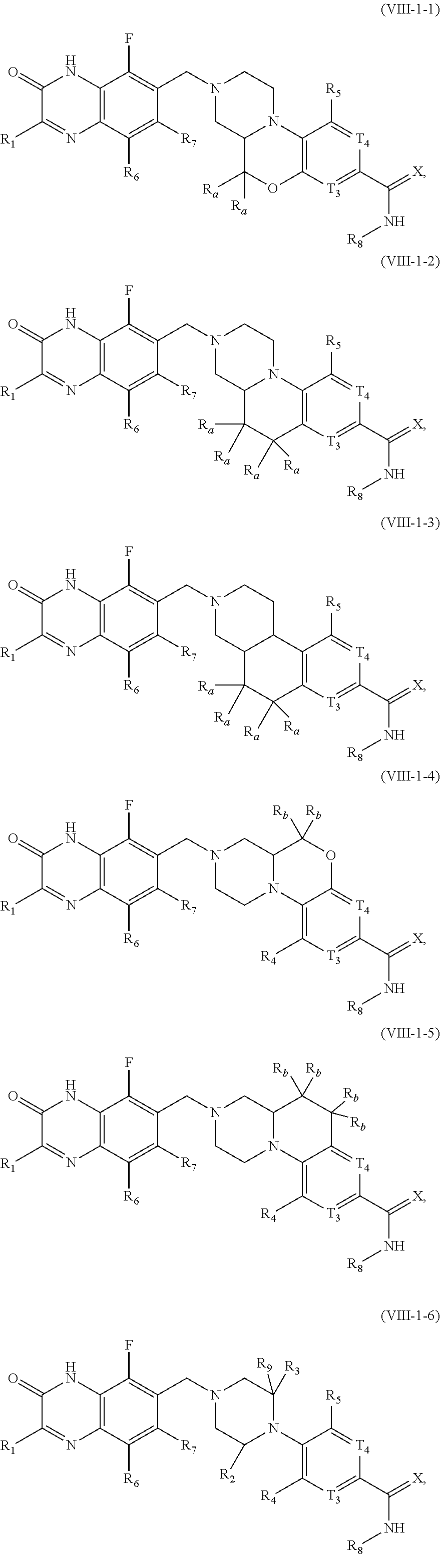

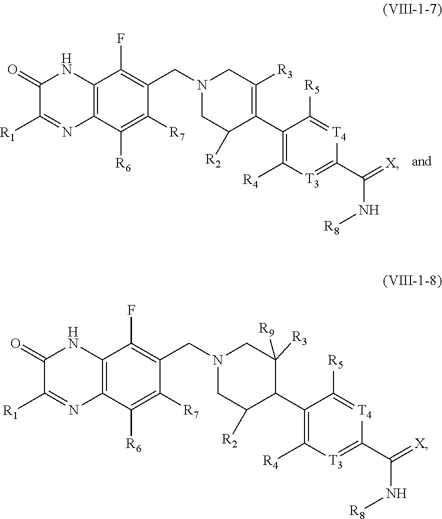

wherein R₁, R₂, R₃, R₄, R₅, R₆, R₇, R₈, R₉, Rₐ, R_{b}, X, T₃, and T₄ are as defined in the present disclosure.

In some embodiments of the present disclosure, the compound or the pharmaceutically acceptable salt thereof is selected from:

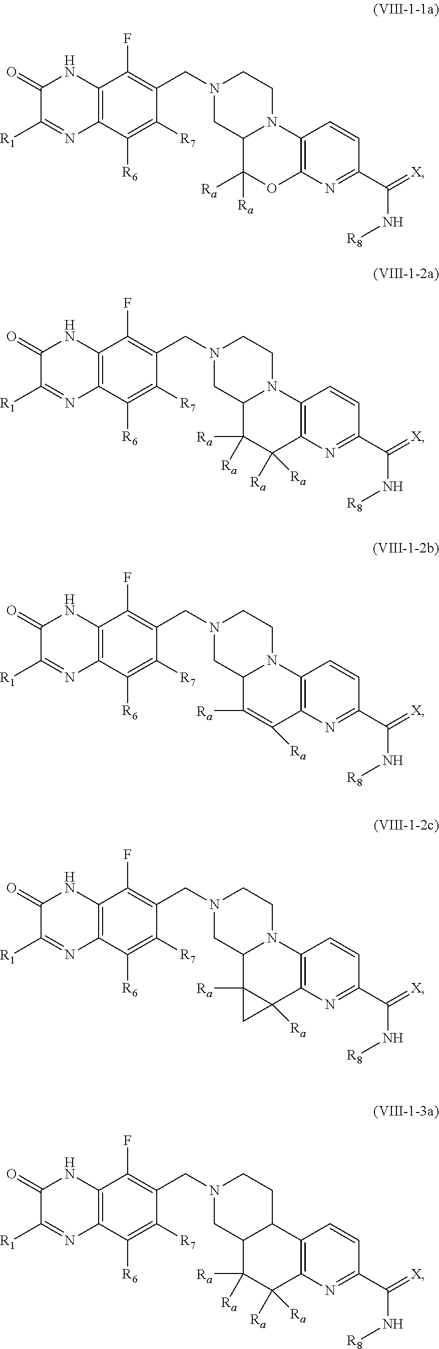

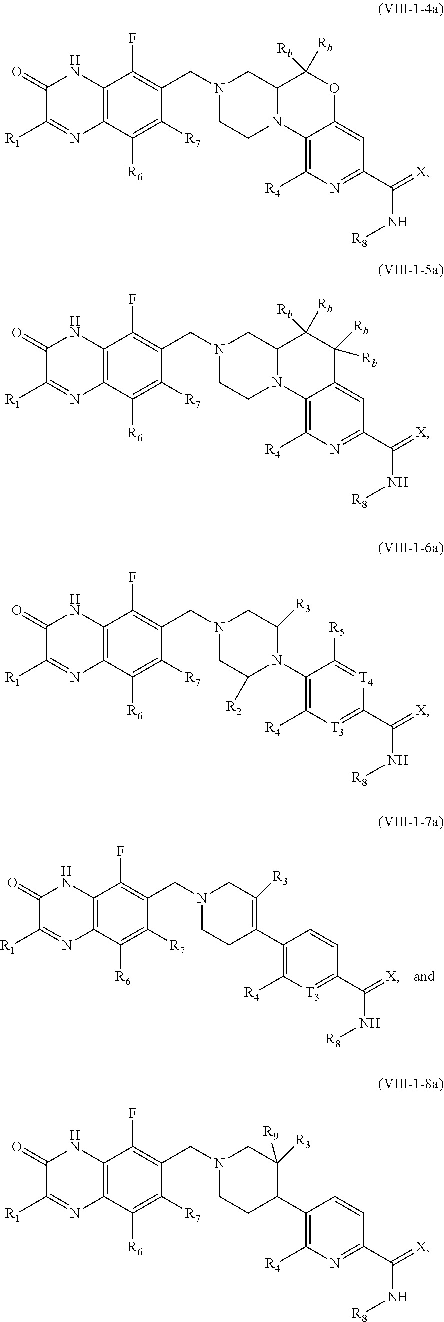

wherein R₁, R₂, R₃, R₄, R₅, R₆, R₇, R₈, R₉, Rₐ, R_{b}, X, T₃, and T₄ are as defined in the present disclosure;
given that
1) when the compound is selected from a compound of formula (VIII-6a), and one of T₃ or T₄ is selected from N, then R₂, R₃, and R₉ are not simultaneously H; or
2) when the compound is selected from a compound of formula (VIII-6a), and one of T₃ or T₄ is selected from N, then R₆ and R₇ are not simultaneously H; or
3) when the compound is selected from a compound of formula (VIII-6a), T₃ is selected from N, and T₄ is selected from CR₁₀, then R₅ is selected from halogen; or
4) when the compound is selected from a compound of formula (VIII-6a), T₄ is selected from N, and T₃ is selected from CR₁₀, then R₄ is selected from halogen; or
5) when the compound is selected from a compound of formula (VIII-7a), and T₃ is selected from N, then R₆ and R₇ are not simultaneously H.

There are still some embodiments of the present disclosure which are obtained by any combination of the above variables.

The present disclosure further provides a compound as follows or a pharmaceutically acceptable salt thereof,

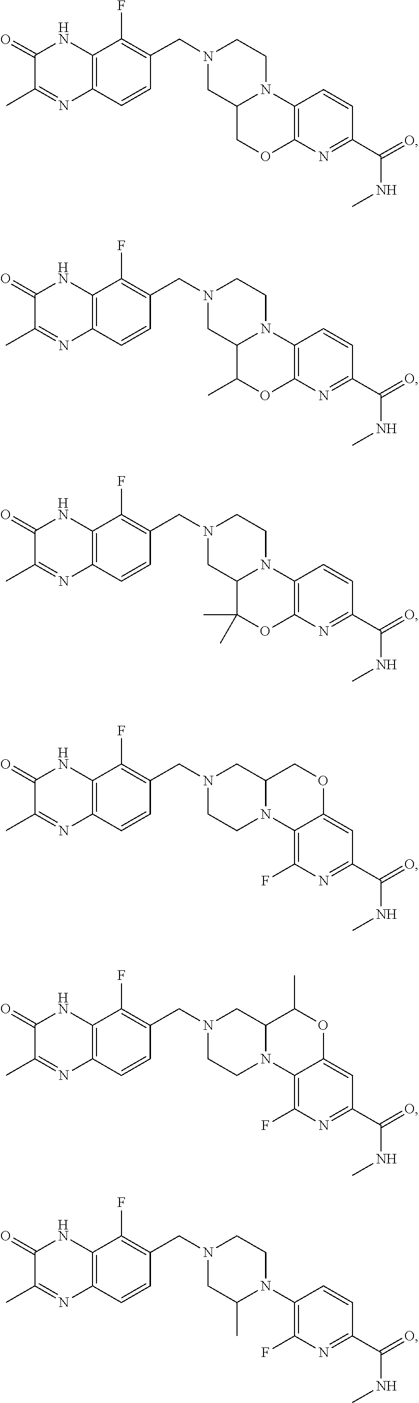 [SMILES: CNC(=O)c1ccc2c(n1)OCC1CN(Cc3ccc4nc(C)c(=O)[nH]c4c3F)CCN21 ; CNC(=O)c1ccc2c(n1)OC(C)C1CN(Cc3ccc4nc(C)c(=O)[nH]c4c3F)CCN21 ; CNC(=O)c1ccc2c(n1)OC(C)(C)C1CN(Cc3ccc4nc(C)c(=O)[nH]c4c3F)CCN21 ; CNC(=O)c1cc2c(c(F)n1)N1CCN(Cc3ccc4nc(C)c(=O)[nH]c4c3F)CC1CO2 ; CNC(=O)c1cc2c(c(F)n1)N1CCN(Cc3ccc4nc(C)c(=O)[nH]c4c3F)CC1C(C)O2 ; CNC(=O)c1ccc(N2CCN(Cc3ccc4nc(C)c(=O)[nH]c4c3F)CC2C)c(F)n1]

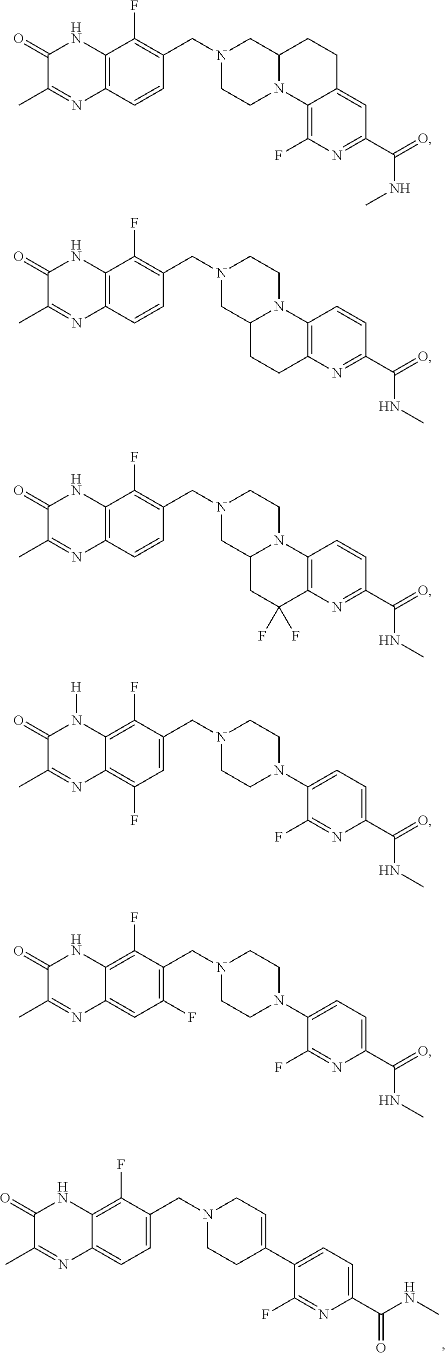

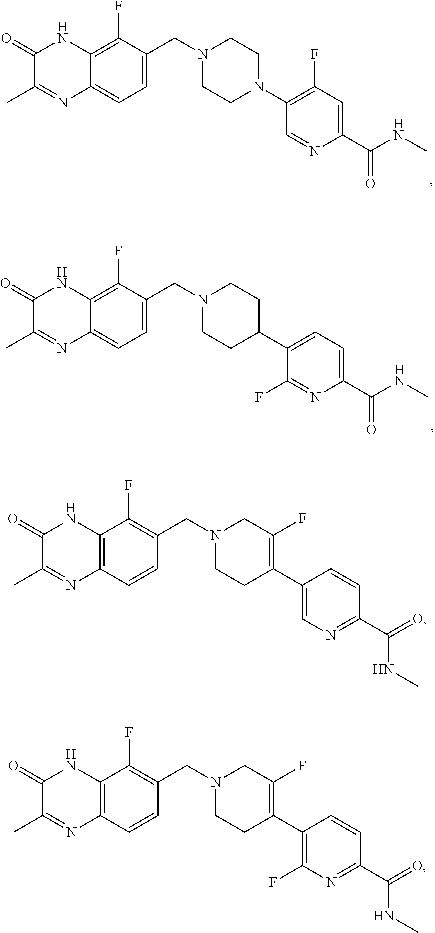

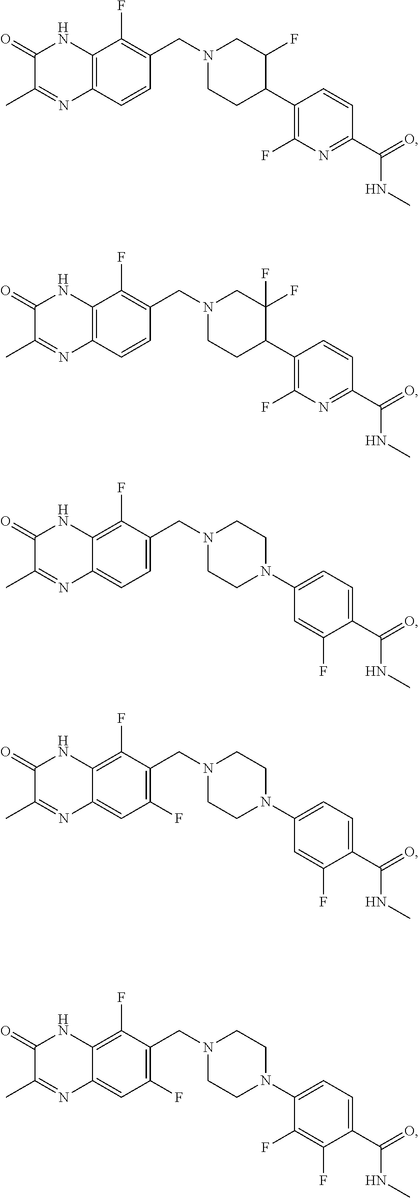

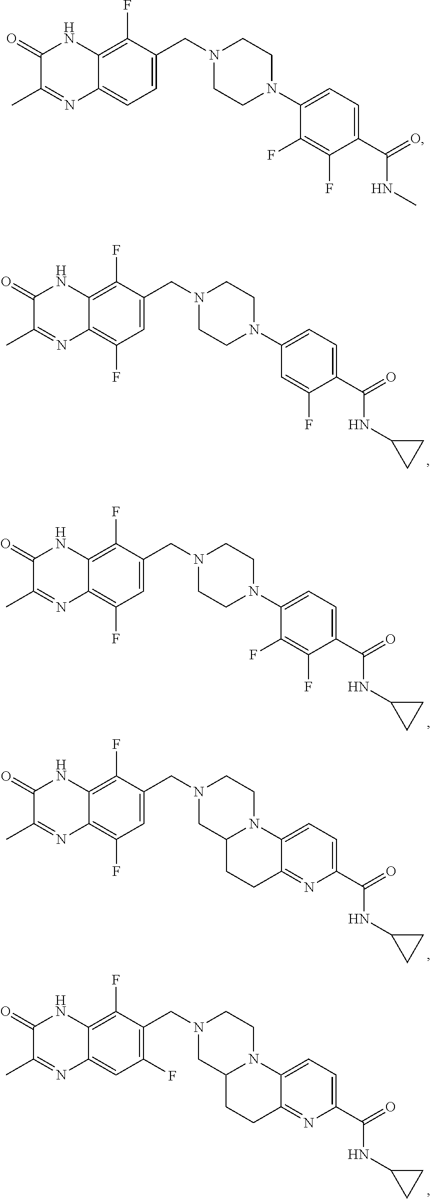

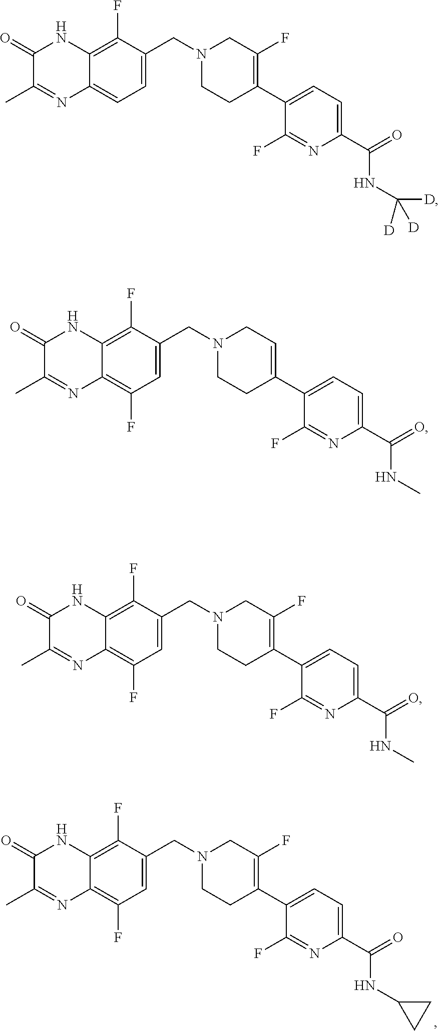

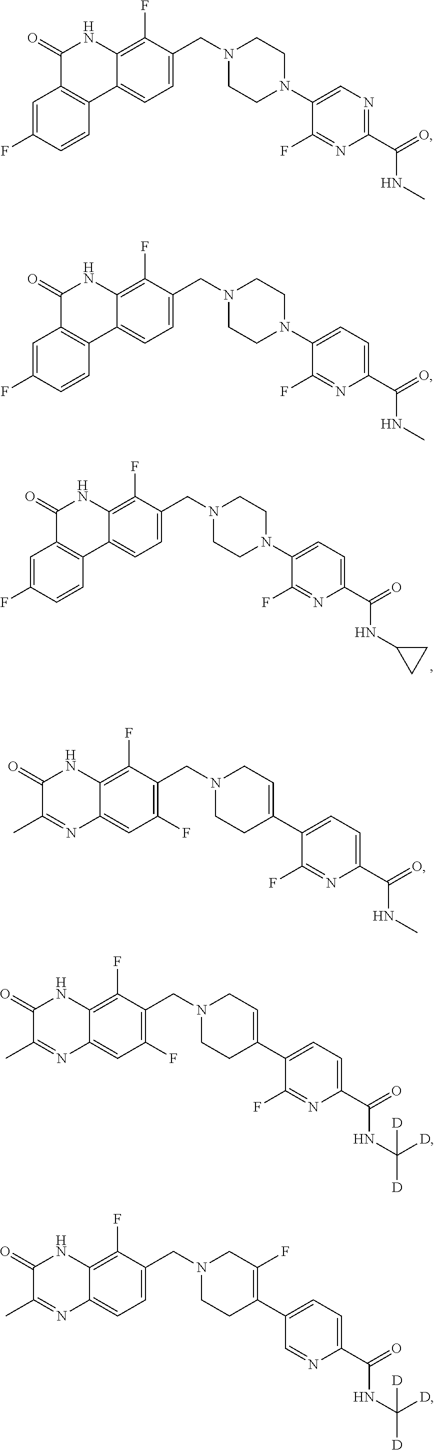

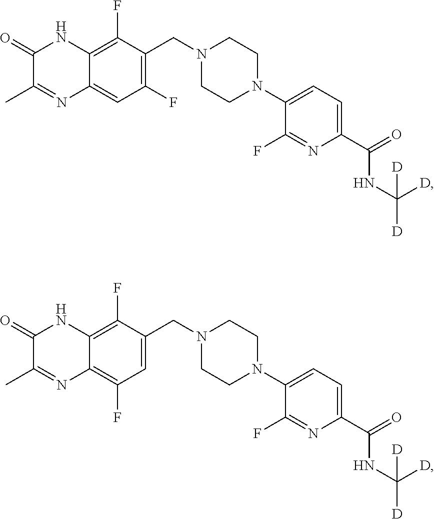

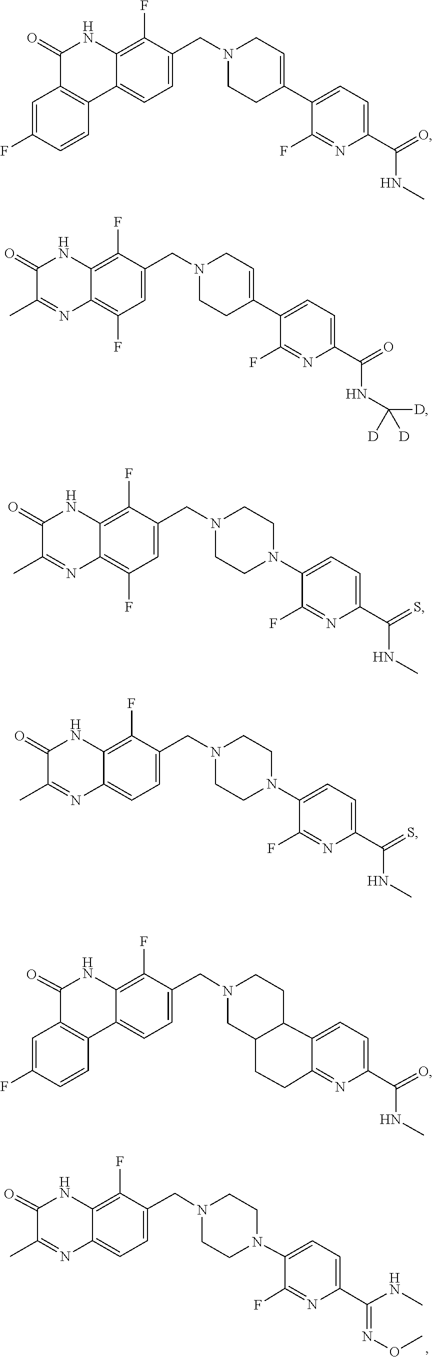

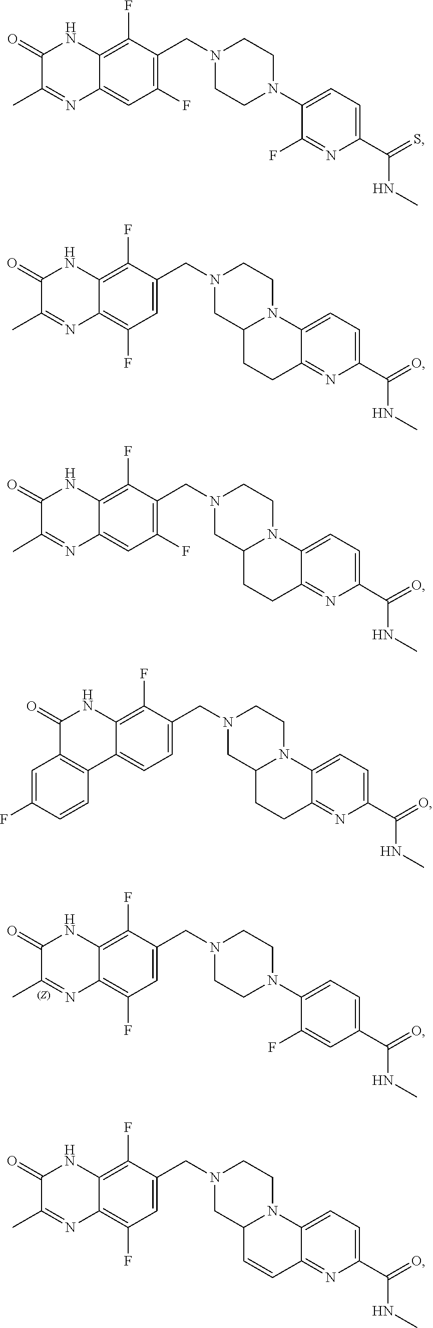

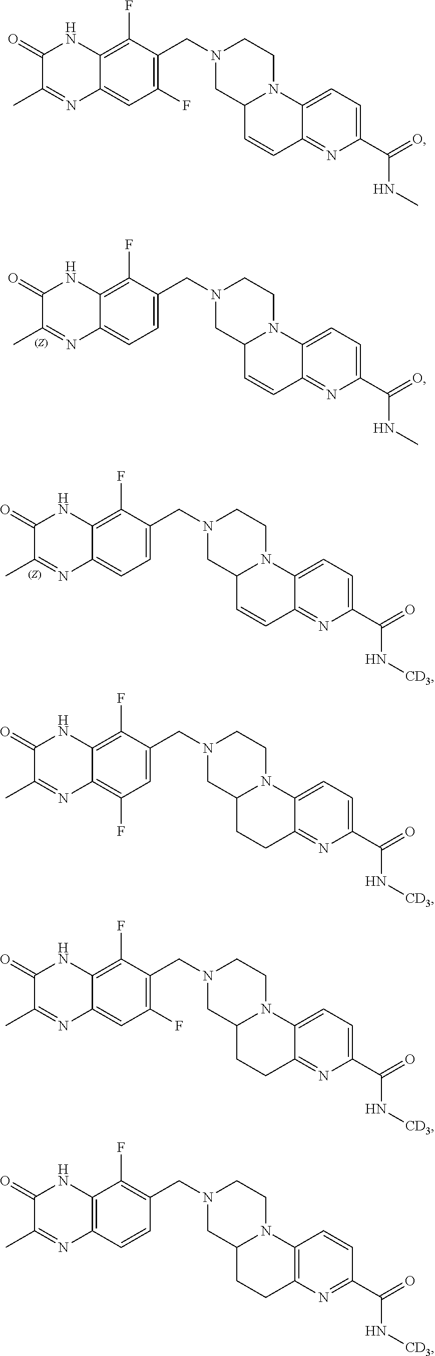

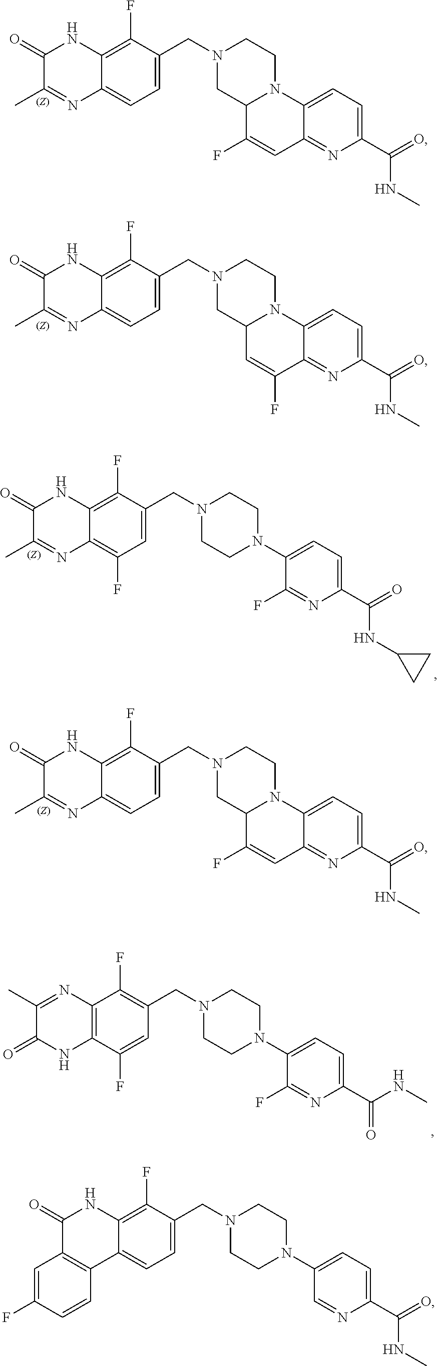

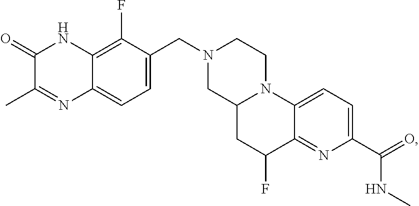

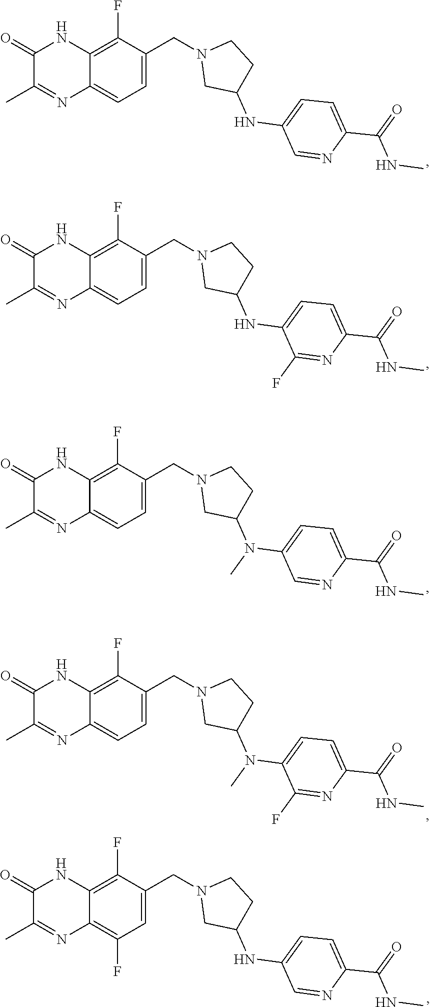

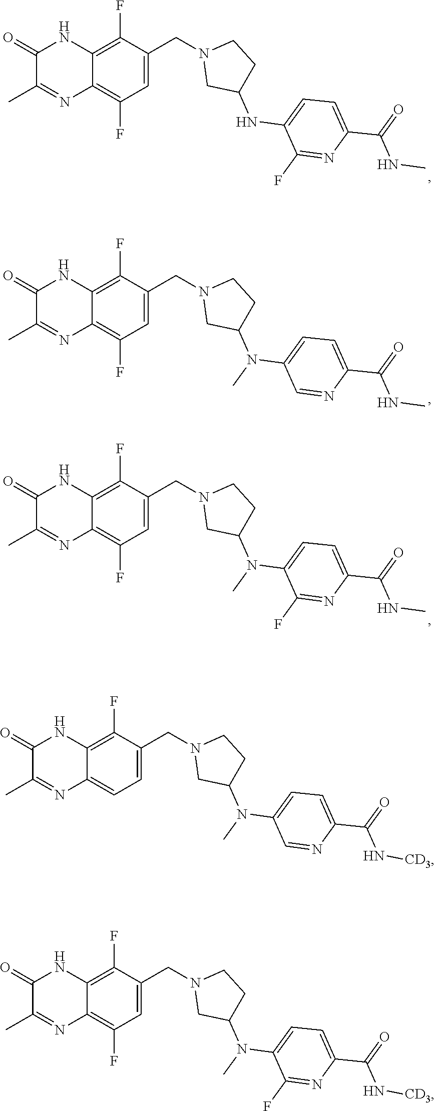

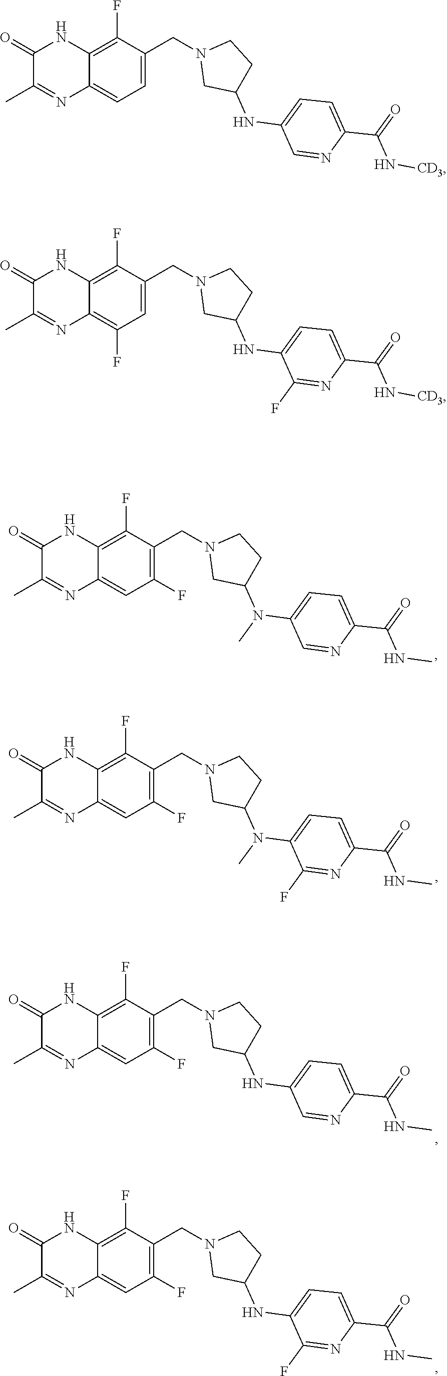

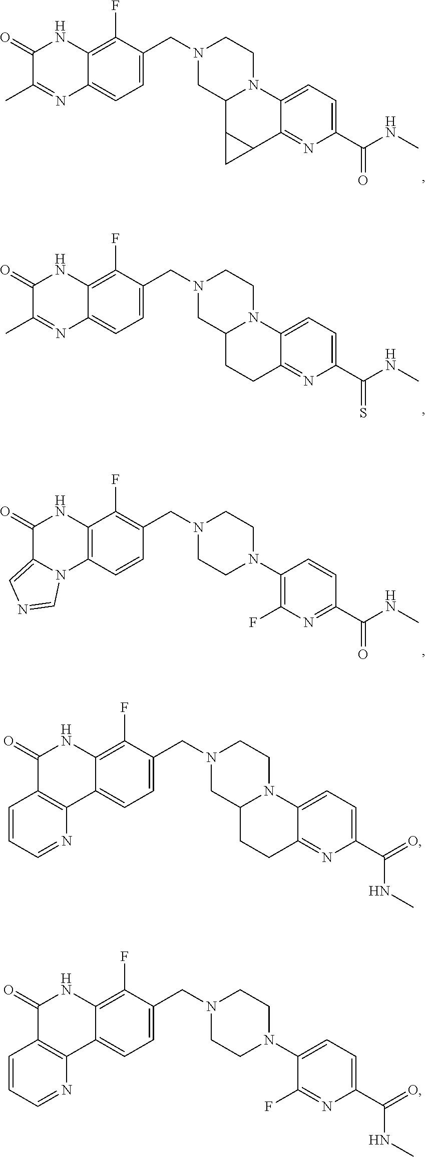

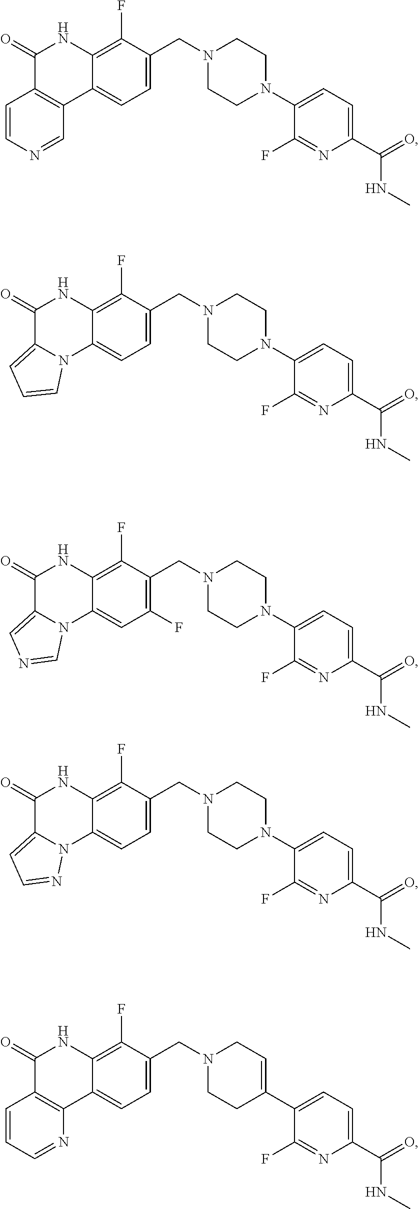 [SMILES: CNC(=O)c1ccc(N2CCN(Cc3ccc4c([nH]c(=O)c5ccncc54)c3F)CC2)c(F)n1 ; CNC(=O)c1ccc(N2CCN(Cc3ccc4c([nH]c(=O)c5cccn54)c3F)CC2)c(F)n1 ; CNC(=O)c1ccc(N2CCN(Cc3c(F)cc4c([nH]c(=O)c5cncn54)c3F)CC2)c(F)n1 ; CNC(=O)c1ccc(N2CCN(Cc3ccc4c([nH]c(=O)c5ccnn54)c3F)CC2)c(F)n1 ; CNC(=O)c1ccc(C2=CCN(Cc3ccc4c([nH]c(=O)c5cccnc54)c3F)CC2)c(F)n1]

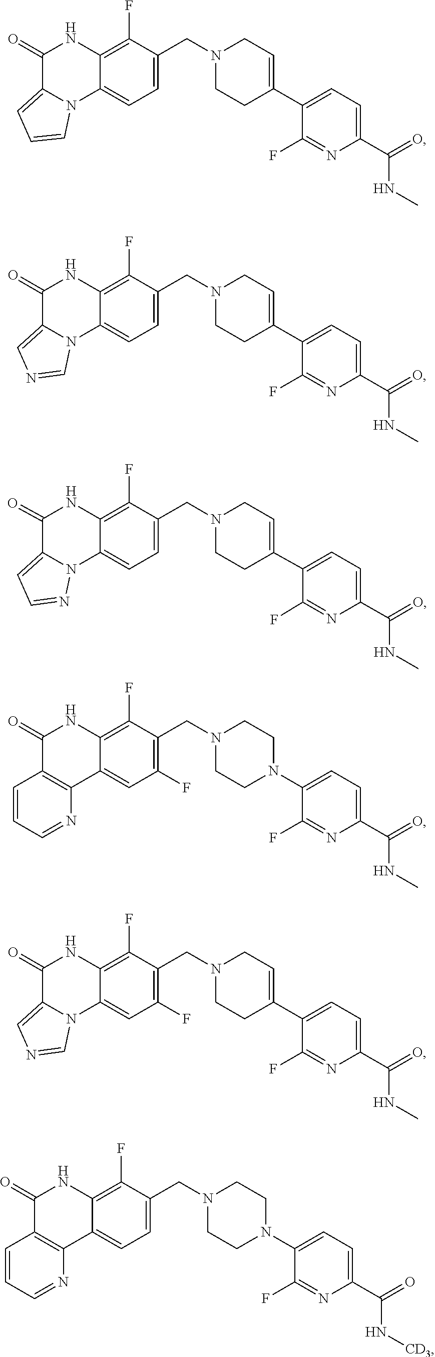 [SMILES: CNC(=O)c1ccc(C2=CCN(Cc3ccc4c([nH]c(=O)c5cccn54)c3F)CC2)c(F)n1 ; CNC(=O)c1ccc(C2=CCN(Cc3ccc4c([nH]c(=O)c5cncn54)c3F)CC2)c(F)n1 ; CNC(=O)c1ccc(C2=CCN(Cc3ccc4c([nH]c(=O)c5ccnn54)c3F)CC2)c(F)n1 ; CNC(=O)c1ccc(N2CCN(Cc3c(F)cc4c([nH]c(=O)c5cccnc54)c3F)CC2)c(F)n1 ; CNC(=O)c1ccc(C2=CCN(Cc3c(F)cc4c([nH]c(=O)c5cncn54)c3F)CC2)c(F)n1 ; [2H]C([2H])([2H])NC(=O)c1ccc(N2CCN(Cc3ccc4c([nH]c(=O)c5cccnc54)c3F)CC2)c(F)n1]

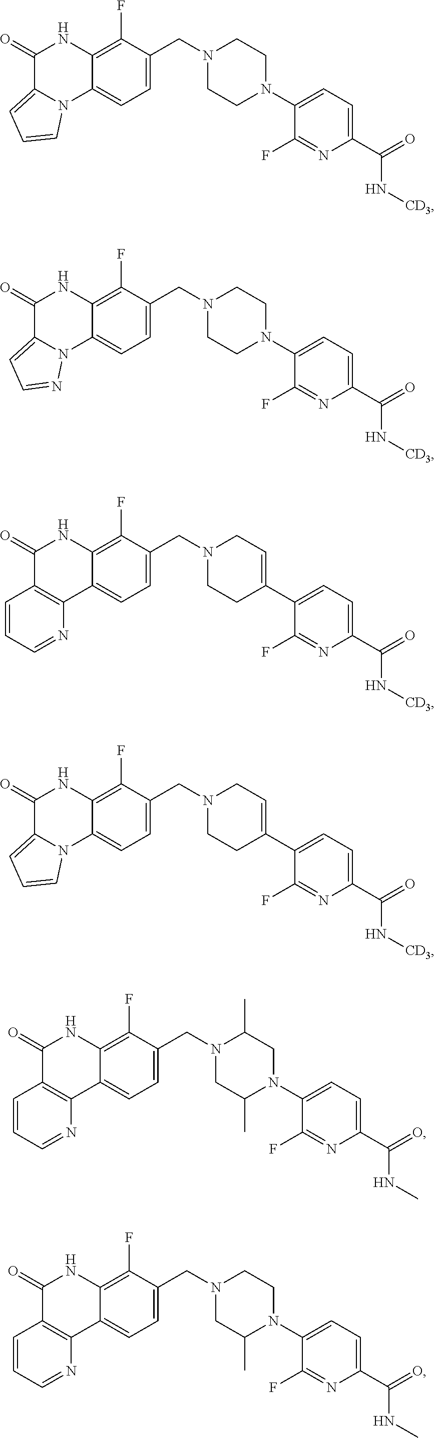 [SMILES: [2H]C([2H])([2H])NC(=O)c1ccc(N2CCN(Cc3ccc4c([nH]c(=O)c5cccn54)c3F)CC2)c(F)n1 ; [2H]C([2H])([2H])NC(=O)c1ccc(N2CCN(Cc3ccc4c([nH]c(=O)c5ccnn54)c3F)CC2)c(F)n1 ; [2H]C([2H])([2H])NC(=O)c1ccc(C2=CCN(Cc3ccc4c([nH]c(=O)c5cccnc54)c3F)CC2)c(F)n1 ; [2H]C([2H])([2H])NC(=O)c1ccc(C2=CCN(Cc3ccc4c([nH]c(=O)c5cccn54)c3F)CC2)c(F)n1 ; CNC(=O)c1ccc(N2CC(C)N(Cc3ccc4c([nH]c(=O)c5cccnc54)c3F)CC2C)c(F)n1 ; CNC(=O)c1ccc(N2CCN(Cc3ccc4c([nH]c(=O)c5cccnc54)c3F)CC2C)c(F)n1]

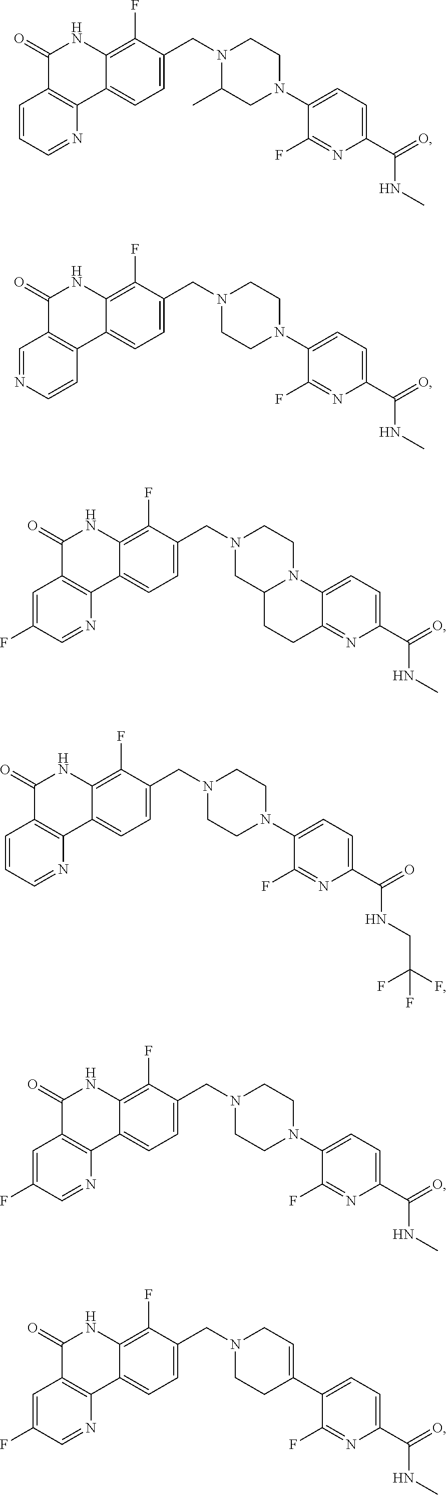 [SMILES: CNC(=O)c1ccc(N2CCN(Cc3ccc4c([nH]c(=O)c5cccnc54)c3F)C(C)C2)c(F)n1 ; CNC(=O)c1ccc(N2CCN(Cc3ccc4c([nH]c(=O)c5cnccc54)c3F)CC2)c(F)n1 ; CNC(=O)c1ccc2c(n1)CCC1CN(Cc3ccc4c([nH]c(=O)c5cc(F)cnc54)c3F)CCN21 ; O=C(NCC(F)(F)F)c1ccc(N2CCN(Cc3ccc4c([nH]c(=O)c5cccnc54)c3F)CC2)c(F)n1 ; CNC(=O)c1ccc(N2CCN(Cc3ccc4c([nH]c(=O)c5cc(F)cnc54)c3F)CC2)c(F)n1 ; CNC(=O)c1ccc(C2=CCN(Cc3ccc4c([nH]c(=O)c5cc(F)cnc54)c3F)CC2)c(F)n1]

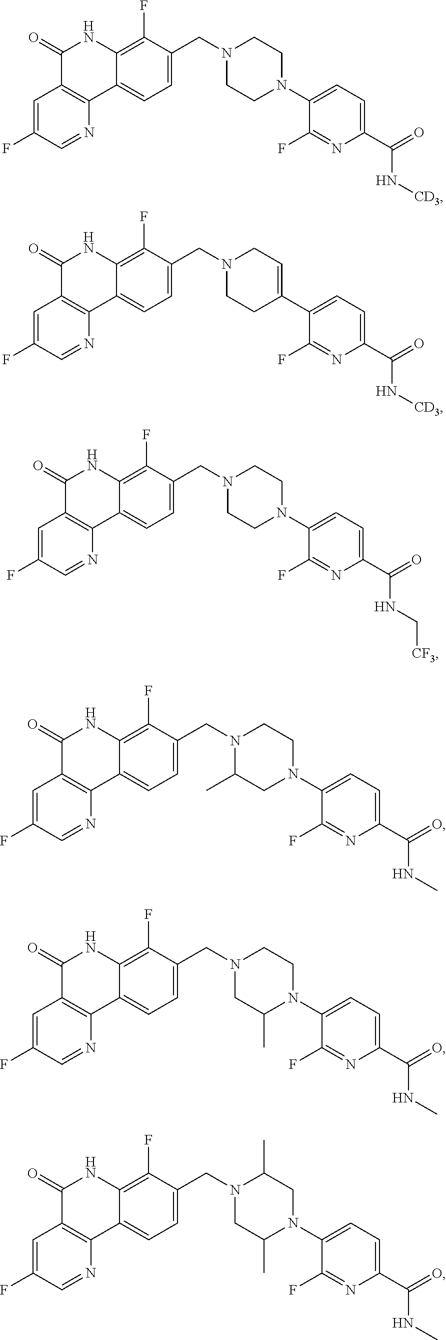 [SMILES: [2H]C([2H])([2H])NC(=O)c1ccc(N2CCN(Cc3ccc4c([nH]c(=O)c5cc(F)cnc54)c3F)CC2)c(F)n1 ; [2H]C([2H])([2H])NC(=O)c1ccc(C2=CCN(Cc3ccc4c([nH]c(=O)c5cc(F)cnc54)c3F)CC2)c(F)n1 ; O=C(NCC(F)(F)F)c1ccc(N2CCN(Cc3ccc4c([nH]c(=O)c5cc(F)cnc54)c3F)CC2)c(F)n1 ; CNC(=O)c1ccc(N2CCN(Cc3ccc4c([nH]c(=O)c5cc(F)cnc54)c3F)C(C)C2)c(F)n1 ; CNC(=O)c1ccc(N2CCN(Cc3ccc4c([nH]c(=O)c5cc(F)cnc54)c3F)CC2C)c(F)n1 ; CNC(=O)c1ccc(N2CC(C)N(Cc3ccc4c([nH]c(=O)c5cc(F)cnc54)c3F)CC2C)c(F)n1]

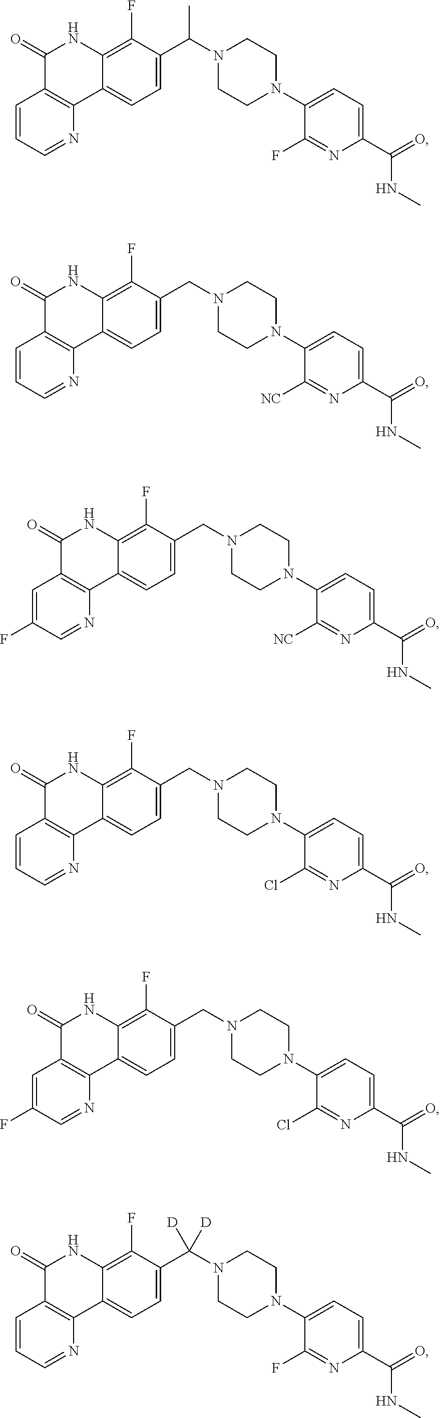 [SMILES: CNC(=O)c1ccc(N2CCN(C(C)c3ccc4c([nH]c(=O)c5cccnc54)c3F)CC2)c(F)n1 ; CNC(=O)c1ccc(N2CCN(Cc3ccc4c([nH]c(=O)c5cccnc54)c3F)CC2)c(C#N)n1 ; CNC(=O)c1ccc(N2CCN(Cc3ccc4c([nH]c(=O)c5cc(F)cnc54)c3F)CC2)c(C#N)n1 ; CNC(=O)c1ccc(N2CCN(Cc3ccc4c([nH]c(=O)c5cccnc54)c3F)CC2)c(Cl)n1 ; CNC(=O)c1ccc(N2CCN(Cc3ccc4c([nH]c(=O)c5cc(F)cnc54)c3F)CC2)c(Cl)n1 ; [2H]C([2H])(c1ccc2c([nH]c(=O)c3cccnc32)c1F)N1CCN(c2ccc(C(=O)NC)nc2F)CC1]

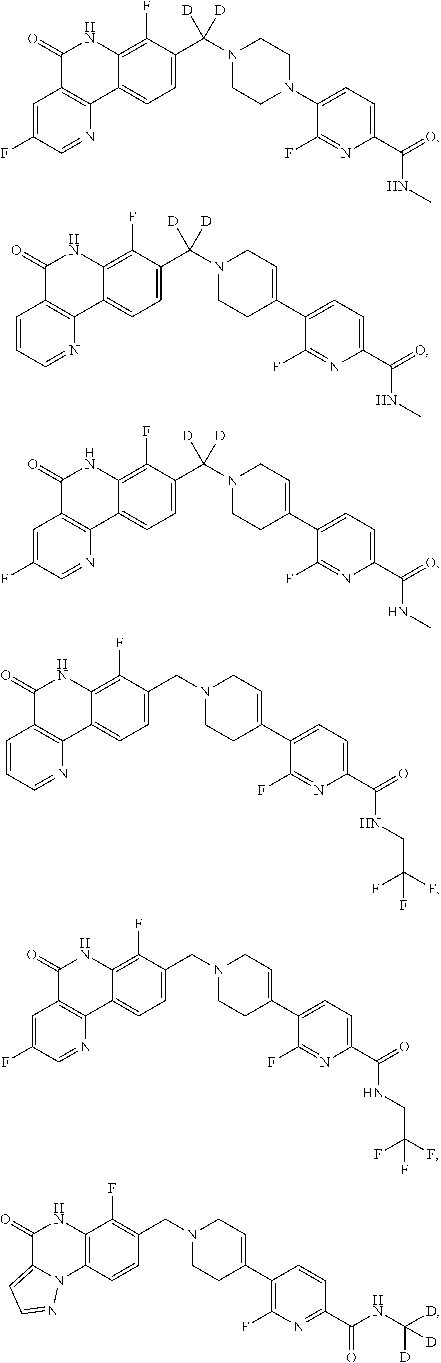

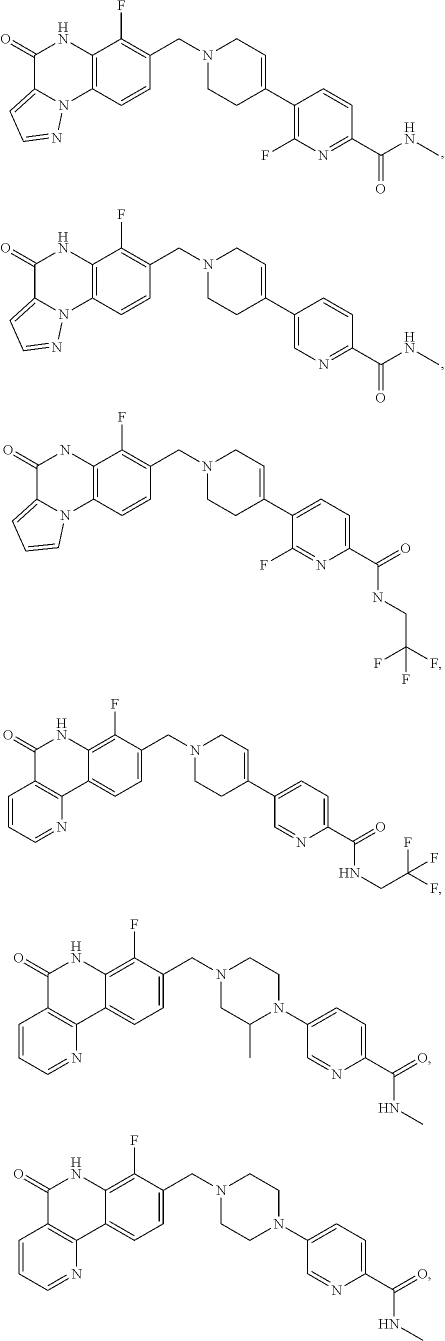

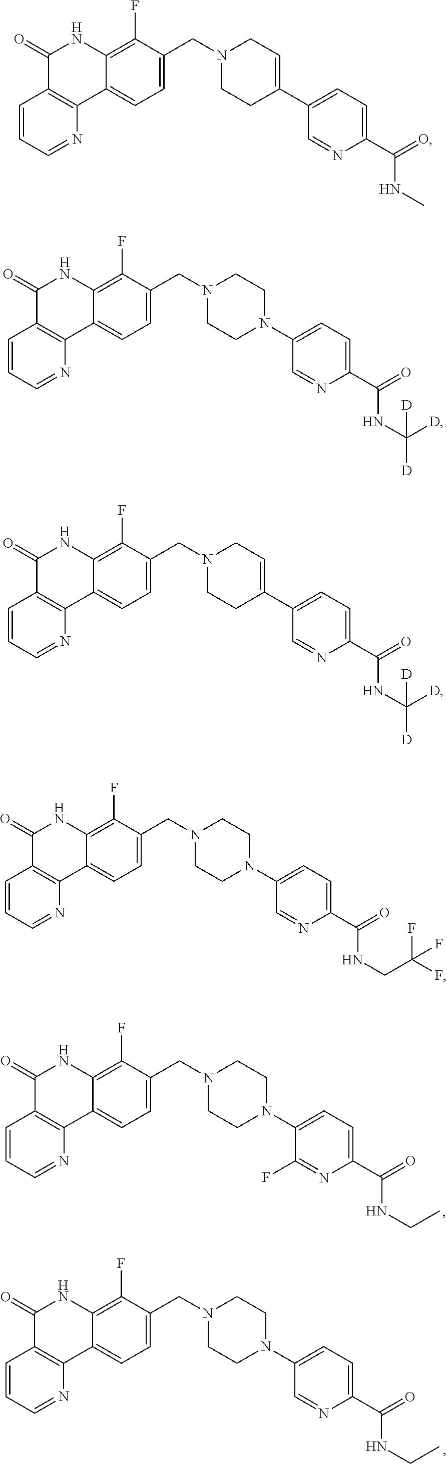

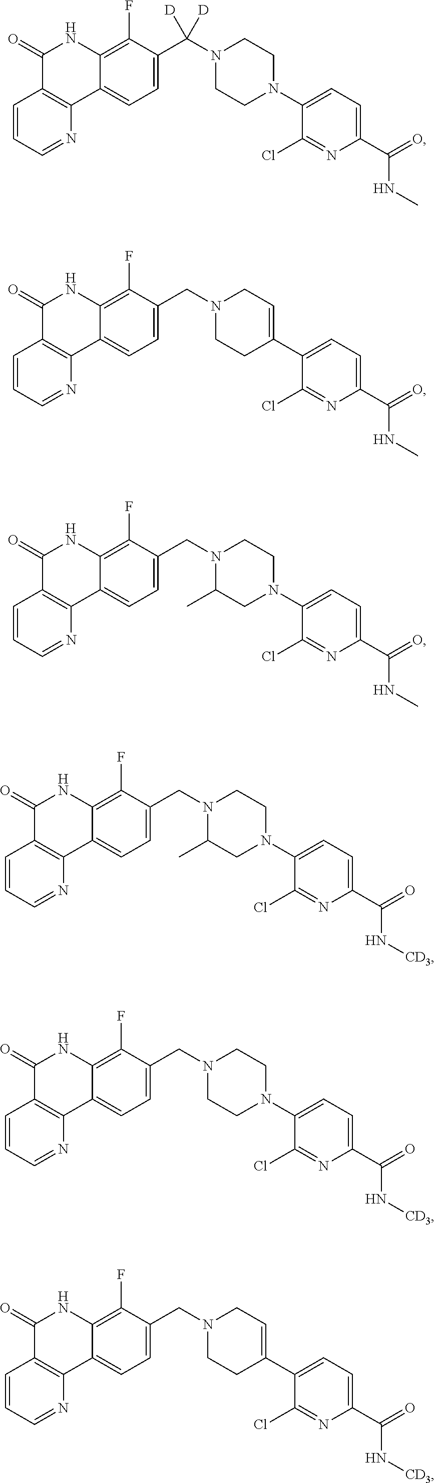 [SMILES: [2H]C([2H])(c1ccc2c([nH]c(=O)c3cccnc32)c1F)N1CCN(c2ccc(C(=O)NC)nc2Cl)CC1 ; CNC(=O)c1ccc(C2=CCN(Cc3ccc4c([nH]c(=O)c5cccnc54)c3F)CC2)c(Cl)n1 ; CNC(=O)c1ccc(N2CCN(Cc3ccc4c([nH]c(=O)c5cccnc54)c3F)C(C)C2)c(Cl)n1 ; [2H]C([2H])([2H])NC(=O)c1ccc(N2CCN(Cc3ccc4c([nH]c(=O)c5cccnc54)c3F)C(C)C2)c(Cl)n1 ; [2H]C([2H])([2H])NC(=O)c1ccc(N2CCN(Cc3ccc4c([nH]c(=O)c5cccnc54)c3F)CC2)c(Cl)n1 ; [2H]C([2H])([2H])NC(=O)c1ccc(C2=CCN(Cc3ccc4c([nH]c(=O)c5cccnc54)c3F)CC2)c(Cl)n1]

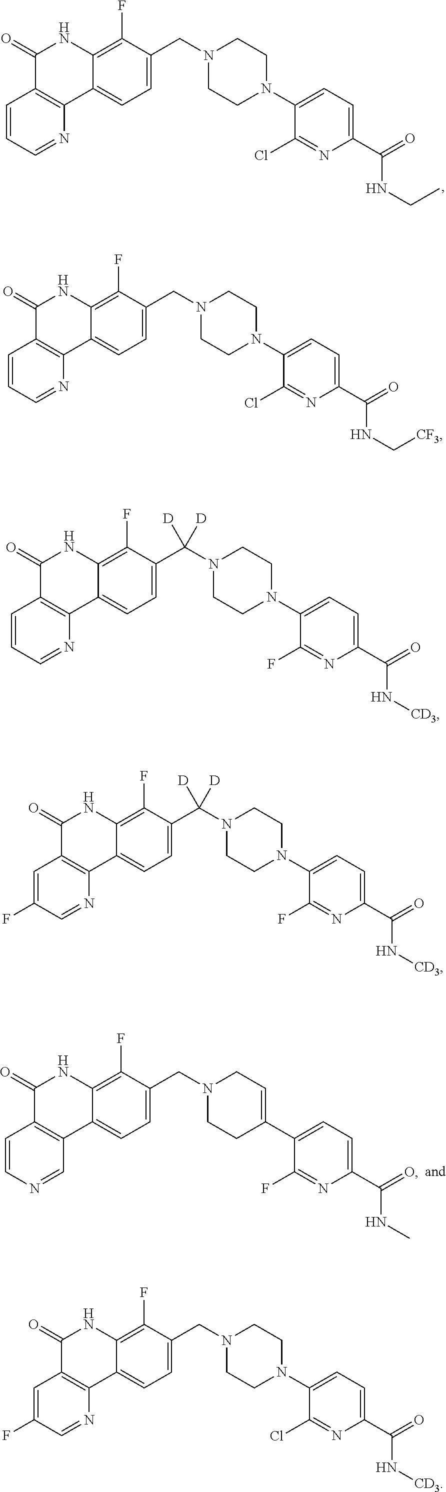 [SMILES: CCNC(=O)c1ccc(N2CCN(Cc3ccc4c([nH]c(=O)c5cccnc54)c3F)CC2)c(Cl)n1 ; O=C(NCC(F)(F)F)c1ccc(N2CCN(Cc3ccc4c([nH]c(=O)c5cccnc54)c3F)CC2)c(Cl)n1 ; [2H]C([2H])([2H])NC(=O)c1ccc(N2CCN(C([2H])([2H])c3ccc4c([nH]c(=O)c5cccnc54)c3F)CC2)c(F)n1 ; [2H]C([2H])([2H])NC(=O)c1ccc(N2CCN(C([2H])([2H])c3ccc4c([nH]c(=O)c5cc(F)cnc54)c3F)CC2)c(F)n1 ; CNC(=O)c1ccc(C2=CCN(Cc3ccc4c([nH]c(=O)c5ccncc54)c3F)CC2)c(F)n1 ; [2H]C([2H])([2H])NC(=O)c1ccc(N2CCN(Cc3ccc4c([nH]c(=O)c5cc(F)cnc54)c3F)CC2)c(Cl)n1]

In some embodiments of the present disclosure, the compound or the pharmaceutically acceptable salt thereof is provided, and the compound is selected from:

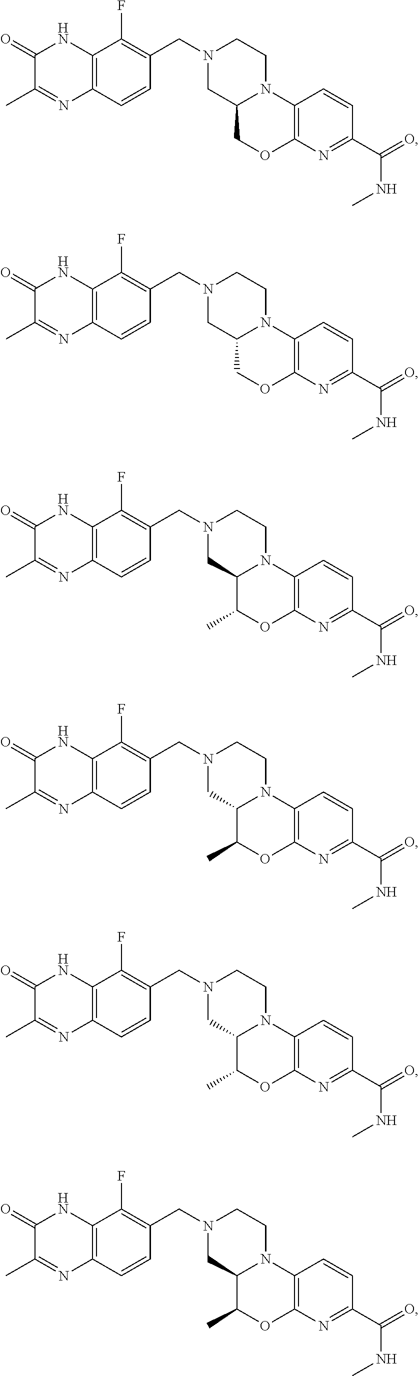 [SMILES: CNC(=O)c1ccc2c(n1)OC[C@H]1CN(Cc3ccc4nc(C)c(=O)[nH]c4c3F)CCN21 ; CNC(=O)c1ccc2c(n1)OC[C@@H]1CN(Cc3ccc4nc(C)c(=O)[nH]c4c3F)CCN21 ; CNC(=O)c1ccc2c(n1)O[C@H](C)[C@H]1CN(Cc3ccc4nc(C)c(=O)[nH]c4c3F)CCN21 ; CNC(=O)c1ccc2c(n1)O[C@@H](C)[C@@H]1CN(Cc3ccc4nc(C)c(=O)[nH]c4c3F)CCN21 ; CNC(=O)c1ccc2c(n1)O[C@H](C)[C@@H]1CN(Cc3ccc4nc(C)c(=O)[nH]c4c3F)CCN21 ; CNC(=O)c1ccc2c(n1)O[C@@H](C)[C@H]1CN(Cc3ccc4nc(C)c(=O)[nH]c4c3F)CCN21]

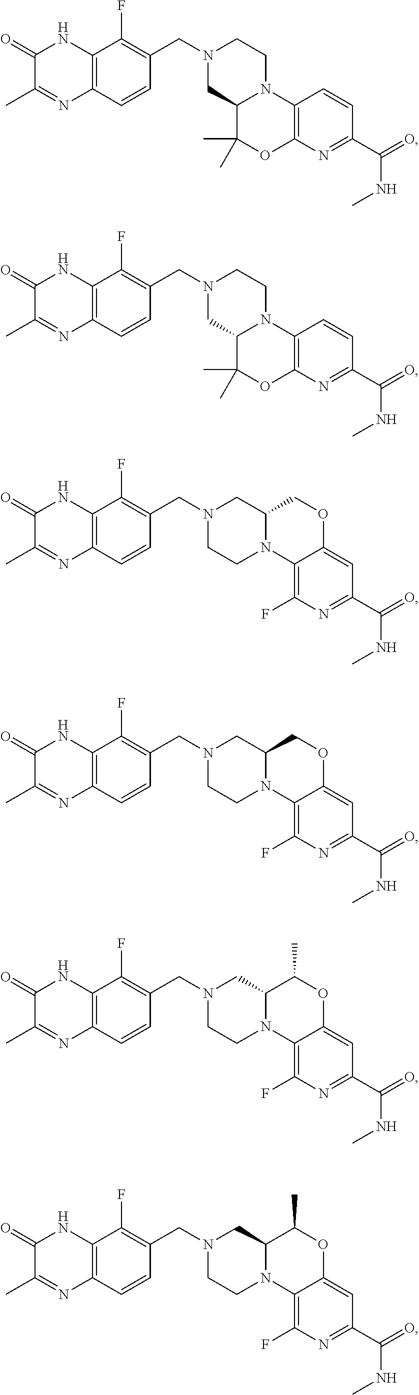 [SMILES: CNC(=O)c1ccc2c(n1)OC(C)(C)[C@H]1CN(Cc3ccc4nc(C)c(=O)[nH]c4c3F)CCN21 ; CNC(=O)c1ccc2c(n1)OC(C)(C)[C@@H]1CN(Cc3ccc4nc(C)c(=O)[nH]c4c3F)CCN21 ; CNC(=O)c1cc2c(c(F)n1)N1CCN(Cc3ccc4nc(C)c(=O)[nH]c4c3F)C[C@@H]1CO2 ; CNC(=O)c1cc2c(c(F)n1)N1CCN(Cc3ccc4nc(C)c(=O)[nH]c4c3F)C[C@H]1CO2 ; CNC(=O)c1cc2c(c(F)n1)N1CCN(Cc3ccc4nc(C)c(=O)[nH]c4c3F)C[C@@H]1[C@H](C)O2 ; CNC(=O)c1cc2c(c(F)n1)N1CCN(Cc3ccc4nc(C)c(=O)[nH]c4c3F)C[C@H]1[C@@H](C)O2]

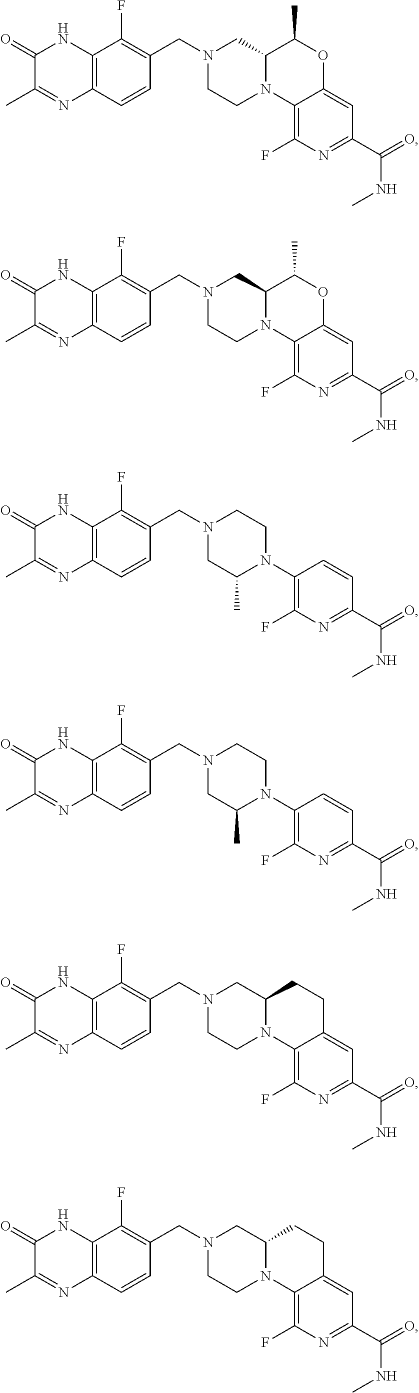 [SMILES: CNC(=O)c1cc2c(c(F)n1)N1CCN(Cc3ccc4nc(C)c(=O)[nH]c4c3F)C[C@@H]1[C@@H](C)O2 ; CNC(=O)c1cc2c(c(F)n1)N1CCN(Cc3ccc4nc(C)c(=O)[nH]c4c3F)C[C@H]1[C@H](C)O2 ; CNC(=O)c1ccc(N2CCN(Cc3ccc4nc(C)c(=O)[nH]c4c3F)C[C@H]2C)c(F)n1 ; CNC(=O)c1ccc(N2CCN(Cc3ccc4nc(C)c(=O)[nH]c4c3F)C[C@@H]2C)c(F)n1 ; CNC(=O)c1cc2c(c(F)n1)N1CCN(Cc3ccc4nc(C)c(=O)[nH]c4c3F)C[C@H]1CC2 ; CNC(=O)c1cc2c(c(F)n1)N1CCN(Cc3ccc4nc(C)c(=O)[nH]c4c3F)C[C@@H]1CC2]

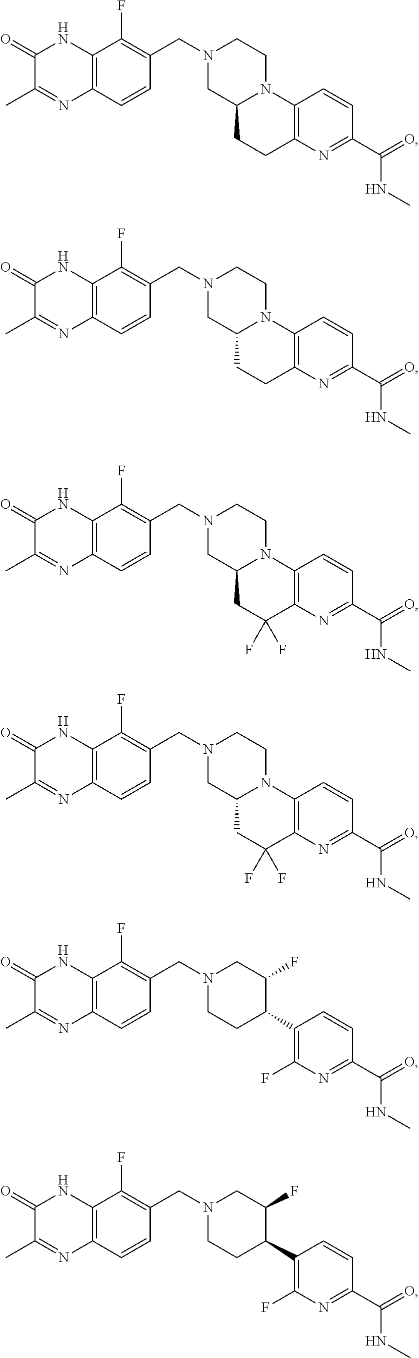 [SMILES: CNC(=O)c1ccc2c(n1)CC[C@H]1CN(Cc3ccc4nc(C)c(=O)[nH]c4c3F)CCN21 ; CNC(=O)c1ccc2c(n1)CC[C@@H]1CN(Cc3ccc4nc(C)c(=O)[nH]c4c3F)CCN21 ; CNC(=O)c1ccc2c(n1)C(F)(F)C[C@H]1CN(Cc3ccc4nc(C)c(=O)[nH]c4c3F)CCN21 ; CNC(=O)c1ccc2c(n1)C(F)(F)C[C@@H]1CN(Cc3ccc4nc(C)c(=O)[nH]c4c3F)CCN21 ; CNC(=O)c1ccc([C@@H]2CCN(Cc3ccc4nc(C)c(=O)[nH]c4c3F)C[C@@H]2F)c(F)n1 ; CNC(=O)c1ccc([C@H]2CCN(Cc3ccc4nc(C)c(=O)[nH]c4c3F)C[C@H]2F)c(F)n1]

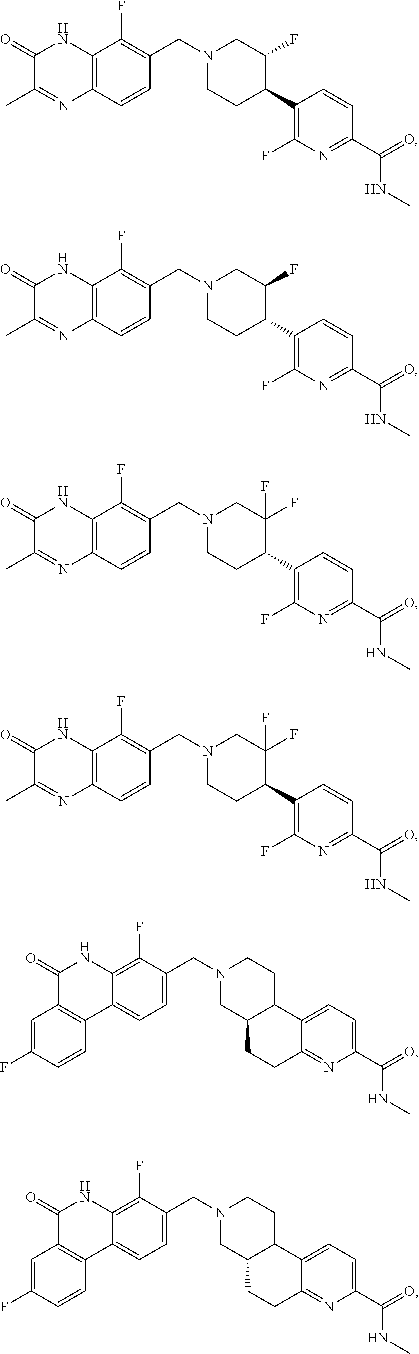

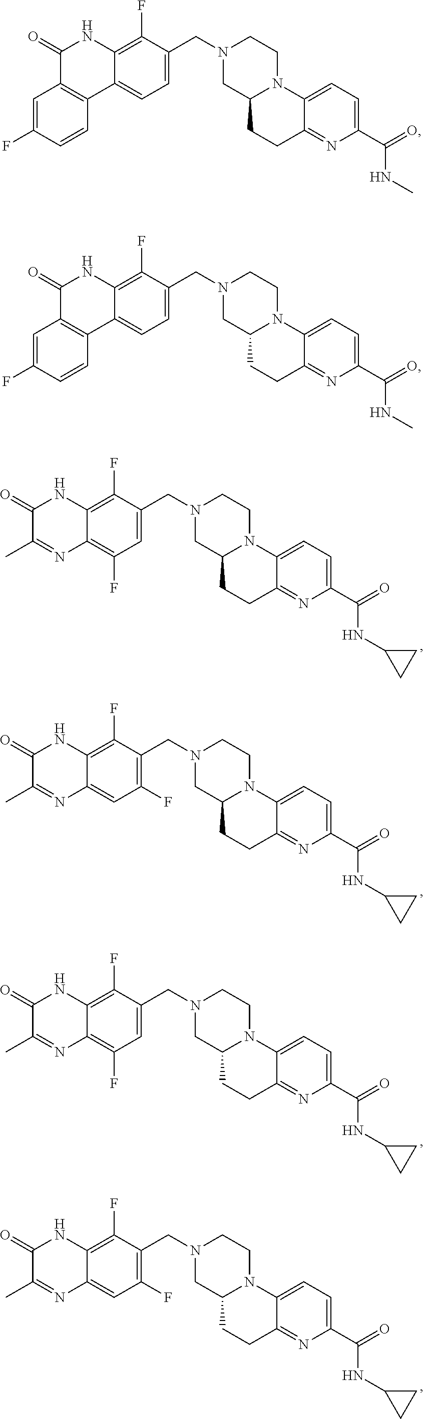 [SMILES: CNC(=O)c1ccc2c(n1)CC[C@H]1CN(Cc3ccc4c([nH]c(=O)c5cc(F)ccc54)c3F)CCN21 ; CNC(=O)c1ccc2c(n1)CC[C@@H]1CN(Cc3ccc4c([nH]c(=O)c5cc(F)ccc54)c3F)CCN21 ; Cc1nc2c(F)cc(CN3CCN4c5ccc(C(=O)NC6CC6)nc5CC[C@H]4C3)c(F)c2[nH]c1=O ; Cc1nc2cc(F)c(CN3CCN4c5ccc(C(=O)NC6CC6)nc5CC[C@H]4C3)c(F)c2[nH]c1=O ; Cc1nc2c(F)cc(CN3CCN4c5ccc(C(=O)NC6CC6)nc5CC[C@@H]4C3)c(F)c2[nH]c1=O ; Cc1nc2cc(F)c(CN3CCN4c5ccc(C(=O)NC6CC6)nc5CC[C@@H]4C3)c(F)c2[nH]c1=O]

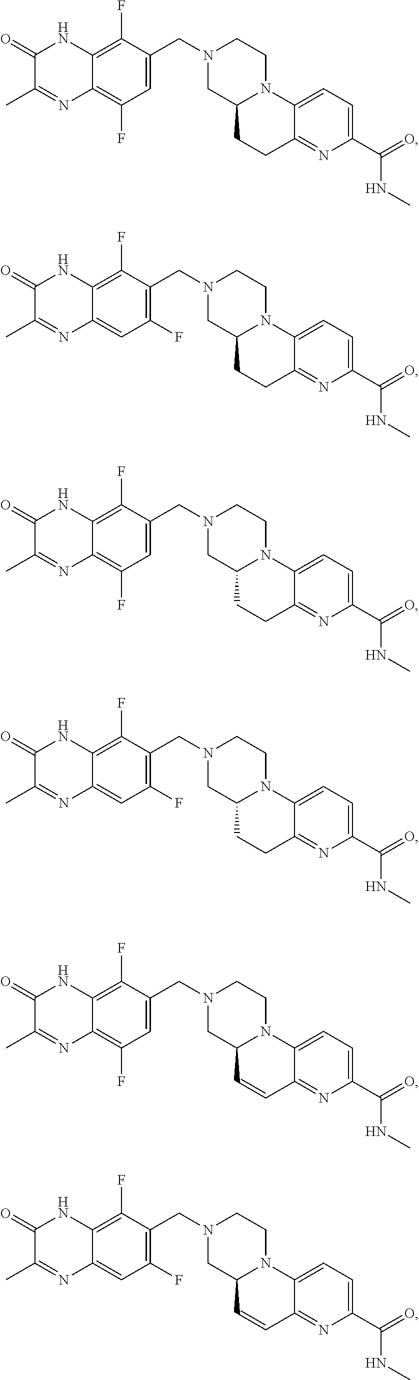 [SMILES: CNC(=O)c1ccc2c(n1)CC[C@H]1CN(Cc3cc(F)c4nc(C)c(=O)[nH]c4c3F)CCN21 ; CNC(=O)c1ccc2c(n1)CC[C@H]1CN(Cc3c(F)cc4nc(C)c(=O)[nH]c4c3F)CCN21 ; CNC(=O)c1ccc2c(n1)CC[C@@H]1CN(Cc3cc(F)c4nc(C)c(=O)[nH]c4c3F)CCN21 ; CNC(=O)c1ccc2c(n1)CC[C@@H]1CN(Cc3c(F)cc4nc(C)c(=O)[nH]c4c3F)CCN21 ; CNC(=O)c1ccc2c(n1)C=C[C@H]1CN(Cc3cc(F)c4nc(C)c(=O)[nH]c4c3F)CCN21 ; CNC(=O)c1ccc2c(n1)C=C[C@H]1CN(Cc3c(F)cc4nc(C)c(=O)[nH]c4c3F)CCN21]

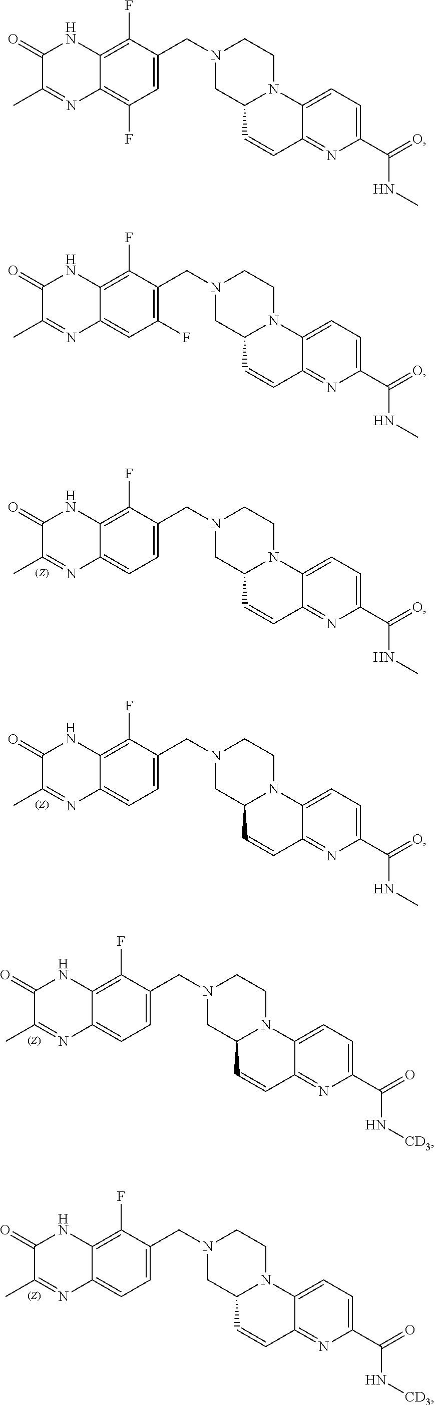 [SMILES: CNC(=O)c1ccc2c(n1)C=C[C@@H]1CN(Cc3cc(F)c4nc(C)c(=O)[nH]c4c3F)CCN21 ; CNC(=O)c1ccc2c(n1)C=C[C@@H]1CN(Cc3c(F)cc4nc(C)c(=O)[nH]c4c3F)CCN21 ; CNC(=O)c1ccc2c(n1)C=C[C@@H]1CN(Cc3ccc4nc(C)c(=O)[nH]c4c3F)CCN21 ; CNC(=O)c1ccc2c(n1)C=C[C@H]1CN(Cc3ccc4nc(C)c(=O)[nH]c4c3F)CCN21 ; [2H]C([2H])([2H])NC(=O)c1ccc2c(n1)C=C[C@H]1CN(Cc3ccc4nc(C)c(=O)[nH]c4c3F)CCN21 ; [2H]C([2H])([2H])NC(=O)c1ccc2c(n1)C=C[C@@H]1CN(Cc3ccc4nc(C)c(=O)[nH]c4c3F)CCN21]

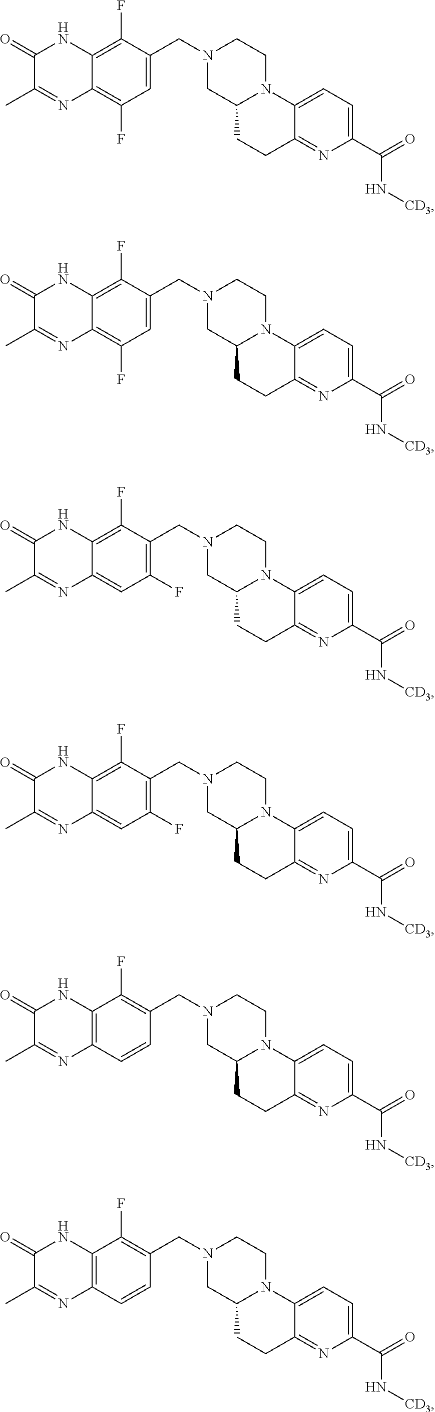 [SMILES: [2H]C([2H])([2H])NC(=O)c1ccc2c(n1)CC[C@@H]1CN(Cc3cc(F)c4nc(C)c(=O)[nH]c4c3F)CCN21 ; [2H]C([2H])([2H])NC(=O)c1ccc2c(n1)CC[C@H]1CN(Cc3cc(F)c4nc(C)c(=O)[nH]c4c3F)CCN21 ; [2H]C([2H])([2H])NC(=O)c1ccc2c(n1)CC[C@@H]1CN(Cc3c(F)cc4nc(C)c(=O)[nH]c4c3F)CCN21 ; [2H]C([2H])([2H])NC(=O)c1ccc2c(n1)CC[C@H]1CN(Cc3c(F)cc4nc(C)c(=O)[nH]c4c3F)CCN21 ; [2H]C([2H])([2H])NC(=O)c1ccc2c(n1)CC[C@H]1CN(Cc3ccc4nc(C)c(=O)[nH]c4c3F)CCN21 ; [2H]C([2H])([2H])NC(=O)c1ccc2c(n1)CC[C@@H]1CN(Cc3ccc4nc(C)c(=O)[nH]c4c3F)CCN21]

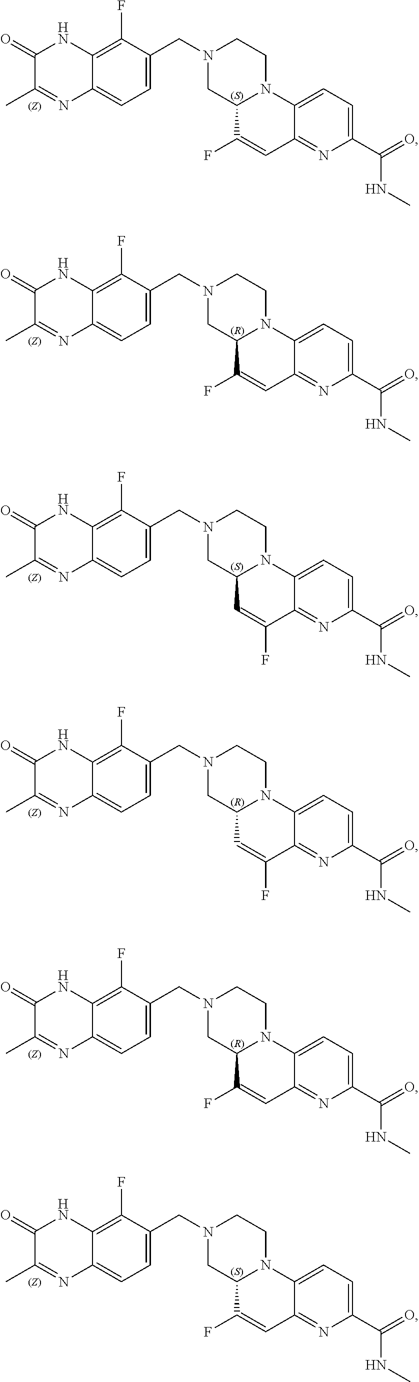

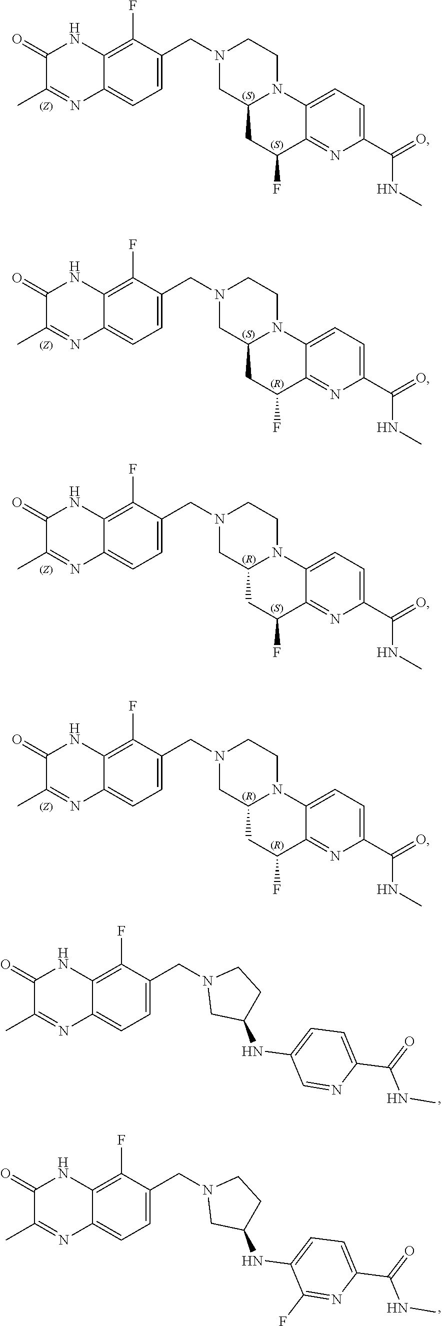

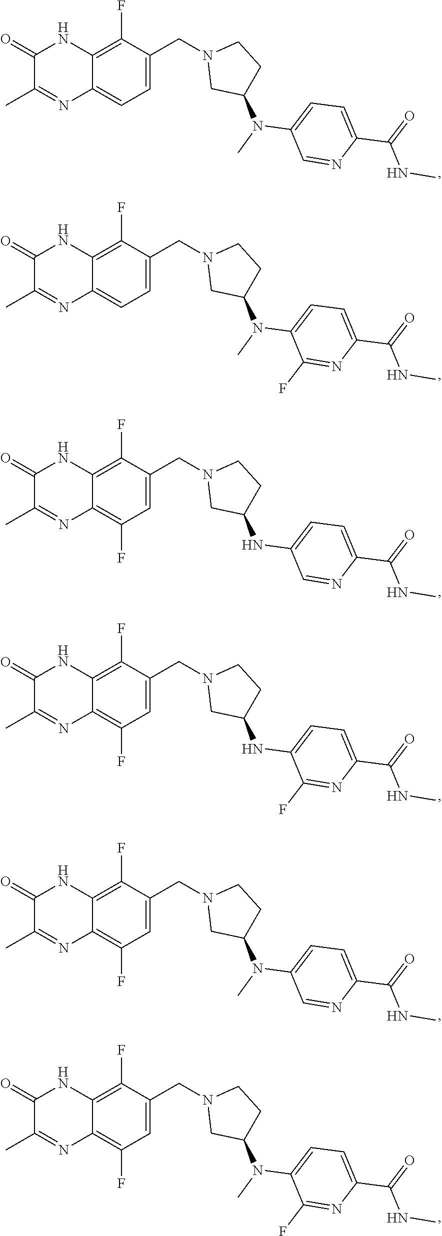 [SMILES: CNC(=O)c1ccc(N(C)[C@@H]2CCN(Cc3ccc4nc(C)c(=O)[nH]c4c3F)C2)cn1 ; CNC(=O)c1ccc(N(C)[C@@H]2CCN(Cc3ccc4nc(C)c(=O)[nH]c4c3F)C2)c(F)n1 ; CNC(=O)c1ccc(N[C@@H]2CCN(Cc3cc(F)c4nc(C)c(=O)[nH]c4c3F)C2)cn1 ; CNC(=O)c1ccc(N[C@@H]2CCN(Cc3cc(F)c4nc(C)c(=O)[nH]c4c3F)C2)c(F)n1 ; CNC(=O)c1ccc(N(C)[C@@H]2CCN(Cc3cc(F)c4nc(C)c(=O)[nH]c4c3F)C2)cn1 ; CNC(=O)c1ccc(N(C)[C@@H]2CCN(Cc3cc(F)c4nc(C)c(=O)[nH]c4c3F)C2)c(F)n1]

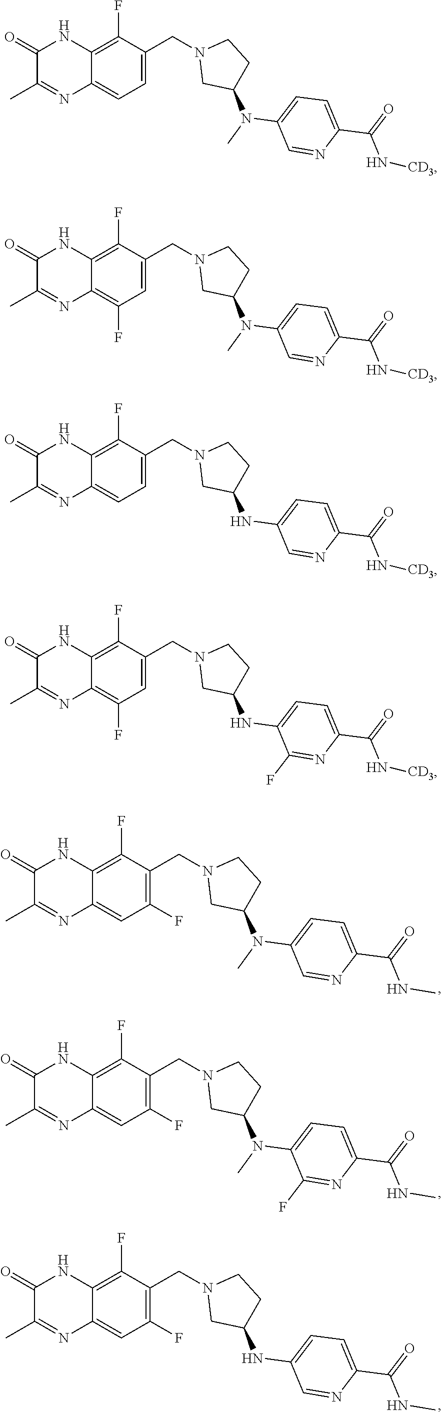

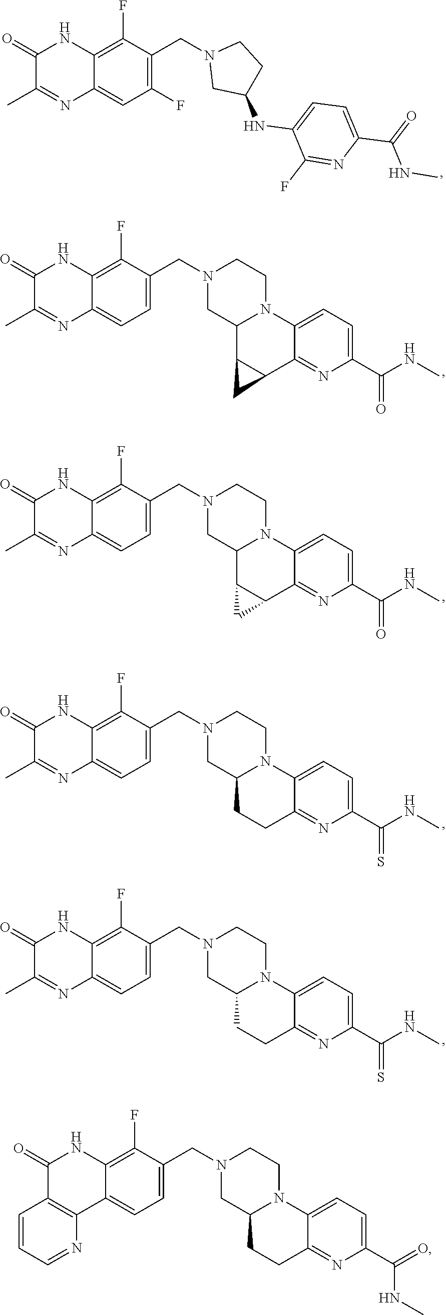 [SMILES: CNC(=O)c1ccc(N[C@@H]2CCN(Cc3c(F)cc4nc(C)c(=O)[nH]c4c3F)C2)c(F)n1 ; CNC(=O)c1ccc2c(n1)[C@H]1C[C@H]1C1CN(Cc3ccc4nc(C)c(=O)[nH]c4c3F)CCN21 ; CNC(=O)c1ccc2c(n1)[C@@H]1C[C@@H]1C1CN(Cc3ccc4nc(C)c(=O)[nH]c4c3F)CCN21 ; CNC(=S)c1ccc2c(n1)CC[C@H]1CN(Cc3ccc4nc(C)c(=O)[nH]c4c3F)CCN21 ; CNC(=S)c1ccc2c(n1)CC[C@@H]1CN(Cc3ccc4nc(C)c(=O)[nH]c4c3F)CCN21 ; CNC(=O)c1ccc2c(n1)CC[C@H]1CN(Cc3ccc4c([nH]c(=O)c5cccnc54)c3F)CCN21]

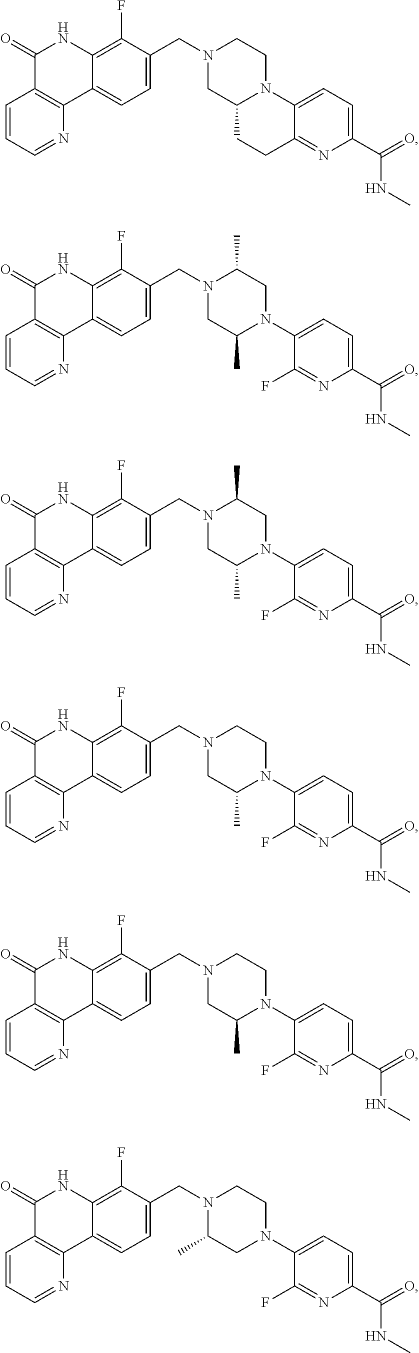 [SMILES: CNC(=O)c1ccc2c(n1)CC[C@@H]1CN(Cc3ccc4c([nH]c(=O)c5cccnc54)c3F)CCN21 ; CNC(=O)c1ccc(N2C[C@@H](C)N(Cc3ccc4c([nH]c(=O)c5cccnc54)c3F)C[C@@H]2C)c(F)n1 ; CNC(=O)c1ccc(N2C[C@H](C)N(Cc3ccc4c([nH]c(=O)c5cccnc54)c3F)C[C@H]2C)c(F)n1 ; CNC(=O)c1ccc(N2CCN(Cc3ccc4c([nH]c(=O)c5cccnc54)c3F)C[C@H]2C)c(F)n1 ; CNC(=O)c1ccc(N2CCN(Cc3ccc4c([nH]c(=O)c5cccnc54)c3F)C[C@@H]2C)c(F)n1 ; CNC(=O)c1ccc(N2CCN(Cc3ccc4c([nH]c(=O)c5cccnc54)c3F)[C@@H](C)C2)c(F)n1]

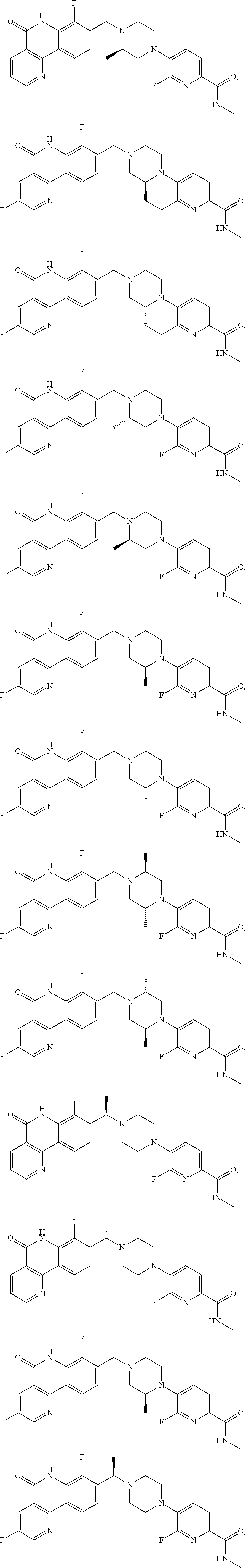

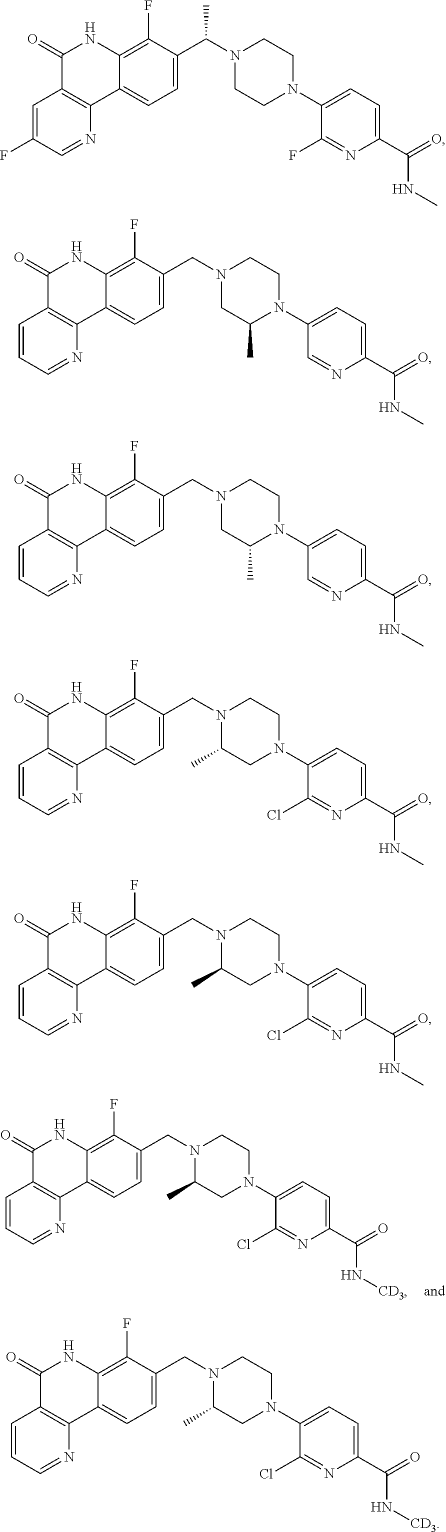

The present disclosure further provides a pharmaceutical composition comprising a therapeutically effective amount of the compound or the pharmaceutically acceptable salt thereof as defined in the present disclosure, and a pharmaceutically acceptable carrier, diluent, or excipient.

The present disclosure further provides a use of the compound, the pharmaceutically acceptable salt thereof, or the composition in the manufacture of a medicament for treating solid tumors.

In some embodiments of the present disclosure, the solid tumors refer to solid tumors such as ovarian cancer, breast cancer, prostate cancer, and glioma.

The present disclosure further provides the following synthesis methods:

### Method 1

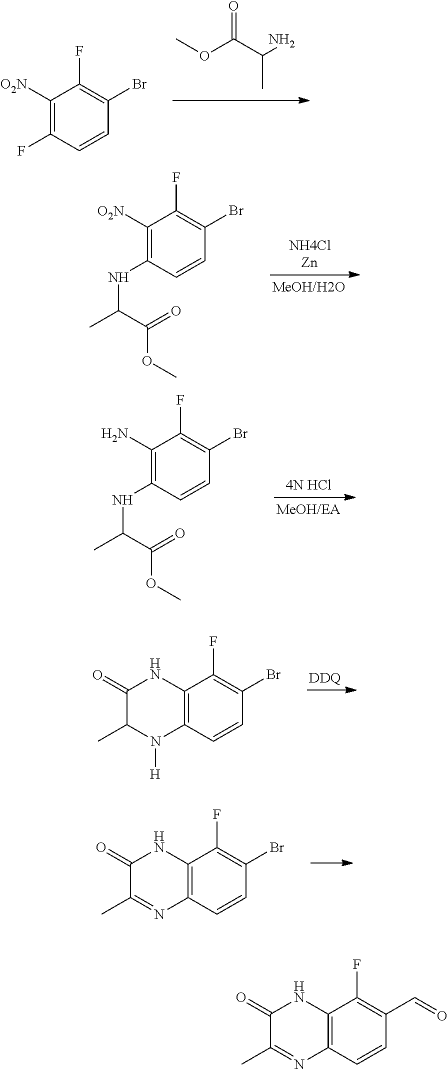

### Method 2

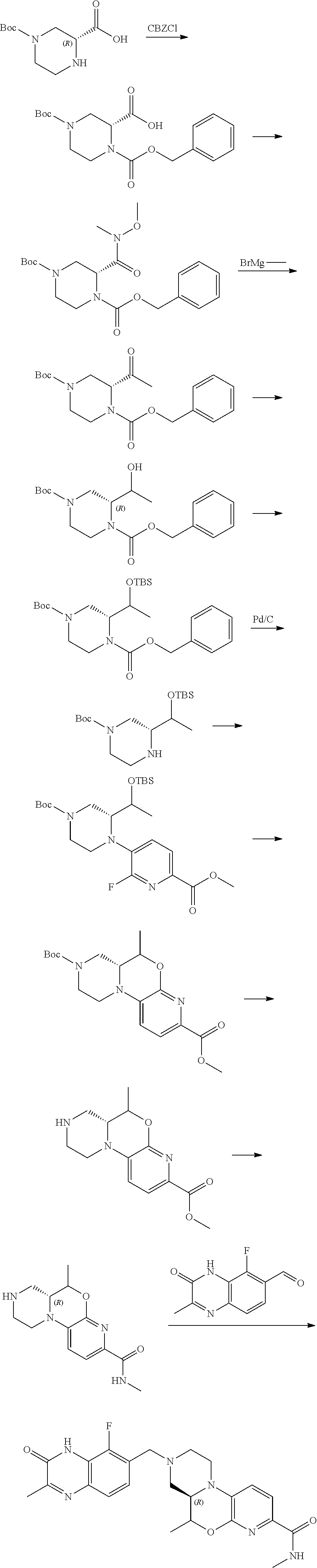

### Method 3

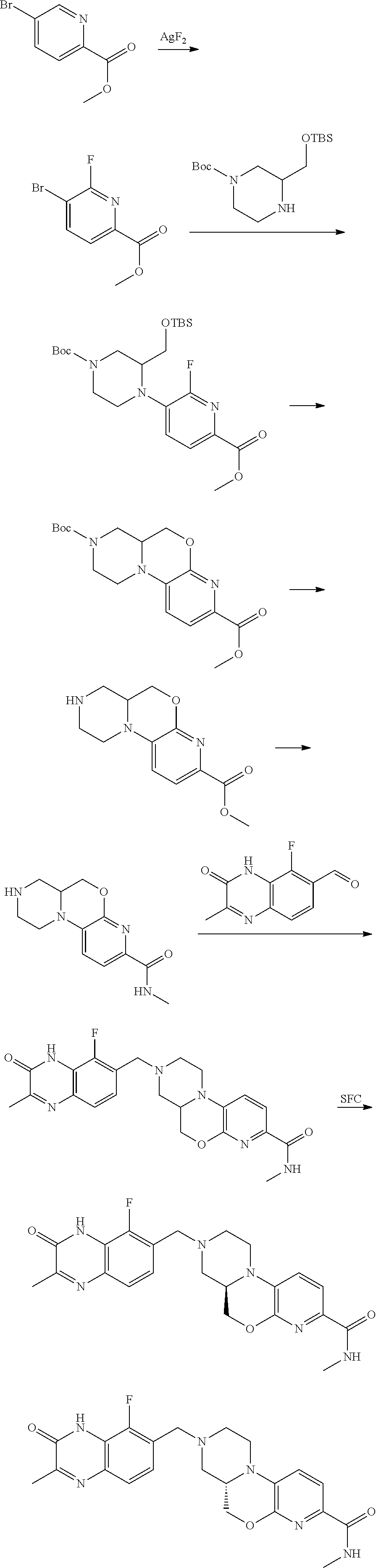

### Method 4

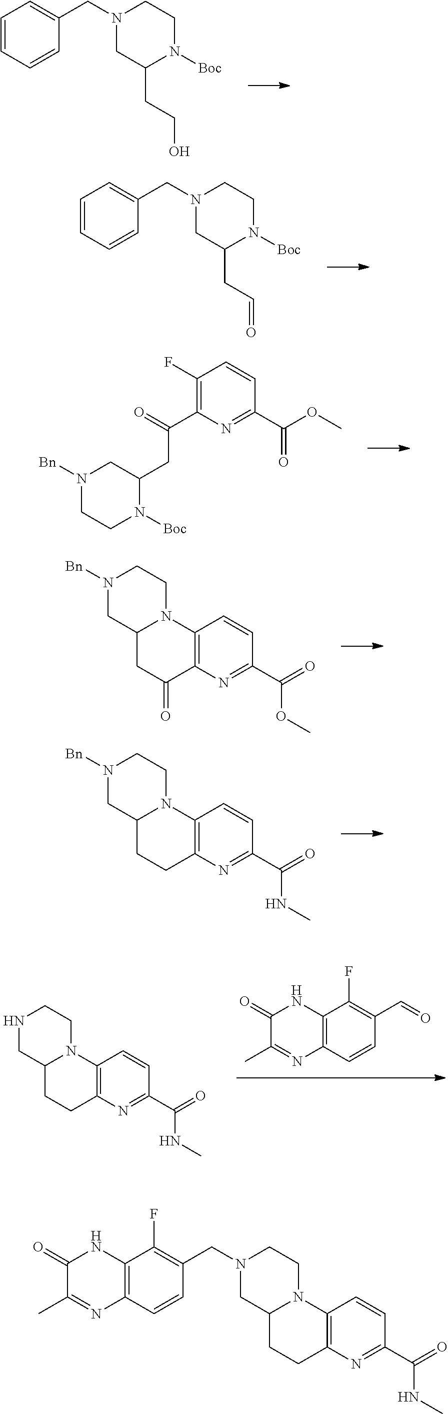

### Method 5

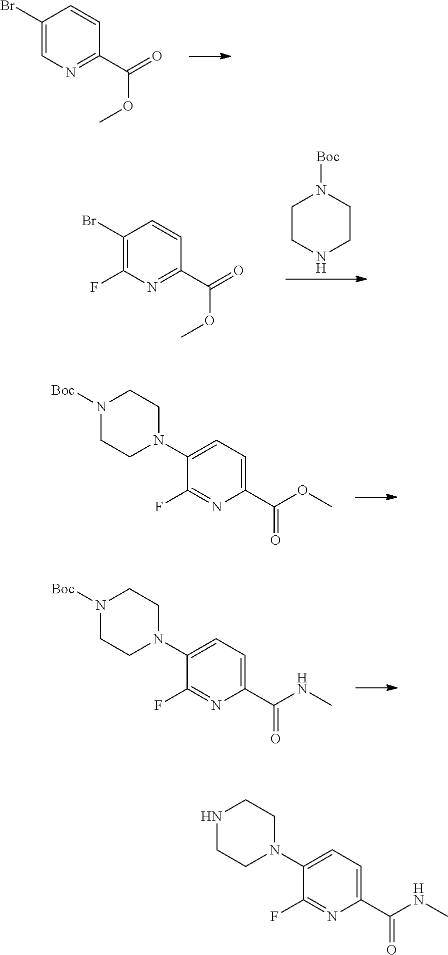 [SMILES: COC(=O)c1ccc(Br)cn1 ; COC(=O)c1ccc(Br)c(F)n1 ; CC(C)(C)OC(=O)N1CCNCC1 ; COC(=O)c1ccc(N2CCN(C(=O)OC(C)(C)C)CC2)c(F)n1 ; CNC(=O)c1ccc(N2CCN(C(=O)OC(C)(C)C)CC2)c(F)n1 ; CNC(=O)c1ccc(N2CCNCC2)c(F)n1]

### Method 6

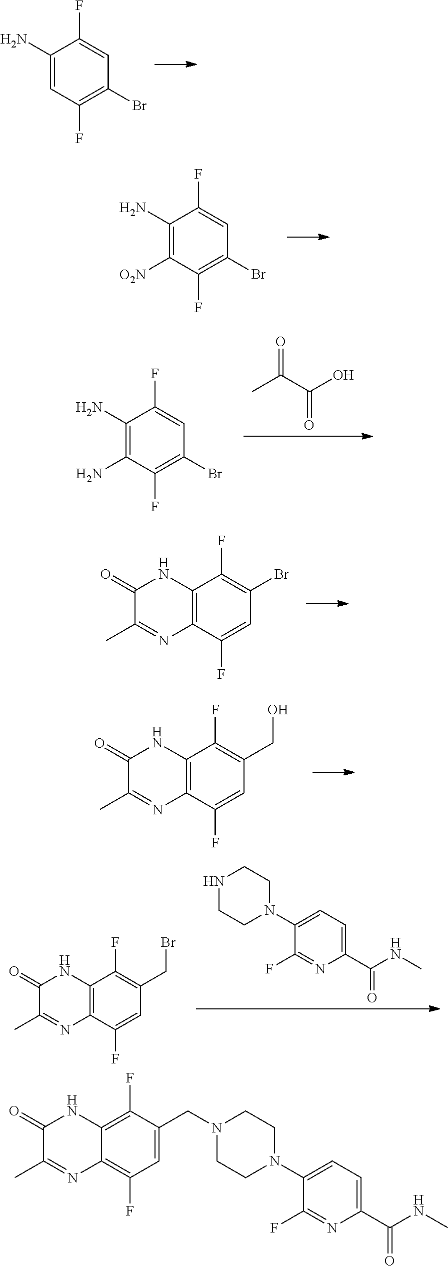

### Method 7

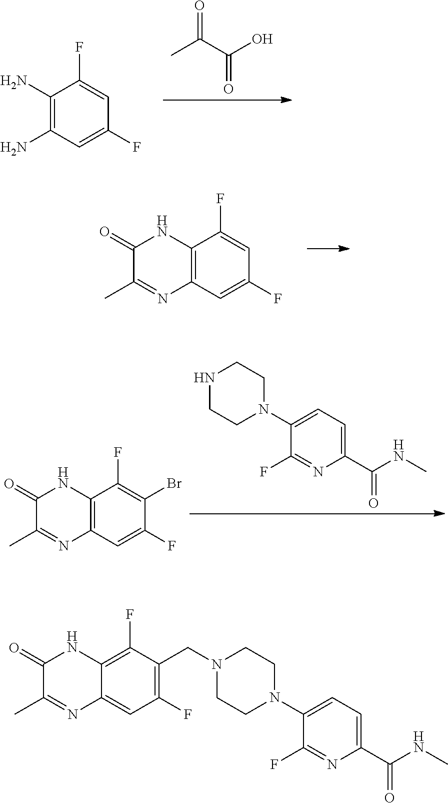

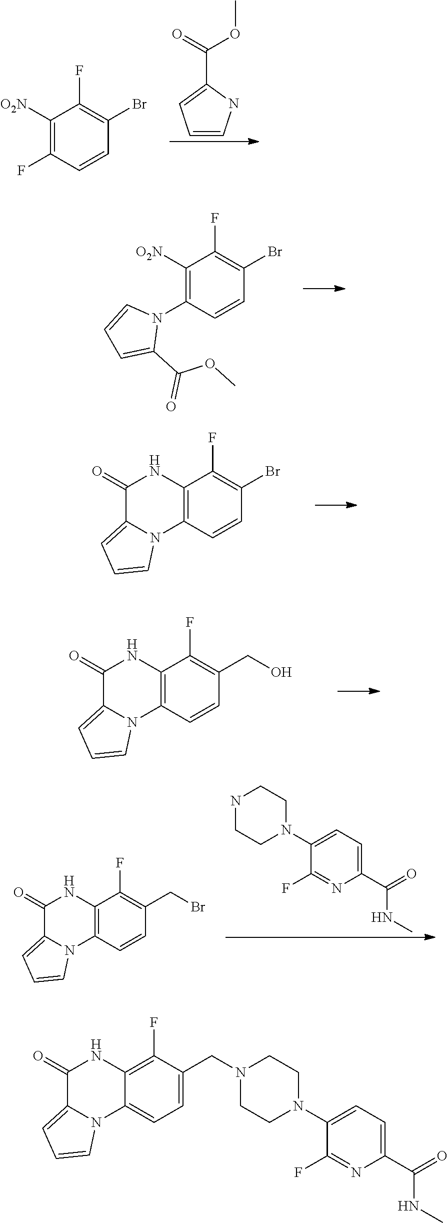

### Method 9

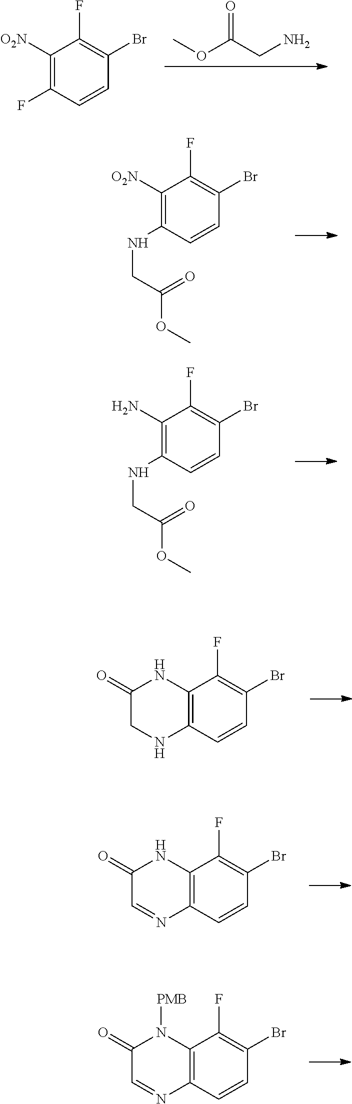

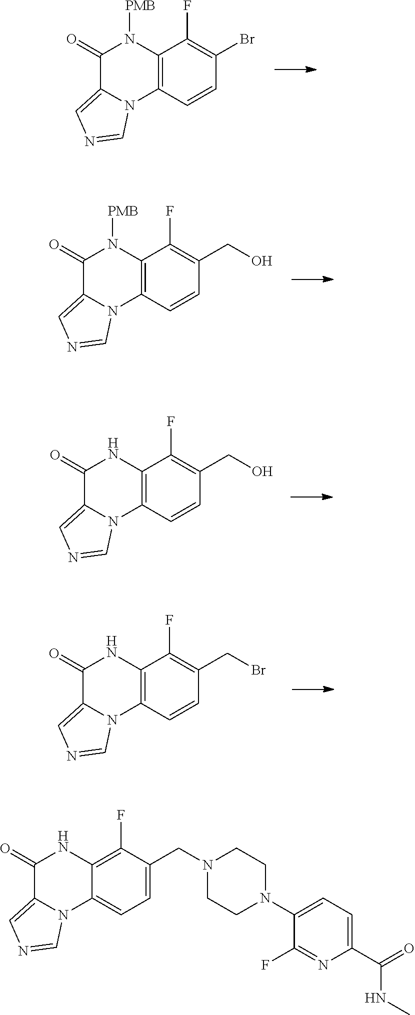

### Method 10

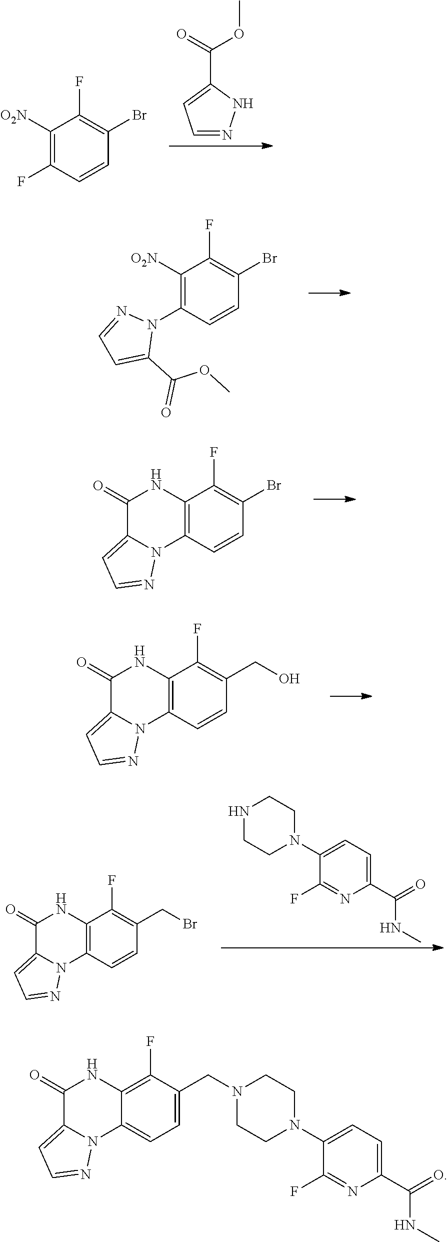

The present disclosure further provides the following bioassay methods:

### Test Method 1: PARP1 Enzyme Activity Assay

### 1. Experimental Materials:

PARP1 chemiluminescent assay kit purchased from BPS Bioscience; EnVision multimode microplate reader (PerkinElmer).

### 2. Experimental Steps:

### Reagent Preparation:

Preparation of PBST buffer: 1×PBS containing 0.05% Tween-20 was prepared, that is, 5 μL of 100% Tween-20 added to 10 mL of PBS.

Preparation of 1× assay buffer: The 10×PARP assay buffer was diluted 10-fold with double-distilled water.

### Compound Preparation:

Preparation of compound solutions: The test compound was diluted 5-fold using 100% DMSO to the 8^{th} concentration, from 1000 μM to 12.8 nM. The internal control compound was diluted 5-fold using 100% DMSO to the 8th concentration, from 200 μM to 2.56 nM. The test compound at each concentration gradient was further diluted using 1× assay buffer to prepare a working solution with a final DMSO concentration of 10%.

### Experimental Methods:

a) The histone solution from the kit was diluted 5-fold with 1×PBS. Then, 25 μL of the diluted solution was added to each well of the microplate, which was incubated overnight at 4° C.

b) After the incubation, the liquid in the wells was discarded. The wells were washed three times with 100 μL of PBST per well, and the remaining liquid in the wells was discarded.

c) 100 μL of blocking solution was added to each well of the microplate and incubated at 25° C. for 90 minutes. After the incubation, the liquid in the wells was discarded. The wells were washed three times with 100 μL of PBST per well, and the remaining liquid in the wells was discarded.

d) 12.5 μL of substrate mixture solution (1.25 μL of 10×PARP assay buffer, 1.25 μL of 10×PARP assay mix, 2.5 μL of activated DNA, and 7.5 μL of double-distilled water) was added to each well of the microplate.

e) 2.5 μL of the compound working solution was added to each well of the microplate, with the experiment set up in duplicate wells.

f) PARP1 enzyme was diluted to a concentration of 2 ng/μL, and 10 μL of the diluted PARP1 enzyme solution was added to each well of the microplate. At this point, the final concentration gradient of the test compound ranged from 10 μM to 0.128 nM, while the final concentration gradient of the internal control compound ranged from 2 μM to 0.0256 nM. The reaction system, with PARP1 at 20 ng/well, was incubated at 25° C. for 60 minutes.

g) After the incubation, the liquid in the wells was discarded. The wells were washed three times with 100 μL of PBST per well, and the remaining liquid in the wells was discarded.

h) Streptavidin-HRP was diluted 50-fold with blocking solution, and 25 μL of this diluted solution was added to each well of the microplate, which was incubated at 25° C. for 30 minutes.

i) After the incubation, the liquid in the wells was discarded. The wells were washed three times with 100 μL of PBST per well, and the remaining liquid in the wells was discarded.

j) ELISA ECL substrate A and ELISA ECL substrate B were mixed at a ratio of 1:1 (v/v), and 50 μL of this mixture was added to each well of the microplate. The chemiluminescence values were then measured.

### 3. Method for Processing Experimental Data

Using the equation (Sample−Min)/(Max−Min)×100% to convert the raw data into enzyme activity, the IC₅₀ value might be obtained by curve fitting with four parameters (obtained by “log (inhibitor) vs. response—Variable slope” mode in GraphPad Prism).

Max: containing 1% DMSO, PARP1, and the substrate mixture solution.

Min: without PARP1 enzyme.

### Test Method 2: Study on the Binding Affinity of the Compounds of the Present Disclosure to PARP1 and PARP2

PARP1 experimental procedures:

The surface plasmon resonance (SPR) experiment was conducted on a Biacore 8K instrument (GE Healthcare).

First, under the condition of 25° C., the biotinylated PARP1 protein (sequence: 655-end) was coupled to a streptavidin-coated SA chip (Cytiva, 29699622). The specific steps were as follows: the chip surface was activated using 1 mM NaCl/50 mM NaOH. The PARP1 protein was diluted with a coupling buffer (50 mM Tris-HCl, pH 8.0, 150 mM NaCl, 10 mM MgCl₂, 0.05% P20) to prepare a ligand solution at a concentration of 10 μg/mL. This solution was injected over the chip surface (with an injection time of 50 seconds and an injection flow rate of 5 μL/min), coupling the PARP1 protein to the chip surface. Any excess active sites on the chip were blocked using a solution of 50% isopropanol/1 M NaCl/50 mM NaOH. The final coupling level in the experiment was 2000-3000 RU (Response Units).

Small molecule compounds were diluted by gradients in a buffer (50 mM Tris, pH 8.0, 150 mM NaCl, 10 mM MgCl₂, 0.05% Tween 20) to obtain solutions with different compound concentrations. These solutions were injected over the chip surface, which was already coupled with the PARP1 protein. The injection flow rate was 50 μL/min, the injection time was 60 seconds, and the dissociation time was 20 minutes. The instrument detected the binding and dissociation curves of the protein-small molecule compounds.

The data from both the sample channel and the reference channel were analyzed using the Biacore 8K evaluation software to generate sensorgrams. The data were fitted based on a 1:1 binding model.

PARP2 experimental procedures:

The surface plasmon resonance (SPR) experiment was conducted on a Biacore 8K instrument (GE Healthcare).

First, under the condition of 25° C., the biotinylated PARP2 protein (sequence: 223-end) was coupled to a streptavidin-coated SA chip (Cytiva, 29699622). The specific steps were as follows: the chip surface was activated using 1 mM NaCl/60 mM NaOH. The PARP2 protein was diluted with a coupling buffer (50 mM Tris-HCl, pH 8.0, 150 mM NaCl, 10 mM MgCl₂, 0.05% P20) to prepare a ligand solution at a concentration of 10 μg/mL. This solution was injected over the chip surface (with an injection time of 50 seconds and an injection flow rate of 10 μL/min), coupling the PARP2 protein to the chip surface. Any excess active sites on the chip were blocked using a solution of 50% isopropanol/1 M NaCl/50 mM NaOH. The final coupling level in the experiment was 3000-4000 RU (Response Units).

Small molecule compounds were diluted by gradients in a buffer (50 mM Tris, pH 8.0, 150 mM NaCl, 10 mM MgCl₂, 0.05% Tween 20) to obtain solutions with different compound concentrations. These solutions were injected over the chip surface, which was already coupled with the PARP2 protein. The injection flow rate was 30 μL/min, the injection time was 60 seconds, and the dissociation time was 400 seconds. The instrument detected the binding and dissociation curves of the protein-small molecule compounds.

The data from both the sample channel and the reference channel were analyzed using the Biacore 8K evaluation software to generate sensorgrams. The data were fitted based on a 1:1 binding model.

### Technical Effect

The compounds of the present disclosure exhibit excellent binding activity to PARP1, selectively inhibiting the PARP1 protein while demonstrating weak binding activity to PARP2. These compounds also show superior antiproliferative activity against BRCA1-mutant MDA-MB-436 cells and BRCA2-knockout DLD1 cells, along with excellent membrane permeability. In vitro, the compounds of the present disclosure demonstrate excellent stability in both human and mouse liver microsomes. The compounds of the present disclosure exhibit excellent metabolic stability in vivo, demonstrating excellent oral absorption and drug exposure. Oral administration also leads to high drug concentration in brain tissue, with a high brain-to-blood ratio, and displays relatively significant anti-tumor activity.

### Related Definitions

Unless otherwise specified, the following terms and phrases when used herein have the following meanings. A specific term or phrase should not be considered indefinite or unclear in the absence of a particular definition, but should be understood in the ordinary sense. When a trading name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, an allergic reaction, or other problems or complications, commensurate with a reasonable benefit/risk ratio.

The term “pharmaceutically acceptable salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by contacting such compounds with a sufficient amount of base in a pure solution or a suitable inert solvent. The pharmaceutically acceptable base addition salt includes a salt of sodium, potassium, calcium, ammonium, organic amine, magnesium, or similar salts. When the compound of the present disclosure contains a relatively basic functional group, an acid addition salt can be obtained by contacting the compound with a sufficient amount of acid in a pure solution or a suitable inert solvent. Certain specific compounds of the present disclosure contain both basic and acidic functional groups, thus can be converted to any base or acid addition salt.

The pharmaceutically acceptable salt of the present disclosure can be prepared from the parent compound that contains an acidic or basic moiety by conventional chemical method. Generally, such salt can be prepared by reacting the free acid or base form of the compound with a stoichiometric amount of an appropriate base or acid in water or an organic solvent or a mixture thereof.

The compounds of the present disclosure may exist in specific geometric or stereoisomeric forms. The present disclosure contemplates all such compounds, including cis and trans isomers, (−)- and (+)-enantiomers, (R)- and(S)-enantiomers, diastereoisomers, (D)-isomers, (L)-isomers, racemic, and other mixtures thereof, such as enantiomers or diastereomer enriched mixtures, all of which are within the scope of the present disclosure. Additional asymmetric carbon atoms may be present in substituents such as alkyl. All these isomers and their mixtures are included within the scope of the present disclosure.

Unless otherwise specified, the term “enantiomer” or “optical isomer” refers to stereoisomers that are mirror images of each other.

Unless otherwise specified, the term “cis-trans isomer” or “geometric isomer” is caused by the inability to rotate freely of double bonds or single bonds of ring-forming carbon atoms.

Unless otherwise specified, the term “diastereomer” refers to a stereoisomer in which a molecule has two or more chiral centers and the relationship between the molecules is not mirror images.

Unless otherwise specified, “(+)” refers to dextrorotation, “(−)” refers to levorotation, and or “(±)” refers to racemic.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) or a straight dashed bond ( [symbol] ).

Unless otherwise specified, when a double bond structure, such as carbon-carbon double bond, carbon-nitrogen double bond, and nitrogen-nitrogen double bond, exists in the compound, and each of the atoms on the double bond is connected to two different substituents (including the condition where a double bond contains a nitrogen atom, the lone pair of electrons attached on the nitrogen atom is regarded as a substituent connected), if the atom on the double bond in the compound is connected to its substituent by a wave line ( [symbol] ), this refers to the (Z) isomer, (E) isomer or a mixture of two isomers of the compound. For example, the following formula (A) means that the compound exists as a single isomer of formula (A-1) or formula (A-2) or as a mixture of two isomers of formula (A-1) and formula (A-2); the following formula (B) means that the compound exists in the form of a single isomer of formula (B-1) or formula (B-2) or in the form of a mixture of two isomers of formula (B-1) and formula (B-2). The following formula (C) means that the compound exists as a single isomer of formula (C-1) or formula (C-2) or as two a mixture of two isomers of formula (C-1) and formula (C-2).

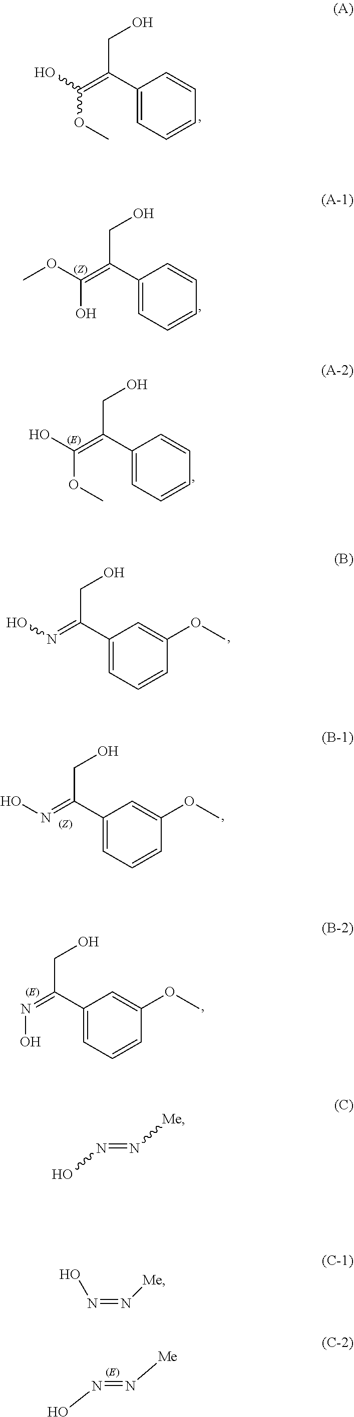

Unless otherwise specified, the term “tautomer” or “tautomeric form” means that at room temperature, the isomers of different functional groups are in dynamic equilibrium and can be transformed into each other quickly. If tautomers possibly exist (such as in solution), the chemical equilibrium of tautomers can be reached. For example, proton tautomer (also called prototropic tautomer) includes interconversion through proton migration, such as keto-enol isomerization and imine-enamine isomerization. Valence tautomer includes some recombination of bonding electrons for mutual transformation. A specific example of keto-enol tautomerization is the tautomerism between two tautomers of pentane-2,4-dione and 4-hydroxypent-3-en-2-one.

Unless otherwise specified, the terms “enriched in one isomer”, “enriched in isomers”, “enriched in one enantiomer” or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomeric excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

The compound of the present disclosure may contain an unnatural proportion of atomic isotope at one or more than one atom that constitutes the compound. For example, the compound can be radiolabeled with a radioactive isotope, such as tritium (³H), iodine-125 (¹²⁵I), or C-14 (¹⁴C). For another example, deuterated drugs can be formed by replacing hydrogen with deuterium, the bond formed by deuterium and carbon is stronger than that of ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “optional” or “optionally” means that the subsequent event or condition may occur but not requisite, that the term includes the instance in which the event or condition occurs and the instance in which the event or condition does not occur.

The term “substituted” means one or more than one hydrogen atom on a specific atom is substituted by the substituent, including deuterium and hydrogen variables, as long as the valence of the specific atom is normal and the substituted compound is stable. When the substituent is an oxygen (i.e., ═O), it means two hydrogen atoms are substituted. The term “optionally substituted” means an atom can be substituted with a substituent or not, unless otherwise specified, the type and number of the substituent may be arbitrary as long as being chemically achievable.

When any variable (such as R) occurs in the constitution or structure of the compound more than once, the definition of the variable at each occurrence is independent. Thus, for example, if a group is substituted by 0 to −2 R, the group can be optionally substituted with up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When one of the variables is selected from a single bond, it means that the two groups linked by the single bond are connected directly. For example, when Lin A-L-Z represents a single bond, the structure of A-L-Z is actually A-Z.

Unless otherwise specified, when a group has one or more than one linkable site, any one or more than one site of the group can be linked to other groups through chemical bonds. When the linking site of the chemical bond is not positioned, and there is an H atom at the linkable site, then the number of H atoms at the site will decrease correspondingly with the number of the chemical bonds linking thereto so as to meet the corresponding valence. The chemical bond between the site and other groups can be represented by a straight solid bond ( [symbol] ), a straight dashed bond ( [symbol] ), or a wavy line ( [symbol] ). For example, the straight solid bond in —OCH₃ means that it is linked to other groups through the oxygen atom in the group; the straight dashed bond in

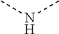

means that it is linked to other groups through the two ends of the nitrogen atom in the group; the wave lines in

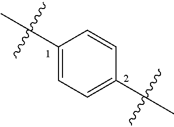 [SMILES: *c1ccc(*)cc1]

means that the phenyl group is linked to other groups through carbon atoms at position 1 and position 2.

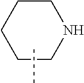

means that it can be linked to other groups through any linkable sites on the piperidinyl by one chemical bond, including at least four types of linkage, including

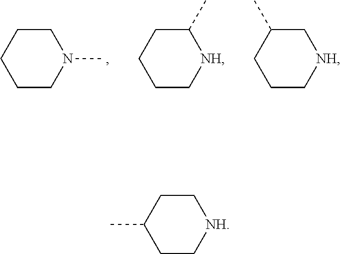

Even though the H atom is drawn on the —N—,

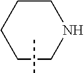

still includes the linkage of

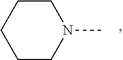

merely when one chemical bond was connected, the H of this site will be reduced by one to the corresponding monovalent piperidinyl.

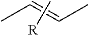

indicates that R can be connected to either end of the double bond, meaning it represents both

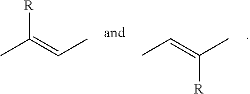

Unless otherwise specified, the phrase “two Rₐ on adjacent atoms, together with the atom to which they are attached, form a double bond” in the general formula means that the two Rₐ on adjacent carbon atoms form a double bond along with the single bond between the two carbon atoms. For example, in the structural moiety

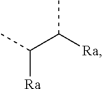

the two Rₐ form a double bond with the single bond between the two carbon atoms, resulting in the formation of the structural moiety

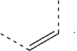

Unless otherwise specified, the term “halogen element” or “halogen” by itself or as part of another substituent refers to fluorine, chlorine, bromine, or iodine atom.

Unless otherwise specified, the term “C₁₋₃ alkyl” refers to a linear or branched saturated hydrocarbon group consisting of 1 to 3 carbon atoms. The C₁₋₃ alkyl includes C₁₋₂ and C₂₋₃ alkyl, etc.; it can be monovalent (such as methyl), divalent (such as methylene), or multivalent (such as methine). Examples of C₁₋₃ alkyl include, but are not limited to, methyl (Me), ethyl (Et), propyl (including n-propyl and isopropyl), etc.

Unless otherwise specified, the term “C₁₋₃ alkoxy” refers to an alkyl group containing 1 to 3 carbon atoms that are connected to the rest of the molecule through an oxygen atom. The C₁₋₃ alkoxy includes C₁₋₂, C₂₋₃, C₃, C₂ alkoxy, etc. Examples of C₁₋₃ alkoxy include, but are not limited to, methoxy, ethoxy, propoxy (including n-propoxy and isopropoxy), etc.

Unless otherwise specified, “C₃₋₅ cycloalkyl” refers to a saturated cyclic hydrocarbon group consisting of 3 to 5 carbon atoms, which is a monocyclic system, and the C₃₋₅ cycloalkyl includes C₃₋₄, C₄₋₅ cycloalkyl, etc.; it can be monovalent, divalent, or multivalent. Examples of C₃₋₅ cycloalkyl include, but are not limited to, cyclopropyl, cyclobutyl, cyclopentyl, etc.

Unless otherwise specified, the terms “5- to 6-membered heteroaryl ring” and “5- to 6-membered heteroaryl” are used interchangeably. The term “5- to 6-membered heteroaryl” refers to a monocyclic group consisting of 5 to 6 ring atoms with a conjugated π-electron system, in which 1, 2, 3, or 4 ring atoms are heteroatoms independently selected from O, S, and N, and the rest are carbon atoms, Here, the nitrogen atom is optionally quaternized, and nitrogen and sulfur heteroatoms can be optionally oxidized (i.e., NO and S(O)ₚ, wherein p is 1 or 2). The 5- to 6-membered heteroaryl can be linked to the rest of the molecule through a heteroatom or a carbon atom. The 5- to 6-membered heteroaryl includes 5-membered and 6-membered heteroaryl. Examples of the 5- to 6-membered heteroaryl include, but are not limited to, pyrrolyl (including N-pyrrolyl, 2-pyrrolyl, and 3-pyrrolyl, etc.), pyrazolyl (including 2-pyrazolyl and 3-pyrazolyl, etc.), imidazolyl (including N-imidazolyl, 2-imidazolyl, 4-imidazolyl, and 5-imidazolyl, etc.), oxazolyl (including 2-oxazolyl, 4-oxazolyl, and 5-oxazolyl, etc.), triazolyl (including 1H-1,2,3-triazolyl, 2H-1,2,3-triazolyl, 1H-1,2,4-triazolyl, and 4H-1,2,4-triazolyl, etc.), tetrazolyl, isoxazolyl (including 3-isoxazolyl, 4-isoxazolyl, and 5-isoxazolyl, etc.), thiazolyl (including 2-thiazolyl, 4-thiazolyl, and 5-thiazolyl, etc.), furyl (including 2-furyl and 3-furyl, etc.), thienyl (including 2-thienyll and 3-thienyl, etc.), pyridyl (including 2-pyridyl, 3-pyridyl, and 4-pyridyl, etc.), pyrazinyl, pyrimidinyl (including 2-pyrimidinyl, 4-pyrimidinyl, etc.).

Unless otherwise specified, the terms “6-membered heteroaryl ring” and “6-membered heteroaryl” in the present disclosure can be used interchangeably, and the term “6-membered heteroaryl” refers to a monocyclic group consisting of 6 ring atoms with a conjugated π-electron system, wherein 1, 2, 3, or 4 ring atoms are heteroatoms independently selected from O, S, and N, and the rest are carbon atoms. Here, the nitrogen atom is optionally quaternized, and nitrogen and sulfur heteroatoms can be optionally oxidized (i.e., NO and S(O)ₚ, wherein p is 1 or 2). The 5- to 6-membered heteroaryl can be linked to the rest of the molecule through a heteroatom or a carbon atom.

Unless otherwise specified, Cₙ₋ₙ₊ₘ or Cₙ-Cₙ₊ₘ includes any specific case of n to n+m carbons, for example, C₁₋₁₂ includes C₁, C₂, C₃, C₄, C₅, C₆, C₇, C₈, C₉, C₁₀, C₁₁, and C₁₂, and any range from n to n+m is also included, for example C₁₋₁₂ includes C₁₋₃, C₁₋₆, C₁₋₉, C₃₋₆, C₃₋₉, C₃₋₁₂, C₆₋₉, C₆₋₁₂, C₉₋₁₂, etc.; similarly, n-membered to n+m-membered means that the number of atoms on the ring is from n to n+m, for example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring, and any range from n to n+m is also included, for example, 3- to 12-membered ring includes 3- to 6-membered ring, 3- to 9-membered ring, 5- to 6-membered ring, 5- to 7-membered ring, 6- to 7-membered ring, 6- to 8-membered ring, 6- to 10-membered ring, etc.

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, and preferred embodiments include, but are not limited to, the examples of the present disclosure.

The structure of the compounds of the present disclosure can be confirmed by conventional methods known to those skilled in the art, and if the present disclosure involves an absolute configuration of a compound, then the absolute configuration can be confirmed by means of conventional techniques in the art. For example, in the case of single crystal X-ray diffraction (SXRD), the absolute configuration can be confirmed by collecting diffraction intensity data from the cultured single crystal using a Bruker D8 venture diffractometer with CuKα radiation as the light source and scanning mode: p/o scan, and after collecting the relevant data, the crystal structure can be further analyzed by the direct method (Shelxs97).

The solvents used in the present disclosure are commercially available. The present disclosure uses the following abbreviations: hr represents hours; min represents minutes; DIEA represents N,N-diisopropylethylamine; DDQ represents 2,3-dichloro-5,6-dicyanobenzoquinone; XPHOS-PD-G2 represents chloro(2-dicyclohexylphosphino-2,4,6-triisopropyl-1,1′-biphenyl) [2-(2-amino-1,1′-biphenyl)]palladium (II); TBSCl represents tert-butyldimethylsilyl chloride; DMAP represents 4-dimethylaminopyridine; Cs₂CO₃ represents cesium carbonate; RuPhos represents 2-dicyclohexylphosphino-2,6-diisopropoxy-1,1′-biphenyl; Pd₂(dba)₃ represents tris(dibenzylideneacetone) dipalladium; TBAF represents tetrabutylammonium fluoride; Et₃N represents triethylamine; Na₂CO₃ represents sodium carbonate; NaHCO₃ represents sodium bicarbonate; H₂O represents water; PE represents petroleum ether; EA represents ethyl acetate; DMF represents N,N-dimethylformamide; MeOH represents methanol; EtOH represents ethanol; THF represents tetrahydrofuran; DCM represents dichloromethane; NBS represents N-bromosuccinimide; Dess-Martin represents Dess-Martin periodinane, CAS No. 87413-09-0; KHMDS represents potassium bis(trimethylsilyl)amide; Pd(OAc)₂ represents palladium acetate; PPh₃ represents triphenylphosphine; Pd/C represents palladium on carbon; HATU represents 2-(7-azabenzotriazol-1-yl)-N,N,N′,N′-tetramethyluronium hexafluorophosphate; (Boc) 20 represents di-tert-butyl dicarbonate; LiAlH₄ represents lithium aluminum hydride; N-Boc-piperazine represents 1-tert-butoxycarbonylpiperazine; Lawesson's reagent represents the compound with CAS No. 19172-47-5; DMSO represents dimethyl sulfoxide; NaBH(OAc)₃ represents sodium triacetoxyborohydride; KI represents potassium iodide; NH₄Cl represents ammonium chloride; HCl represents hydrochloric acid; dioxane represents 1,4-dioxane; Pd(dppf)Cl₂ represents [1,1′-bis(diphenylphosphino) ferrocene]dichloropalladium.

### BRIEF DESCRIPTION OF THE DRAWINGS

FIG. 1: Diagram showing tumor growth volumes.

FIG. 2: Diagram showing body weight changes of mice during drug administration.

### DETAILED DESCRIPTION OF THE PREFERRED EMBODIMENT

The present disclosure is described in detail by the examples below, but it does not mean that there are any adverse restrictions on the present disclosure. The present disclosure has been described in detail herein, and its specific examples have also been disclosed; for those skilled in the art, it is obvious to make various modifications and improvements to the specific examples of the present disclosure without departing from the spirit and scope of the present disclosure.

### Example 1

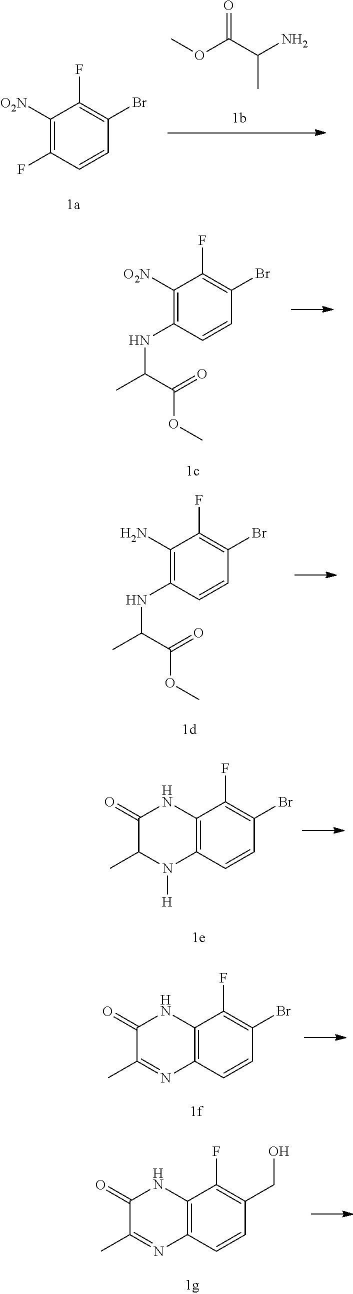

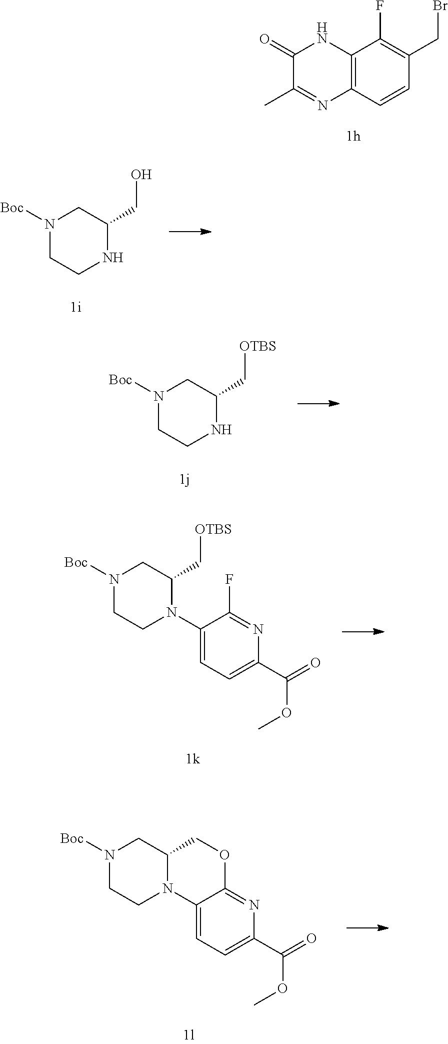 [SMILES: Cc1nc2ccc(CBr)c(F)c2[nH]c1=O ; CC(C)(C)OC(=O)N1CCN[C@@H](CO)C1 ; CC(C)(C)OC(=O)N1CCN[C@@H](CO[Si](C)(C)C(C)(C)C)C1 ; COC(=O)c1ccc(N2CCN(C(=O)OC(C)(C)C)C[C@@H]2CO[Si](C)(C)C(C)(C)C)c(F)n1 ; COC(=O)c1ccc2c(n1)OC[C@H]1CN(C(=O)OC(C)(C)C)CCN21]

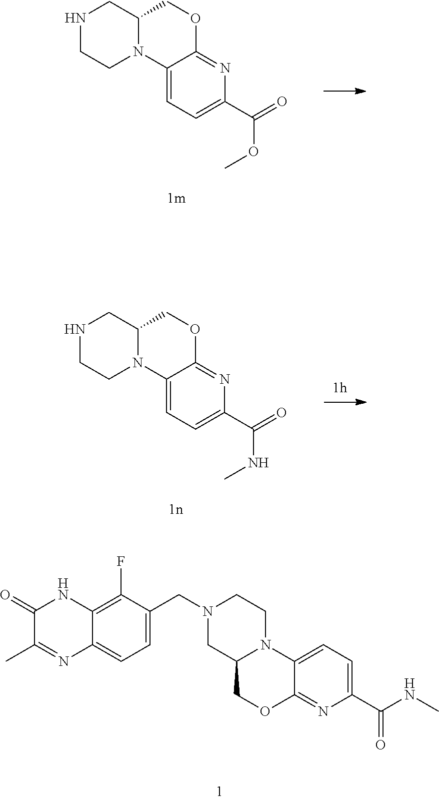 [SMILES: COC(=O)c1ccc2c(n1)OC[C@H]1CNCCN21 ; CNC(=O)c1ccc2c(n1)OC[C@H]1CNCCN21 ; CNC(=O)c1ccc2c(n1)OC[C@H]1CN(Cc3ccc4nc(C)c(=O)[nH]c4c3F)CCN21]

### Step 1: Synthesis of Intermediate 1c

Compound 1a (9.5 g, 39.92 mmol), DIEA (15.48 g, 119.75 mmol, 20.86 mL), and compound 1b (6.13 g, 43.91 mmol, HCl) were added to DMF (100 mL) and the reaction mixture was stirred at 25° C. for 18 hours. Water (1000 mL) was then added to the reaction mixture. The mixture was extracted with ethyl acetate (3× 100 mL). The organic phases were combined, dried, and concentrated to obtain a crude product. The crude product was subjected to column chromatography (eluent: PE:EA=4:1, v/v) to obtain intermediate 1c. MS m/z: 320.8, 322.8 [M+H]⁺.

### Step 2: Synthesis of Intermediate 1d

At 0° C., intermediate 1c (7.4 g, 23.05 mmol), NH₄Cl (9.86 g, 184.37 mmol), and zinc powder (7.7 g, 117.76 mmol) were added to a mixture of MeOH (100 mL) and H₂O (2 mL). The reaction mixture was stirred for 2 hours. The reaction mixture was then filtered, and the filtrate was evaporated under reduced pressure to remove methanol. Water (100 mL) was added thereto, and the mixture was extracted with ethyl acetate (2×30 mL). The organic phases were combined, dried, and filtered. The filtrate was concentrated under reduced pressure to obtain intermediate 1d, which was used directly in the next step. MS m/z: 290.7, 292.7 [M+H]⁺.

### Step 3: Synthesis of Intermediate 1e

At 0° C., intermediate 1d (6.7 g, 23.01 mmol) was added to a mixed solvent of 4 M HCl in dioxane (1.81 mL), ethyl acetate (70 mL), and methanol (70 mL). The reaction mixture was stirred for 2 hours. The reaction mixture was then filtered, and the filtrate was concentrated under reduced pressure to obtain intermediate 1e (5.5 g, 21.23 mmol). ¹H NMR (400 MHz, DMSO-d₆) δ ppm 10.46 (s, 1H) 7.42-7.57 (m, 1H) 7.00 (dd, J=8.53, 7.28 Hz, 1H) 6.49 (d, J=8.03 Hz, 1H) 3.82-3.86 (m, 1H) 1.25 (d, J=6.53 Hz, 3H).

### Step 4: Synthesis of Intermediate 1f

At 0° C., DDQ (5.26 g, 23.16 mmol) was added in batches to a solution of intermediate 1e (5.0 g, 19.30 mmol) in DCM (250 mL). The temperature was then raised to 25° C., and the reaction mixture was stirred for 2 hours and evaporated under reduced pressure to remove the solvent. A saturated NaHCO₃ aqueous solution (200 mL) was slowly added to quench the reaction, and the mixture was stirred overnight. The reaction mixture was extracted with ethyl acetate (3×50 mL), and the organic phases were combined and concentrated to obtain the target product, which was subjected to column chromatography (eluent: DCM:MeOH=100:1, v/v) to obtain intermediate 1f. MS m/z: 256.7, 258.7 [M+H]⁺, ¹H NMR (400 MHz, DMSO-d₆) δ ppm 12.61 (br s, 1H) 7.45-7.55 (m, 2H) 2.40 (s, 3H).

### Step 5: Synthesis of Intermediate 1g

Intermediate 1f (1.0 g, 3.89 mmol), 1-(tributylstannyl) methanol (1.50 g, 4.67 mmol), and XPHOS-PD-G2 (153.04 mg, 194.51 μmol) were added to 1,4-dioxane (20 mL). The reaction mixture was stirred at 80° C. under a nitrogen atmosphere for 4 hours. The reaction mixture was then concentrated under reduced pressure to obtain a crude product. The crude product was purified by column chromatography (eluent: DCM/MeOH=20/1, v/v) to obtain intermediate 1g. MS m/z: 209.0 [M+1]⁺.

### Step 6: Synthesis of Intermediate 1h

Under a nitrogen atmosphere at 0° C., tributylphosphine (388.72 mg, 1.92 mmol, 474.05 μL) was slowly added dropwise to a solution of 1,2-dibromotetrachloroethane (688.23 mg, 2.11 mmol, 253.96 μL) and intermediate 1g (200 mg, 960.67 μmol) in DCM (5 mL). The reaction mixture was stirred at 20° C. for 3 hours. The reaction mixture was then concentrated under reduced pressure to obtain a crude product. A mixed solvent of DCM and MeOH (20:1, 5 mL) was added to the crude product. The mixture was stirred for 30 minutes and filtered. The filter cake was concentrated under reduced pressure to obtain intermediate 1h. ¹H NMR (400 MHz, DMSO-d₆) δ ppm 12.55 (s, 1H) 7.47-7.58 (m, 1H) 7.31-7.43 (m, 1H) 4.80-4.90 (m, 2H) 2.42 (s, 3H).

### Step 7: Synthesis of Intermediate 1j

Triethylamine (1.87 g, 18.49 mmol) was added to a solution of intermediate 1i (2 g, 9.25 mmol), TBSCl (2.09 g, 13.87 mmol), and DMAP (112.97 mg, 924.74 μmol) in DCM (40 mL). The reaction mixture was stirred at 20° C. for 16 hours. The reaction mixture was then concentrated under reduced pressure to obtain a crude product. The crude product was purified by column chromatography (eluent: DCM/MeOH=20/1 to 10/1, v/v) to obtain intermediate 1j. ¹H NMR (400 MHz, CD₃OD) δ ppm 3.88-4.12 (m, 2H) 3.63 (d, J=5.52 Hz, 2H) 3.01 (d, J=12.30 Hz, 1H) 2.54-2.94 (m, 4H) 1.48 (s, 9H) 0.95 (s, 9H) 0.12 (s, 6H).

### Step 8: Synthesis of Intermediate 1k

Cs₂CO₃ (197.15 mg, 605.08 μmol), RuPhos (14.12 mg, 30.25 μmol), intermediate 1j (0.1 g, 302.54 μmol), methyl 5-bromo-6-fluoro-pyridine-2-carboxylate (70.80 mg, 302.54 μmol), and Pd₂(dba)₃ (55.41 mg, 60.51 μmol) were added to a toluene solution (4 mL). The reaction mixture was stirred at 100° C. for 24 hours. The reaction mixture was subjected to suction filtration under reduced pressure, and the filtrate was concentrated under reduced pressure to obtain a crude product. The crude product was separated by column chromatography (eluent: PE:EA=1:1, v/v) to obtain intermediate 1k. MS m/z: 484.3 [M+H]⁺.

### Step 9: Synthesis of Intermediate 11

TBAF (1 M in THE, 258.45 μL, 2.5 eq) and intermediate 1k (50 mg, 103.38 μmol) were added to a THF solution (4 mL). The reaction mixture was stirred at 25° C. for 16 hours. The reaction mixture was then concentrated under reduced pressure to obtain intermediate 1l. MS m/z: 350.2 [M+H]⁺.

### Step 10: Synthesis of Hydrochloride of Intermediate 1m

Intermediate 11 (30 mg, 85.87 μmol) was added to an EA solution (2 mL). HCl/EA (4 M, 85.87 μL) was added dropwise to the reaction mixture. The mixture was stirred at 25° C. for 2 hours. The reaction mixture was concentrated under reduced pressure to obtain the hydrochloride of intermediate 1m. MS m/z: 250.2 [M+H]⁺.

### Step 11: Synthesis of Intermediate 1n

Under a nitrogen atmosphere, the hydrochloride of intermediate 1m (20 mg) was added to an EtOH solution (2 mL). Methylamine (2.26 mg, 72.84 μmol) was then added thereto. The mixture was stirred at 20° C. for 1 hour. The reaction mixture was concentrated under reduced pressure to obtain intermediate 1n. MS m/z: 249.1 [M+H]⁺.

### Step 12: Synthesis of Trifluoroacetate of Compound 1

Intermediate 1n (10.50 mg, 36.89 μmol) was added to DMF (2 mL). Et₃N (7.47 mg, 73.78 μmol, 10.27 μL) was then added thereto. The mixture was stirred at 25° C. for 30 minutes. KI (2.70 mg, 16.24 μmol, 5.68 μL) and intermediate 1h (10 mg, 36.89 μmol) were then added to the reaction mixture, and the mixture was stirred at 50° C. for 2 hours. The reaction mixture was concentrated under reduced pressure using an oil pump at 60° C. to obtain a crude product. The crude product was purified by preparative HPLC (column: Welch Xtimate C18, 100×40 mm×3 um; mobile phase: [water (trifluoroacetic acid)-acetonitrile]; acetonitrile %: 0%-23% over 8 minutes) to obtain the trifluoroacetate of compound 1. MS m/z: 439.2 [M+H]⁺, ¹H NMR (400 MHz, CD₃OD) δ ppm 7.63-7.73 (m, 2H) 7.36-7.50 (m, 2H) 4.44-4.54 (m, 3H) 4.22 (dd, J=11.26, 7.63 Hz, 1H) 4.13 (d, J=11.01 Hz, 1H) 3.51-3.64 (m, 3H) 3.11-3.22 (m, 2H) 2.92 (s, 3H) 2.85-2.91 (m, 1H) 2.56 (s, 3H).

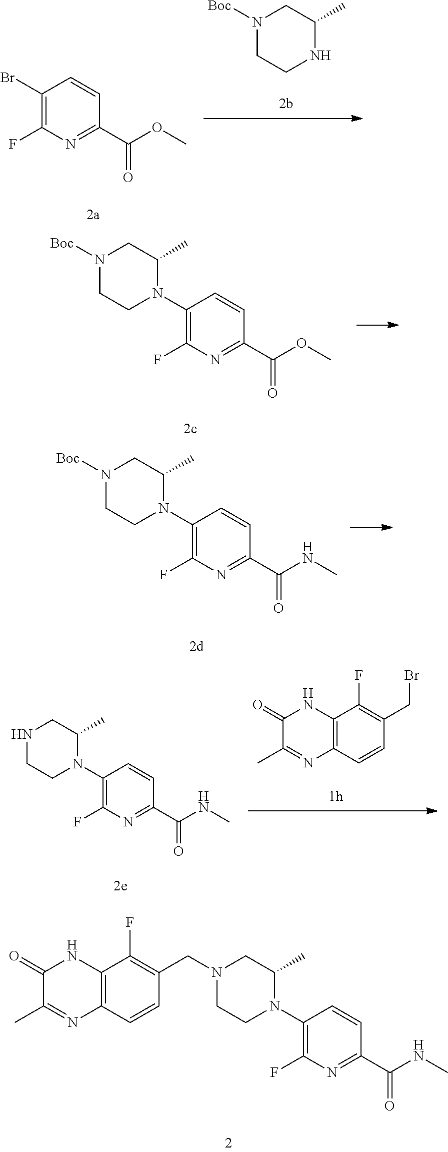

### Step 1: Synthesis of Intermediate 2c

Compound 2a (0.5 g, 2.14 mmol) and compound 2b (642.89 mg, 3.21 mmol) were added to toluene (10 mL). Cs₂CO₃ (2.09 g, 6.42 mmol), RuPhos (199.72 mg, 428.00 μmol), and Pd₂(dba)₃ (195.96 mg, 214.00 μmol) were then added thereto. The reaction mixture was purged with nitrogen three times and stirred at 100° C. for 16 hours. The reaction mixture was subjected to suction filtration under reduced pressure, and the filtrate was concentrated under reduced pressure to obtain a crude product. The crude product was purified by column chromatography (eluent: PE/EA=4/1 to 2/1, v/v) to obtain intermediate 2c. MS m/z: 354.2 [M+1]⁺, ¹H NMR (400 MHz, CDCl₃) δ ppm 7.99 (d, J=8.03 Hz, 1H) 7.22-7.29 (m, 1H) 4.01 (d, J=6.53 Hz, 1H) 4.00-4.24 (m, 1H) 3.98 (s, 3H) 3.81 (d, J=13.30 Hz, 1H) 3.29-3.44 (m, 2H) 3.07-3.25 (m, 2H) 1.50 (s, 9H) 1.08 (d, J=6.53 Hz, 3H).

### Step 2: Synthesis of Intermediate 2d

Intermediate 2c (0.4 g, 1.13 mmol) and methylamine in THF (2 M, 7.27 mL) were added to EtOH (10 mL), and the reaction mixture was stirred at 20° C. for 16 hours. The reaction mixture was concentrated under reduced pressure to obtain the crude intermediate 2d, which was used directly in the next step. MS m/z: 353.0 [M+H]⁺.

### Step 3: Synthesis of Hydrochloride of Intermediate 2e

Intermediate 2d (0.4 g, 1.14 mmol) was added to EA (5 mL). HCl in ethyl acetate (4 M, 1.42 mL) was then added thereto. The reaction mixture was stirred at 20° C. for 16 hours. The reaction mixture was concentrated under reduced pressure to obtain the hydrochloride of the crude intermediate 2e, which was used directly in the next step. MS m/z: 253.0 [M+H]⁺.

### Step 4: Synthesis of Trifluoroacetate of Compound 2

Intermediate 2e (21.30 mg) was added to DMF (2 mL). Then, Et₃N (14.93 mg, 147.56 μmol, 20.54 μL) was added thereto. The reaction mixture was stirred at 25° C. for 30 minutes. KI (5.39 mg, 32.49 μmol) and intermediate 1h (20 mg, 73.78 μmol) were then thereto. The reaction mixture was stirred at 50° C. for 2 hours. The reaction mixture was concentrated under reduced pressure to obtain a crude product. The crude product was purified by preparative HPLC (column: Welch Xtimate C18, 100×40 mm×3 um; mobile phase: [water (trifluoroacetic acid)-acetonitrile]; acetonitrile %: 0%-26% over 8 minutes) to obtain the trifluoroacetate of compound 2. MS m/z: 443.1 [M+H]⁺, ¹H NMR (400 MHz, DMSO-d₆) δ ppm 7.19 (d, J=7.28 Hz, 1H) 6.99 (s, 1H) 6.90 (d, J=8.78 Hz, 1H) 6.67 (d, J=6.78 Hz, 1H) 3.80 (s, 2H) 2.78 (s, 1H) 2.70 (s, 3H) 2.39-2.48 (m, 3H) 2.14 (s, 3H) 1.76 (s, 3H) 0.32 (s, 3H).

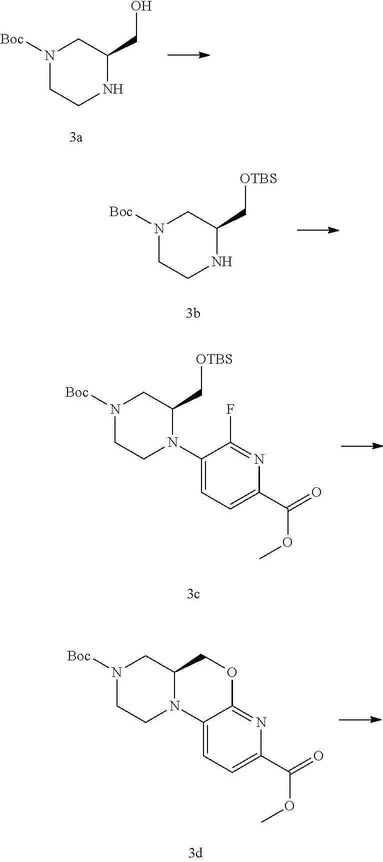 [SMILES: CC(C)(C)OC(=O)N1CCN[C@H](CO)C1 ; CC(C)(C)OC(=O)N1CCN[C@H](CO[Si](C)(C)C(C)(C)C)C1 ; COC(=O)c1ccc(N2CCN(C(=O)OC(C)(C)C)C[C@H]2CO[Si](C)(C)C(C)(C)C)c(F)n1 ; COC(=O)c1ccc2c(n1)OC[C@@H]1CN(C(=O)OC(C)(C)C)CCN21]

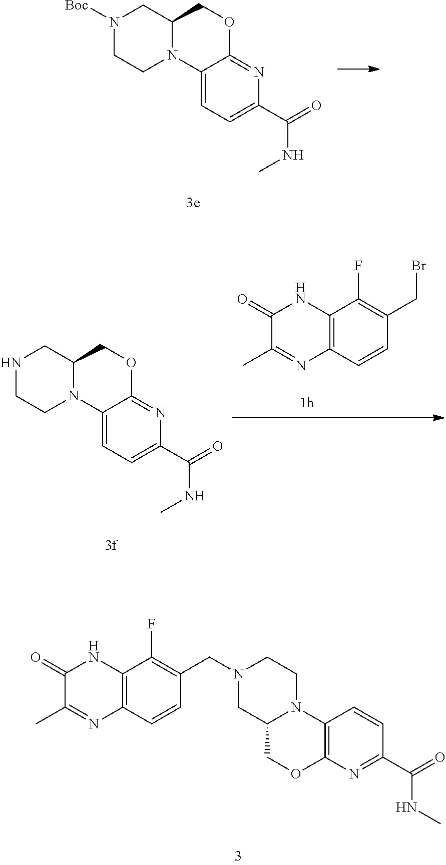 [SMILES: CNC(=O)c1ccc2c(n1)OC[C@@H]1CN(C(=O)OC(C)(C)C)CCN21 ; CNC(=O)c1ccc2c(n1)OC[C@@H]1CNCCN21 ; Cc1nc2ccc(CBr)c(F)c2[nH]c1=O ; CNC(=O)c1ccc2c(n1)OC[C@@H]1CN(Cc3ccc4nc(C)c(=O)[nH]c4c3F)CCN21]

### Step 1: Synthesis of Intermediate 3b

Compound 3a (1 g, 4.62 mmol) was added to DCM (20 mL), and the reaction mixture was cooled to 0° C. Et₃N (935.74 mg, 9.25 mmol, 1.29 mL), TBSCl (1.05 g, 6.94 mmol, 849.87 μL), and DMAP (56.49 mg, 462.37 μmol) were then added thereto. The reaction mixture was stirred at 20° C. for 16 hours. The reaction mixture was concentrated under reduced pressure to obtain a crude product. The crude product was purified by column chromatography (eluent: DCM/MeOH=20/1 to 10/1, v/v) to obtain intermediate 3b. 1H NMR (400 MHz, CDCl₃) δ ppm 3.86 (s, 1H) 3.52 (s, 1H) 3.41 (dd, J=9.79, 7.28 Hz, 1H) 2.92 (d, J=11.29 Hz, 1H) 2.63-2.84 (m, 3H) 2.48 (s, 1H) 1.90 (s, 1H) 1.28-1.48 (m, 9H) 0.72-0.97 (m, 9H)-0.08-0.08 (m, 6H).

### Step 2: Synthesis of Intermediate 3c

Methyl 5-bromo-6-fluoro-pyridine-2-carboxylate (1 g, 4.27 mmol) and intermediate 3b (987.97 mg, 2.99 mmol) were added to toluene (20 mL). RuPhos (398.80 mg, 854.00 μmol), Pd₂(dba)₃ (391.30 mg, 427.00 μmol), and Cs₂CO₃ (4.17 g, 12.81 mmol) were then added thereto. The reaction mixture was purged with nitrogen three times and stirred at 100° C. for 2 hours. The reaction mixture was subjected to suction filtration under reduced pressure, and the filtrate was concentrated under reduced pressure to obtain a crude product. The crude product was purified by column chromatography (eluent: PE/EA=4/1 to 2/1, v/v) to obtain intermediate 3c. MS m/z: 484.3 [M+H]⁺.

### Step 3: Synthesis of Intermediate 3d

Compound 3c (300 mg, 620.29 μmol) was added to THF (2 mL), and TBAF (1 M in THE, 1.86 mL) was added thereto. The reaction mixture was stirred at 20° C. for 2 hours. The reaction mixture was subjected to suction filtration under reduced pressure, and the filtrate was concentrated under reduced pressure to obtain a crude product 3d, which was used directly in the next step. MS m/z: 350.1 [M+H]⁺.

### Step 4: Synthesis of Intermediate 3e

Compound 3d (200.00 mg) was added to EtOH (5 mL), and a solution of methylamine in ethanol (1 mL, 40%) was added thereto. The reaction mixture was stirred at 20° C. for 32 hours. The reaction mixture was concentrated under reduced pressure to obtain a crude product. The crude product was used directly in the next step, yielding intermediate 3e. MS m/z: 349.1 [M+H]⁺.

### Step 5: Synthesis of Hydrochloride of Intermediate 3f

Compound 3e (0.1 g) was added to EA (5 mL), and HCl/ethyl acetate (4 M, 358.79 μL) was added thereto. The reaction mixture was stirred at 20° C. for 8 hours. The reaction mixture was concentrated under reduced pressure to obtain a crude hydrochloride of compound 3f, which was used directly in the next step. MS m/z: 249.1 [M+H]⁺.

### Step 6: Synthesis of Trifluoroacetate of Compound 3

Compound 3f (10.50 mg, HCl) was added to DMF (2 mL). Then, Et₃N (7.47 mg, 73.78 μmol, 10.27 μL) was added thereto. The reaction mixture was stirred at 25° C. for 30 minutes. KI (2.70 mg, 16.24 μmol, 5.68 μL) and compound 1h (10 mg, 36.89 μmol) were then added thereto. The reaction mixture was stirred at 50° C. for 2 hours. The reaction mixture was concentrated using an oil pump at 60° C. to obtain a crude product. The crude product was purified by preparative HPLC (column: Xtimate C18, 150× 40 mm×5 um; mobile phase: [water (trifluoroacetic acid)-acetonitrile]; acetonitrile %: 1%-30% over 10 minutes) to obtain the trifluoroacetate of compound 3. MS m/z: 439.0 [M+H]⁺, ¹H NMR (400 MHz, CD₃OD) δ ppm 7.66-7.71 (m, 2H) 7.42-7.48 (m, 1H) 7.36-7.42 (m, 1H) 4.50 (dd, J=11.19, 2.56 Hz, 1H) 4.43 (s, 2H) 4.22 (dd, J=11.13, 7.75 Hz, 1H) 4.12 (d, J=10.01 Hz, 1H) 3.56 (br s, 1H) 3.51 (d, J=9.38 Hz, 2H) 3.14 (s, 2H) 2.92 (s, 3H) 2.85 (s, 1H) 2.56 (s, 3H).

### Example 4

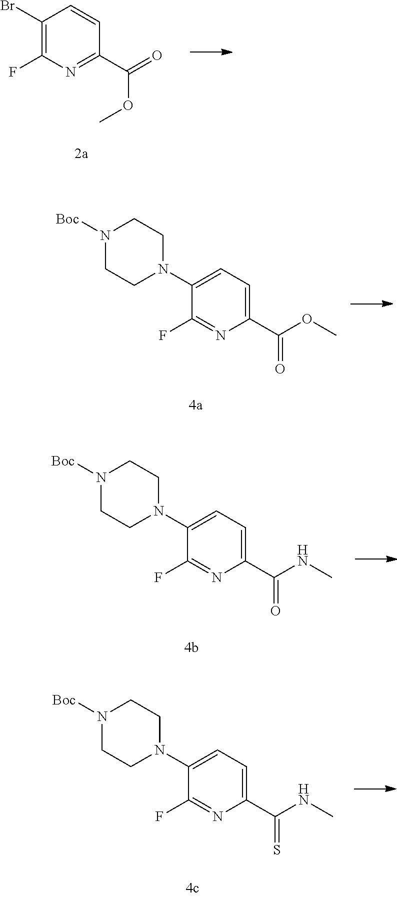 [SMILES: COC(=O)c1ccc(Br)c(F)n1 ; COC(=O)c1ccc(N2CCN(C(=O)OC(C)(C)C)CC2)c(F)n1 ; CNC(=O)c1ccc(N2CCN(C(=O)OC(C)(C)C)CC2)c(F)n1 ; CNC(=S)c1ccc(N2CCN(C(=O)OC(C)(C)C)CC2)c(F)n1]

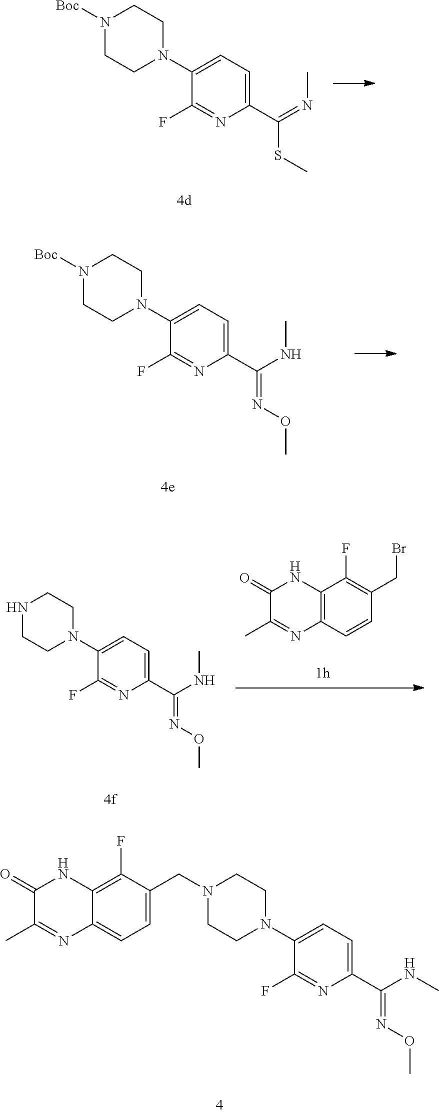 [SMILES: C/N=C(/SC)c1ccc(N2CCN(C(=O)OC(C)(C)C)CC2)c(F)n1 ; CN/C(=N\OC)c1ccc(N2CCN(C(=O)OC(C)(C)C)CC2)c(F)n1 ; CN/C(=N\OC)c1ccc(N2CCNCC2)c(F)n1 ; Cc1nc2ccc(CBr)c(F)c2[nH]c1=O ; CN/C(=N\OC)c1ccc(N2CCN(Cc3ccc4nc(C)c(=O)[nH]c4c3F)CC2)c(F)n1]

### Step 1: Synthesis of Intermediate 4a

Toluene (5 mL) was added to a reaction flask containing compound 2a (500 mg, 2.14 mmol) and N-Boc-piperazine (397.93 mg, 2.14 mmol). RuPhos (199.40 mg, 427.31 μmol), cesium carbonate (1.39 g, 4.27 mmol), and Pd₂(dba)₃ (195.65 mg, 213.65 μmol) were then sequentially added thereto. Under a nitrogen atmosphere, the reaction mixture was heated to 100° C. and stirred for 16 hours. The reaction mixture was filtered directly, and the filtrate was concentrated under reduced pressure to obtain a crude product. The crude product was purified by silica gel column chromatography (gradient elution: PE:EA=100:0 to 80:20) to obtain intermediate 4a. MS m/z: 340.1 [M+H]⁺. ¹H NMR (400 MHz, CDCl₃) δ ppm 7.98-7.98 (m, 1H) 7.91-8.06 (m, 1H) 3.98 (s, 3H) 3.57-3.68 (m, 4H) 3.16-3.31 (m, 4H) 1.51 (s, 9H).

### Step 2: Synthesis of Intermediate 4b

Compound 4a (200 mg, 589.34 μmol) was dissolved in ethanol (8 mL), and a solution of methylamine in ethanol (1.19 g, 17.68 mmol) was added. The reaction mixture was stirred at 25° C. for 16 hours. The reaction mixture was concentrated to obtain intermediate 4b, which was used directly in the next reaction without purification. MS m/z: 339.1 [M+H]⁺. 1H NMR (400 MHz, CDCl₃) δ ppm 1.42 (s, 9H) 2.93 (d, J=5.13 Hz, 3H) 3.03-3.16 (m, 4H) 3.44-3.63 (m, 4H) 7.24 (dd, J=10.01, 8.13 Hz, 1H) 7.44 (br d, J=4.13 Hz, 1H) 7.93 (dd, J=8.00, 1.00 Hz, 1H).

### Step 3: Synthesis of Intermediate 4c

Intermediate 4b (50 mg, 147.76 μmol) was dissolved in toluene (2.5 mL), and Lawesson's reagent (71.72 mg, 177.32 μmol) was added. The reaction mixture was stirred at 120° C. for 16 hours. The reaction mixture was extracted with water (1 mL) and ethyl acetate (3 mL). The organic phase was dried over anhydrous sodium sulfate. The reaction mixture was filtered and concentrated under reduced pressure, and the residue was purified by prep-TLC (PE:EA=5:1) to obtain intermediate 4c. MS m/z: 355.1 [M+H]⁺.

### Step 4: Synthesis of Intermediate 4d

Intermediate 4c (10 mg, 28.21 μmol) was dissolved in DMF (0.5 mL), and sodium hydride (2.82 mg, 70.53 μmol, purity of 60%) was added. The mixture was stirred at 0° C. for 0.5 hours. Methyl iodide (8.01 mg, 56.43 μmol, 3.51 μL) was then added thereto, and the mixture was stirred at 25° C. for 2 hours. The reaction was quenched by adding two drops of ammonium chloride solution. The reaction mixture was extracted with ethyl acetate (5 mL), and the organic phase was washed with saturated brine (2 mL), then dried over anhydrous sodium sulfate, filtered, and concentrated to obtain intermediate 4d, which was used directly in the next step without purification. MS m/z: 369.2 [M+H]⁺.

### Step 5: Synthesis of Intermediate 4e

Intermediate 4d (5 mg, 13.57 μmol) was dissolved in EtOH (0.5 mL). Triethylamine (6.87 mg, 67.85 μmol, 9.44 μL) and methoxyamine hydrochloride (3.40 mg, 40.71 μmol) were added, and the reaction mixture was stirred at 60° C. for 1.5 hours. The reaction mixture was extracted with ethyl acetate (5 mL). The organic phase was washed with water (2 mL), dried over anhydrous sodium sulfate, then filtered and concentrated. The resulting product was purified by prep-TLC (PE:EA=1:1), and the reaction mixture was filtered and concentrated to obtain intermediate 4e. MS m/z: 368.1 [M+H]⁺. ¹H NMR (400 MHz, CDCl₃) δ ppm 7.48-7.56 (m, 1H) 7.15-7.18 (m, 1H) 3.79 (s, 3H) 3.51-3.56 (m, 4H) 3.00-3.05 (m, 4H) 2.92 (s, 3H) 1.42 (s, 9H).

### Step 6: Synthesis of Hydrochloride of Intermediate 4f

Intermediate 4e (10 mg, 27.22 μmol) was dissolved in MeOH (0.5 mL). HCl/dioxane (4 M, 36.74 μL) was added, and the reaction mixture was stirred at 25° C. for 1.5 hours. LCMS analysis indicated that most of the starting material remained. An additional 0.2 mL of HCl/dioxane (4 M, 36.74 μL) was added, and the mixture was stirred for another 1.5 hours. The reaction mixture was then concentrated to obtain the hydrochloride of intermediate 4f, which was used directly in the next step without purification.

### Step 7: Synthesis of Trifluoroacetate of Compound 4

Intermediate 4f (10 mg, hydrochloride) was dissolved in DMF (1 mL). Triethylamine (6.66 mg, 65.84 μmol, 9.16 μL) was added, and the reaction mixture was stirred at 25° C. for 0.5 hours. Intermediate 1h (8.92 mg, 32.92 μmol) and potassium iodide (2.73 mg, 16.46 μmol) were then added. The mixture was stirred at 50° C. for 2 hours. The reaction mixture was filtered to obtain a crude product. The crude product was purified by preparative HPLC (column: Welch Xtimate C18, 100×40 mm×3 um; mobile phase: [water (trifluoroacetic acid)-acetonitrile]; acetonitrile %: 0%-30% over 8 minutes) to obtain the trifluoroacetate of compound 4. MS m/z: 458.2 [M+H]⁺. ¹H NMR (400 MHz, CD₃OD) δ ppm 2.56 (s, 3H) 3.03 (s, 3H) 3.59 (br s, 8H) 3.83-3.94 (m, 3H) 4.64 (s, 2H) 7.46-7.52 (m, 1H) 7.60-7.76 (m, 3H).

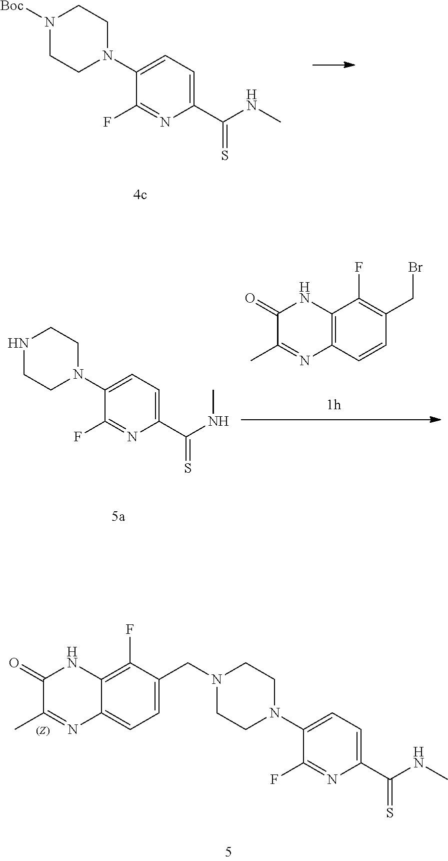 [SMILES: CNC(=S)c1ccc(N2CCN(C(=O)OC(C)(C)C)CC2)c(F)n1 ; CNC(=S)c1ccc(N2CCNCC2)c(F)n1 ; Cc1nc2ccc(CBr)c(F)c2[nH]c1=O ; CNC(=S)c1ccc(N2CCN(Cc3ccc4nc(C)c(=O)[nH]c4c3F)CC2)c(F)n1]

### Step 1: Synthesis of Hydrochloride of Intermediate 5a

Intermediate 4c (19.4 mg, 54.73 μmol) was dissolved in MeOH (1 mL). HCl/dioxane (4 M, 73.89 μL) was added, and the reaction mixture was stirred at 25° C. for 16 hours. The reaction mixture was concentrated to obtain the hydrochloride of intermediate 5a, which was used directly in the next step without purification. MS m/z: 255.0 [M+H]⁺.

### Step 2: Synthesis of Trifluoroacetate of Compound 5

Intermediate 5a (15 mg, hydrochloride) was dissolved in DMF (1 mL). Triethylamine (10.44 mg, 103.17 μmol, 14.36 μL) was added, and the reaction mixture was stirred at 25° C. for 0.5 hours. Methyl iodide (4.28 mg, 25.79 μmol) and intermediate 1h (13.98 mg, 51.58 μmol) were then added, and the mixture was stirred at 50° C. for 2 hours. The reaction mixture was filtered to obtain a crude product. The crude product was purified by preparative HPLC (column: Welch Xtimate C18, 100×40 mm×3 um; mobile phase: [water (trifluoroacetic acid)-acetonitrile]; gradient: 3%-33% acetonitrile over 8 minutes) to obtain the trifluoroacetate of compound 5. MS m/z: 445.1 [M+H]⁺. ¹H NMR (400 MHz, CD₃OD) δ ppm 2.56 (s, 3H) 3.27-3.29 (m, 3H) 3.54 (br d, J=3.76 Hz, 8H) 4.58 (s, 2H) 7.47 (t, J=7.91 Hz, 1H) 7.59 (dd, J=10.16, 8.41 Hz, 1H) 7.70 (d, J=8.28 Hz, 1H) 8.47 (d, J=8.03 Hz, 1H) 10.18-10.34 (m, 1H).

### Example 6

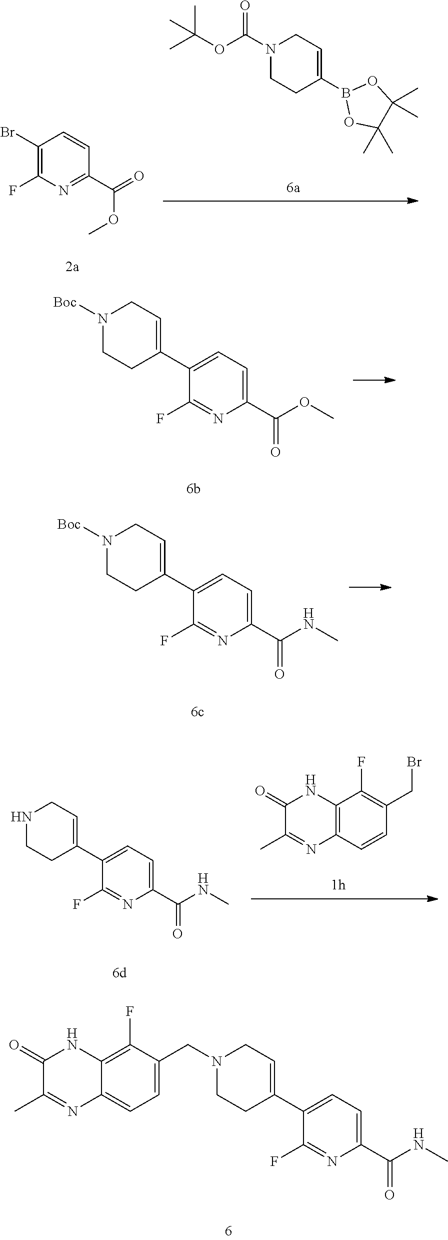

### Step 1: Synthesis of Intermediate 6b

Dioxane (1 mL), water (0.2 mL), intermediate 2a (30 mg, 128.19 μmol), and compound 6a (43.60 mg, 141.01 μmol) were added to a 10 mL vial and stirred. After nitrogen purging, potassium phosphate (54.42 mg, 256.39 μmol) and [1,1′-bis(diphenylphosphino) ferrocene]dichloropalladium (II) (9.38 mg, 12.82 μmol) were added. The reaction mixture was heated to 80° C. and reacted for 2 hours. The reaction mixture was filtered, and the filtrate was concentrated under reduced pressure to obtain a crude product. The crude product was purified by preparative TLC (eluent: PE:EA=3:1) to obtain intermediate 6b. MS m/z: 337.1 [M+H]⁺.

### Step 2: Synthesis of Intermediate 6c

Intermediate 6b (37 mg, 110.00 μmol) was dissolved in EtOH (1.5 mL). A solution of methylamine in ethanol (222.81 mg, 3.30 mmol) was added, and the reaction mixture was stirred at 25° C. for 6 hours. The reaction mixture was directly concentrated to obtain intermediate 6c, which was used directly in the next step without purification. MS m/z: 336.0 [M+H]⁺.

### Step 3: Synthesis of Hydrochloride of Intermediate 6d

Intermediate 6c (17 mg, 50.69 μmol) was dissolved in MeOH (0.5 mL). HCl/dioxane (4 M, 68.43 μL) was added, and the reaction mixture was stirred at 25° C. for 2 hours. The reaction mixture was directly concentrated to obtain the hydrochloride of intermediate 6d, which was used directly in the next step without purification. MS m/z: 236.1 [M+H]⁺.

Step 4: Synthesis of trifluoroacetate of compound 6

Intermediate 6d (10 mg, 36.80 μmol, hydrochloride) was dissolved in DMF (1 mL). Triethylamine (7.45 mg, 73.61 μmol, 10.24 μL) was added, and the reaction mixture was stirred at 25° C. for 0.5 hours. Intermediate 1h (9.98 mg, 36.80 μmol) and potassium iodide (3.05 mg, 18.40 μmol) were then added. The mixture was stirred at 50° C. for 2 hours. The reaction mixture was filtered to obtain a crude product. The crude product was separated by preparative HPLC (column: Welch Xtimate C18, 100× 40 mm×3 um; mobile phase: [water (trifluoroacetic acid)-acetonitrile]; gradient: 0%-30% acetonitrile over 8 minutes) to obtain the trifluoroacetate of compound 6. MS m/z: 426.2 [M+H]⁺. ¹H NMR (400 MHz, CDCl₃) δ ppm 8.12 (dd, J=7.78, 1.51 Hz, 1H) 7.83-7.91 (m, 1H) 7.70-7.79 (m, 1H) 7.57-7.68 (m, 2H) 6.13 (br s, 1H) 4.53 (s, 2H) 3.74-4.00 (m, 2H) 3.36-3.66 (m, 2H) 3.04-3.09 (m, 3H) 2.91-2.99 (m, 1H) 2.65 (s, 3H).

### Example 7

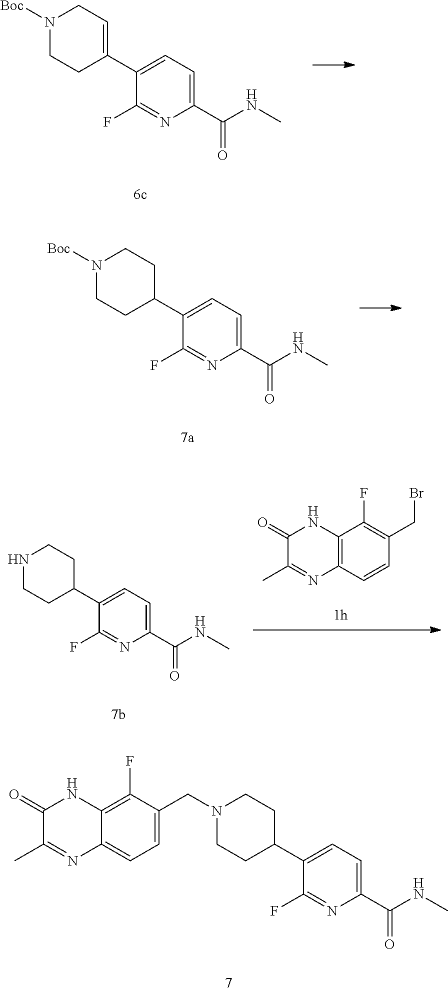 [SMILES: CNC(=O)c1ccc(C2=CCN(C(=O)OC(C)(C)C)CC2)c(F)n1 ; CNC(=O)c1ccc(C2CCN(C(=O)OC(C)(C)C)CC2)c(F)n1 ; CNC(=O)c1ccc(C2CCNCC2)c(F)n1 ; Cc1nc2ccc(CBr)c(F)c2[nH]c1=O ; CNC(=O)c1ccc(C2CCN(Cc3ccc4nc(C)c(=O)[nH]c4c3F)CC2)c(F)n1]

### Step 1: Synthesis of Intermediate 7a

Intermediate 6c (10 mg, 29.82 μmol) was added to a mixture of MeOH (3 mL) and ethyl acetate (1 mL). Palladium on carbon (2 mg, 29.82 μmol, 10%) was also added thereto. The reaction mixture was purged with argon three times and then with hydrogen three times. The reaction mixture was stirred under a hydrogen atmosphere at 16 psi and 30° C. for 16 hours. The reaction mixture was filtered through diatomite, and the organic phase was concentrated under reduced pressure to obtain intermediate 7a, which was used directly in the next step. MS m/z: 360.2 [M+23]⁺.

### Step 2: Synthesis of Hydrochloride of Intermediate 7b

Intermediate 7a (10 mg, 29.64 μmol) was dissolved in MeOH (0.5 mL). HCl/dioxane (4 M, 37.05 μL) was added thereto. The reaction mixture was stirred at 25° C. for 2 hours. The reaction mixture was directly concentrated under reduced pressure to obtain the hydrochloride of intermediate 7b, which was used directly in the next step. MS m/z: 238.0 [M+1]⁺.

### Step 3: Synthesis of Trifluoroacetate of Compound 7

Triethylamine (6.94 mg, 68.56 μmol) was added to a solution of intermediate 7b (9.38 mg, 34.28 μmol, crude hydrochloride) in DMF (1 mL). The reaction mixture was stirred at 20° C. for 0.5 hours. Intermediate 1h (9.29 mg, 34.28 μmol) and potassium iodide (569.03 μg, 3.43 μmol) were then added thereto. The reaction mixture was stirred at 50° C. for 2 hours. The reaction mixture was concentrated under reduced pressure to obtain a crude product. The crude product was purified by preparative HPLC (column: Welch Xtimate C18, 100×40 mm×3 μm; mobile phase: [water (trifluoroacetic acid)-acetonitrile]; gradient: 0%-30% acetonitrile over 8 minutes) to obtain the trifluoroacetate of compound 7. MS m/z: 428.3 [M+1]⁺. ¹H NMR (400 MHz, CD₃OD) δ ppm 8.75 (s, 1H) 8.01 (s, 2H) 7.71 (d, J=8.28 Hz, 1H) 7.47 (t, J=7.65 Hz, 1H) 4.58 (s, 2H) 3.72 (br s, 2H) 3.18-3.30 (m, 3H) 2.95 (d, J=5.02 Hz, 3H) 2.56 (s, 3H) 2.15-2.26 (m, 2H) 2.07 (d, J=12.80 Hz, 2H).

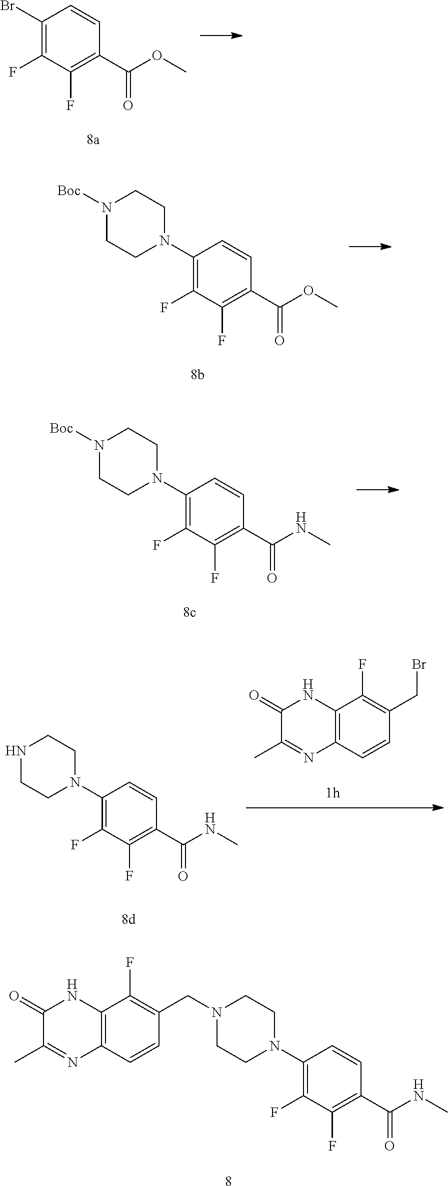

### Step 1: Synthesis of Intermediate 8b

Compound 8a (245.05 mg, 976.20 μmol) and N-Boc-piperazine (200 mg, 1.07 mmol) were added to toluene (5 mL). Pd₂(dba)₃ (8.94 mg, 9.76 μmol), RuPhos (9.11 mg, 19.52 μmol), and Cs₂CO₃ (636.13 mg, 1.95 mmol) were added thereto. The reaction mixture was purged with nitrogen three times and stirred at 100° C. for 16 hours. The reaction mixture was filtered through diatomite, and the organic phase was concentrated under reduced pressure to obtain a crude product. A mixture of ethyl acetate and n-heptane (ethyl acetate/n-heptane=5/1, 10 mL) was added to the crude product, and the resulting mixture was stirred at 25° C. for 0.5 hours and subjected to suction filtration under reduced pressure, and the filter cake was concentrated to obtain intermediate 8b. MS m/z: 301.2 [M−56+1]⁺. ¹H NMR (400 MHz, DMSO-d₆) δ ppm 7.61 (t, J=8.19 Hz, 1H) 6.93 (t, J=8.38 Hz, 1H) 3.83 (s, 3H) 3.50-3.46 (m, 4H) 3.13-3.23 (m, 4H) 1.43 (s, 9H).

### Step 2: Synthesis of Intermediate 8c

Compound 2 (100 mg, 280.61 μmol) and a solution of methylamine in ethanol (87.15 mg, 1.29 mmol) were added to EtOH (2 mL). The mixture was stirred at 25° C. for 16 hours, and then the reaction temperature was increased to 50° C. The mixture was stirred at 50° C. for 16 hours. The reaction mixture was directly concentrated under reduced pressure to obtain a crude product. The crude product was purified by preparative TLC (PE/EA=2/1, R_{f}=0.1) to obtain intermediate 8c. MS m/z: 378.2 [M+23]⁺.

### Step 3: Synthesis of Hydrochloride of Intermediate 8d

Intermediate 8c (50 mg, 140.69 μmol) was dissolved in MeOH (0.5 mL). HCl/dioxane (4 M, 37.05 μL) was added thereto. The reaction mixture was stirred at 25° C. for 2 hours, then directly concentrated under reduced pressure to obtain the hydrochloride of intermediate 8d, which was used directly in the next step. MS m/z: 256.0 [M+1]⁺.

### Step 4: Synthesis of Trifluoroacetate of Compound 8

Triethylamine (10.41 mg, 102.84 μmol) was added to a solution of intermediate 8d (15 mg, 51.42 μmol, crude hydrochloride) in DMF (1 mL). The reaction mixture was stirred at 20° C. for 0.5 hours. Intermediate 1h (13.94 mg, 51.42 μmol) and potassium iodide (853.55 μg, 5.14 μmol) were then added thereto. The reaction mixture was stirred at 50° C. for 2 hours. The reaction mixture was concentrated using an oil pump under reduced pressure to obtain a crude product. The crude product was purified by preparative HPLC (column: Welch Xtimate C18, 100× 40 mm×3 um; mobile phase: [water (trifluoroacetic acid)-acetonitrile]; gradient: 0%-30% acetonitrile over 8 minutes) to obtain the trifluoroacetate of compound 8. MS m/z: 468.1 [M+23]⁺. ¹H NMR (400 MHz, CD₃OD) δ ppm 8.15 (s, 1H) 7.70 (d, J=8.28 Hz, 1H) 7.39-7.57 (m, 2H) 6.89-7.03 (m, 1H) 4.55 (s, 2H) 3.50 (s, 8H) 2.89-2.98 (m, 3H) 2.56 (s, 3H).

### Example 9

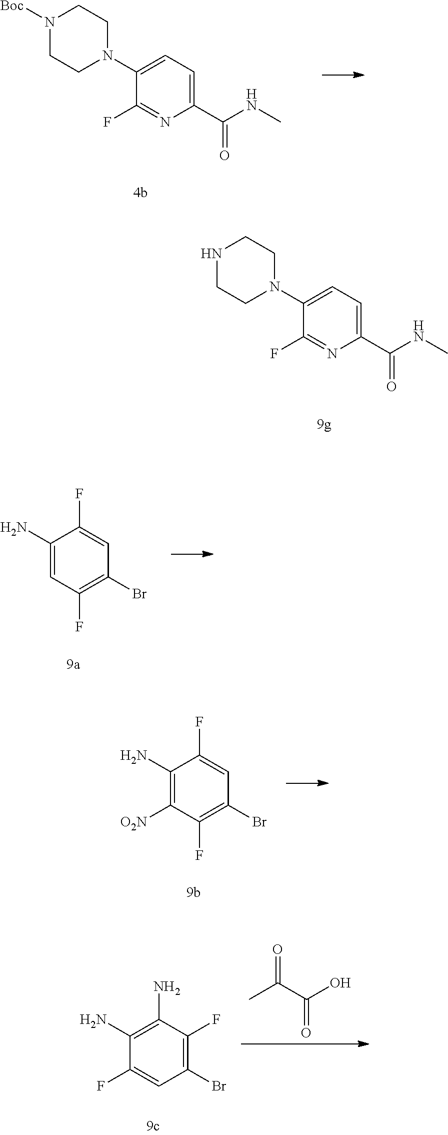

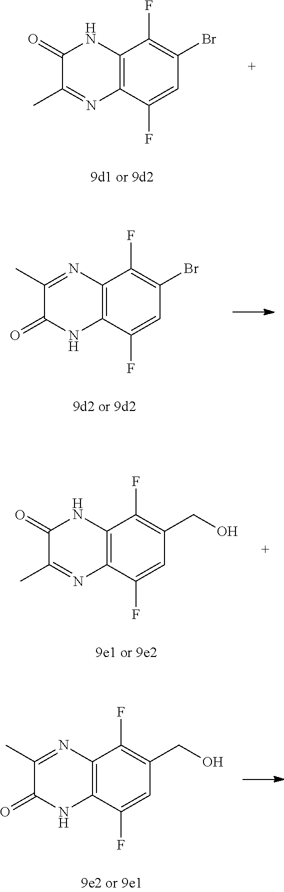 [SMILES: Cc1nc2c(F)cc(Br)c(F)c2[nH]c1=O ; Cc1nc2c(F)c(Br)cc(F)c2[nH]c1=O ; Cc1nc2c(F)cc(CO)c(F)c2[nH]c1=O ; Cc1nc2c(F)c(CO)cc(F)c2[nH]c1=O]

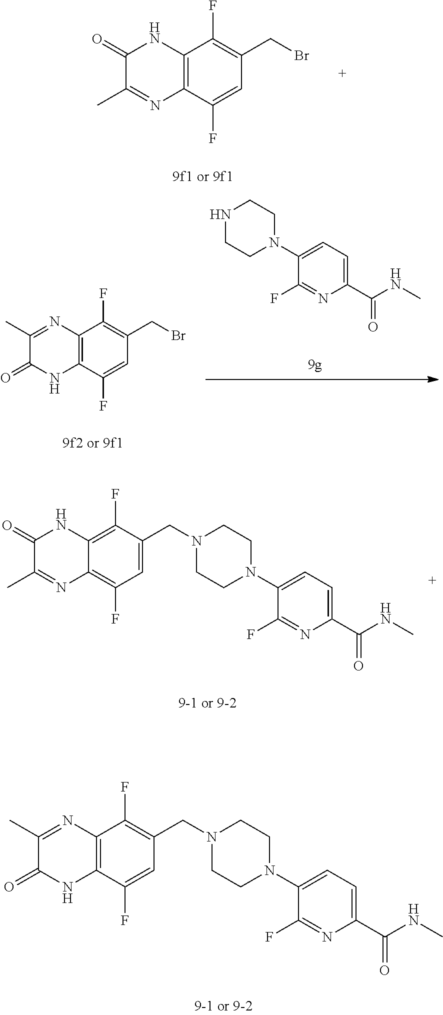

### Step 1: Synthesis of Hydrochloride of Intermediate 9g

Intermediate 4b (50 mg, 147.76 μmol) was dissolved in methanol (0.2 mL). HCl/dioxane (4 M, 199.48 μL) was added, and the reaction mixture was stirred at 25° C. for 2 hours. The reaction mixture was concentrated under reduced pressure without further purification to obtain the hydrochloride of intermediate 9g. MS m/z: 238.9 [M+1]⁺.

### Step 2: Synthesis of Intermediate 9b

At 0° C., compound 9a (1 g, 4.81 mmol) was added to trifluoroacetic acid (10 mL). Potassium nitrate (760 mg, 7.52 mmol) was slowly added in batches. The reaction mixture was then stirred at 20° C. for 16 hours. The reaction mixture was poured into 90 mL of saturated sodium bicarbonate solution, and then extracted with ethyl acetate (100 mL×3). The organic phases was washed with saturated sodium chloride solution (100 mL×2), filtered, and concentrated. The crude product was purified by silica gel column chromatography (EA:PE=0%-10%) to obtain intermediate 9b. ¹H NMR (400 MHz, DMSO-d₆) δ ppm 7.68-7.92 (m, 1H), 7.08 (br s, 2H).

### Step 3: Synthesis of Intermediate 9c

Intermediate 9b (100 mg, 395.26 μmol) was dissolved in EtOH (1 mL) and cooled to 0° C. Acetic acid (237.36 mg, 3.95 mmol, 226.27 μL) was first added, then zinc (240 mg, 3.67 mmol) was added in batches. The reaction was carried out for 6 hours. The reaction mixture was filtered and concentrated to obtain a crude product. The crude product was purified by preparative TLC (PE:EA=3:1) to obtain intermediate 9c. MS m/z: 224.9, 226.9 [M+1]⁺, [M+3]⁺. ¹H NMR (400 MHz, CDCl₃) δ ppm 6.69-6.80 (m, 1H), 3.52 (s, 4H).

### Step 4: Synthesis of Mixture of 9d1 and 9d2

Intermediate 9c (25 mg, 112.10 μmol) was added to EtOH (0.32 mL), then pyruvic acid (11.85 mg, 134.52 μmol) was added. The reaction mixture was reacted at 100° C. for 2 hours, then slowly cooled to 25° C. to crystallize over 14 hours. The reaction mixture was filtered, and the solid was washed once with 0.5 mL of ethanol. A mixture of intermediates 9d1 and 9d2 was obtained without further purification. MS m/z: 274.9 [M+1]⁺.

### Step 5: Synthesis of Mixture of 9e1 and 9e2

The mixture of intermediates 9d1 and 9d2 (95 mg) and XPHOS-PD-G2 (13.59 mg, 17.27 μmol) was dissolved in dioxane (2 mL). 1-(Tributylstannyl) methanol (133.08 mg, 414.47 μmol) was added, and the mixture was purged with nitrogen and reacted at 80° C. for 16 hours. The reaction mixture was purified by preparative TLC (PE:EA=1:1) to obtain a mixture of intermediates 9e1 and 9e2. MS m/z: 227.0 [M+1]⁺.

### Step 6: Synthesis of Mixture of 9f1 and 9f2

Under a nitrogen atmosphere and at 0° C., tributylphosphine (80.50 mg, 397.91 μmol, 98.18 μL) was slowly added dropwise to a solution of intermediates 9f1 and 9f2 (45 mg) and 1,2-dibromotetrachloroethane (142.54 mg, 437.71 μmol) in dichloromethane (1.2 mL). The reaction mixture was reacted at 25° C. for 2 hours. The reaction mixture was purified by preparative TLC (dichloromethane:methanol=15:1) to obtain a mixture of intermediates 9f1 and 9f2.

### Step 7: Synthesis of Mixture of Trifluoroacetates of Compounds 9-1 and 9-2

The mixture of intermediates 9f1 and 9f2 (43 mg) was added to DMF (2 mL). Triethylamine (29.92 mg, 295.65 μmol) was added, and the reaction mixture was stirred at 25° C. for 15 minutes. Potassium iodide (11.04 mg, 66.52 μmol) and intermediate 9g (46 mg, 147.82 μmol) were then added thereto. The mixture was stirred at 50° C. for 2 hours. The reaction mixture was separated by preparative HPLC (column: Welch Xtimate C18, 100×40 mm×3 um; mobile phase: [water (trifluoroacetic acid)-acetonitrile]; acetonitrile %: 0%-30% acetonitrile over 8 minutes). Thus a mixture of trifluoroacetates of compounds 9-1 and 9-2 was obtained. MS m/z: 447.1 [M+1]⁺.

### Example 10

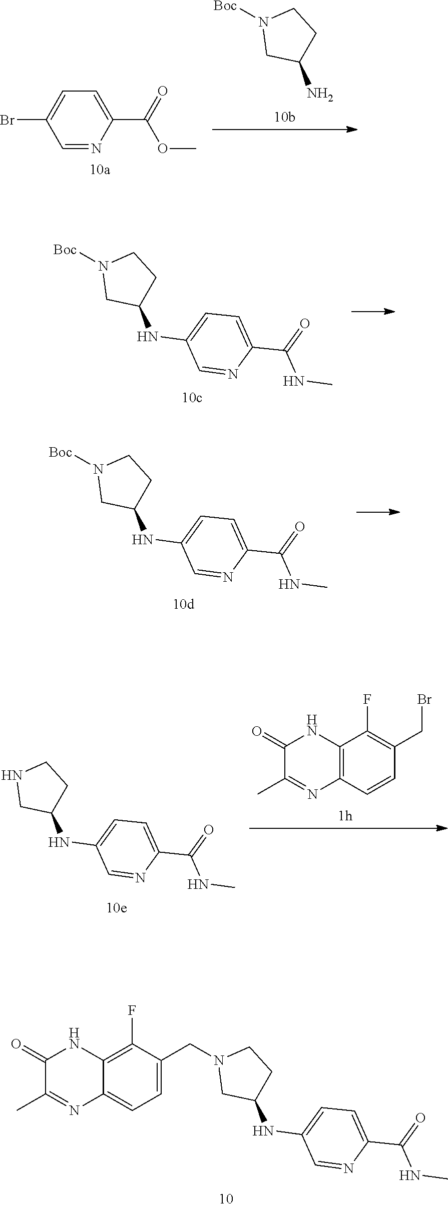

### Step 1: Synthesis of Intermediate 10c

Under a nitrogen atmosphere, 10a (50 mg, 231.45 μmol), compound 10b (51.73 mg, 277.74 μmol), Cs₂CO₃ (150.82 mg, 462.89 μmol), RuPhos (21.60 mg, 46.29 μmol), and Pd₂(dba)₃ (21.19 mg, 23.14 μmol) were added to toluene (1 mL). The mixture was heated to 100° C. and stirred for 16 hours. The reaction mixture was filtered through diatomite and washed with DCM (5 mL). The filtrate was concentrated under reduced pressure to obtain a crude product. The crude product was purified by preparative TLC (PE:EA=0:1) to obtain intermediate 10c. MS m/z: 322.1 [M+1]⁺, ¹H NMR (400 MHz, DMSO-d₆) δ ppm 8.06 (d, J=2.76 Hz, 1H), 7.82 (d, J=8.53 Hz, 1H), 6.93-7.02 (m, 2H), 4.04-4.13 (m, 1H), 3.74-3.80 (m, 3H), 3.53-3.61 (m, 1H), 3.42-3.47 (m, 1H), 3.32-3.35 (m, 1H), 3.04-3.21 (m, 1H), 2.09-2.20 (m, 1H), 1.74-1.86 (m, 1H), 1.40 (br d, J=6.53 Hz, 9H).

### Step 2: Synthesis of Intermediate 10d

Intermediate 10c (60 mg, 186.70 μmol) was added to EtOH (1 mL), then a solution of methylamine in ethanol (1.23 g, 11.88 mmol) was added thereto. The reaction mixture was stirred at 25° C. for 16 hours. The reaction mixture was concentrated under reduced pressure to obtain intermediate 10d, which was used directly in the next step. MS m/z: 321.2 [M+1]⁺.

### Step 3: Synthesis of Hydrochloride of Intermediate 10e

Intermediate 10d (25 mg, 78.03 μmol) was added to MeOH (0.5 mL), then HCl/dioxane (4 M, 105.34 μL) was added. The reaction mixture was stirred at 25° C. for 2 hours. The reaction mixture was concentrated under reduced pressure to obtain the hydrochloride of intermediate 10e, which was used directly in the next step. MS m/z: 221.1 [M+1]⁺, ¹H NMR (400 MHz, DMSO-d₆) δ 9.35 (br s, 3H), 8.01 (d, J=2.51 Hz, 1H), 7.92 (dd, J=8.66, 4.39 Hz, 1H), 7.21 (dd, J=8.41, 2.38 Hz, 1H), 4.20-4.30 (m, 1H), 3.42-3.51 (m, 1H), 3.24-3.37 (m, 2H), 3.04-3.13 (m, 1H), 2.79 (d, J=4.52 Hz, 3H), 2.19-2.31 (m, 1H), 1.86-1.98 (m, 1H).

### Step 4: Synthesis of Compound 10

Triethylamine (15.77 mg, 155.80 μmol, 21.69 μL) was added to a solution of intermediate 10e (20 mg, hydrochloride) in DMF (1 mL). The reaction mixture was stirred at 20° C. for 0.5 hours. Compound 1h (21.12 mg, 77.90 μmol) and potassium iodide (1.29 mg, 7.79 μmol) were then added thereto. The reaction mixture was heated to 50° C. and stirred for 2 hours. The reaction mixture was washed with water (2 mL), extracted three times with a mixture of DCM and MeOH (DCM:MeOH=10:1, 3 mL). The organic phases were combined, dried over anhydrous sodium sulfate, and filtered. The filtrate was concentrated under reduced pressure to obtain a crude product. The crude product was purified by preparative TLC (DCM:MeOH=10:1) to obtain compound 10. MS m/z: 411.1 [M+1]⁺, ¹H NMR (400 MHz, DMSO-d₆) δ ppm 12.44 (s, 1H), 8.27 (br d, J=4.77 Hz, 1H), 7.91 (d, J=2.51 Hz, 1H), 7.72 (d, J=8.53 Hz, 1H), 7.50 (d, J=7.78 Hz, 1H), 7.24-7.32 (m, 1H), 6.94 (dd, J=8.16, 2.64 Hz, 1H), 6.65 (d, J=6.53 Hz, 1H), 3.94-4.02 (m, 1H), 3.75 (br s, 2H), 3.37-3.42 (m, 1H), 2.86 (br t, J=8.66 Hz, 1H), 2.75 (d, J=4.77 Hz, 3H), 2.65-2.71 (m, 1H), 2.43-2.47 (m, 1H), 2.41 (s, 3H), 2.20-2.28 (m, 1H), 1.54-1.65 (m, 1H).

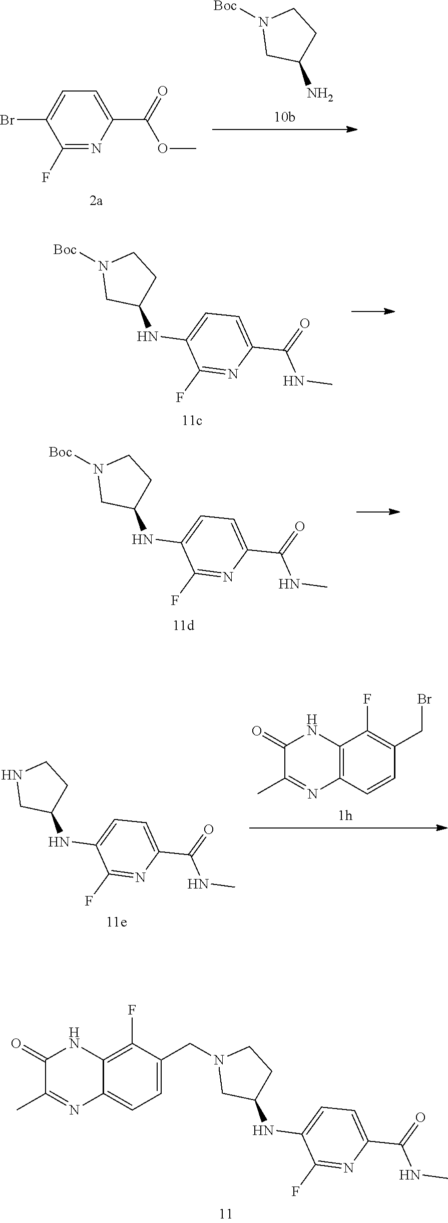

### Step 1: Synthesis of Intermediate 11c

Under a nitrogen atmosphere, 2a (50 mg, 231.45 μmol), compound 10b (51.73 mg, 277.74 μmol), Cs₂CO₃ (150.82 mg, 462.89 μmol), RuPhos (21.60 mg, 46.29 μmol), and Pd₂(dba)₃ (21.19 mg, 23.14 μmol) were added to toluene (1 mL). The reaction mixture was heated to 100° C. and stirred for 16 hours. The reaction mixture was filtered through diatomite, washed with DCM (5 mL). The filtrate was concentrated under reduced pressure to obtain a crude product. The crude product was purified by preparative TLC (PE:EA=0:1) to obtain intermediate 11c. MS m/z: 340.1 [M+1]⁺, ¹H NMR (400 MHz, DMSO-d₆) δ ppm 7.84 (d, J=8.00 Hz, 1H), 7.24 (br t, J=9.26 Hz, 1H), 6.88 (br d, J=6.38 Hz, 1H), 4.07-4.18 (m, 1H), 3.79 (s, 3H), 3.56-3.64 (m, 1H), 3.38-3.49 (m, 2H), 3.20 (br dd, J=11.01, 4.75 Hz, 1H), 2.11-2.22 (m, 1H), 1.86-1.96 (m, 1H), 1.40 (br d, J=5.38 Hz, 9H).

### Step 2: Synthesis of Intermediate 11d

Intermediate 11c (60 mg, 186.70 μmol) was added to EtOH (1 mL), then a solution of methylamine in ethanol (1.23 g, 11.88 mmol) was added thereto. The reaction mixture was stirred at 25° C. for 16 hours. The reaction mixture was concentrated under reduced pressure to obtain intermediate 11d, which was used directly in the next step. MS m/z: 339.1 [M+1]⁺. ¹H NMR (400 MHz, DMSO-d₆) δ ppm 8.21 (br d, J=4.63 Hz, 1H), 7.75 (d, J=8.25 Hz, 1H), 7.24-7.31 (m, 1H), 6.56 (br d, J=6.50 Hz, 1H), 4.04-4.18 (m, 1H), 3.56-3.66 (m, 1H), 3.38-3.47 (m, 2H), 3.16-3.24 (m, 1H), 2.75 (d, J=4.75 Hz, 3H), 2.08-2.24 (m, 1H), 1.84-1.97 (m, 1H) 1.40 (br d, J=6.13 Hz, 9H).

### Step 3: Synthesis of Hydrochloride of Intermediate 11e

Intermediate 11d (25 mg, 78.03 μmol) was added to MeOH (0.5 mL), then HCl/dioxane (4 M, 105.34 μL) was added. The reaction mixture was stirred at 25° C. for 4 hours. The reaction mixture was concentrated under reduced pressure to obtain the hydrochloride of intermediate 11e, which was used directly in the next step. MS m/z: 239.1 [M+1]⁺.

### Step 4: Synthesis of Compound 11

Triethylamine (11.05 mg, 109.20 μmol, 15.20 μL) was added to a solution of the hydrochloride of 11e (obtained from step 3, 15 mg) in DMF (1 mL). The reaction mixture was stirred at 20° C. for 0.5 hours. Compound 1h (14.80 mg, 54.60 μmol) and potassium iodide (906.37 μg, 5.46 μmol) were thereto. The reaction mixture was stirred at 50° C. for 2 hours. The reaction mixture was washed with water (2 mL), extracted three times with a mixture of DCM and MeOH (DCM:MeOH=10:1, 3 mL). The organic phases were combined, dried over anhydrous sodium sulfate, and concentrated under reduced pressure to obtain a crude product. The crude product was purified by preparative TLC (DCM:MeOH=10:1) to obtain compound 11. MS m/z: 429.2 [M+1]⁺, ¹H NMR (400 MHz, DMSO-d₆) δ ppm 12.43 (s, 1H), 8.15 (br d, J=4.63 Hz, 1H), 7.72 (d, J=8.00 Hz, 1H), 7.50 (d, J=8.25 Hz, 1H), 7.28 (t, J=7.63 Hz, 1H), 7.16 (dd, J=10.44, 8.32 Hz, 1H), 6.41 (br d, J=5.88 Hz, 1H), 3.96-4.04 (m, 1H), 3.76 (br s, 2H), 3.29 (br s, 1H), 2.92 (br t, J=8.00 Hz, 1H), 2.74 (d, J=4.75 Hz, 3H), 2.64-2.70 (m, 1H), 2.53-2.58 (m, 1H), 2.41 (s, 3H), 2.18-2.27 (m, 1H), 1.73-1.81 (m, 1H).

### Example 12

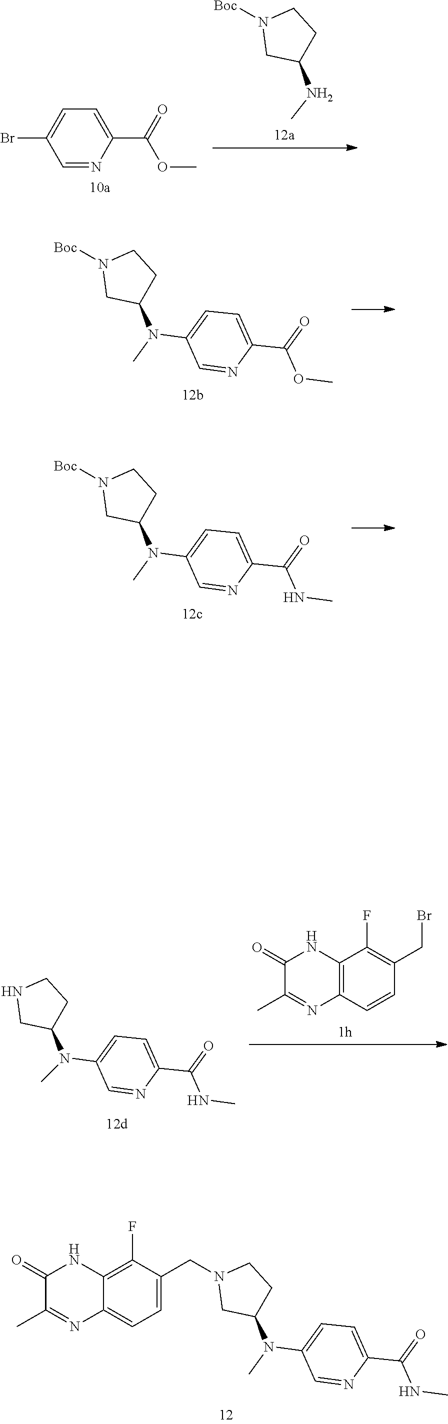

### Step 1: Synthesis of Intermediate 12b

Under a nitrogen atmosphere, 10a (50 mg, 231.45 μmol), compound 12a (51.73 mg, 277.74 μmol), Cs₂CO₃ (150.82 mg, 462.89 μmol), RuPhos (21.60 mg, 46.29 μmol), and Pd₂(dba)₃ (21.19 mg, 23.14 μmol) were added to toluene (1 mL). The reaction mixture was heated to 100° C. and stirred for 16 hours. The reaction mixture was filtered through diatomite, washed with DCM (5 mL). The filtrate was concentrated under reduced pressure to obtain a crude product. The crude product was purified by preparative TLC (PE:EA=0:1) to obtain intermediate 12b. MS m/z: 336.2 [M+1]⁺.

### Step 2: Synthesis of Intermediate 12c

Intermediate 12b (45 mg, 134.17 μmol) was added to EtOH (1 mL), then a solution of methylamine in ethanol (694.48 mg, 6.71 mmol) was added thereto. The reaction mixture was stirred at 25° C. for 16 hours. The reaction mixture was concentrated under reduced pressure to obtain compound 12c. MS m/z: 335.2 [M+1]⁺. ¹H NMR (400 MHz, DMSO-d₆) δ ppm 8.34 (br d, J=4.75 Hz, 1H), 8.18 (d, J=2.88 Hz, 1H), 7.81 (d, J=8.88 Hz, 1H), 7.31 (dd, J=8.88, 3.00 Hz, 1H), 4.61-4.70 (m, 1H), 3.53 (br t, J=8.88 Hz, 1H), 3.41-3.46 (m, 1H), 3.30-3.33 (m, 3H), 3.21-3.25 (m, 1H), 2.78 (d, J=4.88 Hz, 3H), 1.95-2.11 (m, 3H), 1.41 (s, 9H).

### Step 3: Synthesis of Hydrochloride of Intermediate 12d

Intermediate 12c (44 mg, 131.57 μmol) was added to MeOH (0.5 mL), and HCl/dioxane (4 M, 105.34 μL) was then added. The reaction mixture was stirred at 25° C. for 2 hours. The reaction mixture was concentrated under reduced pressure to obtain the hydrochloride of intermediate 12d, which was used directly in the next step. MS m/z: 235.1 [M+1]⁺.

### Step 4: Synthesis of Compound 12

Triethylamine (14.95 mg, 147.73 μmol, 20.56 μL) was added to a solution of the hydrochloride of 12d (20 mg) in DMF (1 mL). The reaction mixture was stirred at 20° C. for 0.5 hours. Compound 1h (20.02 mg, 73.87 μmol) and potassium iodide (1.23 mg, 7.39 μmol) were then added thereto. The reaction mixture was stirred at 50° C. for 2 hours. The reaction mixture was washed with water (2 mL), extracted three times with a mixture of DCM and MeOH (DCM:MeOH=10:1, 3 mL). The organic phases were combined, dried over anhydrous sodium sulfate, and concentrated under reduced pressure to obtain a crude product. The crude product was purified by preparative TLC (DCM:MeOH=10:1) to obtain compound 12. MS m/z: 425.2 [M+1]⁺, ¹H NMR (400 MHz, DMSO-d₆) δ ppm 12.45 (br s, 1H), 8.31 (br d, J=5.02 Hz, 1H), 8.11 (d, J=3.01 Hz, 1H), 7.77 (d, J=8.78 Hz, 1H), 7.52 (d, J=8.28 Hz, 1H), 7.30 (t, J=7.65 Hz, 1H), 7.22 (dd, J=8.78, 3.01 Hz, 1H), 4.56-4.67 (m, 1H), 3.75 (s, 2H), 3.32 (s, 3H), 2.87 (td, J=8.34, 2.89 Hz, 1H), 2.77 (d, J=5.02 Hz, 3H), 2.73 (dd, J=10.04, 3.26 Hz, 1H), 2.62 (t, J=8.78 Hz, 1H), 2.41 (s, 3H), 2.31-2.37 (m, 1H), 2.17-2.28 (m, 1H), 1.66-1.76 (m, 1H).

### Example 14

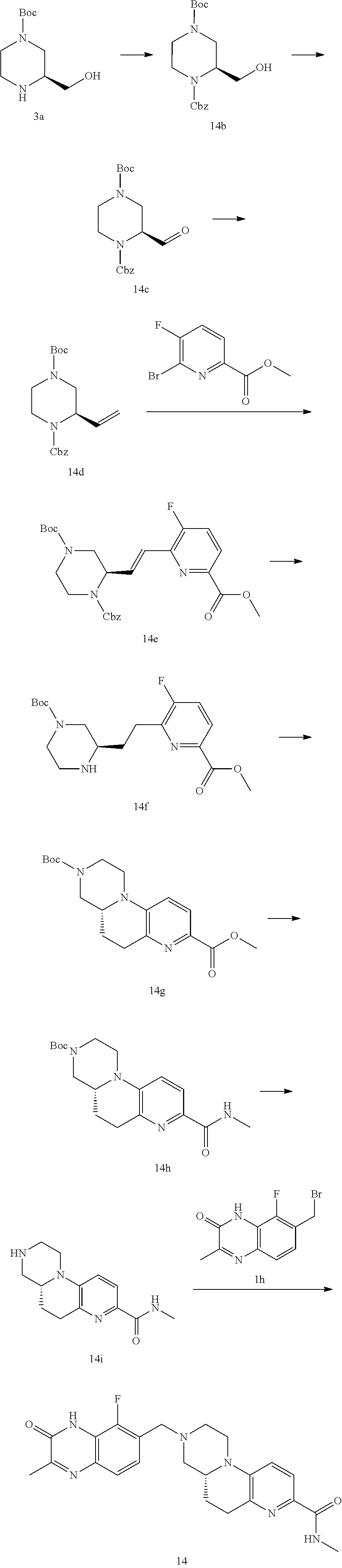

### Step 1: Synthesis of Intermediate 14b

Compound 3a (8 g, 36.99 mmol) was added to a mixture of DCM (80 mL) and H₂O (80 mL). NaHCO₃ (9.32 g, 110.97 mmol) was then added thereto. The reaction mixture was cooled to 0° C., and benzyl chloroformate (9.47 g, 55.48 mmol) was added thereto. The reaction mixture was stirred at 20° C. for 16 hours. An additional 80 mL of DCM was added to the reaction mixture. The mixture was extracted to allow phase separation. The organic phase was dried over anhydrous sodium sulfate, and filtered. The filtrate was concentrated under reduced pressure at 50° C. to obtain a crude product. The crude product was purified by column chromatography (eluent: PE/EA=5/1 to 2/1, v/v) to obtain intermediate 14b.

### Step 2: Synthesis of Intermediate 14c

Intermediate 14b (5 g, 14.27 mmol) was added to DCM (100 mL). At 0° C., Dess-Martin periodinane (9.08 g, 21.40 mmol, 6.63 mL) was added thereto in batches. The reaction mixture was stirred at 0° C. for 3 hours. Calcium hydroxide (6 g) was added to the reaction mixture, and the mixture was stirred at 0° C. for 1 hour. The mixture was filtered, and the filter cake was washed with DCM (100 mL×3). The organic phase was dried over anhydrous sodium sulfate and concentrated under reduced pressure at 50° C. to obtain a crude product. The crude product was purified by flash silica gel column chromatography (eluent: PE:EA=90:10 to 80:20, v/v) to obtain intermediate 14c.

### Step 3: Synthesis of Intermediate 14d

Under a nitrogen atmosphere, KHMDS (1 M, 3.44 mL) was added dropwise to a solution of methyltriphenylphosphonium bromide (1.23 g, 3.44 mmol) in THF (4 mL) at 20° C. The reaction mixture was stirred at 20° C. for 1 hour. The temperature was then lowered to −78° C., and a solution of intermediate 14c (0.4 g, 1.15 mmol) in THF (4 mL) was slowly added dropwise to the reaction mixture with stirring over 10 minutes. The mixture was warmed to 20° C. and stirred for 1 hour at that temperature. Methanol (72 mL) was added to the reaction mixture, then a saturated potassium sodium tartrate solution (36 mL) was added. The mixture was extracted with ethyl acetate (180 mL×3). The organic phases was dried over anhydrous sodium sulfate, and filtered. The filtrate was concentrated under reduced pressure at 45° C. to obtain a crude product. The crude product was purified by silica gel column chromatography (eluent: PE/EA=90:10 to 80:20, v/v) to obtain intermediate 14d.

### Step 4: Synthesis of Intermediate 14e

Methyl 6-bromo-5-fluoropicolinate (100 mg, 427.31 μmol), intermediate 14d (148.03 mg, 427.31 μmol), Pd(OAc)₂ (9.59 mg, 42.73 μmol), PPh₃ (22.42 mg, 85.46 μmol), and Na₂CO₃ (90.58 mg, 854.62 μmol) were added to DMF (5 mL). The reaction mixture was purged with nitrogen and then heated to 130° C. and stirred for 6 hours. 5 mL of water was added to the reaction mixture, and the mixture was extracted with ethyl acetate (10 mL) three times. The organic phases were combined, dried over anhydrous sodium sulfate, and concentrated under reduced pressure to obtain a crude product. The crude product was purified by preparative TLC (PE:EA=2:1) to obtain intermediate 14e. MS m/z: 500.2 [M+1]⁺.

### Step 5: Synthesis of Intermediate 14f

Intermediate 14e (20 mg, 40.04 μmol) was added to methanol (4 mL), then Pd/C (42.61 mg) was added to the reaction mixture. The reaction mixture was purged with argon three times, then with hydrogen three times. The reaction mixture was stirred at 30° C. under a hydrogen pressure of 30 psi for 16 hours. The reaction mixture was cooled to 20° C., and subjected to suction filtration under reduced pressure. The filtrate was concentrated under reduced pressure to obtain intermediate 14f, which was used directly in the next step. MS m/z: 368.2 [M+1]⁺.

### Step 6: Synthesis of Intermediate 14g

Potassium carbonate (11.28 mg, 81.65 μmol) was added to a solution of intermediate 14f (15 mg, 40.83 μmol) in DMF (1 mL). The reaction mixture was heated to 50° C. and stirred for 6 hours. Water (5 mL) was then added to the reaction mixture, and the mixture was extracted with EA (10 mL) three times. The organic phases were combined, dried over anhydrous sodium sulfate, and concentrated under reduced pressure to obtain a crude product. The crude product was purified by preparative TLC (PE:EA=1:1) to obtain intermediate 14g. MS m/z: 348.2 [M+1]⁺; ¹H NMR (400 MHz, CDCl₃) δ ppm 7.82 (d, J=8.63 Hz, 1H), 6.94 (d, J=8.76 Hz, 1H), 3.98-4.13 (m, 2H), 3.87 (s, 3H), 3.62-3.74 (m, 1H), 3.14 (tt, J=10.33, 3.42 Hz, 1H), 2.94-3.06 (m, 2H), 2.84-2.92 (m, 1H), 2.57-2.70 (m, 1H), 2.04 (dq, J=13.35, 4.39 Hz, 1H), 1.67-1.82 (m, 1H), 1.52-1.63 (m, 1H), 1.42 (s, 9H).

### Step 7: Synthesis of Intermediate 14h

A solution of methylamine in ethanol (0.41 g, 3.96 mmol) was added to a solution of intermediate 14g (20 mg, 57.57 μmol) in EtOH (1 mL). The reaction mixture was stirred at 25° C. for 16 hours. The reaction mixture was concentrated under reduced pressure to obtain intermediate 14h. MS m/z: 347.2 [M+1]⁺.

### Step 8: Synthesis of Hydrochloride of Intermediate 14i

Intermediate 14h (18 mg, 51.96 μmol) was dissolved in MeOH (0.5 mL). HCl/dioxane (4 M, 64.95 μL) was then added thereto, and the reaction mixture was stirred at 25° C. for 2 hours. The reaction mixture was concentrated under reduced pressure to obtain the hydrochloride of intermediate 14i. MS m/z: 247.2 [M+1]⁺.

### Step 9: Synthesis of Compound 14

Triethylamine (70.73 μmol, 9.84 μL) was added to a solution of the hydrochloride of intermediate 14i (10 mg) in DMF (1 mL). The reaction mixture was stirred at 20° C. for 0.5 hours. Then, intermediate 1h (9.59 mg, 35.36 μmol) and potassium iodide (587.06 μg, 3.54 μmol) were added, and the reaction mixture was stirred at 50° C. for 2 hours. The reaction mixture was filtered, and the residue was separated by preparative HPLC (column: Welch Xtimate C18, 100× 40 mm×3 um; mobile phase: [water (trifluoroacetic acid)-acetonitrile]; gradient: 0%-30% acetonitrile over 8 minutes) to obtain a crude product. A solution of methylamine in ethanol (0.30 g) was added thereto. The reaction mixture was stirred at 25° C. for 24 hours, then concentrated to obtain compound 14. MS m/z: 437.2 [M+1]⁺; ¹H NMR (400 MHz, CD₃OD) δ ppm 7.63 (d, J=8.53 Hz, 1H), 7.45 (d, J=8.53 Hz, 1H), 7.29 (t, J=7.65 Hz, 1H), 7.10 (d, J=8.78 Hz, 1H), 4.50 (br s, 1H), 3.75 (br d, J=11.29 Hz, 1H), 3.67 (s, 2H), 3.03-3.14 (m, 1H), 2.87-2.95 (m, 2H), 2.81 (s, 3H), 2.37-2.43 (m, 2H), 2.16-2.27 (m, 1H), 2.09 (t, J=7.53 Hz, 1H), 1.88-1.95 (m, 3H), 1.65-1.76 (m, 1H), 1.46-1.54 (m, 1H).

### Example 15

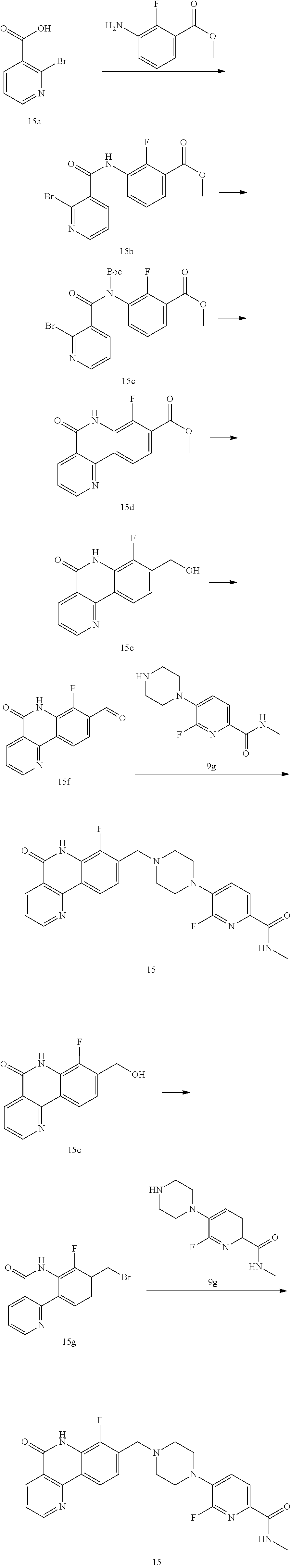

### Step 1: Synthesis of Intermediate 15b

Compound 15a (0.8 g, 3.96 mmol) and methyl 3-amino-2-fluorobenzoate (803.87 mg, 4.75 mmol) were added to DMF (10 mL). HATU (1.81 g, 4.75 mmol) and triethylamine (1.20 g, 11.88 mmol, 1.65 mL) were then added to the reaction mixture. The reaction mixture was stirred at 25° C. for 16 hours. The reaction mixture was added to 5 mL of water, and the mixture was extracted with ethyl acetate (10 mL×3). The organic phases were combined, dried over anhydrous sodium sulfate, and filtered. The filtrate was concentrated under reduced pressure at 45° C. to obtain a crude product. The crude product was purified by preparative TLC (PE/EA=2/1, R_{f}=0.14) to obtain intermediate 15b. MS m/z: 352.7, 354.7 [M+1]⁺; ¹H NMR (400 MHz, CDCl₃) δ ppm 8.53-8.62 (m, 1H) 8.43 (dd, J=4.75, 1.88 Hz, 2H) 8.00 (dd, J=7.63, 1.88 Hz, 1H) 7.65 (td, J=7.41, 1.69 Hz, 1H) 7.36 (dd, J=7.63, 4.75 Hz, 1H) 7.13-7.24 (m, 1H) 3.84-3.91 (m, 3H).

### Step 2: Synthesis of Intermediate 15c

Intermediate 15b (700 mg, 1.98 mmol) was added to DCM (15 mL). Et₃N (5.95 mmol, 827.70 μL), (Boc) 20 (519.14 mg, 2.38 mmol), and DMAP (48.43 mg, 396.44 μmol) were then added to the reaction mixture. The reaction mixture was stirred at 25° C. for 4 hours. The reaction mixture was added to 5 mL of water, and the mixture was extracted with ethyl acetate (10 mL×3). The organic phases were combined, dried over anhydrous sodium sulfate, and concentrated under reduced pressure at 45° C. to obtain a crude product. The crude product was purified by column chromatography (eluent: PE/EA=5/1 to 2/1, v/v) to obtain intermediate 15c. MS m/z: 452.9, 454.9 [M+1]⁺.

### Step 3: Synthesis of Intermediate 15d

Intermediate 15c (20 mg, 44.12 μmol) was added to DMF (3 mL). Tributylphosphine (8.93 mg, 44.12 μmol), 1,3-bis(diphenylphosphino) propane (7.28 mg, 17.65 μmol), Pd(OAc)₂ (3.96 mg, 17.65 μmol), and potassium carbonate (18.30 mg, 132.37 μmol) were then added to the reaction mixture. The reaction was carried out under microwave irradiation at 140° C. for 10 minutes. The reaction mixture was concentrated under reduced pressure at 60° C. to obtain a crude product. The crude product was purified by preparative TLC (DCM/MeOH=20/1) to obtain intermediate 15d. MS m/z: 272.8 [M+1]⁺.

### Step 4: Synthesis of Intermediate 15e

Intermediate 15d (20 mg, 73.47 μmol) was added to THF (2 mL). At 0° C., LiAlH₄ (2.79 mg, 73.47 μmol) was added to the reaction mixture. The mixture was stirred at 0° C. for 30 minutes. The reaction mixture was added with saturated potassium sodium tartrate solution (2 mL), and extracted with ethyl acetate (10 mL×3). The organic phases were combined, dried over anhydrous sodium sulfate, and concentrated under reduced pressure at 45° C. to obtain a crude product. The crude product was purified by preparative TLC (PE/EA=2/1) to obtain intermediate 15e. MS m/z: 244.8 [M+1]⁺.

### Step 5: Synthesis of Intermediate 15f

Intermediate 15e (11.5 mg, 47.09 μmol) was added to THF (4 mL). Manganese dioxide (40.94 mg, 470.89 μmol) was then added to the reaction mixture at 20° C. The mixture was reacted at 20° C. for 2 hours, then filtered. The filtrate was concentrated under reduced pressure at 45° C. to obtain a crude product 15f. MS m/z: 243.0 [M+1]⁺.

### Step 6: Synthesis of Trifluoroacetate of Compound 15

Intermediate 9g (12.26 mg, hydrochloride) was added to DMSO (2 mL), then Et₃N (9.03 mg, 89.22 μmol) was added to the reaction mixture. The reaction mixture was stirred at 25° C. for 0.5 hours. Intermediate 15f (10 mg, 41.29 μmol) was then added to the reaction mixture, and the pH of the mixture was adjusted to 6 using acetic acid. The mixture was stirred at 25° C. for 2 hours. NaBH(OAc)₃ (18.91 mg, 89.22 μmol) was added to the reaction mixture, which was stirred at 25° C. for 1 hour. The reaction mixture was added with water (2 mL), and extracted with dichloromethane (5 mL×3). The organic phases were combined, dried over anhydrous sodium sulfate, and concentrated under reduced pressure at 45° C. to obtain a crude product. The crude product was purified by preparative HPLC (column: Welch Xtimate C18, 100× 40 mm×3 um; mobile phase: [water (trifluoroacetic acid)-acetonitrile]; gradient: 2%-32% acetonitrile over 8 minutes) to obtain the trifluoroacetate of compound 15. MS m/z: 487.2 [M+Na]⁺. ¹H NMR (400 MHz, DMSO-d₆) δ ppm 12.19 (br s, 1H), 10.03-10.15 (m, 1H), 9.16 (d, J=2.40 Hz, 1H), 8.72 (d, J=3.20 Hz, 1H), 8.43-8.62 (m, 2H), 7.92 (d, J=4.42 Hz, 1H), 7.71-7.84 (m, 2H), 7.45-7.52 (m, 1H), 4.66 (s, 2H), 3.70-3.81 (m, 4H), 3.19-3.25 (m, 4H), 2.81 (d, J=2.40 Hz, 3H).

### Step 7: Synthesis of Intermediate 15g

Intermediate 15e (50 mg, 204.73 μmol) was dissolved in DCM (3 mL). 1,2-Dibromotetrachloroethane (146.67 mg, 450.41 μmol) was added, and at 0° C., tributylphosphine (82.84 mg, 409.47 μmol) was added. The reaction mixture was stirred at 25° C. for 3 hours. The reaction mixture was filtered to obtain the filter cake, which was intermediate 15g. 1H NMR (400 MHz, DMSO-d₆) δ ppm 12.02 (s, 1H), 9.10 (d, J=4.52 Hz, 1H), 8.65 (d, J=8.03 Hz, 1H), 8.39-8.52 (m, 1H), 7.69-7.80 (m, 1H), 7.34-7.54 (m, 1H), 4.78-5.01 (m, 2H).

### Step 8: Synthesis of Compound 15

Intermediate 9g (38.02 mg, hydrochloride) was dissolved in DMF (1 mL). Triethylamine (32.95 mg, 325.61 μmol) was added, and the reaction mixture was stirred at 25° C. for 0.5 hours. Intermediate 15g (50 mg, 162.80 μmol) and KI (2.70 mg, 16.28 μmol) were then added. The reaction mixture was stirred at 50° C. for 3 hours. The reaction mixture was added with water (2 mL), and stirred for 10 minutes, resulting in the precipitation of a solid. The reaction mixture was filtered to obtain the filter cake, which was compound 15 g. MS m/z: 465.1 [M+1]⁺. ¹H NMR (400 MHz, DMSO-d₆) δ ppm 11.90 (s, 1H), 9.09 (dd, J=4.63, 1.75 Hz, 1H), 8.65 (dd, J=7.94, 1.81 Hz, 1H), 8.39-8.49 (m, 2H), 7.84 (dd, J=8.13, 1.25 Hz, 1H), 7.72 (dd, J=8.00, 4.50 Hz, 1H), 7.57 (dd, J=10.76, 8.13 Hz, 1H), 7.37 (dd, J=7.94, 6.44 Hz, 1H), 3.75 (s, 2H), 3.15-3.24 (m, 4H), 2.76 (d, J=4.75 Hz, 3H), 2.61-2.65 (m, 4H).

### Example 16

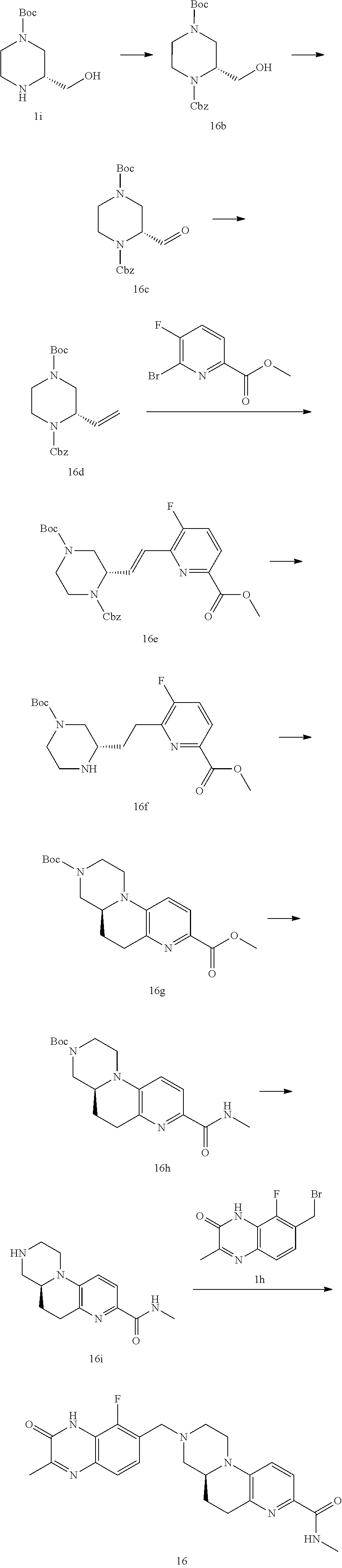

### Step 1: Synthesis of Intermediate 16b

Compound 1i (5 g, 23.12 mmol) was added to a mixture of H₂O (40 mL) and DCM (40 mL). NaHCO₃ (5.83 g, 69.36 mmol) was then added to the reaction mixture. The mixture was cooled to 0° C., and benzyl chloroformate (5.92 g, 34.68 mmol, 4.95 mL) was added to the reaction mixture. The reaction mixture was stirred at 20° C. for 16 hours. DCM (40 mL) was added to the reaction mixture, resulting in separation of phases. The organic phase was dried over anhydrous sodium sulfate, and concentrated under reduced pressure at 50° C. to obtain a crude product. The crude product was purified by column chromatography (eluent: PE/EA=5:1 to 2:1, v/v) to obtain intermediate 16b.

### Step 2: Synthesis of Intermediate 16c

Intermediate 16b (8 g, 22.83 mmol) was added to DCM (120 mL). At 0° C., Dess-Martin periodinane (14.52 g, 34.25 mmol, 10.61 mL) was added to the reaction mixture in batches. The mixture was stirred at 0° C. for 3 hours. Calcium hydroxide (10 g) was added to the reaction mixture, and the mixture was stirred at 0° C. for 1 hour. The mixture was then filtered, and the filter cake was washed with dichloromethane (60 mL×3). The filtrate was dried over anhydrous sodium sulfate and concentrated under reduced pressure at 50° C. to obtain a crude product. The crude product was purified by column chromatography (eluent: ethyl acetate/n-heptane=5/1 to 2/1, v/v) to obtain intermediate 16c.

### Step 3: Synthesis of Intermediate 16d

Under a nitrogen atmosphere, KHMDS (1 M, 51.67 mL) was added dropwise to a solution of methyltriphenylphosphonium bromide (18.46 g, 51.67 mmol) in THF (50 mL) at 20° C. The reaction mixture was stirred at 20° C. for 1 hour. The temperature was then lowered to −70° C., and a solution of intermediate 16c (6 g, 17.22 mmol) in THF (50 mL) was slowly added dropwise to the reaction mixture with stirring over 10 minutes. The reaction mixture was warmed to 20° C. and stirred for 3 hours at that temperature. The reaction mixture was added with 50 mL of methanol, then 50 mL of saturated potassium sodium tartrate solution was added thereto. The mixture was extracted with ethyl acetate (100 mL×3). The organic phase was dried over anhydrous sodium sulfate and filtered. The filtrate was concentrated under reduced pressure at 45° C. to obtain a crude product. The crude product was purified by column chromatography (eluent: PE/EA=5/1 to 1/1, v/v) to obtain intermediate 16d. 1H NMR (400 MHz, CDCl₃) δ ppm 7.14-7.28 (m, 5H) 5.61 (ddd, J=17.39, 10.76, 4.50 Hz, 1H) 5.02-5.11 (m, 2H) 5.00 (d, J=2.63 Hz, 2H) 4.58 (br s, 1H) 3.67-3.96 (m, 3H) 2.58-3.04 (m, 3H) 1.30 (s, 9H).

### Step 4: Synthesis of Intermediate 16e

Intermediate 16d (500.00 mg, 1.44 mmol), methyl 6-bromo-5-fluoropicolinate (337.77 mg, 1.44 mmol), PPh₃ (75.71 mg, 288.67 μmol), Na₂CO₃ (458.93 mg, 4.33 mmol), and Pd(OAc)₂ (32.40 mg, 144.33 μmol) were added to DMF (5 mL). The reaction mixture was purged with nitrogen three times and stirred at 130° C. for 12 hours. The reaction mixture was added with 10 mL of water, then extracted with ethyl acetate (10 mL×3). The organic phase was dried over anhydrous sodium sulfate, and concentrated under reduced pressure at 50° C. to obtain a crude product. The crude product was purified by column chromatography (eluent: PE/EA=4/1 to 2/1, v/v) to obtain intermediate 16e. MS m/z: 522.1 [M+23]⁺.

### Step 5: Synthesis of Intermediate 16f

Intermediate 16e (0.2 g, 400.38 μmol) was added to a hydrogenation flask containing methanol (10 mL). The flask was purged with argon once, and Pd/C (200 mg) was added into the reaction flask. The reaction mixture was purged with hydrogen three times and stirred under a hydrogen atmosphere (30 psi) at 30° C. for 16 hours. The reaction mixture was subjected to suction filtration under reduced pressure through diatomite, and the filtrate was concentrated under reduced pressure at 40° C. to obtain intermediate 16f. MS m/z: 368.1 [M+1]⁺.

### Step 6: Synthesis of Intermediate 16g

Intermediate 16f (100 mg, 272.17 μmol) was added to DMF (4 mL), then potassium carbonate (75.23 mg, 544.34 μmol) was added to the reaction mixture. The reaction mixture was stirred at 50° C. for 2 hours, and subjected to suction filtration under reduced pressure. The filtrate was concentrated under reduced pressure to obtain intermediate 16g.

MS m/z: 348.2 [M+1]⁺.

### Step 7: Synthesis of Intermediate 16h

Intermediate 16g (80 mg, 230.28 μmol) was added to ethanol (2 mL). Then a solution of methylamine in ethanol (2.53 g, 24.44 mmol) was added thereto. The reaction mixture was stirred at 30° C. for 16 hours, and concentrated under reduced pressure to obtain intermediate 16h. MS m/z: 347.1 [M+1]⁺.

### Step 8: Synthesis of Hydrochloride of Intermediate 16i

Intermediate 16h (40 mg, 115.47 μmol) was added to MeOH (2 mL). HCl/dioxane solution (4 M, 144.33 μL) was then added thereto. The reaction mixture was stirred at 30° C. for 4 hours, and concentrated under reduced pressure to obtain the hydrochloride of intermediate 16i. MS m/z: 269.1 [M+23]⁺.

### Step 9: Synthesis of Trifluoroacetate of Compound 16

Et₃N (10.74 mg, 106.09 μmol, 14.77 μL) was added to a solution of the hydrochloride of intermediate 16i (15 mg) in DMF (1 mL). The reaction mixture was stirred at 20° C. for 0.5 hours. Intermediate 1h (14.38 mg, 53.05 μmol) and potassium iodide (880.58 μg, 5.30 μmol) were then added thereto. The reaction mixture was stirred at 50° C. for 2 hours, and concentrated under reduced pressure to obtain a crude product. The crude product was purified by preparative HPLC (column: Welch Xtimate C18, 100×40 mm×3 um; mobile phase: [water (trifluoroacetic acid)-acetonitrile]; gradient: 24%-54% acetonitrile) to obtain the trifluoroacetate of compound 16. MS m/z: 437.2 [M+1]⁺; ¹H NMR (400 MHz, CD₃OD) δ ppm 7.82 (d, J=8.53 Hz, 1H) 7.71 (d, J=8.28 Hz, 1H) 7.44-7.51 (m, 1H) 7.37 (d, J=8.78 Hz, 1H) 4.59 (s, 2H) 4.23 (br d, J=14.56 Hz, 1H) 3.61-3.75 (m, 2H) 3.38-3.54 (m, 1H) 3.08-3.23 (m, 3H) 2.99-3.06 (m, 2H) 2.94 (s, 3H) 2.56 (s, 3H) 2.14-2.24 (m, 1H) 1.85-1.98 (m, 1H).

### Example 17

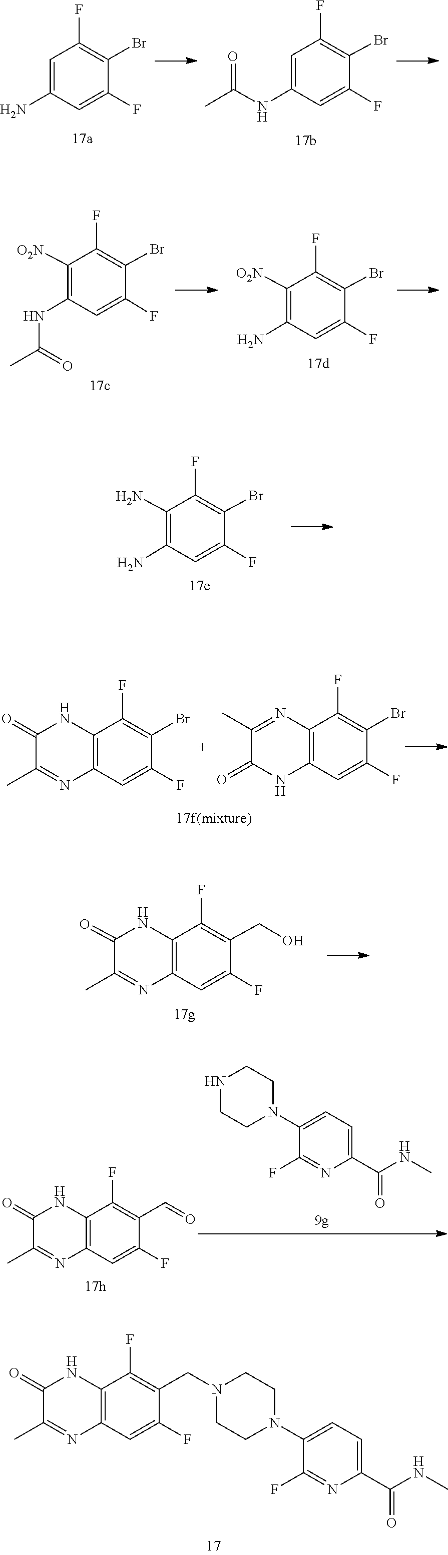

### Step 1: Synthesis of Intermediate 17b

At 0° C., triethylamine (19.46 g, 192.30 mmol) was added dropwise to a solution of compound 17a (10 g, 48.08 mmol) in DCM (50 mL). The mixture was stirred for 30 minutes. Subsequently, acetyl chloride (6.04 g, 76.92 mmol, 5.47 mL) was slowly added dropwise thereto. The reaction mixture was then warmed to 25° C. and reacted for 3 hours. The reaction mixture was added to water (100 mL). The mixture was extracted with dichloromethane (40 mL×5). The organic phases were combined, washed with saturated brine (100 mL). The organic phase was dried over anhydrous sodium sulfate, and filtered. The filtrate was concentrated under reduced pressure to obtain a crude product. The crude product was purified by column chromatography (eluent: EA/PE=0%-10%, v/v) to obtain intermediate 17b. MS m/z: 249.9, 251.9 [M+1]⁺; ¹H NMR (400 MHz, DMSO-d₆) δ ppm 10.41 (s, 1H) 7.43-7.45 (m, 2H) 2.07 (s, 3H).

### Step 2: Synthesis of Intermediate 17c

Fuming nitric acid (71.99 mmol, 3.24 mL) was added to a three-neck flask and cooled to 0° C. Intermediate 17b was added in batches, then sulfuric acid (334.75 mmol, 17.84 mL) was slowly added dropwise thereto while maintaining the temperature at 0° C. The dropwise addition was completed within 1 hour, and the mixture was then warmed to 25° C. and reacted for 1 hour. The reaction mixture was poured into 100 mL of 3M sodium hydroxide solution at 0° C., and the pH was adjusted to approximately 7. The mixture was extracted with ethyl acetate (30 mL×5). The organic phases were combined, washed with saturated brine (100 mL×2), dried over anhydrous sodium sulfate, filtered, and concentrated under reduced pressure to obtain intermediate 17c. ¹H NMR (400 MHz, DMSO-d₆) δ ppm 10.55 (s, 1H) 7.50-7.53 (m, 1H) 2.08 (s, 3H).

### Step 3: Synthesis of Intermediate 17d

Intermediate 17c (9.50 g, 32.20 mmol) was dissolved in sulfuric acid (90 mL) and reacted at 50° C. for 2 hours. The reaction mixture was poured into 100 mL of ice water, and the pH was adjusted to 7-8 using 3 M NaOH solution. The mixture was filtered, and the filter cake was washed with 10 mL of water, dried under vacuum to obtain intermediate 17d. 1H NMR (400 MHz, DMSO-d₆) δ ppm 7.45 (br, s, 2H) 6.74-6.77 (m, 1H).

### Step 4: Synthesis of Intermediate 17e

Intermediate 17d (6.2 g, 24.51 mmol) was dissolved in EtOH (70 mL) and cooled to 0° C. Acetic acid (14.72 g, 245.06 mmol, 14.03 mL) was added, then zinc (14.88 g, 227.56 mmol) was added in batches. The reaction was carried out for 1 hour. The reaction mixture was poured into 500 mL of water and extracted with ethyl acetate (80 mL×5). The organic phase was washed with 300 mL of saturated brine, dried over anhydrous sodium sulfate, and concentrated under reduced pressure to obtain intermediate 17e. ¹H NMR (400 MHz, DMSO-d6) δ ppm 6.37-6.40 (m, 1H) 5.21 (br, s, 2H) 4.65 (br, s, 2H).

### Step 5: Synthesis of Intermediate 17f

Intermediate 17e (3.15 g, 14.12 mmol) was added to EtOH (30 mL). Then pyruvic acid (1.49 g, 16.95 mmol, 1.19 mL) was dropwise added. The reaction mixture was reacted at 100° C. for 2 hours and concentrated under reduced pressure. 10 mL of ethanol was added, and the mixture was stirred for 20 minutes. The mixture was filtered, and the filter cake was collected to obtain a mixture of intermediate 17f. MS m/z: 274.9, 276.9 [M+1]⁺.

### Step 6: Synthesis of Intermediate 17g

The mixture of intermediate 17f (3.05 g, 11.09 mmol) and 1-(tributylstannyl) methanol (4.27 g, 13.31 mmol) were dissolved in dioxane (30 mL). Chloro(2-dicyclohexylphosphino-2′,4′,6′-triisoporpyl-1, l′-biphenyl) [2-(2′-amino-1,1′-biphenyl)]palladium (II) (436.24 mg, 554.45 μmol) was then added thereto. The reaction mixture was purged with nitrogen and reacted at 110° C. for 3 hours. The reaction mixture was concentrated under reduced pressure. The reaction mixture was purified by flash silica gel chromatography (EA/PE=0% to 30%, MeOH/DCM=0% to 5%) to obtain a crude product. The crude product was further separated by preparative HPLC (column: Welch Xtimate C18, 100×40 mm×3 um; mobile phase: [water (trifluoroacetic acid)-acetonitrile]; gradient: 0%-26% acetonitrile over 8 minutes) to obtain intermediate 17g. MS m/z: 227.1 [M+1]⁺; ¹H NMR (400 MHz, DMSO-d₆) δ ppm 12.51 (s, 1H) 7.35-7.37 (m, 1H) 5.25 (s, 1H) 4.57 (s, 2H) 2.41 (s, 3H).

### Step 7: Synthesis of Intermediate 17h

At 25° C., Dess-Martin periodinane (84.39 mg, 198.96 μmol) was added to a solution of intermediate 17g (30 mg, 132.64 μmol) in DCM (2 mL). The reaction mixture was stirred for 2 hours. The reaction mixture was added with calcium hydroxide (0.057 g), and stirred at 25° C. for 1 hour. The mixture was filtered, and the filter cake was washed with dichloromethane (10 mL×3). The filtrate was concentrated under reduced pressure at 50° C. to obtain a crude product. The crude product was purified by preparative TLC (PE:EA=1:1) to obtain intermediate 17h. ¹H NMR (400 MHz, DMSO-d₆) δ ppm 12.72 (s, 1H), 10.27 (s, 1H), 7.53-7.63 (m, 1H), 2.47 (s, 3H).

### Step 8: Synthesis of Compound 17

Intermediate 9g (21.86 mg, hydrochloride) was dissolved in DMSO (1 mL). Triethylamine (13.54 mg, 133.83 μmol) was added to the reaction mixture with stirring, and the mixture was stirred at 25° C. for 30 minutes. Intermediate 17h (15 mg, 66.92 μmol) was then added thereto, and the pH was adjusted to 6-7 using acetic acid. The reaction mixture was stirred at 25° C. for 2 hours. NaBH(OAc)₃ (28.36 mg, 133.83 μmol) was added thereto, and the mixture was stirred at 25° C. for 16 hours. The reaction mixture was added with water (2 mL) and extracted with EA (5 mL) three times. The organic phases were combined, dried over anhydrous sodium sulfate, and concentrated under reduced pressure to obtain a crude product. The crude product was purified by preparative HPLC (column: Welch Xtimate C18, 100× 40 mm×3 um; mobile phase: [water (trifluoroacetic acid)-acetonitrile]; gradient: 0%-30% acetonitrile over 8 minutes). The resulting product was basified with triethylamine (3 μL) and further purified by preparative TLC (100% EA) to obtain compound 17. MS m/z: 447.1 [M+1]⁺; ¹H NMR (400 MHz, CDCl₃) δ ppm 7.88 (dd, J=8.16, 1.13 Hz, 1H), 7.49 (dd, J=10.29, 8.03 Hz, 1H), 7.37 (dd, J=9.79, 1.76 Hz, 1H), 3.87 (s, 2H), 3.22-3.25 (m, 4H), 2.91 (s, 3H), 2.71-2.77 (m, 4H), 2.53 (s, 3H).

### Example 18

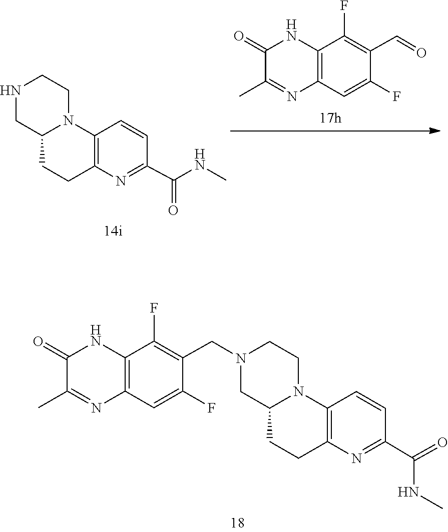 [SMILES: CNC(=O)c1ccc2c(n1)CC[C@@H]1CNCCN21 ; Cc1nc2cc(F)c(C=O)c(F)c2[nH]c1=O ; CNC(=O)c1ccc2c(n1)CC[C@@H]1CN(Cc3c(F)cc4nc(C)c(=O)[nH]c4c3F)CCN21]

### Step 1: Synthesis of Compound 18

Intermediate 14i (19.87 mg, hydrochloride) was dissolved in DMSO (1 mL). Triethylamine (133.83 μmol, 18.63 μL) was added to the reaction mixture with stirring, and the mixture was stirred at 25° C. for 30 minutes. Intermediate 17h (15.00 mg, 66.92 μmol) was then added thereto, and the pH was adjusted to 6-7 using acetic acid. The mixture was stirred at 25° C. for 2 hours. NaBH(OAc)₃ (28.36 mg, 133.83 μmol) was added thereto, and the reaction mixture was stirred at 25° C. for 16 hours. The reaction mixture was added with water (2 mL) and extracted with EA (5 mL×3). The organic phases were combined, dried over anhydrous sodium sulfate, and concentrated under reduced pressure to obtain a crude product. The crude product was separated by preparative HPLC (column: Welch Xtimate C18, 100× 40 mm×3 um; mobile phase: [water (trifluoroacetic acid)-acetonitrile]; gradient: 0%-30% acetonitrile over 8 minutes) to obtain a further crude product. The crude product was added with triethylamine (3 μL), and purified by preparative TLC (100% EA) to obtain compound 18. MS m/z: 455.2 [M+1]⁺; ¹H NMR (400 MHz, CD₃OD) δ ppm 7.64 (d, J=8.53 Hz, 1H), 7.06-7.16 (m, 2H), 3.76 (br d, J=12.55 Hz, 1H), 3.64 (s, 2H), 3.06-3.15 (m, 1H), 2.83-2.95 (m, 4H), 2.81-2.83 (m, 3H), 2.43 (s, 3H), 2.23 (td, J=11.54, 3.26 Hz, 1H), 1.92-2.00 (m, 2H), 1.64-1.78 (m, 1H), 1.22-1.27 (m, 1H).

### Example 19

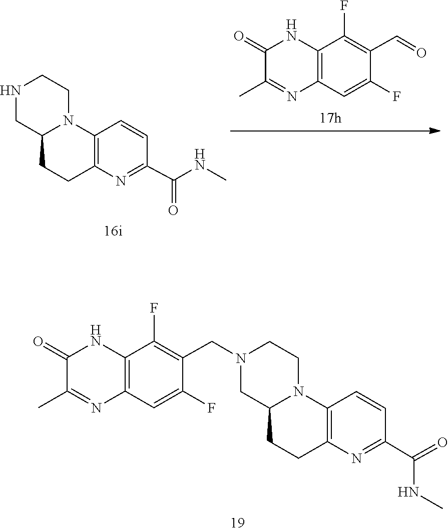 [SMILES: CNC(=O)c1ccc2c(n1)CC[C@H]1CNCCN21 ; Cc1nc2cc(F)c(C=O)c(F)c2[nH]c1=O ; CNC(=O)c1ccc2c(n1)CC[C@H]1CN(Cc3c(F)cc4nc(C)c(=O)[nH]c4c3F)CCN21]

### Step 1: Synthesis of Trifluoroacetate of Compound 19

Intermediate 16i (12.5 mg, hydrochloride) was dissolved in DMSO (1 mL). Triethylamine (74.58 μmol, 10.38 μL) was added, and the reaction mixture was stirred at 20° C. for 0.5 hours. 17h (8.36 mg, 37.29 μmol) and the pH was adjusted to 6-7 using acetic acid. The mixture was stirred at 20° C. for 2 hours. Subsequently, NaBH(OAc)₃ (15.81 mg, 74.58 μmol) was added, and the mixture was stirred for 16 hours. The reaction mixture was extracted with water (3 mL) and ethyl acetate (3 mL×3). The organic phases were combined, washed with 3 mL of saturated brine, dried over anhydrous sodium sulfate, and filtered to obtain a crude product. The crude product was separated by preparative HPLC (column: Welch Xtimate C18, 100× 40 mm×3 um; mobile phase: [water (trifluoroacetic acid)-acetonitrile]; gradient: 0%-30% acetonitrile over 8 minutes) to obtain the trifluoroacetate of compound 19. MS m/z: 455.1 [M+1]⁺; ¹H NMR (400 MHz, CD₃OD) δ ppm 1.63-1.77 (m, 1H) 1.93-2.05 (m, 2H) 2.18-2.31 (m, 1H) 2.42 (s, 3H) 2.81 (s, 3H) 2.82-2.86 (m, 2H) 2.93 (br t, J=10.42 Hz, 2H) 3.07 (s, 2H) 3.72-3.81 (m, 3H) 7.09 (d, J=8.78 Hz, 1H) 7.27 (dd, J=9.54, 1.76 Hz, 1H) 7.59-7.68 (m, 1H).

### Example 20

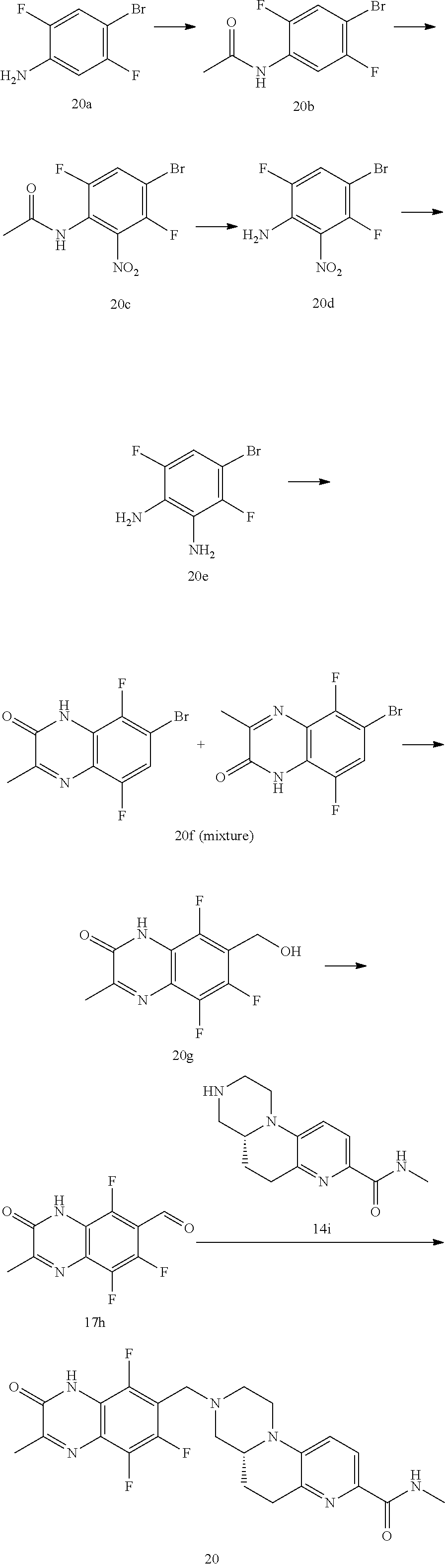

### Step 1: Synthesis of Intermediate 20b

At 0° C., triethylamine (7.78 g, 76.92 mmol, 10.71 mL) was added dropwise to a solution of compound 20a (4 g, 19.23 mmol) in DCM (20 mL). The mixture was stirred for 0.5 hours. Subsequently, acetyl chloride (2.42 g, 30.77 mmol, 2.19 mL) was slowly added dropwise thereto. The reaction mixture was then warmed to 18° C. and reacted for 3 hours. The reaction mixture was slowly added dropwise into 100 mL of water. The mixture was extracted with dichloromethane (20 mL×5). The organic phase was washed with 100 mL of saturated brine, dried over anhydrous sodium sulfate, and concentrated under reduced pressure to obtain a crude product. The crude product was purified by flash silica gel column chromatography (EA/PE=0%-10%) to obtain intermediate 20b. MS m/z: 250.0 [M+1]⁺; ¹H NMR (400 MHz, DMSO-d₆) δ ppm 10.03 (br s, 1H) 8.02-8.17 (m, 1H) 7.77 (dd, J=10.16, 6.40 Hz, 1H) 2.12 (s, 3H).

### Step 2: Synthesis of Intermediate 20c

At 0° C., intermediate 20b (2.52 g, 10.08 mmol) was suspended in nitric acid (1.27 g, 20.16 mmol, 907.24 μL). Sulfuric acid (9.19 g, 93.73 mmol, 5.00 mL) was then slowly added dropwise to the mixture. After the dropwise addition was complete, the mixture was reacted at 25° C. for 1 hour. The reaction mixture was added dropwise into water, resulting in the precipitation of a solid. The pH was adjusted to 6-7 using 2 M NaOH solution. The mixture was filtered, and the filter cake was dried to obtain intermediate 20c. ¹H NMR (400 MHz, DMSO-d₆) δ ppm 10.48 (s, 1H) 8.21-8.38 (m, 1H) 2.07 (s, 3H).

### Step 3: Synthesis of Intermediate 20d

Intermediate 20c (2.28 g, 7.73 mmol) was dissolved in sulfuric acid (22 mL) and stirred at 50° C. for 2 hours. The reaction mixture was added into 400 ml of water, and the pH was adjusted to 6-7 using 2 M NaOH solution, resulting in the precipitation of a solid. The mixture was filtered to obtain the filter cake, and the filter cake was dried under vacuum to obtain intermediate 20d. ¹H NMR (400 MHz, DMSO-d₆) δ ppm 7.76-7.89 (m, 1H) 7.09 (s, 2H).

### Step 4: Synthesis of Intermediate 20e

Intermediate 20d (1.49 g, 5.89 mmol) was dissolved in EtOH (15 mL). At 0° C., zinc powder (3.66 g, 55.95 mmol) and acetic acid (3.54 g, 58.89 mmol, 3.37 mL) were added in batches. The mixture was stirred at 40° C. for 1 hour. The reaction mixture was filtered, and the filtrate was concentrated under reduced pressure to obtain a crude product. The crude product was purified by column chromatography (eluent: PE:EA=100:0 to 90:10, v/v) to obtain intermediate 20e. MS m/z: 223.0 [M+1]⁺; ¹H NMR (400 MHz, DMSO-d₆) δ ppm 6.65 (dd, J=10.04, 6.02 Hz, 1H) 5.06 (s, 2H) 4.90 (s, 2H).

### Step 5: Synthesis of Intermediate 20f

Intermediate 20e (1.14 g, 5.11 mmol) was dissolved in EtOH (12 mL), and pyruvic acid (540.17 mg, 6.13 mmol, 432.14 μL) was then added. The mixture was stirred at 100° C. for 2 hours, then slowly cooled to 25° C. to crystallize over 14 hours. The reaction mixture was filtered to obtain the filter cake, which was intermediate 20f (mixture). MS m/z: 274.9 [M+1]⁺.

### Step 6: Synthesis of Intermediate 20g

Under a nitrogen atmosphere, intermediate 20f (500 mg, 1.82 mmol) was dissolved in dioxane (5 mL). 1-(Tributylstannyl) methanol (700.42 mg, 2.18 mmol) and chloro(2-dicyclohexylphosphino-2′,4′,6′-triisoporpyl-1,1′-biphenyl) [2-(2′-amino-1,1′-biphenyl)]palladium (II) (71.51 mg, 90.89 μmol) were added. The reaction mixture was stirred at 110° C. for 4 hours. The reaction mixture was concentrated under reduced pressure and purified by column chromatography (eluent: PE:EA=90:10 to 50:50, V/V) to obtain a crude product. The crude product was purified by preparative HPLC (column: Welch Xtimate C18, 100× 40 mm×3 um; mobile phase: [water (trifluoroacetic acid)-acetonitrile]; gradient: 0%-26% acetonitrile) to obtain intermediate 20g. MS m/z: 226.8 [M+1]⁺; ¹H NMR (400 MHz, DMSO-d6) δ ppm 12.58 (br s, 1H) 6.99-7.25 (m, 1H) 4.62 (br d, J=3.51 Hz, 2H) 2.43 (br s, 3H).

### Step 7: Synthesis of Intermediate 20h

Intermediate 20g (100 mg, 442.13 μmol) was dissolved in DCM (2 mL). Dess-Martin periodinane (281.29 mg, 663.19 μmol, 205.47 μL) was added, and the reaction mixture was stirred at 20° C. for 2 hours. The reaction mixture was added with 0.1899 g of calcium hydroxide and stirred at 20° C. for 1 hour. The reaction mixture was filtered to obtain a crude product. The crude product was purified by preparative TLC (DCM:MeOH=20:1) to obtain intermediate 20h. ¹H NMR (400 MHz, DMSO-d₆) δ ppm 12.96 (br, s, 1H), 10.26 (s, 1H), 7.40-7.44 (m, 1H), 2.49 (s, 3H).

### Step 8: Synthesis of Compound 20

Intermediate 14i (13.25 mg, hydrochloride) was dissolved in DMSO (1 mL). Triethylamine (9.03 mg, 89.22 μmol, 12.42 μL) was added to the reaction mixture with stirring, and the mixture was stirred at 25° C. for 30 minutes. Intermediate 20h (10 mg, 44.61 μmol) was added to the reaction mixture, and the pH was adjusted to 6-7 using acetic acid. The mixture was stirred at 25° C. for 2 hours. NaBH(OAc)₃ (18.91 mg, 89.22 μmol) was added thereto, and the reaction mixture was stirred at 25° C. for 16 hours. The reaction mixture was added with water (2 mL) and extracted with EA (5 mL×3). The organic phases were combined, dried over anhydrous sodium sulfate, and concentrated under reduced pressure to obtain a crude product. The crude product was separated by preparative HPLC (column: Welch Xtimate C18, 100× 40 mm×3 um; mobile phase: [water (trifluoroacetic acid)-acetonitrile]; gradient: 0%-30% acetonitrile) to obtain a crude product. The crude product was added with triethylamine (3 μL), and purified by preparative TLC (100% EA) to obtain compound 20. MS m/z: 455.2 [M+1]⁺; ¹H NMR (400 MHz, CD₃OD) δ ppm 7.75 (d, J=8.78 Hz, 1H), 7.38 (dd, J=9.41, 1.63 Hz, 1H), 7.21 (d, J=8.78 Hz, 1H), 4.59 (s, 1H), 3.83-3.90 (m, 3H), 3.14-3.23 (m, 1H), 3.01-3.11 (m, 2H), 2.94-2.99 (m, 1H), 2.92-2.94 (m, 3H), 2.85-2.92 (m, 1H), 2.54 (s, 3H), 2.31-2.41 (m, 1H), 1.93-2.23 (m, 2H), 1.75-1.88 (m, 1H).

### Example 21

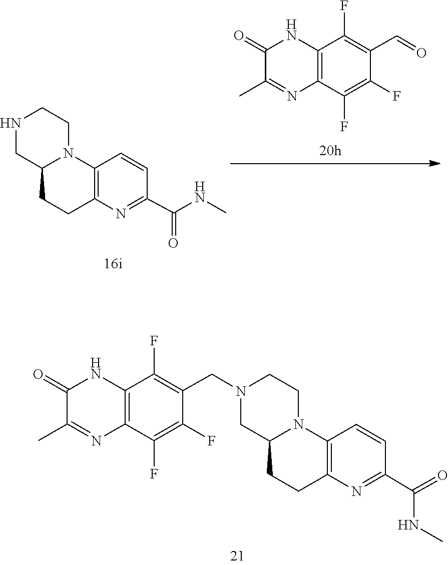 [SMILES: CNC(=O)c1ccc2c(n1)CC[C@H]1CNCCN21 ; Cc1nc2c(F)c(F)c(C=O)c(F)c2[nH]c1=O ; CNC(=O)c1ccc2c(n1)CC[C@H]1CN(Cc3c(F)c(F)c4nc(C)c(=O)[nH]c4c3F)CCN21]

### Step 1: Synthesis of Compound 21

Intermediate 16i (15 mg, hydrochloride) was dissolved in DMSO (1 mL). Triethylamine (9.06 mg, 89.50 μmol, 12.46 μL) was added, and the reaction mixture was stirred at 20° C. for 0.5 hours. Intermediate 20h (12.54 mg, 44.75 μmol) was then added thereto and the pH was adjusted to 6-7 using acetic acid. The mixture was stirred at 20° C. for 2 hours. Subsequently, NaBH(OAc)₃ (18.97 mg, 89.50 μmol) was added, and the mixture was stirred for 16 hours. The reaction mixture was extracted with water (3 mL) and ethyl acetate (3 mL×3). The organic phases were combined, washed with 10 mL of saturated brine, then dried over anhydrous sodium sulfate, and filtered to obtain a crude product. The crude product was separated by preparative HPLC (column: Welch Xtimate C18, 100× 40 mm×3 um; mobile phase: [water (trifluoroacetic acid)-acetonitrile]; gradient: 0%-30% acetonitrile over 8 minutes) to obtain a crude product. The crude product was added with 3 μL of triethylamine and purified by preparative TLC (EA=100%) to obtain compound 21. MS m/z: 455.0 [M+1]⁺; ¹H NMR (400 MHz, CD₃OD) δ ppm 7.58-7.69 (m, 1H) 7.10 (d, J=8.78 Hz, 2H) 3.76 (br d, J=11.80 Hz, 1H) 3.64 (d, J=1.51 Hz, 2H) 3.04-3.17 (m, 1H) 2.82-2.96 (m, 5H) 2.82 (s, 3H) 2.43 (s, 3H) 2.15-2.30 (m, 1H) 1.88-2.01 (m, 2H) 1.61-1.79 (m, 1H).

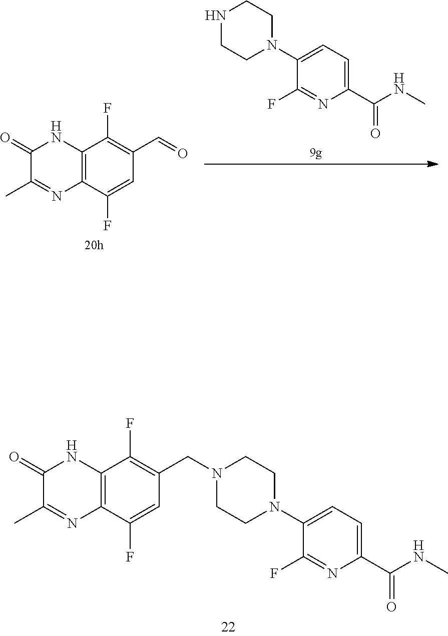

### Step 1: Synthesis of Trifluoroacetate of Compound 22

Intermediate 9g (7.29 mg, hydrochloride) was dissolved in DMSO (1 mL). Triethylamine (44.61 μmol, 6.21 μL) was added, and the reaction mixture was stirred at 20° C. for 0.5 hours. Intermediate 20h (5 mg, 22.31 μmol) was then added thereto and the pH was adjusted to 6-7 using acetic acid. The mixture was stirred for 2 hours. Subsequently, NaBH(OAc)₃ (9.45 mg, 44.61 μmol) was added, and the mixture was stirred for 4 hours. The reaction mixture was extracted with water (1 mL) and ethyl acetate (1 mL×3). The organic phase was washed with 10 mL of saturated brine, dried, and filtered to obtain a crude product. The crude product was separated by preparative HPLC (column: Welch Xtimate C18, 100×40 mm×3 um; mobile phase: [water (trifluoroacetic acid)-acetonitrile]; gradient: 1%-31% acetonitrile over 8 minutes) to obtain the trifluoroacetate of compound 22. MS m/z: 447.1 [M+1]⁺; ¹H NMR (400 MHz, CD₃OD) δ ppm 7.92-7.99 (m, 1H) 7.54-7.68 (m, 1H) 7.27 (dd, J=10.16, 5.40 Hz, 1H) 4.22-4.39 (m, 2H) 3.42-3.51 (m, 4H) 3.23-3.31 (m, 4H) 2.93 (s, 3H) 2.57 (s, 3H).

### Example 23

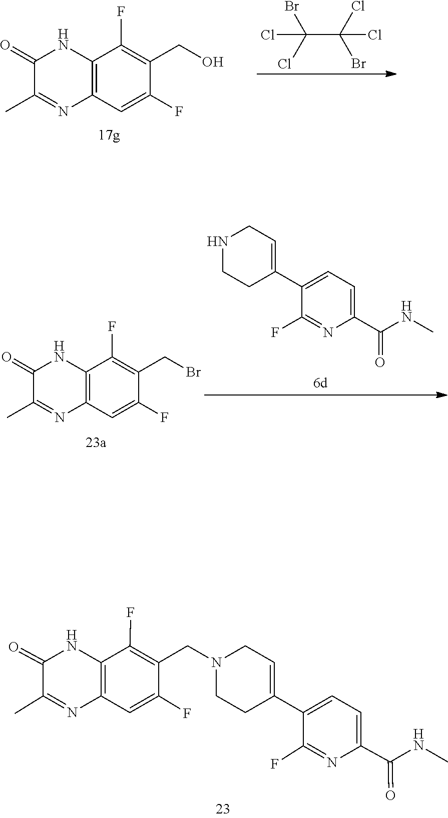 [SMILES: Cc1nc2cc(F)c(CO)c(F)c2[nH]c1=O ; ClC(Cl)(Br)C(Cl)(Cl)Br ; Cc1nc2cc(F)c(CBr)c(F)c2[nH]c1=O ; CNC(=O)c1ccc(C2=CCNCC2)c(F)n1 ; CNC(=O)c1ccc(C2=CCN(Cc3c(F)cc4nc(C)c(=O)[nH]c4c3F)CC2)c(F)n1]

### Step 1: Synthesis of Intermediate 23a

Compound 17g (100.00 mg, 442.13 μmol) and 1,2-dibromo-1,1,2,2-tetrachloroethane (316.74 mg, 972.68 μmol) were dissolved in DCM (5 mL). At 0° C., tributylphosphine (178.90 mg, 884.25 μmol) was added thereto. After the addition was complete, the mixture was warmed to 25° C. and stirred for 12 hours. The reaction mixture was filtered to obtain the filter cake, which was intermediate 23a. ¹H NMR (400 MHz, DMSO-d₆) δ ppm 12.63 (br s, 1H), 7.33-7.72 (m, 1H), 4.71-4.94 (m, 2H), 2.43 (s, 3H).

### Step 2: Synthesis of Compound 23

Triethylamine (21.00 mg, 207.56 μmol) was added to a solution of intermediate 6d (25.64 mg, hydrochloride) in DMF (2 mL). The mixture was stirred at 25° C. for 30 minutes. Intermediate 23a (30 mg, 103.78 μmol) and KI (1.72 mg, 10.38 μmol) were then added, and the reaction mixture was stirred at 50° C. for 2 hours. The reaction mixture was filtered, and the filtrate was concentrated to obtain a crude product. The crude product was purified by preparative HPLC (column: Welch Xtimate C18, 100×40 mm×3 μm; mobile phase: [water (trifluoroacetic acid)-acetonitrile]; gradient: 0%-30% acetonitrile) to obtain a further crude product. The crude product was added with triethylamine (20 μL), and purified by preparative TLC (eluent ratio: DCM:MeOH=10:1) to obtain compound 23. MS m/z: 444.1 [M+1]⁺; ¹H NMR (400 MHz, DMSO-d₆) δ ppm 12.54 (br s, 1H), 8.63 (br d, J=5.00 Hz, 1H), 8.05 (dd, J=9.82, 7.82 Hz, 1H), 7.91 (dd, J=7.69, 1.69 Hz, 1H), 7.46 (d, J=8.88 Hz, 1H), 6.14-6.27 (m, 1H), 3.80 (s, 2H), 3.13-3.26 (m, 3H), 2.79 (d, J=4.88 Hz, 3H), 2.60-2.76 (m, 3H), 2.44 (s, 3H).

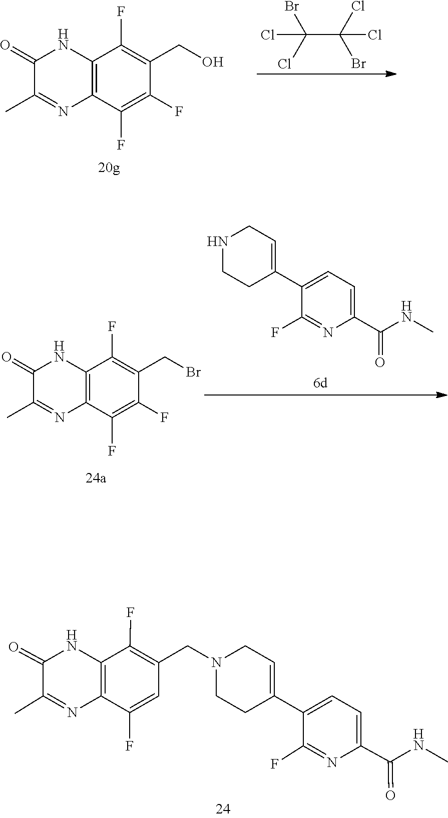 [SMILES: Cc1nc2c(F)c(F)c(CO)c(F)c2[nH]c1=O ; ClC(Cl)(Br)C(Cl)(Cl)Br ; Cc1nc2c(F)c(F)c(CBr)c(F)c2[nH]c1=O ; CNC(=O)c1ccc(C2=CCNCC2)c(F)n1 ; CNC(=O)c1ccc(C2=CCN(Cc3cc(F)c4nc(C)c(=O)[nH]c4c3F)CC2)c(F)n1]

### Step 1: Synthesis of Intermediate 24a

Compound 20g (100.00 mg, 442.13 μmol) and 1,2-dibromo-1,1,2,2-tetrachloroethane (316.74 mg, 972.68 μmol) were dissolved in DCM (5 mL). At 0° C., tributylphosphine (178.90 mg, 884.25 μmol) was added thereto. After the addition was complete, the mixture was warmed to 25° C. and stirred for 8 hours. The reaction mixture was filtered to obtain the filter cake, which was intermediate 24a. ¹H NMR (400 MHz, DMSO-d₆) δ ppm 12.72 (br s, 1H), 7.32 (dd, J=10.54, 5.77 Hz, 1H), 4.76 (s, 2H), 2.44 (s, 3H).

### Step 2: Synthesis of Compound 24

Triethylamine (21.00 mg, 207.56 μmol) was added to a solution of intermediate 6d (25.64 mg, hydrochloride) in DMF (2 mL). The mixture was stirred at 25° C. for 30 minutes. Intermediate 24a (30 mg, 103.78 μmol) and KI (1.72 mg, 10.38 μmol) were then added, and the reaction mixture was stirred at 50° C. for 2 hours. The reaction mixture was filtered, and the filtrate was concentrated to obtain a crude product. The crude product was purified by preparative HPLC (column: Welch Xtimate C18, 100×40 mm×3 um; mobile phase: [water (trifluoroacetic acid)-acetonitrile]; gradient: 0%-30% acetonitrile) to obtain a further crude product. The crude product was added with triethylamine (20 μL), and purified by preparative TLC (eluent ratio: DCM:MeOH=10:1) to obtain compound 24. MS m/z: 444.1 [M+1]⁺; ¹H NMR (400 MHz, DMSO-d₆) δ ppm 12.63 (br s, 1H), 8.64 (br d, J=4.63 Hz, 1H), 8.01-8.17 (m, 1H), 7.92 (br d, J=7.38 Hz, 1H), 7.07-7.28 (m, 1H), 6.25 (br s, 1H), 3.75 (s, 2H), 3.18-3.25 (m, 3H), 2.80 (br d, J=4.63 Hz, 3H), 2.65-2.76 (m, 3H), 2.44 (s, 3H).

### Example 25

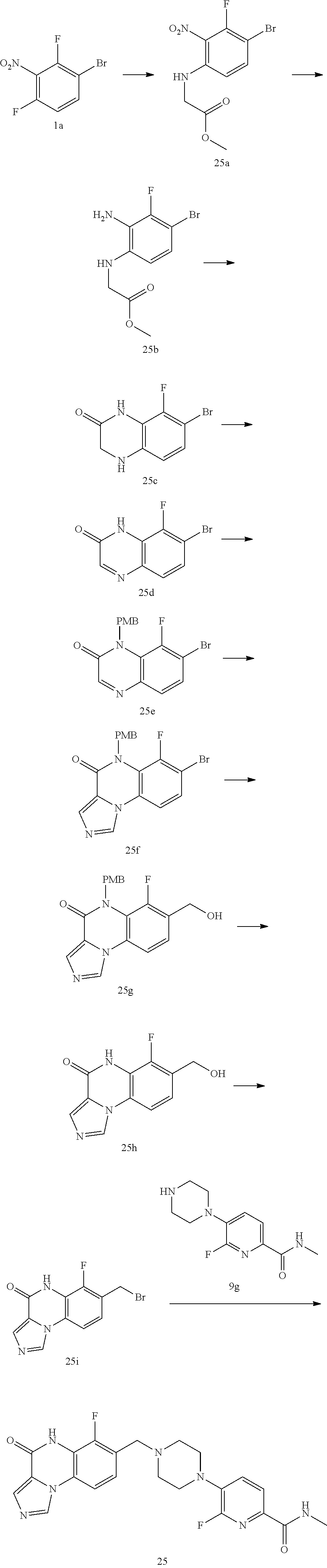

### Step 1: Synthesis of Intermediate 25a

Compound 1a (6.1 g, 25.63 mmol), methyl aminoacetate (3.54 g, 28.19 mmol, HCl), and N,N-diisopropylethylamine (9.94 g, 76.90 mmol) were added to DMF (60 mL). The reaction mixture was reacted at 25° C. for 18 hours. The reaction mixture was poured into 300 mL of water and extracted with ethyl acetate (100 mL×5). The organic phase was washed with 200 mL of saturated brine, dried over anhydrous sodium sulfate, filtered, and concentrated under reduced pressure to obtain a crude product. The crude product was purified by flash silica gel column chromatography (EA:PE=0%-10%) to obtain intermediate 25a. ¹H NMR (400 MHz, DMSO-d₆) δ ppm 7.54-7.68 (m, 2H), 6.70 (d, J=7.8 Hz, 1H), 4.20 (d, J=4.2 Hz, 2H), 3.68 (s, 3H).

### Step 2: Synthesis of Intermediate 25b

Intermediate 25a (1.82 g, 5.93 mmol) and NH₄Cl (2.54 g, 47.42 mmol) were added to a mixed solution of MeOH (26 mL) and H₂O (0.468 mL). Zinc (1.98 g, 30.29 mmol) was then added. The reaction was carried out at 0° C. for 2 hours. The reaction mixture was filtered, and 50 mL of water was added to the filtrate. The mixture was extracted with ethyl acetate (30 mL×3). The organic phases were combined, washed with 50 mL of saturated brine, dried over anhydrous sodium sulfate, and filtered. The filtrate was concentrated under reduced pressure to obtain intermediate 25b. MS m/z: 276.9, 278.9 [M+1]⁺.

### Step 3: Synthesis of Intermediate 25c

At 0° C., intermediate 25b (1.35 g, 4.87 mmol) was added to a mixed system of HCl/dioxane (4 M, 15.64 mL), EA (13.5 mL), and MeOH (13.5 mL). The reaction was carried out for 2 hours. The reaction mixture was directly concentrated under reduced pressure to obtain intermediate 25c. MS m/z: 245.0, 247.0 [M+1]⁺.

### Step 4: Synthesis of Intermediate 25d

At 0° C., DDQ (1.56 g, 6.86 mmol) was added to a solution of intermediate 25c (1.4 g, 5.71 mmol) in DCM (14 mL). The mixture was warmed to 25° C. and reacted for 4 hours. The reaction mixture was concentrated under reduced pressure, and 70 mL of saturated sodium bicarbonate solution was slowly added thereto. The mixture was stirred overnight. The mixture was extracted with ethyl acetate (50 mL×3). The organic phases were combined, washed with 70 mL of saturated brine, dried over anhydrous sodium sulfate, filtered, and concentrated under reduced pressure to obtain a crude product. The crude product was purified by flash silica gel column chromatography (eluent ratio: MeOH/DCM=0%-5%) to obtain intermediate 25d. MS m/z: 243.0, 245.0 [M+1]⁺. ¹H NMR (400 MHz, DMSO-d₆) δ ppm 12.75 (br s, 1H), 8.24 (s, 1H), 7.53-7.60 (m, 2H).

### Step 5: Synthesis of Intermediate 25e

Intermediate 25d (660 mg, 2.72 mmol) was dissolved in DMF (1 mL). Potassium carbonate (563.00 mg, 4.07 mmol), potassium iodide (67.62 mg, 407.35 μmol), and p-methoxybenzyl chloride (510.36 mg, 3.26 mmol) were added thereto, and the mixture was stirred at 20° C. for 3 hours. The reaction system was added with water (15 mL), and extracted with ethyl acetate (50 mL). The phases were separated. The organic phase was washed with water, dried over anhydrous sodium sulfate, and filtered. The filtrate was concentrated to obtain a crude product. The crude product was purified by flash silica gel column chromatography (PE:EA=3:1) to obtain intermediate 25e. MS m/z: 362.8, 364.8 [M+1]⁺, ¹H NMR (400 MHz, DMSO-d₆) δ ppm 8.43 (s, 1H), 7.50-7.64 (m, 2H), 7.24 (d, J=4.2 Hz, 2H), 6.85 (d, J=4.2 Hz, 2H), 5.60 (s, 2H), 3.72 (s, 3H).

### Step 6: Synthesis of Intermediate 25f

At 0° C., a solution of p-toluenesulfonylmethyl isocyanide (451.57 mg, 2.31 mmol) in THF (10 mL) was added to sodium hydride (154.19 mg, 3.85 mmol). The mixture was stirred for 10 minutes, and intermediate 25e (700.00 mg, 1.93 mmol) was then added thereto. The reaction mixture was stirred at 25° C. for 2 hours. The reaction mixture was added with water (5 mL), and extracted with ethyl acetate (30 mL). The organic phase was dried over anhydrous sodium sulfate and filtered. The filtrate was concentrated to obtain a crude product. The crude product was purified by flash silica gel column chromatography (first using PE:EA=3:1 to 1:1, then DCM:MeOH=10:1) to obtain intermediate 25f. MS m/z: 402.0, 404.0 [M+1]⁺, ¹H NMR (400 MHz, CDCl₃) δ ppm 8.44 (s, 1H), 8.13 (s, 1H), 7.40-7.51 (m, 2H), 7.22 (d, J=4.2 Hz, 2H), 6.85 (d, J=4.2 Hz, 2H), 5.61 (s, 2H), 3.79 (s, 3H).

### Step 7: Synthesis of Intermediate 25g

1-(Tributylstannyl) methanol (191.59 mg, 596.69 μmol) and intermediate 25f (200 mg, 497.24 μmol) were dissolved in 1,4-dioxane (5 mL). Chloro(2-dicyclohexylphosphino-2′,4′,6′-triisoporpyl-1,1′-biphenyl) [2-(2′-amino-1,1′-biphenyl)]palladium (II) (39.12 mg, 49.72 μmol) was added thereto, and the mixture was stirred at 80° C. for 1.5 hours. 1-(Tributylstannyl) methanol (31.93 mg, 99.44 μmol) was then added thereto. The reaction mixture was stirred at 80° C. for 2 hours, and heated to 100° C. and stirred for another 2 hours. The reaction mixture was directly concentrated under reduced pressure to obtain a crude product. The crude product was purified by flash silica gel column chromatography (eluent ratio: PE:EA=1:1; DCM:MeOH=10:1) to obtain intermediate 25g. MS m/z: 354.1 [M+1]⁺.

### Step 8: Synthesis of Intermediate 25h

At 15 to 25° C., intermediate 25g (80 mg, 226.41 μmol) was added to a reaction flask. Trifluoroacetic acid (3.07 g, 26.93 mmol, 2.00 mL) and trifluoromethanesulfonic acid (4.52 mmol, 400.00 μL) were added thereto. The reaction mixture was stirred for 2 hours. The reaction mixture was added with water (5 mL) and methanol (5 mL). The pH was adjusted to 10 with the addition of solid potassium carbonate. The mixture was stirred for another 1 hour. The reaction mixture was extracted with dichloromethane (20 mL). The organic phases were combined, washed with water (15 mL×2), then dried and filtered. The filtrate was concentrated under reduced pressure to obtain intermediate 25h. MS m/z: 234.1 [M+1]⁺.

### Step 9: Synthesis of Intermediate 25i

Intermediate 25h (80 mg, 343.06 μmol) and 1,2-dibromotetrachloroethane (245.77 mg, 754.72 μmol) were added to DCM (1.5 mL). Tributylphosphine (138.81 mg, 686.11 μmol) was then added, and the mixture was stirred at 20° C. for 3 hours. The reaction mixture was concentrated directly under reduced pressure to obtain the crude intermediate 25i.

### Step 10: Synthesis of Trifluoroacetate of Compound 25

Intermediate 25i (30 mg) and compound 9g (28.97 mg, hydrochloride) were dissolved in DMF (1 mL). Et₃N (10.25 mg, 101.32 μmol) and KI (16.82 mg, 101.32 μmol) were added thereto, and the mixture was stirred at 50° C. for 2 hours. The reaction system was added with water (5 mL), and extracted with ethyl acetate (10 mL×3). The organic phases were combined, dried over anhydrous sodium sulfate, and concentrated to obtain a crude product. The crude product was first purified by preparative TLC (eluent ratio: DCM:MeOH=10:1), then separated by preparative HPLC (column: Welch Xtimate C18, 100×40 mm×3 um; mobile phase: [water (trifluoroacetic acid)-acetonitrile]; gradient: 0%-30% acetonitrile) to obtain the trifluoroacetate of compound 25. MS m/z: 454.2 [M+1]⁺, ¹H NMR (400 MHz, CD₃OD) δ ppm 8.99 (s, 1H), 8.05 (d, J=4.4 Hz, 1H), 8.02 (s, 1H), 7.93 (d, J=4.4 Hz, 1H), 7.58-7.63 (m, 1H), 7.46-7.49 (m, 1H), 4.46 (s, 2H), 3.71-3.75 (m, 4H), 3.40-3.51 (m, 4H), 2.92 (s, 3H).

### Example 26

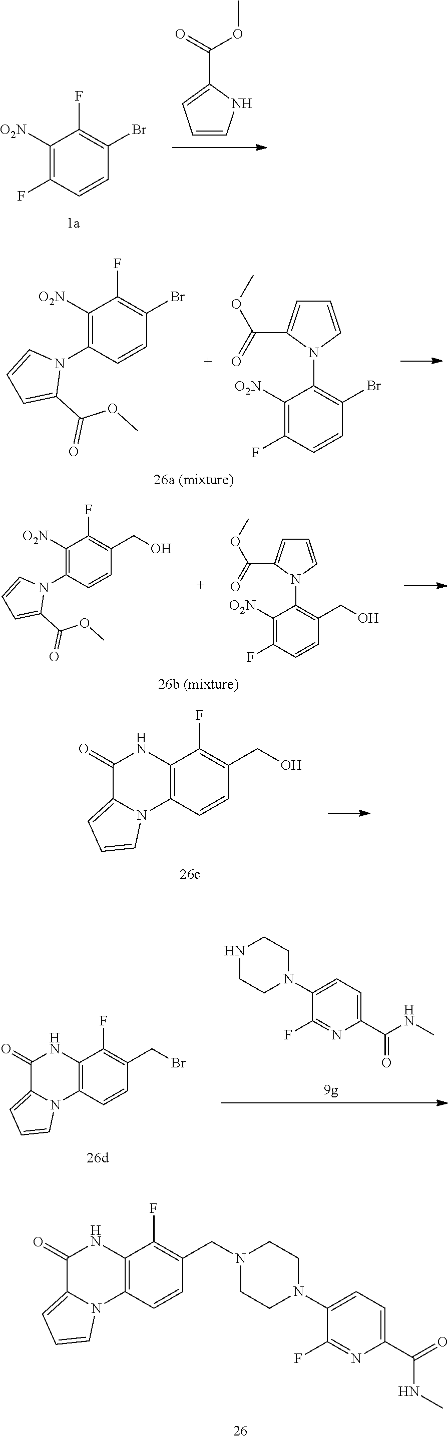

### Step 1: Synthesis of Intermediate 26a

Methyl 2-pyrrolecarboxylate (262.88 mg, 2.10 mmol) and compound 1a (500 mg, 2.10 mmol) were dissolved in DMF (10 mL). Sodium hydride (126.05 mg, 3.15 mmol, purity of 60%) was added in batches, and the mixture was stirred at room temperature (20° C.) for 3 hours. The reaction mixture was slowly added dropwise into 20 mL of saturated ammonium chloride solution. The mixture was extracted with ethyl acetate (30 mL×2). The organic phases were combined, washed with 20 mL of saturated brine, dried over anhydrous sodium sulfate, and filtered. The filtrate was concentrated under reduced pressure to obtain a crude product. The crude product was purified by flash silica gel column chromatography (eluent: PE:EA=90:10, V/V) to obtain the mixture intermediate 26a (in an approximate ratio of 2:1). MS m/z: 342.8, 344.8 [M+1]⁺.

### Step 2: Synthesis of Intermediate 26b

The mixture intermediate compound 26a (370 mg) was dissolved in dioxane (10 mL). 1-(Tributylstannyl) methanol (415.51 mg, 1.29 mmol) and chloro(2-dicyclohexylphosphino-2′,4′,6′-triisoporpyl-1,1′-biphenyl) [2-(2′-amino-1,1′-biphenyl)]palladium (II) (42.42 mg, 53.92 μmol) were added thereto, and the mixture was stirred at 110° C. for 4 hours. The reaction mixture was concentrated under reduced pressure to obtain a crude product. The crude product was purified by flash silica gel column chromatography (gradient elution: PE:EA=100:0 to 60:40) to obtain the mixture intermediate 26b. MS m/z: 294.9 [M+1]⁺.

### Step 3: Synthesis of Intermediate 26c

The mixture intermediate 26b (185 mg) was dissolved in MeOH (3 mL) and purged with argon. Palladium hydroxide (8.83 mg, 62.87 μmol) was added. The reaction flask was purged with hydrogen, and the mixture was stirred at 30° C. under 50 psi pressure for 16 hours. The reaction mixture was filtered, and the filtrate was concentrated under reduced pressure. DMF (2 mL) and 5 drops of triethylamine were added, and the mixture was stirred at 90° C. for 16 hours. DMF was removed using an oil pump to obtain a crude product, and a mixed solution of DCM and MeOH (DCM:MeOH=1:1, 3 mL) was added to the crude product. The mixture was filtered to obtain the filter cake, which was intermediate 26c. MS m/z: 232.8 [M+1]⁺. ¹H NMR (400 MHz, DMSO-d₆) δ ppm 11.22 (br s, 1H), 8.18-8.19 (m, 1H), 7.87 (d, J=4.40 Hz 1H), 7.26 (t, J=8.20 Hz, 1H), 7.07-7.08 (m, 1H), 6.70-6.72 (m, 1H), 5.35 (t, J=5.60 Hz, 1H), 4.60 (d, J=2.80 Hz, 2H).

### Step 4: Synthesis of Intermediate 26d

Intermediate 26c (20 mg, 86.13 μmol) and 1,2-dibromotetrachloroethane (28.05 mg, 86.13 μmol) were dissolved in DCM (0.5 mL). At 0° C., tributylphosphine (17.43 mg, 86.13 μmol) was added. After the addition was complete, the mixture was stirred at room temperature (20° C.) for 16 hours. An additional portion of 1,2-dibromotetrachloroethane (28.05 mg, 86.13 μmol) and tributylphosphine (17.43 mg, 86.13 μmol) was added thereto. The reaction mixture was stirred for another 1 hour. The reaction mixture was filtered to obtain the filter cake, which was intermediate 26d. ¹H NMR (400 MHz, DMSO-d₆) δ ppm 11.41 (br s, 1H), 8.20-8.21 (m, 1H), 7.85 (d, J=4.40 Hz 1H), 7.36 (t, J=8.20 Hz, 1H), 7.10-7.11 (m, 1H), 6.73-6.75 (m, 1H), 4.79 (s, 2H).

### Step 5: Synthesis of Compound 26

Intermediate 9g (11.83 mg, hydrochloride) was dissolved in DMF (1 mL). Triethylamine (17.43 mg, 172.26 μmol) was added, and the reaction mixture was stirred at 25° C. for 0.5 hours. Intermediate 26d (10 mg, 43.06 μmol) and potassium iodide (3.57 mg, 21.53 μmol) were then added. The mixture was stirred at 50° C. for 2 hours. The reaction mixture was added with water (2 mL), and extracted with ethyl acetate (3 mL×3). The organic phases were combined, dried over anhydrous sodium sulfate, and filtered. The filtrate was concentrated under reduced pressure to obtain a crude product. The crude product was purified by preparative TLC (DCM:MeOH=30:1) to obtain compound 26. MS m/z: 453.0 [M+1]⁺.

### Example 27

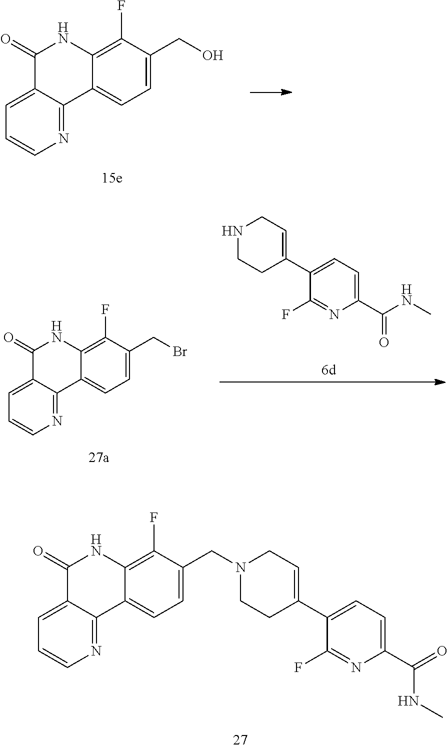 [SMILES: O=c1[nH]c2c(F)c(CO)ccc2c2ncccc12 ; O=c1[nH]c2c(F)c(CBr)ccc2c2ncccc12 ; CNC(=O)c1ccc(C2=CCNCC2)c(F)n1 ; CNC(=O)c1ccc(C2=CCN(Cc3ccc4c([nH]c(=O)c5cccnc54)c3F)CC2)c(F)n1]

### Step 1: Synthesis of Intermediate 27a

Intermediate 15e (61.9 mg, 253.46 μmol) and 1,2-dibromotetrachloroethane (82.54 mg, 253.46 μmol) were dissolved in DCM (2 mL). At 0° C., tributylphosphine (51.28 mg, 253.46 μmol) was added. After the addition was complete, the mixture was stirred at room temperature (20° C.) for 19 hours. The reaction mixture was filtered to obtain the filter cake, which was intermediate 27a. ¹H NMR (400 MHz, DMSO-d₆) δ ppm 12.02 (s, 1H) 9.05-9.13 (m, 1H) 8.61-8.68 (m, 1H) 8.41-8.49 (m, 1H) 7.77 (br d, J=2.01 Hz, 1H) 7.35-7.50 (m, 1H) 4.94 (s, 2H).

### Step 2: Synthesis of Compound 27

Intermediate 6d (21.23 mg, 78.15 μmol, HCl) was dissolved in DMF (2 mL). Triethylamine (19.77 mg, 195.36 μmol) was added, and the mixture was stirred at 25° C. for 0.5 hours. Intermediate 27a (30 mg, 97.68 μmol) and potassium iodide (1.62 mg, 9.77 μmol) were then added, and the mixture was stirred at 50° C. for 2 hours. The reaction mixture was added with 2 mL of water, and extracted three times with 5 mL of a mixed solution (DCM:MeOH=10:1). The organic phases were combined, dried over anhydrous sodium sulfate, and concentrated under reduced pressure to obtain a crude product. The crude product was stirred with EA (0.5 mL) for 10 minutes and filtered to obtain the filter cake, which was compound 27. MS m/z: 462.1 [M+1]⁺. ¹H NMR (400 MHz, DMSO-d₆) δ ppm 11.90 (s, 1H), 9.09 (dd, J=4.82, 1.81 Hz, 1H), 8.65 (br dd, J=8.00, 1.88 Hz, 2H), 8.43 (d, J=8.50 Hz, 1H), 8.05-8.12 (m, 1H), 7.88-7.96 (m, 1H), 7.72 (dd, J=7.88, 4.88 Hz, 1H), 7.39 (br t, J=7.44 Hz, 1H), 6.26 (br s, 1H), 3.80 (s, 2H), 3.21-3.22 (m, 2H), 2.79 (d, J=4.75 Hz, 3H), 2.72-2.76 (m, 4H).

### Example 28

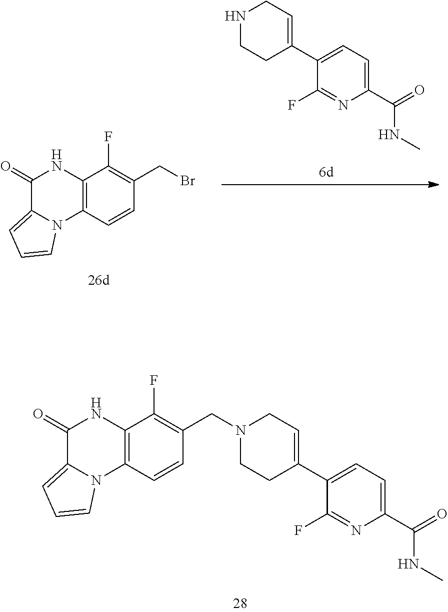 [SMILES: O=c1[nH]c2c(F)c(CBr)ccc2n2cccc12 ; CNC(=O)c1ccc(C2=CCNCC2)c(F)n1 ; CNC(=O)c1ccc(C2=CCN(Cc3ccc4c([nH]c(=O)c5cccn54)c3F)CC2)c(F)n1]

### Step 1: Synthesis of Compound 28

Compound 26d (25 mg, 84.72 μmol) was dissolved in DMF (1 mL). Triethylamine (34.29 mg, 338.86 μmol) was added, and the mixture was stirred at 25° C. for 0.5 hours. Compound 6d (23 mg, hydrochloride) and potassium iodide (7.03 mg, 42.36 μmol) were then added, and the mixture was stirred at 50° C. for 2 hours. The reaction mixture was added with water (2 mL), and extracted with ethyl acetate (3 mL×3). The organic phases were combined, dried over anhydrous sodium sulfate, and filtered. The filtrate was concentrated under reduced pressure to obtain a crude product. The crude product was stirred with 0.5 mL of ethyl acetate for 10 minutes and filtered to obtain the filter cake, which was compound 28. MS m/z: 450.0 [M+1]⁺, ¹H NMR (400 MHz, DMSO-d₆) δ ppm 11.32 (s, 1H), 8.64 (br d, J=4.77 Hz, 1H), 8.20 (s, 1H), 8.01-8.14 (m, 1H), 7.83-7.97 (m, 2H), 7.26 (t, J=7.78 Hz, 1H), 7.09 (d, J=3.01 Hz, 1H), 6.72 (t, J=3.26 Hz, 1H), 6.25 (br s, 1H), 3.72 (s, 2H), 3.32 (s, 2H), 3.13-3.22 (m, 2H), 2.79 (d, J=4.77 Hz, 3H), 2.66-2.75 (m, 2H).

### Example 29

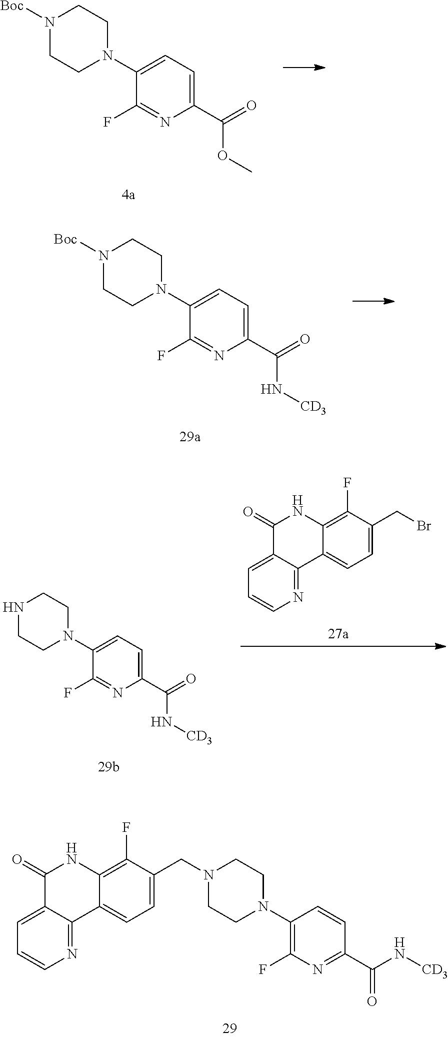 [SMILES: COC(=O)c1ccc(N2CCN(C(=O)OC(C)(C)C)CC2)c(F)n1 ; [2H]C([2H])([2H])NC(=O)c1ccc(N2CCN(C(=O)OC(C)(C)C)CC2)c(F)n1 ; [2H]C([2H])([2H])NC(=O)c1ccc(N2CCNCC2)c(F)n1 ; O=c1[nH]c2c(F)c(CBr)ccc2c2ncccc12 ; [2H]C([2H])([2H])NC(=O)c1ccc(N2CCN(Cc3ccc4c([nH]c(=O)c5cccnc54)c3F)CC2)c(F)n1]

### Step 1: Synthesis of Intermediate 29a

Deuterated methylamine hydrochloride (124.71 mg, 1.77 mmol) was added to H₂O (1 mL). Sodium tert-butoxide (84.95 mg, 884.01 μmol) was then added to the reaction mixture, and the mixture was stirred at 25° C. for 1 hour. A solution of compound 4a (30 mg, 88.40 μmol) in MeOH (2 mL) was added thereto. The reaction mixture was then heated to 60° C. and reacted for 4 hours. The reaction mixture was directly concentrated under reduced pressure to obtain a crude product, which was intermediate 29a. MS m/z: 342.1 [M+1]⁺.

### Step 2: Synthesis of Hydrochloride of Intermediate 29b

Intermediate 29a (20 mg) was dissolved in MeOH (0.6 mL). HCl/dioxane (4 M, 0.2 mL) was added thereto, and the mixture was stirred at 20° C. for 2 hours. The reaction mixture was directly concentrated under reduced pressure to obtain the hydrochloride of intermediate 29b. MS m/z: 242.2 [M+1]⁺.

### Step 3: Synthesis of Compound 29

Intermediate 29b (19.22 mg, hydrochloride) was dissolved in DMF (1 mL). Triethylamine (162.80 μmol, 22.66 μL) was added, and the mixture was stirred at 25° C. for 30 minutes. Compound 27a (25 mg, 81.40 μmol) and potassium iodide (1.35 mg, 8.14 μmol) were then added, and the mixture was heated to 50° C. and stirred for 3 hours. The reaction mixture was added with 2 mL of water, and filtered to obtain the filter cake, which was compound 29. MS m/z: 468.2 [M+1]⁺. ¹H NMR (400 MHz, DMSO-d₆) δ ppm 11.90 (s, 1H), 9.09 (dd, J=4.63, 1.75 Hz, 1H), 8.65 (dd, J=8.00, 1.75 Hz, 1H), 8.43 (d, J=8.13 Hz, 1H), 8.38 (s, 1H), 7.85 (d, J=7.88 Hz, 1H), 7.72 (dd, J=8.00, 4.50 Hz, 1H), 7.57 (dd, J=10.57, 8.19 Hz, 1H), 7.37 (t, J=7.32 Hz, 1H), 3.75 (s, 2H), 3.15-3.22 (m, 4H), 2.62-2.65 (m, 4H).

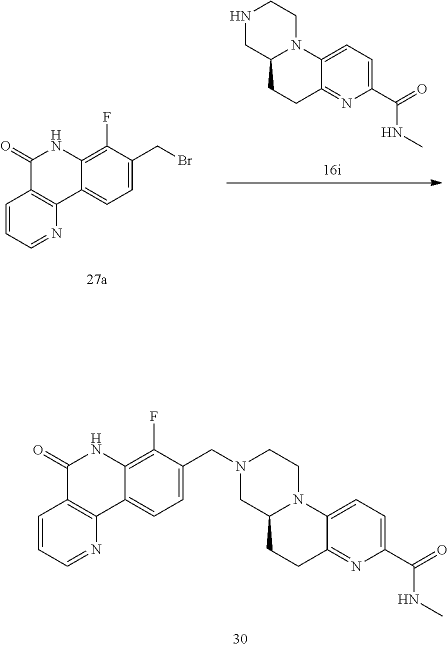 [SMILES: O=c1[nH]c2c(F)c(CBr)ccc2c2ncccc12 ; CNC(=O)c1ccc2c(n1)CC[C@H]1CNCCN21 ; CNC(=O)c1ccc2c(n1)CC[C@H]1CN(Cc3ccc4c([nH]c(=O)c5cccnc54)c3F)CCN21]

### Step 1: Synthesis of Compound 30

Intermediate 16i (29.46 mg, hydrochloride) was dissolved in DMF (2 mL). Triethylamine (52.72 mg, 520.97 μmol) was added, and the reaction mixture was stirred at 25° C. for 0.5 hours. Intermediate 27a (40 mg, 130.24 μmol) and KI (21.62 mg, 130.24 μmol) were then added. The mixture was stirred at 50° C. for 2 hours. The reaction mixture was added with 2 mL of water, and extracted with ethyl acetate (3 mL×3). The organic phase was dried over anhydrous sodium sulfate, filtered, and concentrated under reduced pressure to obtain a crude product. The crude product was stirred with 0.5 mL of ethyl acetate for 10 minutes and filtered to obtain the filter cake. The filter cake was further purified by preparative TLC (DCM:MeOH=20:1) to obtain compound 30. MS m/z: 473.0 [M+1]⁺. ¹H NMR (400 MHz, CDCl₃) δ ppm 8.95-9.02 (m, 1H), 8.73-8.79 (m, 1H), 8.68 (dd, J=8.16, 1.88 Hz, 1H), 8.46 (br d, J=8.03 Hz, 1H), 7.77-7.88 (m, 1H), 7.65-7.75 (m, 1H), 7.48-7.54 (m, 1H), 7.34-7.43 (m, 1H), 6.98 (d, J=8.53 Hz, 1H), 3.63-3.86 (m, 3H), 2.97-3.33 (m, 2H), 2.92 (d, J=5.27 Hz, 3H), 2.82-2.89 (m, 2H), 2.03-2.43 (m, 1H), 1.90-1.94 (m, 1H), 1.68-1.83 (m, 1H), 1.33-1.39 (m, 1H), 1.23-1.31 (m, 2H).

### Example 31

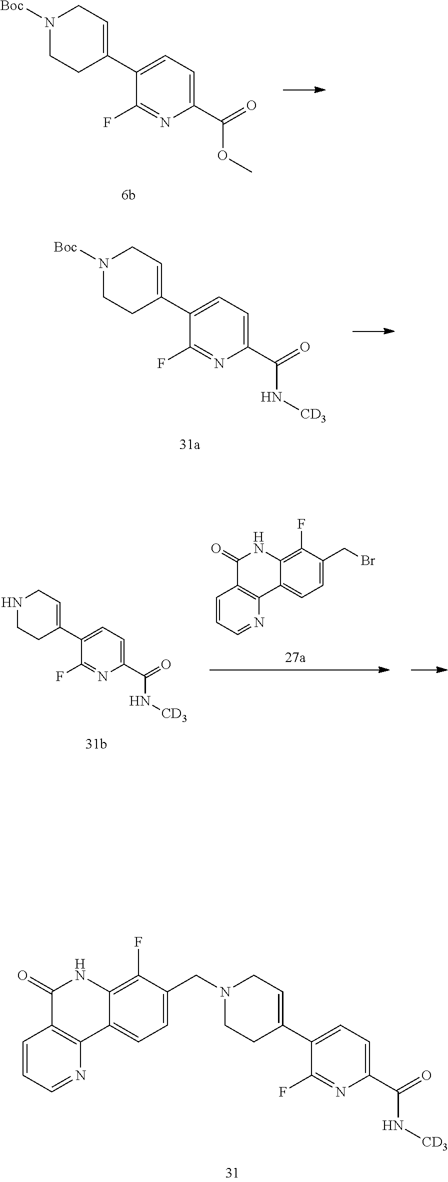 [SMILES: COC(=O)c1ccc(C2=CCN(C(=O)OC(C)(C)C)CC2)c(F)n1 ; [2H]C([2H])([2H])NC(=O)c1ccc(C2=CCN(C(=O)OC(C)(C)C)CC2)c(F)n1 ; [2H]C([2H])([2H])NC(=O)c1ccc(C2=CCNCC2)c(F)n1 ; O=c1[nH]c2c(F)c(CBr)ccc2c2ncccc12 ; [2H]C([2H])([2H])NC(=O)c1ccc(C2=CCN(Cc3ccc4c([nH]c(=O)c5cccnc54)c3F)CC2)c(F)n1]

### Step 1: Synthesis of Intermediate 31a

Deuterated methylamine hydrochloride (167.77 mg, 2.38 mmol) was added to H₂O (1 mL). Potassium tert-butoxide (133.44 mg, 1.19 mmol) was then added to the reaction mixture, and the mixture was stirred at 25° C. for 1 hour. A solution of compound 6b (40 mg, 118.92 μmol) in MeOH (2 mL) was added thereto. The reaction mixture was then heated to 60° C. and reacted for 16 hours. The reaction mixture was directly concentrated under reduced pressure to obtain a crude product, which was intermediate 31a. MS m/z: 339.1 [M+1]⁺.

### Step 2: Synthesis of Hydrochloride of Intermediate 31b

Intermediate 31a was dissolved in MeOH (1 mL). HCl/dioxane (4 M, 99.74 μL) was added thereto, and the mixture was stirred at 25° C. for 2 hours. The reaction mixture was directly concentrated under reduced pressure to obtain the hydrochloride of intermediate 31b. MS m/z: 238.9 [M+1]⁺.

### Step 3: Synthesis of Compound 31

Intermediate 31b (15 mg, hydrochloride) was dissolved in DMF (1 mL). Triethylamine (27.62 mg, 272.99 μmol, 38.00 μL) was added, and the reaction mixture was stirred at 25° C. for 0.5 hours. Intermediate 27a (20.96 mg, 68.25 μmol) and potassium iodide (5.66 mg, 34.12 μmol) were then added. The mixture was stirred at 50° C. for 2 hours. After cooling, the reaction mixture was added with 2 mL of water, resulting in the precipitation of a solid. The mixture was filtered to obtain the filter cake, which was compound 31. MS m/z: 465.1 [M+1]⁺, ¹H NMR (400 MHz, DMSO-d₆) δ ppm 11.89 (br d, J=1.76 Hz, 1H), 9.02-9.14 (m, 1H), 8.56-8.70 (m, 2H), 8.43 (d, J=8.03 Hz, 1H), 8.04-8.14 (m, 1H), 7.89-7.96 (m, 1H), 7.72 (dd, J=8.03, 4.52 Hz, 1H), 7.39 (t, J=7.53 Hz, 1H), 6.26 (br s, 1H), 3.80 (s, 2H), 3.31 (s, 2H), 3.18-3.24 (m, 2H), 2.74 (br t, J=5.27 Hz, 2H).

### Example 32

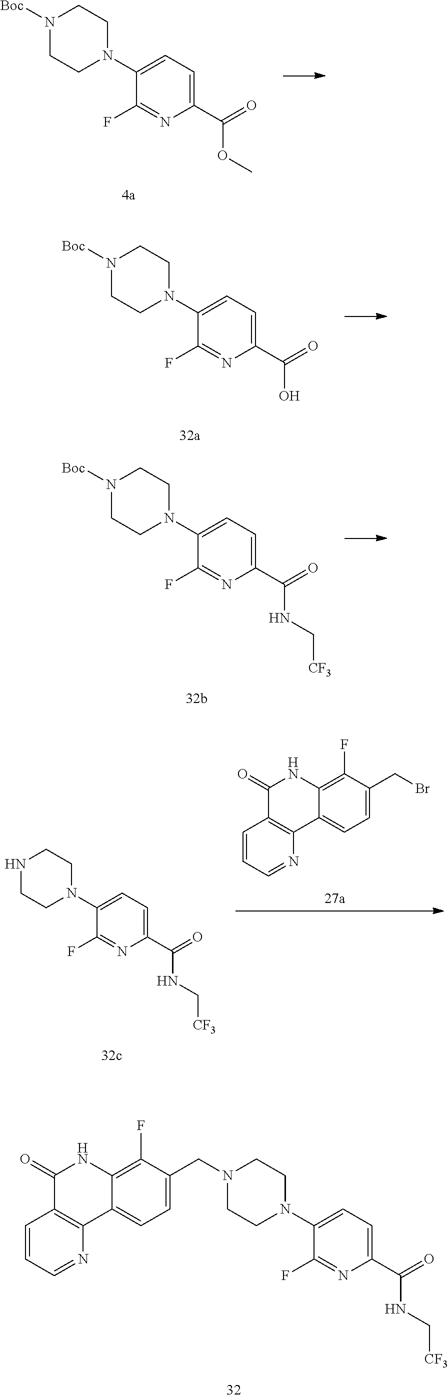 [SMILES: COC(=O)c1ccc(N2CCN(C(=O)OC(C)(C)C)CC2)c(F)n1 ; CC(C)(C)OC(=O)N1CCN(c2ccc(C(=O)O)nc2F)CC1 ; CC(C)(C)OC(=O)N1CCN(c2ccc(C(=O)NCC(F)(F)F)nc2F)CC1 ; O=C(NCC(F)(F)F)c1ccc(N2CCNCC2)c(F)n1 ; O=c1[nH]c2c(F)c(CBr)ccc2c2ncccc12 ; O=C(NCC(F)(F)F)c1ccc(N2CCN(Cc3ccc4c([nH]c(=O)c5cccnc54)c3F)CC2)c(F)n1]

### Step 1: Synthesis of Intermediate 32a

Intermediate 4a (0.2 g, 589.34 μmol) was added to a mixture of THF (5 mL) and H₂O (1 mL). With stirring, lithium hydroxide monohydrate (123.65 mg, 2.95 mmol) was added thereto, and the reaction mixture was stirred at 20° C. for 4 hours. The pH of the reaction mixture was adjusted to 3-4 using dilute hydrochloric acid (1 mol/L). The mixture was extracted with ethyl acetate (5 mL×2). The organic phases were combined, dried over anhydrous sodium sulfate, concentrated under reduced pressure at 45° C. to obtain intermediate 32a. MS m/z: 347.9 [M+23]⁺.

### Step 2: Synthesis of Intermediate 32b

Under a nitrogen atmosphere, intermediate 32a (0.1 g, 307.38 μmol), 2,2,2-trifluoroethylamine (45.67 mg, 461.06 μmol), 1H-benzotriazol-1-yltetramethyluronium tetrafluoroborate (197.39 mg, 614.75 μmol), and diisopropylethylamine (119.18 mg, 922.13 μmol) were added to DMF (2 mL). The reaction mixture was stirred at 40° C. for 4 hours. The reaction mixture was concentrated under reduced pressure to obtain a crude product. The crude product was purified by preparative TLC (PE/EA=2/1) to obtain intermediate 32b. MS m/z: 351.1 [M−56+1]⁺.

### Step 3: Synthesis of Hydrochloride of Intermediate 32c

Intermediate 32b (57.66 mg, 141.88 μmol) was added to MeOH (2 mL). With stirring, HCl/dioxane (4 M, 191.54 μL) was added thereto, and the reaction mixture was stirred at 25° C. for 2 hours. The reaction mixture was concentrated under reduced pressure at 45° C. to obtain a crude product, which was the hydrochloride of intermediate 32c. MS m/z: 306.9 [M+1]⁺.

### Step 4: Synthesis of Compound 32

Triethylamine (156.29 μmol, 21.75 μL) was added to a solution of intermediate 32c (26.78 mg, hydrochloride) in DMF (1 mL). The mixture was stirred at 20° C. for 0.5 hours. Intermediate 27a (30 mg, 78.15 μmol) and potassium iodide (1.30 mg, 7.81 μmol) were added thereto. The reaction mixture was stirred at 50° C. for 2 hours, and filtered. The filtrate was concentrated under reduced pressure at 60° C. to obtain a crude product. The crude product was purified by preparative TLC (EA: 100%) to obtain compound 32. MS m/z: 555.1 [M+23]⁺. ¹H NMR (400 MHz, CDCl₃) δ ppm 8.92-9.04 (m, 1H), 8.62-8.76 (m, 2H), 8.44 (br d, J=8.25 Hz, 1H), 7.93 (d, J=8.63 Hz, 1H), 7.71 (s, 1H), 7.45-7.55 (m, 1H), 7.24-7.35 (m, 1H), 3.92-4.07 (m, 2H), 3.68-3.78 (m, 2H), 3.21 (m, 4H), 2.66 (m, 4H).

### Example 33

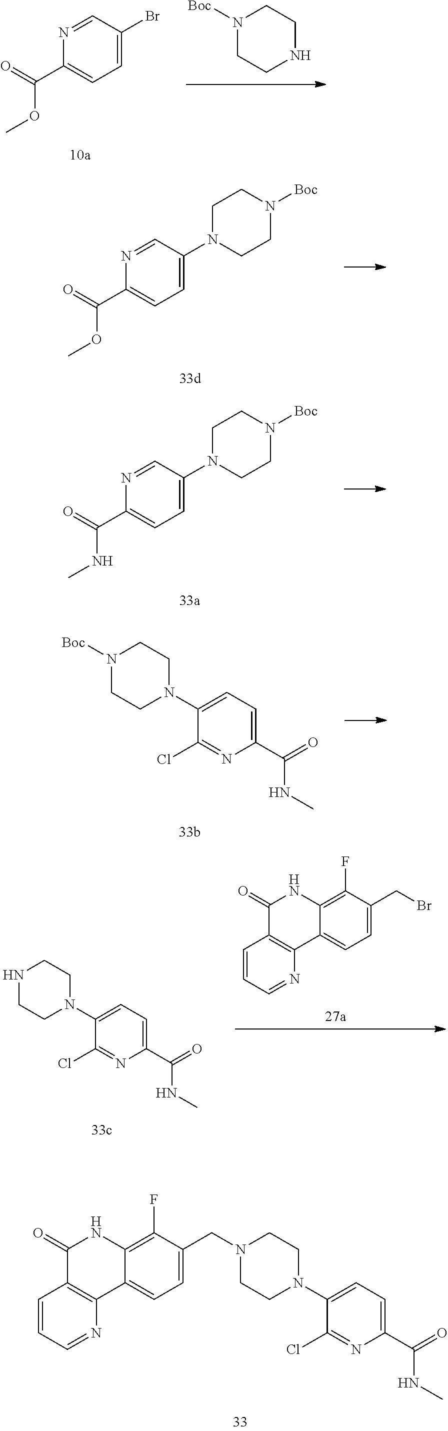

### Step 1: Synthesis of Intermediate 33d

10a (3 g, 13.89 mmol) was dissolved in toluene (30 mL). 1-(tert-Butoxycarbonyl) piperazine (2.59 g, 13.89 mmol), RuPhos (648.01 mg, 1.39 mmol), Pd₂(dba)₃ (381.49 mg, 416.61 μmol), and Cs₂CO₃ (13.57 g, 41.66 mmol) were added. The reaction mixture was stirred under a nitrogen atmosphere at 100° C. for 16 hours. The reaction mixture was filtered, and the filtrate was concentrated under reduced pressure to obtain a crude product. The crude product was purified by column chromatography (eluent: PE:EA=100:0 to 90:10 to 75:25, V/V) to obtain intermediate 33d. MS m/z: 321.9 [M+1]⁺.

### Step 2: Synthesis of Intermediate 33a

At 25° C., intermediate 33d (1 g, 3.11 mmol) was added to a solution of methylamine in ethanol (322.13 mg, 3.11 mmol). The reaction mixture was stirred at 25° C. for 1 hour. The reaction mixture was concentrated under reduced pressure to obtain a crude product. The crude product was dissolved in 10 mL of dichloromethane and washed with 10 ml of water. The mixture was extracted, and the phases were separated to collect the organic phase. The organic phase was dried over anhydrous sodium sulfate, and filtered. The filtrate was concentrated under reduced pressure to obtain intermediate 33a. MS m/z: 320.9 [M+1]⁺. ¹H NMR (400 MHz, CDCl₃) δ ppm 8.16 (d, J=2.25 Hz, 1H), 8.08 (d, J=8.76 Hz, 1H), 7.85 (br s, 1H), 7.24 (dd, J=2.50, 8.75 Hz, 1H), 3.56-3.69 (m, 4H), 3.30 (br d, J=4.63 Hz, 4H), 3.01 (d, J=5.00 Hz, 3H), 1.49 (s, 9H).

### Step 3: Synthesis of Intermediate 33b

Compound 33a (0.3 g, 936.37 μmol) was dissolved in DMF (1.5 mL). N-Chlorosuccinimide (137.54 mg, 1.03 mmol) was added at 20° C., and the mixture was stirred for 14 hours. The reaction mixture was added with water (5 mL) and extracted with ethyl acetate (20 mL×2). The organic phases were combined, washed with water (20 mL), then dried over anhydrous sodium sulfate, and filtered. The filtrate was concentrated under reduced pressure to obtain compound 33b. MS m/z: 355.1, 357.1 [M+1]⁺. ¹H NMR (400 MHz, CDCl₃) δ ppm 8.10 (d, J=4.2 Hz, 1H), 7.69 (br, s, 1H), 7.40 (d, J=4.2 Hz, 1H), 3.63-3.66 (m, 4H), 3.09-3.15 (m, 4H), 3.03 (d, J=2.4 Hz, 3H), 1.51 (s, 9H).

### Step 4: Synthesis of Hydrochloride of Intermediate 33c

Intermediate 33b (350 mg, 986.39 μmol) was dissolved in MeOH (6 mL). HCl/dioxane (4 M, 6 mL) was added, and the mixture was stirred at 20° C. for 4 hours. The reaction mixture was concentrated under reduced pressure at 60° C. to obtain the hydrochloride of intermediate 33c. MS m/z: 255.1, 257.1 [M+1]⁺.

### Step 5: Synthesis of Compound 33

Intermediate 27a (20 mg, 65.12 μmol), intermediate 33c (18.96 mg, hydrochloride), potassium iodide (5.41 mg, 32.56 μmol), and triethylamine (26.36 mg, 260.49 μmol) were sequentially added to DMF (1 mL). The reaction mixture was heated to 50° C. and stirred for 12 hours. The reaction mixture was added with water (3 mL), and extracted with ethyl acetate (10 mL×2). The organic phases were combined, dried over anhydrous sodium sulfate, and filtered. The filtrate was concentrated under reduced pressure to obtain a crude product. The crude product was stirred with 1.2 mL of methanol for 10 minutes, and the mixture was filtered to obtain the filter cake, which was compound 33. MS m/z: 481.1 [M+1]⁺. ¹H NMR (400 MHz, DMSO-d₆) δ ppm 11.90 (br s, 1H), 9.08-9.09 (m, 1H), 8.65 (d, J=2.40 Hz, 2H), 8.43 (d, J=4.20 Hz, 1H), 7.93 (d, J=3.92 Hz, 1H), 7.66-7.74 (m, 2H), 7.33-7.39 (m, 1H), 3.77 (s, 2H), 3.05-3.15 (m, 4H), 2.79 (d, J=2.40 Hz, 3H), 2.65-2.68 (m, 4H).

### Example 34

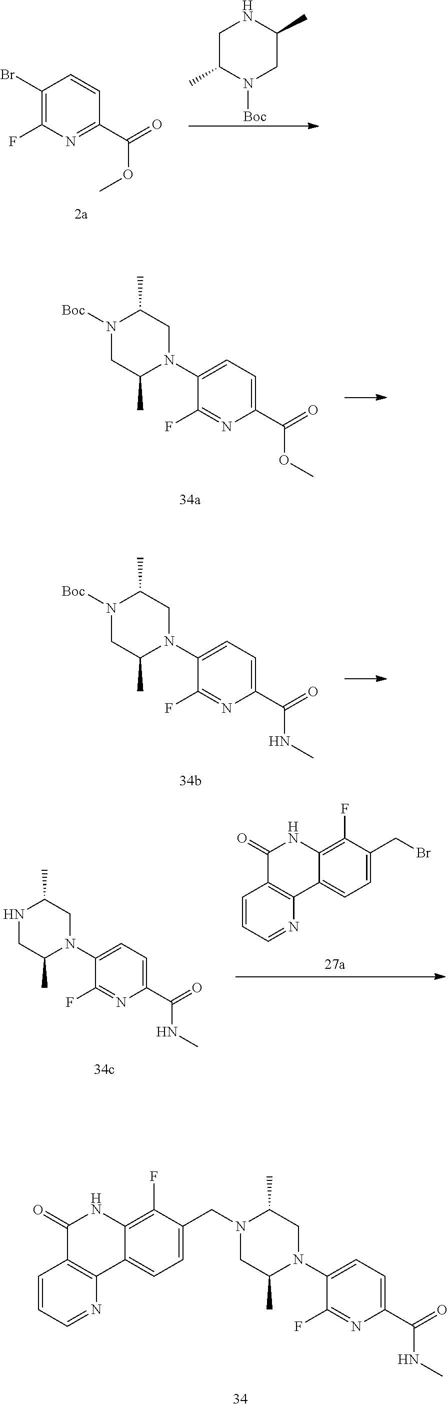

### Step 1: Synthesis of Intermediate 34a

Intermediate 2a (200 mg, 854.62 μmol) and (2R,5S)-1-tert-butoxycarbonyl-2,5-dimethylpiperazine (183.15 mg, 854.62 μmol) were dissolved in toluene (5 mL). RuPhos (79.76 mg, 170.92 μmol), Cs₂CO₃ (556.90 mg, 1.71 mmol), and Pd₂(dba)₃ (78.26 mg, 85.46 μmol) were sequentially added. The reaction mixture was stirred under a nitrogen atmosphere at 100° C. for 16 hours. The reaction mixture was filtered, and the filtrate was concentrated under reduced pressure to obtain a crude product. The crude product was purified by column chromatography (eluent: PE:EA=100:0 to 90:10 to 75:25, v/v) to obtain intermediate 34a. MS m/z: 368.0 [M+1]⁺, ¹H NMR (400 MHz, CDCl₃) δ ppm 7.97 (dd, J=8.03, 1.25 Hz, 1H), 7.20 (dd, J=10.04, 8.03 Hz, 1H), 4.38-4.45 (m, 1H), 4.05 (br d, J=2.26 Hz, 1H), 3.97 (s, 3H), 3.74-3.83 (m, 1H), 3.47-3.52 (m, 2H), 3.10 (d, J=13.05 Hz, 1H), 1.51 (s, 9H), 1.29-1.32 (m, 3H), 1.12 (d, J=5.27 Hz, 3H).

### Step 2: Synthesis of Intermediate 34b

Intermediate 34a (187.4 mg, 510.05 μmol) was dissolved in EtOH (2 mL). A solution of methylamine in ethanol (792.03 mg, 7.63 mmol, 30%) was added, and the mixture was stirred at 25° C. for 16 hours. The reaction mixture was directly concentrated under reduced pressure to obtain intermediate 34b. MS m/z: 367.0 [M+1]⁺, ¹H NMR (400 MHz, CDCl₃) δ ppm 7.99 (dd, J=8.03, 1.26 Hz, 1H), 7.47-7.54 (m, 1H), 7.24 (dd, J=10.16, 8.16 Hz, 1H), 4.39-4.52 (m, 1H), 3.93-4.02 (m, 1H), 3.73-3.78 (m, 1H), 3.50 (dd, J=12.17, 3.89 Hz, 2H), 2.95-3.02 (m, 4H), 1.51 (s, 9H), 1.31 (d, J=6.78 Hz, 3H), 1.09 (d, J=6.53 Hz, 3H).

### Step 3: Synthesis of hydrochloride of intermediate 34c

Intermediate 34b (170 mg, 463.94 μmol) was dissolved in MeOH (2 mL). HCl/dioxane (4 M, 0.62 mL) was added, and the mixture was stirred at 25° C. for 3 hours. The reaction mixture was concentrated under reduced pressure to obtain the hydrochloride of intermediate 34c. MS m/z: 266.9 [M+1]⁺.

### Step 4: Synthesis of Trifluoroacetate of Compound 34

Intermediate 34c (26.51 mg, hydrochloride) was dissolved in DMF (1 mL). Triethylamine (39.54 mg, 390.73 μmol) was added, and the mixture was stirred at 25° C. for 0.5 hours. Intermediate 27a (30 mg, 97.68 μmol) and potassium iodide (8.11 mg, 48.84 μmol) were then added, and the mixture was stirred at 50° C. for 2 hours. The reaction mixture was filtered, and the filtrate was separated by preparative HPLC (column: Xtimate C18, 100× 30 mm×3 μm; mobile phase: [water (trifluoroacetic acid)-acetonitrile]; gradient: 14%-44% acetonitrile) to obtain the trifluoroacetate of compound 34. MS m/z: 493.1 [M+1]⁺. ¹H NMR (400 MHz, CD₃OD) δ ppm 9.11 (dd, J=4.52, 1.76 Hz, 1H), 8.78 (dd, J=8.03, 1.76 Hz, 1H), 8.69 (d, J=8.53 Hz, 1H), 8.01 (d, J=7.03 Hz, 1H), 7.85-7.92 (m, 1H), 7.76 (dd, J=8.03, 4.52 Hz, 1H), 7.53-7.56 (m, 1H), 4.98 (br d, J=16.56 Hz, 2H), 4.47 (br d, J=14.31 Hz, 1H), 3.74-3.90 (m, 1H), 3.49-3.57 (m, 2H), 3.07-3.20 (m, 2H), 2.95 (s, 3H), 1.65 (d, J=6.27 Hz, 3H), 1.02 (d, J=6.02 Hz, 3H).

### Example 35

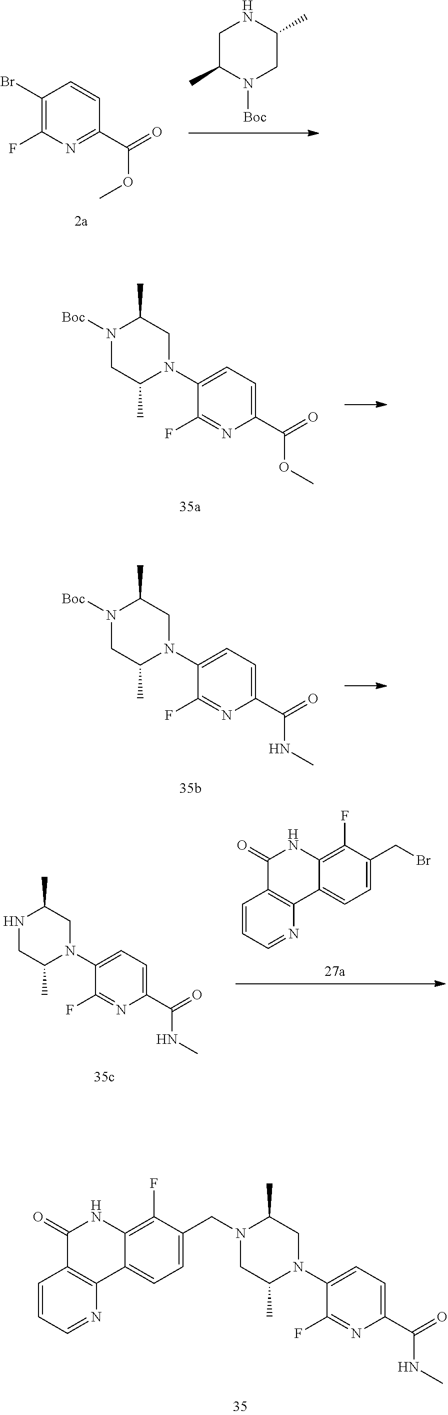

### Step 1: Synthesis of Intermediate 35a

(2S,5R)-1-tert-Butoxycarbonyl-2,5-dimethylpiperazine (180.00 mg, 839.93 μmol) and intermediate 2a (196.56 mg, 839.93 μmol) were dissolved in toluene (10 mL). Cs₂CO₃ (821.00 mg, 2.52 mmol), RuPhos (39.19 mg, 83.99 μmol), and Pd₂(dba)₃ (38.46 mg, 42.00 μmol) were sequentially added. The reaction mixture was stirred under a nitrogen atmosphere at 100° C. for 16 hours. The reaction mixture was filtered while hot, and the filtrate was concentrated under reduced pressure to obtain a crude product. The crude product was added with 2 mL of a mixture of petroleum ether and ethyl acetate (PE/EA=5/1) and stirred at 20° C. for 0.5 hours. The mixture was subjected to suction filtration, and the filter cake was dried under reduced pressure to obtain intermediate 35a. MS m/z: 368.1 [M+1]⁺.

### Step 2: Synthesis of Intermediate 35b

Intermediate 35a (100 mg, 272.17 μmol) was dissolved in EtOH (2 mL). A solution of methylamine in ethanol (1.13 g, 10.89 mmol, 30%) was added, and the mixture was stirred at room temperature (25° C.) for 16 hours. The reaction mixture was concentrated under reduced pressure to obtain intermediate 35b. MS m/z: 367.1 [M+1]⁺.

### Step 3: Synthesis of Hydrochloride of Intermediate 35c

Intermediate 35b (100 mg, 272.90 μmol) was dissolved in MeOH (2 mL). HCl/dioxane (4 M, 0.38 mL) was added, and the mixture was stirred at 25° C. for 2 hours. The reaction mixture was concentrated under reduced pressure to obtain the hydrochloride of intermediate 35c. MS m/z: 267.0 [M+1]⁺.

### Step 4: Synthesis of Trifluoroacetate of Compound 35

Triethylamine (15.8.2 mg, 156.29 μmol) was added to a solution of intermediate 35c (23.66 mg, hydrochloride) in DMF (1 mL). The mixture was stirred at 20° C. for 0.5 hours. Intermediate 27a (30 mg, 78.15 μmol) and KI (1.30 mg, 7.815 μmol) were then added thereto, and the reaction mixture was stirred at 50° C. for 0.5 hours. The reaction mixture was concentrated under reduced pressure to obtain a crude product. The crude product was purified by preparative HPLC (column: Xtimate C18, 100×30 mm×3 um; mobile phase: [water (trifluoroacetic acid)-acetonitrile]; gradient: 14%-44% acetonitrile) to obtain the trifluoroacetate of compound 35. MS m/z: 493.2 [M+1]⁺¹H NMR (400 MHz, CD₃OD) δ ppm 9.11 (dd, J=4.52, 1.76 Hz, 1H), 8.77 (dd, J=8.03, 1.76 Hz, 1H), 8.69 (d, J=7.78 Hz, 1H), 8.01 (d, J=7.78 Hz, 1H), 7.86-7.93 (m, 1H), 7.76 (dd, J=8.03, 4.77 Hz, 1H), 7.55 (dd, J=8.28, 6.78 Hz, 1H), 4.91-5.02 (m, 2H), 4.50 (br d, J=13.55 Hz, 1H), 3.75-3.83 (m, 1H), 3.48-3.58 (m, 2H), 3.10-3.23 (m, 2H), 2.94 (s, 3H), 1.66 (d, J=6.53 Hz, 3H), 1.01 (d, J=6.02 Hz, 3 H).

### Example 36

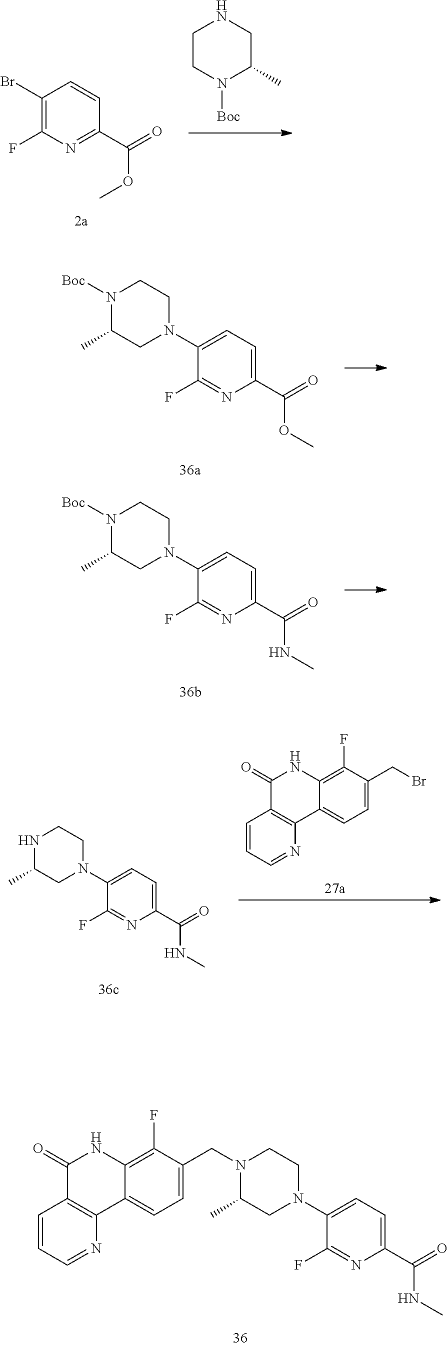

### Step 1: Synthesis of Intermediate 36a

(S)-1-N-tert-Butoxycarbonyl-2-methylpiperazine (128.37 mg, 640.96 μmol) and intermediate 2a (150 mg, 640.96 μmol) were dissolved in toluene (10 mL). RuPhos (59.82 mg, 128.19 μmol), Cs₂CO₃ (417.68 mg, 1.28 mmol), and Pd₂(dba)₃ (58.69 mg, 64.10 μmol) were sequentially added. The reaction mixture was stirred under a nitrogen atmosphere at 100° C. for 16 hours. The reaction mixture was filtered, and the filtrate was concentrated under reduced pressure to obtain a crude product. The crude product was purified by column chromatography (eluent: PE:EA=90:10 to 75:25, v/v) to obtain intermediate 36a. MS m/z: 354.1 [M+1]⁺. ¹H NMR (400 MHz, CDCl₃) δ ppm 7.99 (dd, J=8.03, 1.00 Hz, 1H), 7.26 (dd, J=10.04, 8.28 Hz, 1H), 4.38 (br d, J=5.02 Hz, 1H), 4.02 (br s, 1H), 3.98 (s, 3H), 3.44-3.57 (m, 2H), 3.31 (td, J=12.80, 3.26 Hz, 1H), 3.02 (dd, J=11.92, 3.64 Hz, 1H), 2.84-2.94 (m, 1H), 1.51 (s, 9H), 1.35 (d, J=6.78 Hz, 3H).

### Step 2: Synthesis of Intermediate 36b

Intermediate 36a (100 mg, 272.17 μmol) was dissolved in EtOH (2 mL). A solution of methylamine in ethanol (0.65 g, 6.29 mmol, 30%) was added, and the mixture was stirred at room temperature (25° C.) for 16 hours. The reaction mixture was directly concentrated under reduced pressure to obtain intermediate 36b. MS m/z: 353.1 [M+1]⁺. ¹H NMR (400 MHz, CDCl₃) δ ppm 7.94-8.06 (m, 1H), 7.52 (br d, J=4.27 Hz, 1H), 7.29-7.34 (m, 1H), 4.39 (br s, 1H), 4.01 (br d, J=13.80 Hz, 1H), 3.38-3.49 (m, 2H), 3.30 (td, J=12.74, 3.39 Hz, 1H), 3.02 (d, J=5.02 Hz, 3H), 2.97 (dd, J=11.80, 3.51 Hz, 1H), 2.81-2.89 (m, 1H), 1.51 (s, 9H), 1.36 (d, J=6.78 Hz, 3H).

### Step 3: Synthesis of Hydrochloride of Intermediate 36c

Intermediate 36b (51 mg, 428.49 μmol) was dissolved in MeOH (2 mL). HCl/dioxane (4 M, 0.54 mL) was added, and the mixture was stirred at 25° C. for 2 hours. The reaction mixture was concentrated under reduced pressure to obtain the hydrochloride of intermediate 36c. MS m/z: 253.0 [M+1]⁺.

### Step 4: Synthesis of Compound 36

Triethylamine (32.95 mg, 325.61 μmol) was added to a solution of intermediate 36c (21.18 mg, hydrochloride) in DMF (1 mL). The mixture was stirred at 20° C. for 0.5 hours. Intermediate 27a (25 mg, 81.40 μmol) and KI (6.76 mg, 40.70 μmol) were then added thereto, and the reaction mixture was stirred at 50° C. for 2 hours. The reaction mixture was added with water (2 mL), and extracted with ethyl acetate (3 mL×3). The organic phases were combined, dried over anhydrous sodium sulfate, and filtered. The filtrate was concentrated under reduced pressure to obtain a crude product. The crude product was stirred with 1 mL of methanol for 10 minutes, and the mixture was filtered to obtain the filter cake, which was compound 36. MS m/z: 479.0 [M+1]⁺. ¹H NMR (400 MHz, DMSO-d₆) δ ppm 11.87 (s, 1H), 9.08 (dd, J=4.64, 1.88 Hz, 1H), 8.64 (dd, J=8.03, 1.76 Hz, 1H), 8.34-8.49 (m, 2H), 7.80-7.89 (m, 1H), 7.71 (dd, J=8.03, 4.77 Hz, 1H), 7.56 (dd, J=10.54, 8.28 Hz, 1H), 7.33-7.44 (m, 1H), 4.12 (d, J=13.55 Hz, 1H), 3.50 (d, J=13.80 Hz, 1H), 3.36-3.43 (m, 1H), 2.87-2.98 (m, 1H), 2.74-2.86 (m, 5H), 2.65-2.73 (m, 1H) 2.28-2.47 (m, 2H) 1.22 (d, J=6.02 Hz, 3H).

### Example 37

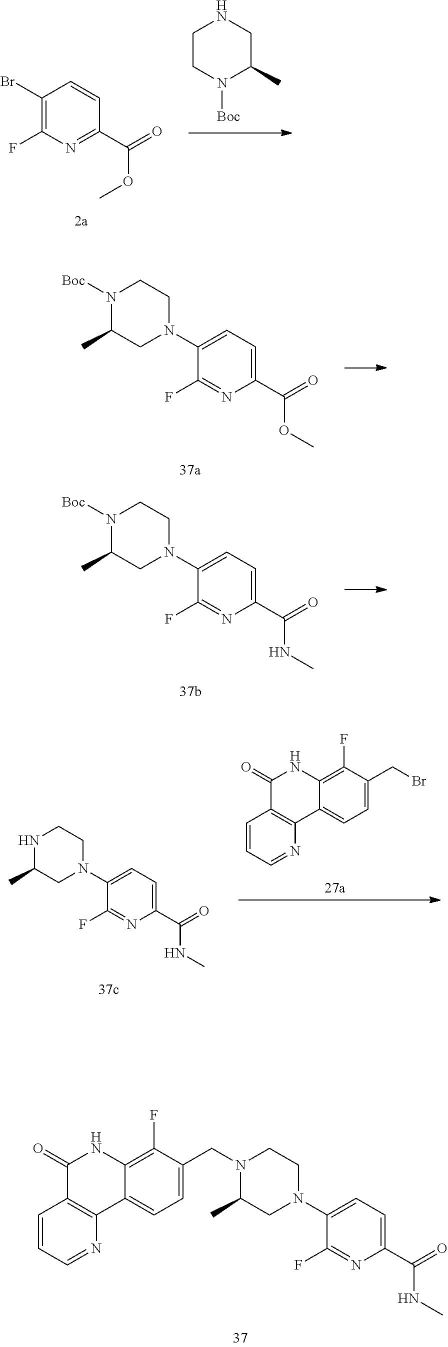

### Step 1: Synthesis of Intermediate 37a

(R)-1-N-tert-Butoxycarbonyl-2-methylpiperazine (0.13 g, 649.10 μmol) and intermediate 2a (151.90 mg, 649.10 μmol) were dissolved in toluene (10 mL). Cs₂CO₃ (634.47 mg, 1.95 mmol), RuPhos (30.29 mg, 64.91 μmol), and Pd₂(dba)₃ (29.72 mg, 32.46 μmol) were sequentially added. The reaction mixture was stirred under a nitrogen atmosphere at 100° C. for 16 hours. The reaction mixture was filtered while hot, and the filtrate was concentrated under reduced pressure to obtain a crude product. The crude product was stirred with 2 mL of a mixture of PE and EA (PE/EA=5/1) at 20° C. for 0.5 hours. The mixture was subjected to suction filtration under reduced pressure, and the filter cake was dried under reduced pressure to obtain intermediate 37a. MS m/z: 376.1 [M+23]⁺.

### Step 2: Synthesis of Intermediate 37b

Intermediate 37a (96.18 mg, 272.17 μmol) was dissolved in EtOH (2 mL). A solution of methylamine in ethanol (1.13 g, 10.89 mmol, 30%) was added, and the mixture was stirred at 20° C. for 16 hours. The reaction mixture was concentrated under reduced pressure to obtain intermediate 37b. MS m/z: 375.1 [M+23]⁺.

### Step 3: Synthesis of Hydrochloride of Intermediate 37c

Intermediate 37b (100 mg, 283.77 μmol) was dissolved in MeOH (2 mL). HCl/dioxane (4 M, 0.38 mL) was added, and the mixture was stirred at 25° C. for 2 hours. The reaction mixture was concentrated under reduced pressure to obtain the hydrochloride of intermediate 37c. MS m/z: 253.0 [M+1]⁺.

### Step 4: Synthesis of Compound 37

Triethylamine (10.54 mg, 104.19 μmol) was added to a solution of intermediate 37c (15.04 mg, hydrochloride) in DMF (1 mL). The mixture was stirred at 20° C. for 0.5 hours. Intermediate 27a (20 mg, 52.10 μmol) and KI (864.82 μg, 5.21 μmol) were then added thereto, and the reaction mixture was stirred at 50° C. for 2 hours. The reaction mixture was concentrated under reduced pressure to obtain a crude product. The crude product was purified by preparative TLC (EA, 100%) to obtain compound 37. MS m/z: 479.1 [M+1]⁺.

### Example 38

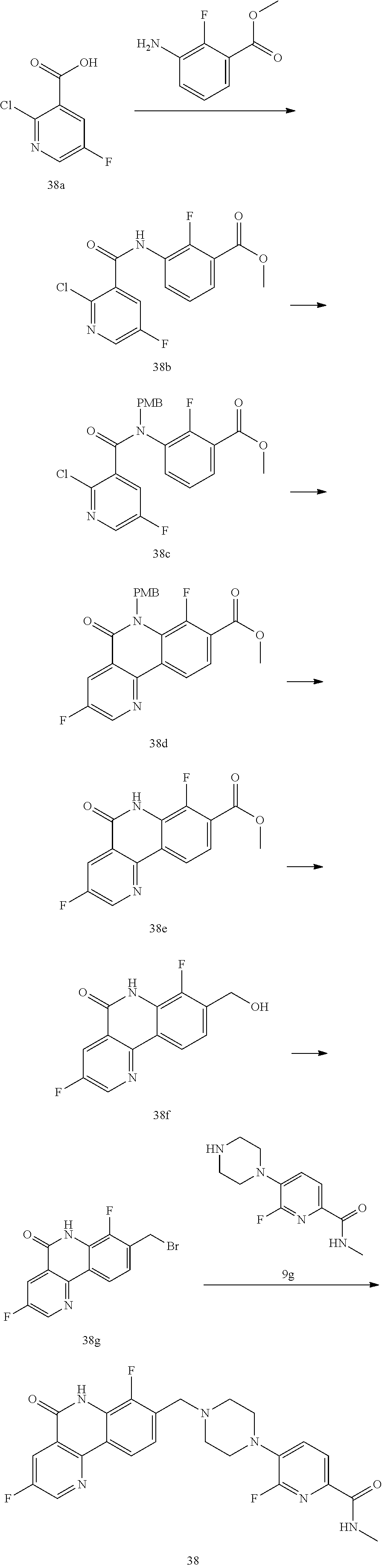

### Step 1: Synthesis of Intermediate 38b

Compound 38a (1 g, 5.70 mmol), methyl 3-amino-2-fluorobenzoate (867.23 mg, 5.13 mmol), and pyridine (2.70 g, 34.18 mmol, 2.76 mL) were added to DCM (10 mL). At 0 to 5° C., phosphorus oxychloride (960.81 mg, 6.27 mmol) was added, and the mixture was stirred at the same temperature for 2 hours. At 0 to 5° C., the reaction mixture was added with 10 mL of water, and extracted with DCM (10 mL×2). The organic phases were combined and concentrated under reduced pressure to obtain a crude product. The crude product was purified by column chromatography (eluent: PE:EA=100:0 to 90:10, v/v) to obtain intermediate 38b. MS m/z: 327.0, 329.0 [M+1]⁺, ¹H NMR (400 MHz, CDCl₃) δ ppm 8.91 (br s, 1H), 8.64-8.72 (m, 1H), 8.45 (d, J=3.00 Hz, 1H), 8.11 (dd, J=7.75, 3.00 Hz, 1H), 7.75-7.80 (m, 1H), 7.29-7.34 (m, 1H), 3.98 (s, 3H).

### Step 2: Synthesis of Intermediate 38c

Intermediate 38b (1.3 g, 3.98 mmol) and potassium carbonate (1.65 g, 11.94 mmol) were added to DMF (15 mL). p-Methoxybenzyl chloride (747.85 mg, 4.78 mmol) was then added, and the mixture was heated to 90° C. and stirred for 2 hours. The reaction mixture was filtered. The filtrate was added with 15 mL of water, and extracted with EA (30 mL×2). The organic phases were combined and concentrated under reduced pressure to obtain a crude product. The crude product was purified by column chromatography (eluent: PE:EA=100:0 to 75:25, v/v) to obtain intermediate 38c. MS m/z: 447.1, 449.1 [M+1]⁺.

### Step 3: Synthesis of Intermediate 38d

Under a nitrogen atmosphere, intermediate 38c (300 mg, 671.40 μmol), tributylphosphine (135.83 mg, 671.40 μmol), 1,3-bis(diphenylphosphino) propane (110.76 mg, 268.56 μmol), Pd(OAc)₂ (60.29 mg, 268.56 μmol), and potassium carbonate (185.58 mg, 1.34 mmol) were added to DMF (8 mL). The reaction was carried out at 120° C. under microwave irradiation for 10 minutes. The reaction mixture was filtered. The filter cake was washed with EA (30 mL), and the filtrate was concentrated to obtain a crude product. The crude product was purified by column chromatography (eluent: PE:EA=100:0 to 90:10, v/v) to obtain intermediate 38d. MS m/z: 411.0 [M+1]⁺. ¹H NMR (400 MHz, DMSO-d₆) δ ppm 9.22 (d, J=2.88 Hz, 1H), 8.69 (d, J=8.63 Hz, 1H), 8.51 (dd, J=8.38, 2.88 Hz, 1H), 7.80 (dd, J=8.38, 6.13 Hz, 1H), 7.15 (d, J=8.50 Hz, 2H), 6.87 (d, J=8.76 Hz, 2H), 5.60 (s, 2H), 3.86 (s, 3H), 3.71 (s, 3H).

### Step 4: Synthesis of Intermediate 38e

Intermediate 38d (140 mg, 341.16 μmol) was added to a mixed solution of trifluoroacetic acid (1 mL) and trifluoromethanesulfonic acid (0.2 mL). The reaction mixture was stirred at 25° C. for 1 hour. The reaction mixture was added with water (4 mL), resulting in the precipitation of a solid. The mixture was filtered to obtain the filter cake, which was intermediate 38e. ¹H NMR (400 MHz, DMSO-d₆) δ ppm 12.23 (br s, 1H), 9.18 (d, J=2.26 Hz, 1H), 8.44-8.46 (m, 2H), 7.70-7.84 (m, 1H), 3.93 (s, 3H).

### Step 5: Synthesis of Intermediate 38f

Under a nitrogen atmosphere at 0° C., LiAlH₄ (23.54 mg, 620.22 μmol) was added to a solution of intermediate 38e (120 mg, 413.48 μmol) in THF (4 mL). The mixture was stirred at this temperature for 1 hour. To the reaction mixture, 24 μL of water, 24 μL of 15% sodium hydroxide solution, and 72 μL of ice water were slowly added in sequence to quench the reaction. The reaction mixture was added with water (2 mL) and extracted with EA (3 mL×2). The organic phases were combined, dried, and concentrated to obtain a crude product. The crude product was stirred with 3 mL of a mixed solution (PE:EA=1:1) for 10 minutes, and filtered to obtain the filter cake, which was intermediate 38f. MS m/z: 263.1 [M+1]⁺. ¹H NMR (400 MHz, DMSO-d₆) δ ppm 12.01 (br s, 1H), 9.12 (d, J=2.76 Hz, 1H), 8.41 (dd, J=8.66, 2.89 Hz, 1H), 8.37 (d, J=8.03 Hz, 1H), 7.37-7.46 (m, 1H), 5.46 (t, J=5.65 Hz, 1H), 4.68 (d, J=5.27 Hz, 2H).

### Step 6: Synthesis of Intermediate 38g

Intermediate 38f (50 mg, 190.69 μmol) and 1,2-dibromotetrachloroethane (136.61 mg, 419.51 μmol) were dissolved in DCM (0.5 mL). At 0° C., tributylphosphine (77.16 mg, 381.37 μmol, 94.10 μL) was added. The mixture was warmed to room temperature (25° C.) and stirred for 3 hours. The reaction mixture was filtered to obtain the filter cake, which was intermediate 38g. ¹H NMR (400 MHz, DMSO-d₆) δ ppm 12.15 (s, 1H), 9.14 (d, J=3.01 Hz, 1H), 8.42 (dd, J=8.53, 3.01 Hz, 1H), 8.36 (d, J=8.28 Hz, 1H), 7.38-7.51 (m, 1H), 4.84 (s, 2H).

### Step 7: Synthesis of Compound 38

At 25° C., triethylamine (15.56 mg, 153.80 μmol) was added to a solution of intermediate 9g (16.90 mg, hydrochloride) in DMF (1 mL). The mixture was stirred for 30 minutes. Intermediate 38g (25 mg, 76.90 μmol) and KI (1.28 mg, 7.69 μmol) were then added, and the reaction mixture was stirred at 50° C. for 3 hours. With stirring, the reaction mixture was added with 2 mL of water, and stirred for about 10 minutes. The mixture was filtered to obtain the filter cake, which was compound 38. MS m/z: 483.1 [M+1]⁺. ¹H NMR (400 MHz, DMSO-d₆) δ ppm 12.05 (s, 1H), 9.14 (d, J=2.76 Hz, 1H), 8.35-8.43 (m, 3H), 7.81-7.88 (m, 1H), 7.57 (dd, J=10.54, 8.03 Hz, 1H), 7.35-7.41 (m, 1H), 3.74 (s, 2H) 3.15-3.23 (m, 4H), 2.76 (d, J=4.77 Hz, 3H), 2.60-2.65 (m, 4H).

### Example 39

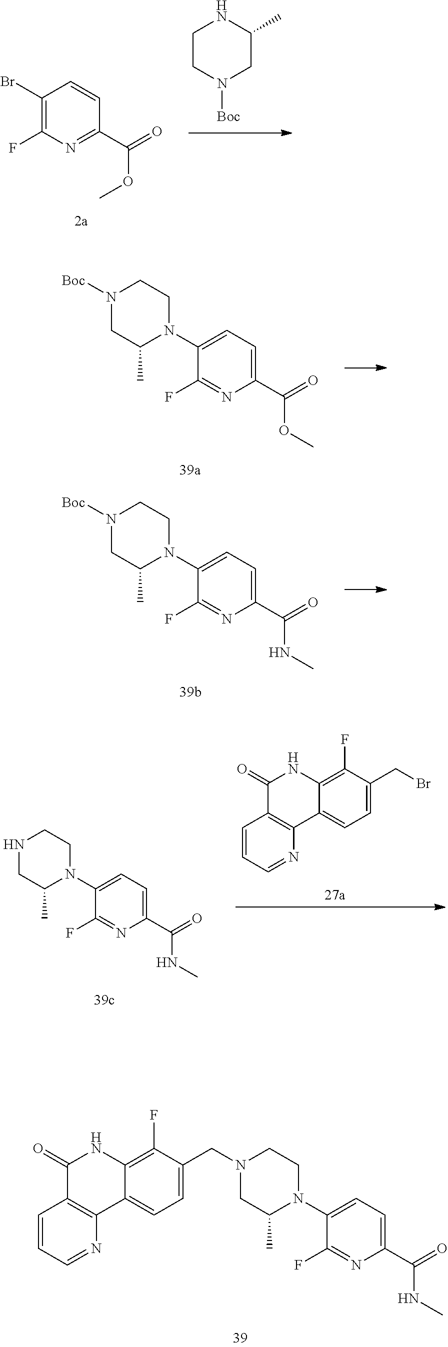

### Step 1: Synthesis of Intermediate 39a

Compound 2a (200 mg, 854.62 μmol) and (R)-4-tert-butoxycarbonyl-2-methylpiperazine (171.16 mg, 854.62 μmol) were dissolved in toluene (5 mL). RuPhos (79.76 mg, 170.92 μmol), Cs₂CO₃ (556.90 mg, 1.71 mmol), and Pd₂(dba)₃ (78.26 mg, 85.46 μmol) were sequentially added. The mixture was stirred under a nitrogen atmosphere at 100° C. for 16 hours. The reaction mixture was filtered, and the filtrate was concentrated under reduced pressure to obtain a crude product. The crude product was purified by column chromatography (eluent: PE:EA=100:0 to 90:10 to 75:25, V/V) to obtain intermediate 39a. MS m/z: 353.9 [M+1]⁺.

### Step 2: Synthesis of Intermediate 39b

Intermediate 39a (150 mg, 424.46 μmol) was dissolved in EtOH (2 mL). A solution of methylamine in ethanol (659.13 mg, 21.22 mmol) was added, and the reaction mixture was stirred at 25° C. for 16 hours. The reaction mixture was concentrated under reduced pressure to obtain intermediate 39b. MS m/z: 353.0 [M+1]⁺.

### Step 3: Synthesis of Hydrochloride of Intermediate 39c

Intermediate 39b (150 mg, 425.65 μmol) was dissolved in MeOH (3 mL). HCl/dioxane (4 M, 3 mL) was added, and the reaction mixture was stirred at room temperature (25° C.) for 3 hours. The reaction mixture was concentrated under reduced pressure to obtain the hydrochloride of intermediate 39c. MS m/z: 253.1 [M+1]⁺

### Step 4: Synthesis of Trifluoroacetate of Compound 39

Intermediate 27a (25 mg, 81.40 μmol), intermediate 39c (20.54 mg, hydrochloride), KI (6.76 mg, 40.70 μmol), and triethylamine (32.95 mg, 325.61 μmol) were dissolved in DMF (1 mL). The mixture was heated to 50° C. and stirred for 2 hours. After cooling, the mixture was added with 3 mL of water and extracted with ethyl acetate (4 mL×2). The organic phases were combined, dried over anhydrous sodium sulfate, and filtered. The filtrate was concentrated to obtain a crude product. The crude product was separated by preparative HPLC (column: C18, 100×40 mm; mobile phase: [water (trifluoroacetic acid)-acetonitrile]; gradient: 3%-33% acetonitrile) to obtain the trifluoroacetate of compound 39. MS m/z: 479.2 [M+1]⁺. ¹H NMR (400 MHz, CD₃OD) δ ppm 9.08 (dd, J=1.76, 4.77 Hz, 1H), 8.74 (d, J=4.82 Hz, 1H), 8.64 (d, J=8.53 Hz, 1H), 7.96 (d, J=8.03 Hz, 1H), 7.73 (dd, J=4.52, 8.03 Hz, 1H), 7.52 (dd, J=6.78, 8.28 Hz, 1H), 7.35 (d, J=8.03 Hz, 1H), 4.54 (s, 2H), 3.85-4.12 (m, 2H), 3.32-3.51 (m, 4H), 3.23-3.28 (m, 1H), 2.93 (s, 3H), 1.13 (d, J=6.27 Hz, 3H).

### Example 40

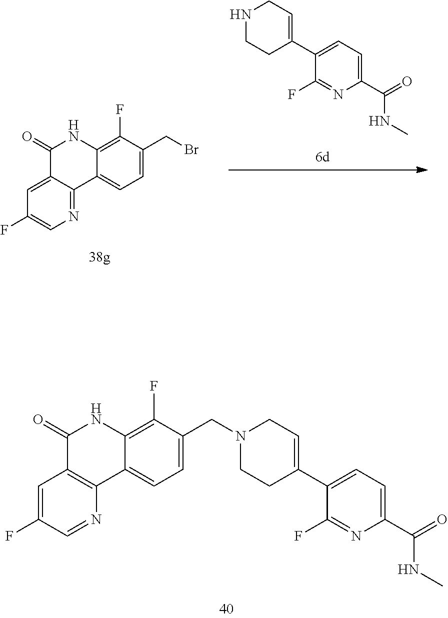 [SMILES: O=c1[nH]c2c(F)c(CBr)ccc2c2ncc(F)cc12 ; CNC(=O)c1ccc(C2=CCNCC2)c(F)n1 ; CNC(=O)c1ccc(C2=CCN(Cc3ccc4c([nH]c(=O)c5cc(F)cnc54)c3F)CC2)c(F)n1]

### Step 1: Synthesis of Trifluoroacetate of Compound 40

At 25° C., triethylamine (15.56 mg, 153.80 μmol) was added to a solution of intermediate 6d (16.72 mg, hydrochloride) in DMF (1 mL). The mixture was stirred for 30 minutes. Intermediate 38g (25 mg, 76.90 μmol) and KI (1.28 mg, 7.69 μmol) were then added, and the reaction mixture was stirred at 50° C. for 3 hours. The reaction mixture was filtered, and the filtrate was separated by preparative HPLC (column: C18, 100×40 mm; mobile phase: [water (trifluoroacetic acid)-acetonitrile]; gradient: 6%-36% acetonitrile) to obtain the trifluoroacetate of compound 40. MS m/z: 480.2 [M+1]⁺. ¹H NMR (400 MHz, DMSO-d₆) δ ppm 12.29 (br s, 1H), 10.26 (s, 1H), 9.18 (d, J=2.63 Hz, 1H), 8.66 (d, J=5.13 Hz, 1H), 8.42-8.56 (m, 2H), 8.13 (t, J=8.63 Hz, 1H), 7.98 (d, J=7.63 Hz, 1H), 7.45-7.61 (m, 1H), 6.27 (s, 1H), 4.66 (s, 2H), 3.90-4.12 (m, 2H), 3.66-3.83 (m, 1H), 2.80 (d, J=4.75 Hz, 3H), 2.74-2.79 (m, 1H), 2.22-2.32 (m, 2H).

### Example 41

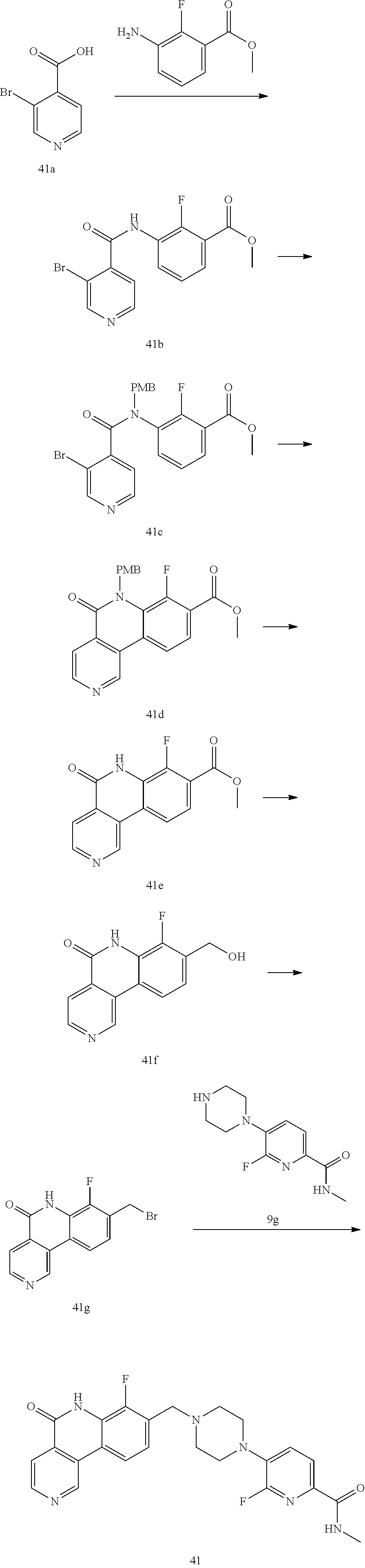

### Step 1: Synthesis of Intermediate 41b

Compound 41a (2 g, 9.90 mmol) and methyl 3-amino-2-fluorobenzoate (867.23 mg, 5.13 mmol) were added to DMF (20 mL). Triethylamine (3.01 g, 29.70 mmol) and HATU (4.52 g, 11.88 mmol) were then added, and the mixture was stirred at 25° C. for 16 hours. The reaction mixture was added with water (20 mL) and extracted with EA (30 mL×3). The organic phases were combined, dried over anhydrous sodium sulfate, and concentrated to obtain a crude product. The crude product was purified by silica gel column chromatography (eluent: DCM:MeOH=100:0 to 99:1, v/v) to obtain intermediate 41b. MS m/z: 352.9, 354.9 [M+1]⁺, ¹H NMR (400 MHz, DMSO-d₆) δ ppm 10.67-10.84 (m, 1H), 8.89 (s, 1H), 8.70 (d, J=5.02 Hz, 1H), 8.06-8.12 (m, 1H), 7.74-7.78 (m, 1H), 7.65 (d, J=4.77 Hz, 1H), 7.38 (t, J=7.91 Hz, 1H), 3.88 (s, 3H).

### Step 2: Synthesis of Intermediate 41c

Intermediate 41b (1.8 g, 5.10 mmol) and potassium carbonate (2.11 g, 15.29 mmol) were added to DMF (20 mL). p-Methoxybenzyl chloride (957.90 mg, 6.12 mmol) was then added, and the mixture was heated to 90° C. and stirred for 3 hours. The reaction mixture was filtered. The filtrate was added with 20 ml of water, and extracted with EA (30 mL×2). The organic phases were combined and concentrated under reduced pressure to obtain a crude product. The crude product was purified by silica gel column chromatography (eluent: PE:EA=2:1, v/v) to obtain intermediate 41c. MS m/z: 473.0, 475.0 [M+1]⁺.

### Step 3: Synthesis of Intermediate 41d

Under a nitrogen atmosphere, intermediate 41c (300 mg, 633.86 μmol), tributylphosphine (135.83 mg, 671.40 μmol), 1,3-bis(diphenylphosphino) propane (104.57 mg, 253.54 μmol), Pd(OAc)₂ (56.92 mg, 253.54 μmol), and potassium carbonate (175.21 mg, 1.27 mmol) were added to DMF (10 mL). The reaction was carried out at 140° C. under microwave irradiation for 10 minutes. The reaction mixture was filtered. The filtrate was added with 20 mL of water and extracted with EA (30 mL) three times. The organic phases were combined, dried over anhydrous sodium sulfate, and concentrated to obtain a crude product. The crude product was purified by column chromatography (eluent: PE:EA=100:0 to 60:40, v/v) to obtain intermediate 41d. MS m/z: 393.1 [M+1]⁺, ¹H NMR (400 MHz, DMSO-d₆) δ 9.99 (s, 1H), 8.95 (d, J=5.13 Hz, 1H), 8.66 (d, J=8.50 Hz, 1H), 8.22 (d, J=5.13 Hz, 1H), 7.76 (dd, J=8.38, 6.25 Hz, 1H), 7.13 (d, J=8.50 Hz, 2H), 6.86 (d, J=8.63 Hz, 2H), 5.58 (s, 2H), 3.86 (s, 3H), 3.71 (s, 3H).

### Step 4: Synthesis of Intermediate 41e

Intermediate 41d (250 mg, 637.14 μmol) was added to a mixed solution of trifluoroacetic acid (1 mL) and trifluoromethanesulfonic acid (0.2 mL). The mixture was stirred at 25° C. for 1 hour. The reaction mixture was added with 2 mL of water and extracted with ethyl acetate (8 mL). The organic phase was dried over anhydrous sodium sulfate, and filtered. The filtrate was concentrated to obtain intermediate 41e. MS m/z: 273.1 [M+1]⁺, ¹H NMR (400 MHz, DMSO-d₆) δ ppm 12.17 (br s, 1H), 9.95 (s, 1H), 8.93 (d, J=5.13 Hz, 1H), 8.52 (d, J=8.50 Hz, 1H), 8.19 (d, J=5.25 Hz, 1H), 7.72 (dd, J=8.44, 6.69 Hz, 1H), 3.93 (s, 3H).

### Step 5: Synthesis of Intermediate 41f

Under a nitrogen atmosphere, at 0° C., LiAlH₄ (2.5 M in tetrahydrofuran solution, 205.71 μL) was added to a solution of intermediate 41e (140 mg, 514.27 μmol) in THF (5 mL). The mixture was stirred at this temperature for 1 hour. To the reaction mixture, 30 μL of water, 30 μL of 15% sodium hydroxide solution, and 90 μL of ice water were slowly added in sequence to quench the reaction. The reaction mixture was added with water (10 mL) and extracted with EA (20 mL×3). The organic phases were combined, dried, and concentrated to obtain a crude product, which was intermediate 41f. MS m/z: 245.1 [M+1]⁺. ¹H NMR (400 MHz, DMSO-d₆) δ ppm 11.96 (br s, 1H), 9.89 (s, 1H), 8.85 (d, J=5.02 Hz, 1H), 8.41 (d, J=8.53 Hz, 1H), 8.14 (d, J=5.02 Hz, 1H), 7.39 (t, J=7.65 Hz, 1H), 5.46 (t, J=5.77 Hz, 1H), 4.68 (d, J=4.77 Hz, 2H).

### Step 6: Synthesis of Intermediate 41g

Intermediate 41f (50 mg, 204.73 μmol) and 1,2-dibromotetrachloroethane (146.67 mg, 450.41 μmol) were dissolved in DCM (0.5 mL). At 0° C., tributylphosphine (82.84 mg, 409.47 μmol) was added. The mixture was warmed to room temperature (25° C.) and stirred for 3 hours. The reaction mixture was filtered to obtain the filter cake, which was intermediate 41g. ¹H NMR (400 MHz, DMSO-d₆) δ ppm 12.16 (s, 1H), 9.90-9.98 (m, 1H), 8.82-9.06 (m, 1H), 8.38-8.48 (m, 1H), 8.21 (d, J=5.02 Hz, 1H), 7.46 (t, J=7.65 Hz, 1H), 4.94 (s, 2H).

### Step 7: Synthesis of Trifluoroacetate of Compound 41

At 25° C., triethylamine (153.80 μmol, 21.41 μL) was added to a solution of intermediate 9g (16.90 mg, hydrochloride) in DMF (1 mL). The mixture was stirred for 30 minutes. Intermediate 41g (25 mg, 81.40 μmol) and KI (1.35 mg, 8.14 μmol) were then added, and the reaction mixture was stirred at 50° C. for 2 hours. The reaction mixture was filtered, and the filtrate was purified by preparative liquid chromatography (column: C18, 100× 40 mm; mobile phase: [water (trifluoroacetic acid)-acetonitrile]; gradient: 0%-30% acetonitrile) to obtain the trifluoroacetate of compound 41. MS m/z: 465.2 [M+1]⁺. ¹H NMR (400 MHz, CD₃OD) δ ppm 9.86 (s, 1H), 8.91 (d, J=5.27 Hz, 1H), 8.49 (d, J=8.53 Hz, 1H), 8.33 (d, J=5.27 Hz, 1H), 7.96 (d, J=8.28 Hz, 1H), 7.64 (dd, J=10.29, 8.28 Hz, 1H), 7.56 (dd, J=8.28, 7.03 Hz, 1H), 4.66 (s, 2H), 3.47-3.72 (m, 8H), 2.93 (s, 3H).

### Example 42

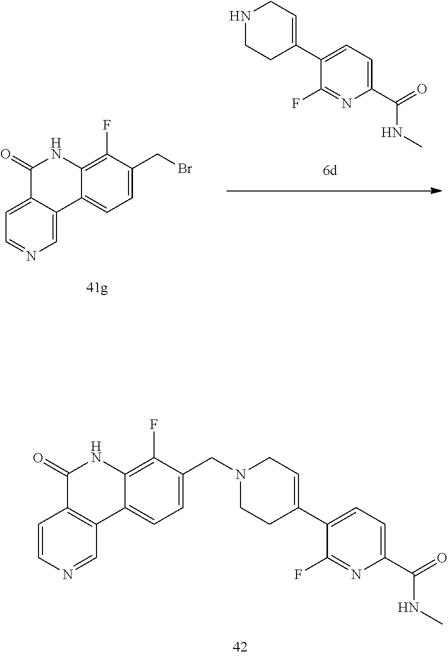 [SMILES: O=c1[nH]c2c(F)c(CBr)ccc2c2cnccc12 ; CNC(=O)c1ccc(C2=CCNCC2)c(F)n1 ; CNC(=O)c1ccc(C2=CCN(Cc3ccc4c([nH]c(=O)c5ccncc54)c3F)CC2)c(F)n1]

### Step 1: Synthesis of Trifluoroacetate of Compound 42

Triethylamine (16.47 mg, 162.80 μmol) was added to a solution of intermediate 6d (17.69 mg, hydrochloride) in DMF (1 mL). The mixture was stirred at 25° C. for 30 minutes. Intermediate 41g (25 mg, 81.40 μmol) and KI (1.35 mg, 8.14 μmol) were then added, and the reaction mixture was heated to 50° C. and stirred for 2 hours. The reaction mixture was filtered, and the filtrate was purified by preparative HPLC (column: C18, 100×40 mm; mobile phase: [water (trifluoroacetic acid)-acetonitrile]; gradient: 0%-30% acetonitrile) to obtain the trifluoroacetate of compound 42. MS m/z: 462.1 [M+1]⁺. ¹H NMR (400 MHz, CD₃OD) δ 9.86 (s, 1H), 8.91 (d, J=5.27 Hz, 1H), 8.50 (d, J=7.78 Hz, 1H), 8.34 (d, J=5.77 Hz, 1H), 8.08-8.15 (m, 1H), 8.02-8.07 (m, 1H), 7.58 (dd, J=8.28, 7.03 Hz, 1H), 6.31 (s, 1H), 4.74 (s, 2H), 4.11 (s, 2H), 3.58-3.85 (m, 2H), 2.96-3.01 (m, 2H), 2.96 (s, 3H).

### Example 43

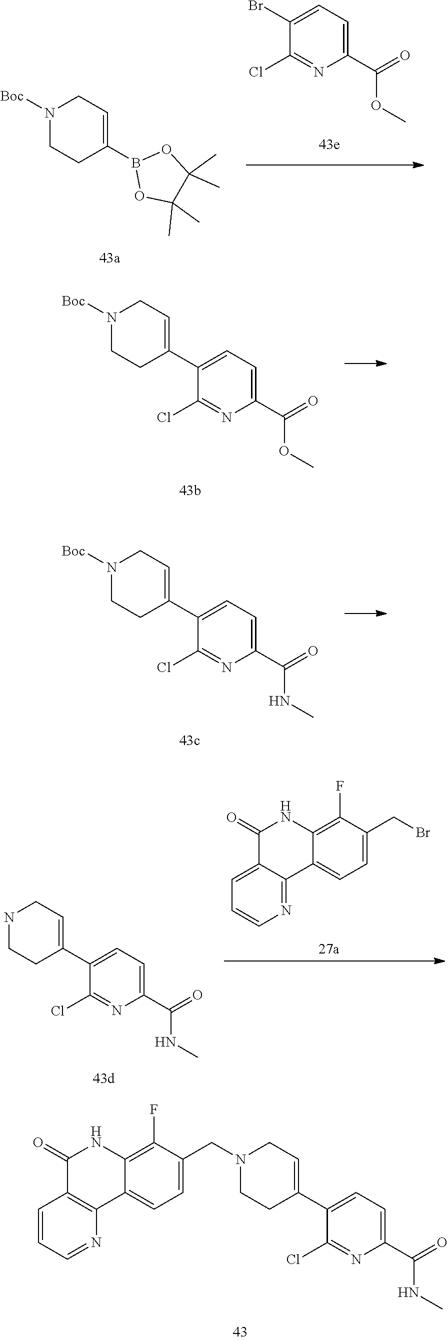

### Step 1: Synthesis of Intermediate 43b

Under a nitrogen atmosphere, 43e (800 mg, 3.19 mmol), compound 43a (1.09 g, 3.51 mmol), Pd(dppf)Cl₂ (233.70 mg, 319.39 μmol), and potassium acetate (626.91 mg, 6.39 mmol) were added to a mixed solution of H₂O (1.6 mL) and THF (8 mL). The mixture was heated to 70° C. and stirred for 16 hours. The reaction mixture was filtered. The filter cake was rinsed with EA (15 mL), and the filtrate was concentrated under reduced pressure to obtain a crude product. The crude product was purified by flash column chromatography (eluent: PE:EA=100:0 to 90:10, v/v) to obtain intermediate 43b. MS m/z: 353.1 [M+1]⁺. ¹H NMR (400 MHz, CDCl₃) δ ppm 8.07 (dd, J=7.78, 1.00 Hz, 1H), 7.64-7.76 (m, 1H), 5.83 (s, 1H), 4.11 (s, 2H), 3.67 (t, J=5.27 Hz, 2H), 2.50 (br s, 2H), 1.52 (s, 9H), 1.26 (s, 3H).

### Step 2: Synthesis of Intermediate 43c

A solution of methylamine in ethanol (9.05 g, 87.42 mmol, 30%) was added to a solution of intermediate 43b (300 mg, 850.31 μmol) in MeOH (1 mL). The mixture was stirred at 40° C. for 16 hours. The reaction mixture was concentrated under reduced pressure to obtain intermediate 43c. MS m/z: 352.1 [M+1]⁺. ¹H NMR (400 MHz, CD₃OD) δ ppm 7.92 (d, J=7.78 Hz, 1H), 7.74 (d, J=7.53 Hz, 1H), 5.75 (s, 1H), 3.98 (s, 2H), 3.53-3.61 (m, 2H), 2.85 (s, 3H), 2.33-2.41 (m, 2H), 1.40 (s, 9H).

### Step 3: Synthesis of Hydrochloride of Intermediate 43d

HCl/dioxane (4 M, 1.06 mL) was added to a solution of intermediate 43c (298 mg, 847.01 μmol) in MeOH (0.5 mL). The mixture was stirred at 25° C. for 1 hour. HCl/dioxane (4 M, 635.26 μL) was further added. The mixture was stirred for another 1 hour. The reaction mixture was concentrated under reduced pressure to obtain the hydrochloride of intermediate 43d. MS m/z: 252.1 [M+1]⁺.

### Step 4: Synthesis of Compound 43

Triethylamine (32.95 mg, 325.61 μmol) was added to a solution of intermediate 43d (37.53 mg, hydrochloride) in DMF (1 mL). The mixture was stirred at 25° C. for 30 minutes. Intermediate 27a (50 mg, 162.80 μmol) and KI (2.70 mg, 16.28 μmol) were then added, and the mixture was heated to 50° C. and stirred for 2.5 hours. The reaction mixture was added with DMF (2 mL), and with water (1 mL), resulting in the precipitation of a solid. The mixture was filtered to obtain the filter cake, which was compound 43. MS m/z: 478.1 [M+1]⁺. ¹H NMR (400 MHz, DMSO-d₆) δ ppm 11.90 (s, 1H), 9.09 (dd, J=4.57, 1.81 Hz, 1H), 8.65 (dd, J=8.07, 1.81 Hz, 1H), 8.61 (d, J=4.88 Hz, 1H), 8.43 (d, J=8.51 Hz, 1H), 7.88-7.93 (m, 2H), 7.72 (dd, J=8.07, 4.57 Hz, 1H), 7.36-7.43 (m, 1H), 5.86 (s, 1H), 3.82 (s, 2H), 3.67-3.74 (m, 2H), 3.18 (d, J=2.50 Hz, 2H), 2.81 (d, J=4.75 Hz, 3H), 2.45 (d, J=1.63 Hz, 2H).

### Example 44

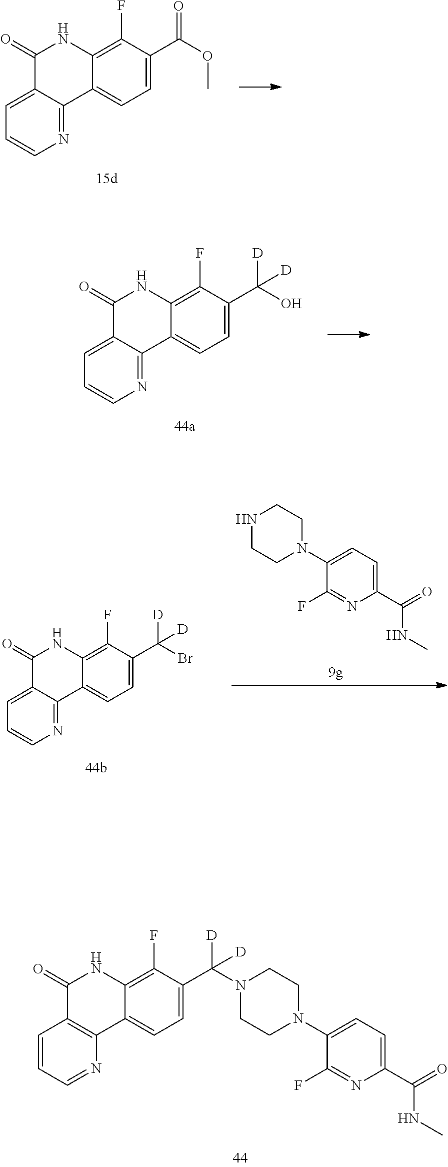 [SMILES: COC(=O)c1ccc2c([nH]c(=O)c3cccnc32)c1F ; [2H]C([2H])(O)c1ccc2c([nH]c(=O)c3cccnc32)c1F ; [2H]C([2H])(Br)c1ccc2c([nH]c(=O)c3cccnc32)c1F ; CNC(=O)c1ccc(N2CCNCC2)c(F)n1 ; [2H]C([2H])(c1ccc2c([nH]c(=O)c3cccnc32)c1F)N1CCN(c2ccc(C(=O)NC)nc2F)CC1]

### Step 1: Synthesis of Intermediate 44a

Intermediate 15d (100 mg, 367.34 μmol) was dissolved in THF (1 mL). At 0° C., deuterated lithium aluminum hydride powder (20.91 mg, 551.00 μmol) was added thereto, and the mixture was stirred at 0° C. for 1 hour. The reaction mixture was added to saturated sodium potassium tartrate solution (5 mL). The mixture was then extracted with ethyl acetate (10 mL×3). The organic phases were combined, dried over anhydrous sodium sulfate, and filtered. The filtrate was concentrated under reduced pressure at 45° C. to obtain intermediate 44a. MS m/z: 246.9 [M+1]⁺. ¹H NMR (400 MHz, DMSO-d₆) δ ppm 11.87 (s, 1H), 9.08 (dd, J=1.8, 4.5 Hz, 1H), 8.64 (dd, J=1.9, 7.9 Hz, 1H), 8.43 (d, J=7.5 Hz, 1H), 7.71 (dd, J=4.8, 8.0 Hz, 1H), 7.40 (dd, J=6.7, 8.2 Hz, 1H), 5.41 (s, 1H).

### Step 2: Synthesis of Intermediate 44b

Intermediate 44a (56 mg, 227.43 μmol) was dissolved in DCM (1 mL). 1,2-Dibromotetrachloroethane (162.93 mg, 500.34 μmol) was added, and at 0° C., tributylphosphine (92.02 mg, 454.85 μmol) was added. The mixture was stirred at 25° C. for 3 hours. The reaction mixture was concentrated under reduced pressure, and methanol (2 mL) was added thereto. The mixture was stirred for 5 minutes and then filtered to obtain the filter cake, which was intermediate 44b. ¹H NMR (400 MHz, DMSO-d₆) δ ppm 12.00 (s, 1H), 9.09 (d, J=4.5 Hz, 1H), 8.71-8.61 (m, 1H), 8.49-8.38 (m, 1H), 7.80-7.69 (m, 1H), 7.51-7.36 (m, 1H).

### Step 3: Synthesis of Compound 44

Intermediate 9g (31.99 mg, hydrochloride) was dissolved in DMF (1 mL). Triethylamine (58.92 mg, 582.28 μmol) was added, and the reaction mixture was stirred at 25° C. for 0.5 hours. Intermediate 44b (45 mg, 145.57 μmol) and KI (12.08 mg, 72.78 μmol) were then added. The mixture was stirred at 50° C. for 2 hours. The reaction mixture was added with water (1.5 mL), and stirred for 10 minutes, resulting in the precipitation of a solid. The mixture was filtered to obtain the filter cake, which was compound 44. MS m/z: 489.0 [M+23]⁺. ¹H NMR (400 MHz, DMSO-d₆) δ ppm 11.90 (s, 1H), 9.09 (dd, J=1.8, 4.5 Hz, 1H), 8.64 (dd, J=1.8, 8.0 Hz, 1H), 8.47-8.37 (m, 2H), 7.84 (d, J=7.8 Hz, 1H), 7.72 (dd, J=4.5, 8.0 Hz, 1H), 7.57 (dd, J=8.3, 10.5 Hz, 1H), 7.37 (dd, J=6.7, 8.2 Hz, 1H), 3.17-3.20 (m, 4H), 2.76 (d, J=4.8 Hz, 3H), 2.57-2.65 (m, 4H).

### Example 45

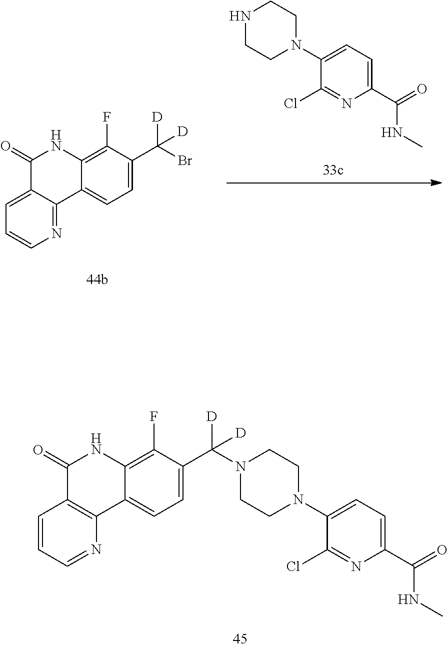 [SMILES: [2H]C([2H])(Br)c1ccc2c([nH]c(=O)c3cccnc32)c1F ; CNC(=O)c1ccc(N2CCNCC2)c(Cl)n1 ; [2H]C([2H])(c1ccc2c([nH]c(=O)c3cccnc32)c1F)N1CCN(c2ccc(C(=O)NC)nc2Cl)CC1]

### Step 1: Synthesis of Compound 45

Intermediate 33c (37.68 mg, hydrochloride) was dissolved in DMF (1 mL). Triethylamine (65.47 mg, 646.98 μmol) was added, and the reaction mixture was stirred at 25° C. for 0.5 hours. Intermediate 44b (50 mg, 161.74 μmol) and KI (13.42 mg, 80.87 μmol) were then added. The mixture was stirred at 50° C. for 1 hour. After cooling, the reaction mixture was added with water (1 mL), and stirred for 10 minutes, resulting in the precipitation of a solid. The mixture was filtered to obtain the filter cake, which was compound 45. MS m/z: 483.1 [M+1]⁺. ¹H NMR (400 MHz, DMSO-d₆) δ ppm 11.90 (s, 1H), 9.09 (dd, J=1.8, 4.5 Hz, 1H), 8.65 (dd, J=1.8, 8.0 Hz, 1H), 8.48-8.38 (m, 2H), 7.94 (d, J=8.3 Hz, 1H), 7.72 (dd, J=4.5, 8.0 Hz, 1H), 7.66 (d, J=8.3 Hz, 1H), 7.37 (dd, J=6.7, 7.9 Hz, 1H), 3.10-3.15 (m, 4H), 2.79 (d, J=4.8 Hz, 3H), 2.62-2.67 (m, 4H).

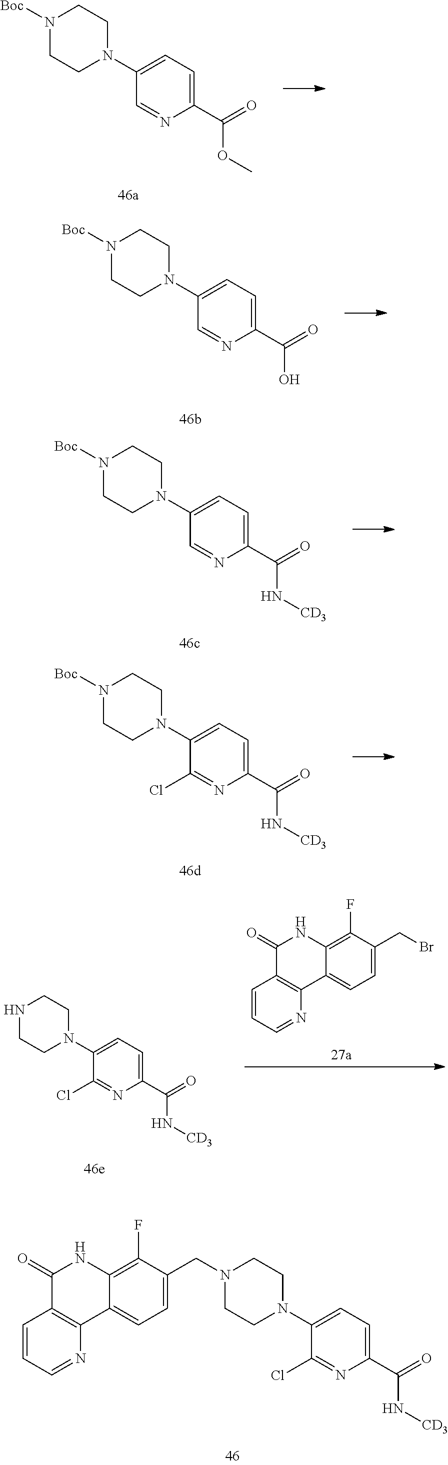

### Step 1: Synthesis of Intermediate 46b

Intermediate 46a (2 g, 6.22 mmol) was dissolved in THF (20 mL) and H₂O (4 mL). Lithium hydroxide monohydrate (1.31 g, 31.12 mmol) was added thereto, and the mixture was stirred at room temperature (25° C.) for 2 hours. The pH of the reaction mixture was adjusted to 3 with dilute hydrochloric acid (1 mol/L). Then the mixture was extracted with ethyl acetate (20 mL×3). The organic phase was washed with 20 mL of saturated brine, dried over anhydrous sodium sulfate, and filtered. The filtrate was concentrated under reduced pressure to obtain intermediate 46b. MS m/z: 307.9 [M+1]⁺. ¹H NMR (400 MHz, DMSO-d₆) δ ppm 8.37 (d, J=2.8 Hz, 1H), 7.88 (d, J=8.8 Hz, 1H), 7.36 (dd, J=3.0, 8.8 Hz, 1H), 3.50-3.46 (m, 4H), 3.39-3.36 (m, 4H), 1.43 (s, 9H).

### Step 2: Synthesis of Intermediate 46c

Intermediate 46b (400 mg, 1.30 mmol) and deuterated methylamine hydrochloride (100.98 mg, 1.43 mmol) were dissolved in EA (4 mL). DIEA (336.41 mg, 2.60 mmol) and n-butyl phosphoric anhydride (50% ethyl acetate solution) (937.74 mg, 2.60 mmol) were added, and the mixture was stirred at 25° C. for 3 hours. An additional 937.74 mg of n-butyl phosphoric anhydride was added, and the mixture was stirred for another 16 hours. The pH of the reaction mixture was adjusted to 10. The mixture was added with 5 mL of water and extracted with ethyl acetate (5 mL×3). The organic phases were combined, dried over anhydrous sodium sulfate, and filtered. The filtrate was concentrated under reduced pressure to obtain intermediate 46c. MS m/z: 324.0 [M+1]⁺. ¹H NMR (400 MHz, DMSO-d₆) δ ppm 8.43-8.37 (m, 1H), 8.28 (d, J=2.8 Hz, 1H), 7.84 (d, J=8.8 Hz, 1H), 7.41 (dd, J=2.8, 8.8 Hz, 1H), 3.52-3.45 (m, 4H), 3.32-3.30 (m, 4H), 1.43 (s, 9H).

### Step 3: Synthesis of Intermediate 46d

Intermediate 46c (314 mg, 970.92 μmol) was dissolved in DMF (3.5 mL). N-Chlorosuccinimide (142.61 mg, 1.07 mmol) was added, and the reaction mixture was stirred at 50° C. for 1 hour. The reaction mixture was added with 10 ml of water and extracted with ethyl acetate (10 mL×3). The organic phases were combined, dried over anhydrous sodium sulfate, and filtered. The filtrate was concentrated under reduced pressure to obtain intermediate 46d. MS m/z: 358.0 [M+1]⁺, 380.0 [M+23]⁺. ¹H NMR (400 MHz, DMSO-d6) δ ppm 8.44 (s, 1H), 7.95 (d, J=8.0 Hz, 1H), 7.68 (d, J=8.0 Hz, 1H), 3.52-3.47 (m, 4H), 3.09-3.01 (m, 4H), 1.43 (s, 9H).

### Step 4: Synthesis of Hydrochloride of Intermediate 46e

Intermediate 46d (355 mg, 992.04 μmol) was dissolved in MeOH (4 mL). HCl/dioxane (4 M, 1.34 mL) was added, and the reaction mixture was stirred at 25° C. for 2 hours. The reaction mixture was concentrated under reduced pressure to obtain the hydrochloride of intermediate 46e. MS m/z: 257.9 [M+1]⁺.

### Step 5: Synthesis of Compound 46

Intermediate 46e (38.32 mg, hydrochloride) was dissolved in DMF (1 mL). Triethylamine (65.90 mg, 651.22 μmol) was added, and the reaction mixture was stirred at 25° C. for 0.5 hours. KI (13.51 mg, 81.40 μmol) and intermediate 27a (50 mg, 162.80 μmol) were then added, and the mixture was stirred at 50° C. for 1 hour. After cooling, the reaction mixture was stirred with 2 mL of water for 10 minutes, resulting in the precipitation of a solid. The mixture was filtered to obtain the filter cake, which was compound 46. MS m/z: 484.0 [M+1]⁺, 506.0 [M+23]⁺. ¹H NMR (400 MHz, DMSO-d₆) δ ppm 11.90 (s, 1H), 9.09 (dd, J=1.8, 4.5 Hz, 1H), 8.64 (dd, J=1.8, 8.0 Hz, 1H), 8.47-8.38 (m, 2H), 7.94 (d, J=8.3 Hz, 1H), 7.72 (dd, J=4.5, 8.0 Hz, 1H), 7.67 (d, J=8.0 Hz, 1H), 7.37 (dd, J=6.8, 8.0 Hz, 1H), 3.76 (s, 2H), 3.16-3.12 (m, 4H), 2.69-2.63 (m, 4H).

### Example 47

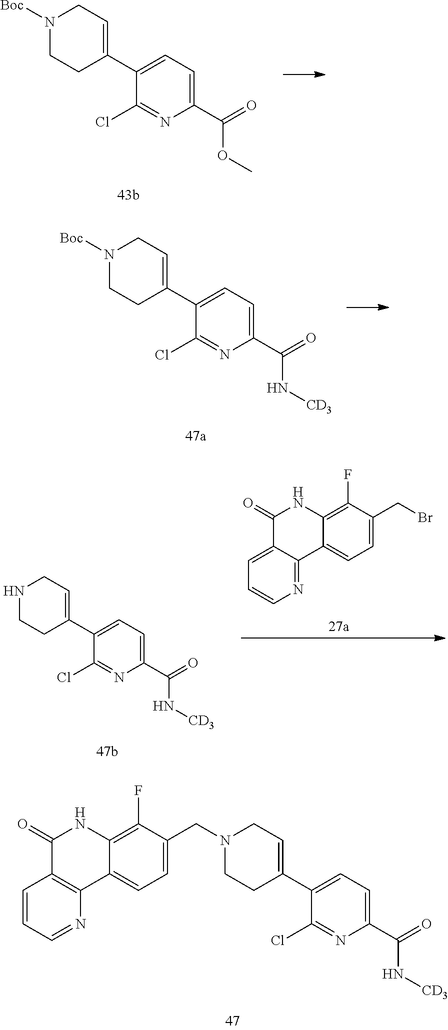 [SMILES: COC(=O)c1ccc(C2=CCN(C(=O)OC(C)(C)C)CC2)c(Cl)n1 ; [2H]C([2H])([2H])NC(=O)c1ccc(C2=CCN(C(=O)OC(C)(C)C)CC2)c(Cl)n1 ; [2H]C([2H])([2H])NC(=O)c1ccc(C2=CCNCC2)c(Cl)n1 ; O=c1[nH]c2c(F)c(CBr)ccc2c2ncccc12 ; [2H]C([2H])([2H])NC(=O)c1ccc(C2=CCN(Cc3ccc4c([nH]c(=O)c5cccnc54)c3F)CC2)c(Cl)n1]

### Step 1: Synthesis of Intermediate 47a

Deuterated methylamine hydrochloride (399.85 mg, 5.67 mmol) was added to H₂O (2 mL). Sodium tert-butoxide (272.39 mg, 2.83 mmol) was then added. The mixture was stirred at 25° C. for 1 hour. A solution of intermediate 43b (100 mg, 283.44 μmol) in MeOH (4 mL) was added. The reaction mixture was heated to 60° C. and stirred for 4 hours. After cooling to room temperature, the mixture was extracted with ethyl acetate (5 mL×3). The organic phases were combined, dried over anhydrous sodium sulfate, and concentrated under reduced pressure to obtain a crude product. The crude product was purified by preparative TLC (eluent: PE:EA=2:1) to obtain intermediate 47a. MS m/z: 355.1, 357.1 [M+1]⁺.

### Step 2: Synthesis of Hydrochloride of Intermediate 47b

At 25° C., HCl/dioxane (4 M, 211.36 μL) was added to a solution of intermediate 47a (60 mg, 169.09 μmol) in MeOH (0.5 mL). The mixture was stirred for 2 hours. The reaction mixture was concentrated under reduced pressure to obtain the hydrochloride of intermediate 47b. MS m/z: 255.1, 257.1 [M+1]⁺. ¹H NMR (400 MHz, DMSO-d₆) δ ppm 9.36 (br s, 2H), 8.63 (s, 1H), 8.03 (d, J=7.78 Hz, 1H), 7.89 (d, J=7.78 Hz, 1H), 5.93 (br s, 1H), 3.76 (br d, J=1.51 Hz, 2H), 3.30 (br d, J=4.52 Hz, 2H), 2.63 (br d, J=1.51 Hz, 2H).

### Step 3: Synthesis of Compound 47

Triethylamine (32.95 mg, 325.61 μmol) was added to a solution of intermediate 47b (37.93 mg, hydrochloride) in DMF (1.5 mL). The mixture was stirred at 25° C. for 0.5 hours. Intermediate 27a (50 mg, 162.80 μmol) and KI (2.70 mg, 16.28 μmol) were then added, and the reaction mixture was stirred at 50° C. for 2.5 hours. After cooling, the reaction mixture was added with 1.5 mL of water, resulting in the precipitation of a solid. The mixture was filtered to obtain the filter cake, which was compound 47. MS m/z: 481.0, 483.0 [M+1]⁺. ¹H NMR (400 MHz, DMSO-d₆) δ ppm 11.89 (s, 1H), 9.09 (dd, J=4.50, 1.75 Hz, 1H), 8.65 (dd, J=8.00, 1.75 Hz, 1H), 8.57 (s, 1H), 8.44 (d, J=8.25 Hz, 1H), 7.95-7.99 (m, 1H), 7.88-7.93 (m, 1H), 7.72 (dd, J=8.00, 4.63 Hz, 1H), 7.36-7.43 (m, 1H), 5.87 (s, 1H), 3.82 (s, 2H), 3.19 (d, J=2.13 Hz, 2H), 2.72-2.78 (m, 2H), 2.44-2.47 (m, 2H).

### Example 48

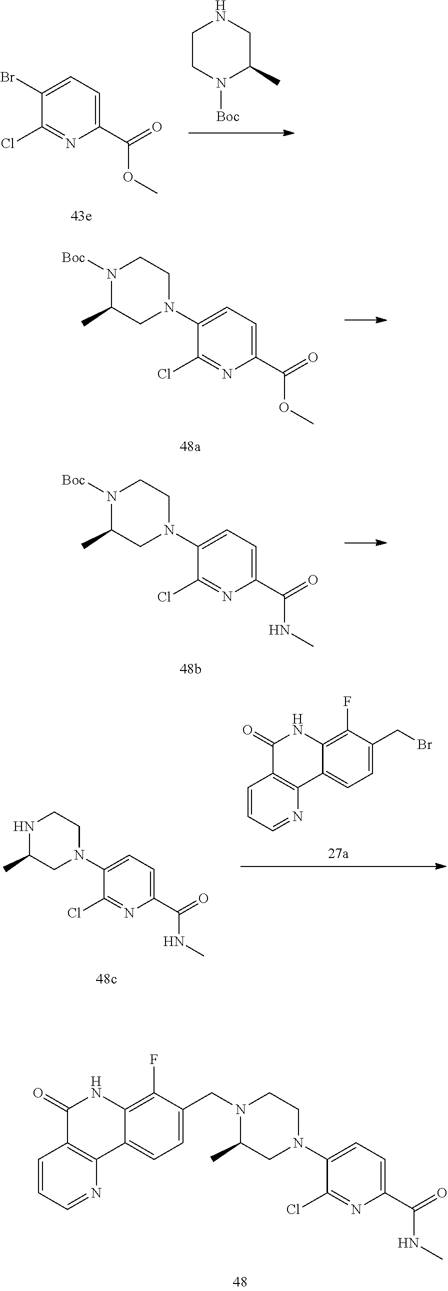

### Step 1: Synthesis of Intermediate 48a

(R)-1-tert-Butoxycarbonyl-2-methylpiperazine (0.2 g, 998.62 μmol), 43e (250.13 mg, 998.62 μmol), Cs₂CO₃ (650.74 mg, 2.00 mmol), RuPhos (93.20 mg, 199.72 μmol), and Pd₂(dba)₃ (91.44 mg, 99.86 μmol) were sequentially added to toluene (4 mL). The mixture was purged with nitrogen three times and stirred at 100° C. for 6 hours. The reaction mixture was filtered under reduced pressure while hot, and the filtrate was concentrated under reduced pressure at 50° C. to obtain a crude product. The crude product was purified by column chromatography (eluent: PE/EA=4/1, v/v) to obtain intermediate 48a. MS m/z: 370.0, 372.0 [M+1]⁺.

### Step 2: Synthesis of Intermediate 48b

Intermediate 48a (183.15 mg, 495.21 μmol) was added to EtOH (2 mL). With stirring, a solution of methylamine in ethanol (2.27 g, 21.93 mmol) was added thereto, and the reaction mixture was stirred at 20° C. for 16 hours. The reaction mixture was concentrated under reduced pressure at 45° C. to obtain a crude product of intermediate 48b. MS m/z: 312.9, 314.9 [M−56]⁺.

### Step 3: Synthesis of Hydrochloride of Intermediate 48c

Intermediate 48b (100 mg, 271.11 μmol) was added to MeOH (2 mL). With stirring, HCl/dioxane (4 M, 383.08 μL) was added thereto, and the reaction mixture was stirred at 25° C. for 2 hours. The reaction mixture was concentrated under reduced pressure at 45° C. to obtain the hydrochloride of intermediate 48c. MS m/z: 268.9, 270.9 [M+1]⁺.

### Step 4: Synthesis of Compound 48

Triethylamine (26.36 mg, 260.49 μmol) was added to a solution of intermediate 48c (39.75 mg, hydrochloride) in DMF (2 mL). The mixture was stirred at 20° C. for 0.5 hours. Intermediate 27a (50 mg, 130.24 μmol) and KI (2.16 mg, 13.02 μmol) were then added thereto, and the reaction mixture was stirred at 50° C. for 2 hours. The reaction mixture was concentrated under reduced pressure to obtain a crude product. The crude product was purified by preparative TLC (100% EA) to obtain compound 48. MS m/z: 495.2, 497.2 [M+1]⁺. ¹H NMR (400 MHz, DMSO-d₆) δ ppm 11.86 (s, 1H), 9.08 (dd, J=4.57, 1.69 Hz, 1H), 8.64 (dd, J=8.00, 1.75 Hz, 1H), 8.36-8.48 (m, 2H), 7.94 (d, J=8.13 Hz, 1H), 7.60-7.76 (m, 2H), 7.33-7.47 (m, 1H), 4.12 (d, J=13.38 Hz, 1H), 3.53 (d, J=13.88 Hz, 1H), 3.20-3.26 (m, 1H), 2.85-2.95 (m, 1H), 2.80-2.85 (m, 1H), 2.79 (d, J=4.75 Hz, 3H), 2.67-2.77 (m, 2H), 2.38-2.45 (m, 2H), 1.24 (d, J=5.50 Hz, 3H).

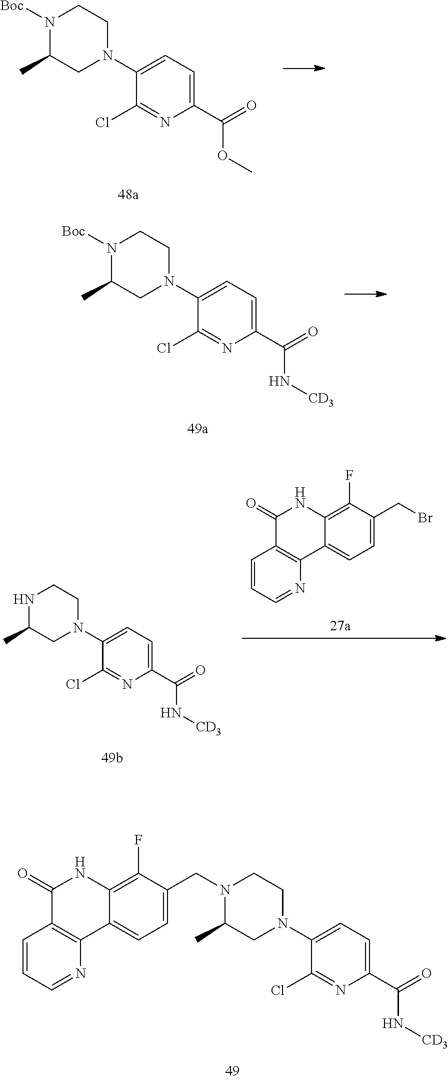

### Step 1: Synthesis of Intermediate 49a

Deuterated methylamine hydrochloride (190.72 mg, 2.70 mmol) was added to H₂O (1 mL). Sodium tert-butoxide (129.92 mg, 1.35 mmol) was then added thereto. The reaction mixture was stirred at 25° C. for 1 hour. A solution of intermediate 48a (50 mg, 135.19 μmol) in MeOH (2 mL) was then added thereto, and the mixture was heated to 60° C. and stirred for 4 hours. The reaction mixture was extracted with ethyl acetate (5 mL×3). The organic phases were combined, dried over anhydrous sodium sulfate, and filtered. The filtrate was concentrated under reduced pressure to obtain intermediate 49a. MS m/z: 394.1, 396.1 [M+23]⁺.

### Step 2: Synthesis of Hydrochloride of Intermediate 49b

Intermediate 49a (50 mg, 134.45 μmol) was added to MeOH (2 mL). With stirring, HCl/dioxane (4 M, 383.08 μL) was added thereto, and the reaction mixture was stirred at 25° C. for 1 hour. The reaction mixture was concentrated under reduced pressure to obtain the hydrochloride of intermediate 49b. MS m/z: 272.0, 274.0 [M+1]⁺.

### Step 3: Synthesis of Compound 49

Triethylamine (26.36 mg, 260.49 μmol) was added to a solution of intermediate 49b (40.14 mg, hydrochloride) in DMF (2 mL). The mixture was stirred at 20° C. for 0.5 hours. Intermediate 27a (50 mg, 130.24 μmol) and KI (2.16 mg, 13.02 μmol) were added to the reaction mixture, and the mixture was stirred at 50° C. for 2 hours. The mixture was concentrated under reduced pressure to obtain a crude product. The crude product was purified by preparative TLC (100% EA) to obtain compound 49. MS m/z: 498.2, 500.2 [M+1]⁺. ¹H NMR (400 MHz, DMSO-d₆) δ ppm 11.86 (s, 1H), 9.08 (dd, J=4.57, 1.69 Hz, 1H), 8.64 (dd, J=7.94, 1.81 Hz, 1H), 8.33-8.50 (m, 2H), 7.94 (d, J=8.25 Hz, 1H), 7.58-7.78 (m, 2H), 7.31-7.48 (m, 1H), 4.12 (d, J=13.38 Hz, 1H), 3.53 (d, J=13.76 Hz, 1H), 3.24 (s, 1H), 2.80-2.93 (m, 2H), 2.70-2.79 (m, 2H), 2.43 (t, J=9.01 Hz, 2H), 1.24 (d, J=5.50 Hz, 3H).

### Example 50

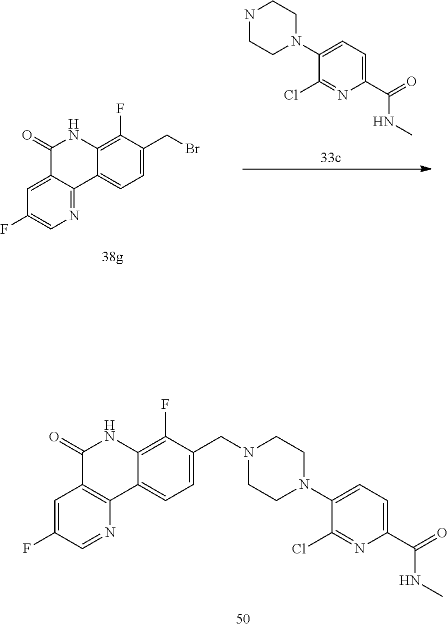 [SMILES: O=c1[nH]c2c(F)c(CBr)ccc2c2ncc(F)cc12 ; CNC(=O)c1ccc(N2CCNCC2)c(Cl)n1 ; CNC(=O)c1ccc(N2CCN(Cc3ccc4c([nH]c(=O)c5cc(F)cnc54)c3F)CC2)c(Cl)n1]

### Step 1: Synthesis of Compound 50

Triethylamine (31.12 mg, 307.59 μmol) was added to a solution of intermediate 33c (35.83 mg, hydrochloride) in DMF (1 mL). The mixture was stirred at 25° C. for 0.5 hours. Intermediate 38g (50 mg, 153.80 μmol) and KI (2.55 mg, 15.38 μmol) were then added, and the reaction mixture was stirred at 50° C. for 2.5 hours. After cooling, the reaction mixture was added with 1 mL of water, resulting in the precipitation of a solid. The mixture was directly filtered to obtain the filter cake, which was compound 50. MS m/z: 499.1, 501.1 [M+1]⁺. ¹H NMR (400 MHz, DMSO-d₆) δ ppm 12.03 (br s, 1H), 9.13 (d, J=2.88 Hz, 1H), 8.39-8.46 (m, 2H), 8.36 (d, J=8.25 Hz, 1H), 7.93 (d, J=8.25 Hz, 1H), 7.66 (d, J=8.25 Hz, 1H), 7.38 (dd, J=8.00, 6.75 Hz, 1H), 3.76 (s, 2H), 3.08-3.16 (m, 4H), 2.79 (d, J=4.88 Hz, 3H), 2.63-2.69 (m, 4H).

### Example 51

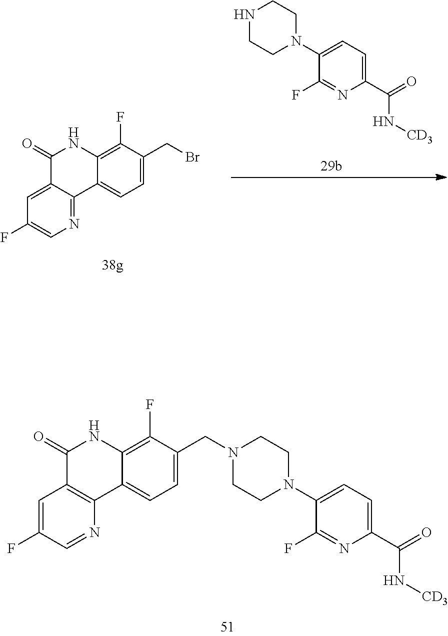 [SMILES: O=c1[nH]c2c(F)c(CBr)ccc2c2ncc(F)cc12 ; [2H]C([2H])([2H])NC(=O)c1ccc(N2CCNCC2)c(F)n1 ; [2H]C([2H])([2H])NC(=O)c1ccc(N2CCN(Cc3ccc4c([nH]c(=O)c5cc(F)cnc54)c3F)CC2)c(F)n1]

### Step 1: Synthesis of Compound 51

Triethylamine (18.67 mg, 184.55 μmol) was added to a solution of intermediate 29b (23.07 mg, hydrochloride) in DMF (1 mL). The mixture was stirred at 25° C. for 0.5 hours. Intermediate 38g (30 mg, 92.28 μmol) and KI (1.53 mg, 9.23 μmol) were then added, and the reaction mixture was stirred at 50° C. for 2.5 hours. After cooling to room temperature, the reaction mixture was added with 1 mL of water, resulting in the precipitation of a solid. The mixture was filtered to obtain the filter cake, which was compound 51. MS m/z: 486.2 [M+1]⁺. ¹H NMR (400 MHz, DMSO-d₆) δ ppm 12.05 (s, 1H), 9.13 (d, J=3.00 Hz, 1H), 8.42 (dd, J=8.57, 2.94 Hz, 1H), 8.34-8.39 (m, 2H), 7.84 (d, J=7.50 Hz, 1H), 7.56 (dd, J=10.57, 8.19 Hz, 1H), 7.34-7.41 (m, 1H), 3.74 (s, 2H), 3.15-3.22 (m, 4H), 2.59-2.65 (m, 4H).

### Example 52

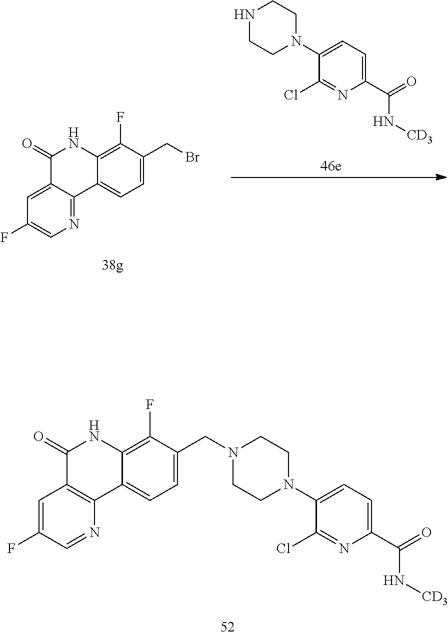 [SMILES: O=c1[nH]c2c(F)c(CBr)ccc2c2ncc(F)cc12 ; [2H]C([2H])([2H])NC(=O)c1ccc(N2CCNCC2)c(Cl)n1 ; [2H]C([2H])([2H])NC(=O)c1ccc(N2CCN(Cc3ccc4c([nH]c(=O)c5cc(F)cnc54)c3F)CC2)c(Cl)n1]

### Step 1: Synthesis of Compound 52

Intermediate 46e (21.72 mg, hydrochloride) was dissolved in DMF (1 mL). Triethylamine (37.35 mg, 369.11 μmol) was added, and the reaction mixture was stirred at 25° C. for 0.5 hours. KI (7.66 mg, 46.14 μmol) and intermediate 38e (30 mg, 92.28 μmol) were then added, and the mixture was stirred at 50° C. for 2 hours. After cooling to room temperature, the reaction mixture was added with 1 mL of water, resulting in the precipitation of a solid. The mixture was filtered to obtain the filter cake, which was compound 52. MS m/z: 502.2 [M+1]⁺. ¹H NMR (400 MHz, DMSO-d₆) δ ppm 12.06 (s, 1H), 9.14 (d, J=3.0 Hz, 1H), 8.44-8.40 (m, 2H), 8.37 (d, J=8.5 Hz, 1H), 7.94 (d, J=8.0 Hz, 1H), 7.66 (d, J=8.3 Hz, 1H), 7.39 (t, J=7.5 Hz, 1H), 3.76 (s, 2H), 3.12 (m, 4H), 2.69-2.62 (m, 4H).

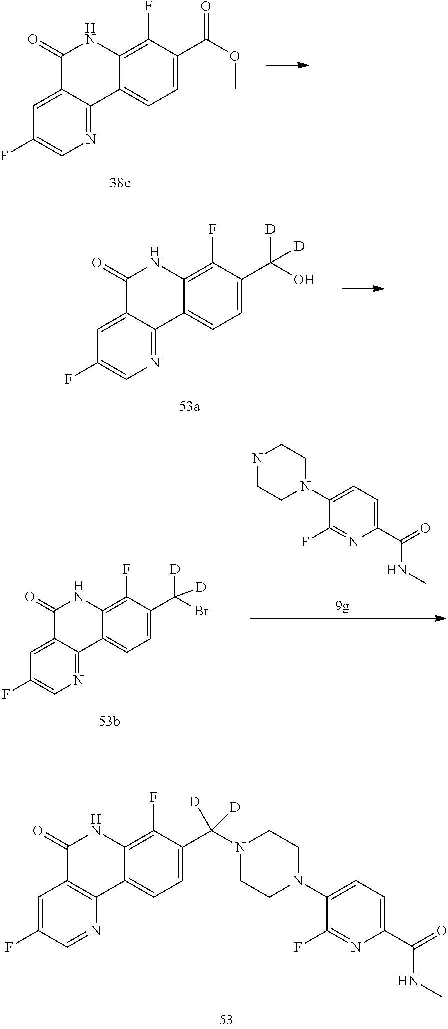 [SMILES: COC(=O)c1ccc2c([nH]c(=O)c3cc(F)cnc32)c1F ; [2H]C([2H])(O)c1ccc2c([nH]c(=O)c3cc(F)cnc32)c1F ; [2H]C([2H])(Br)c1ccc2c([nH]c(=O)c3cc(F)cnc32)c1F ; CNC(=O)c1ccc(N2CCNCC2)c(F)n1 ; [2H]C([2H])(c1ccc2c([nH]c(=O)c3cc(F)cnc32)c1F)N1CCN(c2ccc(C(=O)NC)nc2F)CC1]

### Step 1: Synthesis of Intermediate 53a

Intermediate 38e (150 mg, 516.85 μmol) was dissolved in THF (2 mL). At 0° C., deuterated lithium aluminum hydride powder (29.42 mg, 775.27 μmol) was added thereto, and the mixture was stirred at 0° C. for 1 hour. To the reaction mixture, 30 μL of water, 30 μL of 15% sodium hydroxide solution, and 90 μL of ice water were slowly added in sequence to quench the reaction. The reaction mixture was added with water (5 mL) and extracted with ethyl acetate (10 mL). The organic phase was dried over anhydrous sodium sulfate, and filtered. The filtrate was concentrated under reduced pressure at 45° C. to obtain intermediate 53a. MS m/z: 265.1 [M+1]⁺. ¹H NMR (400 MHz, DMSO-d₆) δ ppm 9.10 (d, J=2.8 Hz, 1H), 8.34-8.41 (m, 2H), 7.36 (t, J=8.0 Hz, 1H), 5.38 (s, 1H).

### Step 2: Synthesis of Intermediate 53b

Intermediate 53a (130 mg, 492.01 μmol) was dissolved in DCM (3 mL). 1,2-Dibromotetrachloroethane (352.48 mg, 1.08 mmol) was added, and at 0° C., tributylphosphine (199.09 mg, 984.01 μmol) was added. The mixture was stirred at 25° C. for 3 hours. The reaction mixture was concentrated under reduced pressure, and methanol (5 mL) was added thereto. The mixture was stirred for 5 minutes and then filtered to obtain the filter cake, which was intermediate 53b. ¹H NMR (400 MHz, DMSO-d₆) δ ppm 12.16 (s, 1H), 9.13 (d, J=2.8 Hz, 1H), 8.41-8.43 (m, 1H), 8.34-8.37 (m, 1H), 7.44 (t, J=8.0 Hz, 1H).

### Step 3: Synthesis of Compound 53

Intermediate 9g (22.68 mg, hydrochloride) was dissolved in DMF (1 mL). Triethylamine (18.56 mg, 183.42 μmol) was added, and the reaction mixture was stirred at 25° C. for 0.5 hours. Intermediate 53b (30 mg, 91.71 μmol) and KI (1.52 mg, 9.17 μmol) were then added. The mixture was stirred at 50° C. for 2 hours. The reaction mixture was added with water (1 mL), and stirred for 10 minutes, resulting in the precipitation of a solid. The mixture was filtered to obtain the filter cake, which was compound 53. MS m/z: 485.1 [M+1]⁺. ¹H NMR (400 MHz, DMSO-d₆) δ ppm 12.05 (s, 1H), 9.14 (d, J=3.01 Hz, 1H), 8.39-8.45 (m, 2H), 8.37 (d, J=8.03 Hz, 1H), 7.84 (d, J=7.28 Hz, 1H), 7.56 (dd, J=10.54, 8.28 Hz, 1H), 7.38 (t, J=7.15 Hz, 1H), 3.14-3.24 (m, 4H), 2.76 (d, J=4.77 Hz, 3H), 2.60-2.66 (m, 4H).

### Example 54

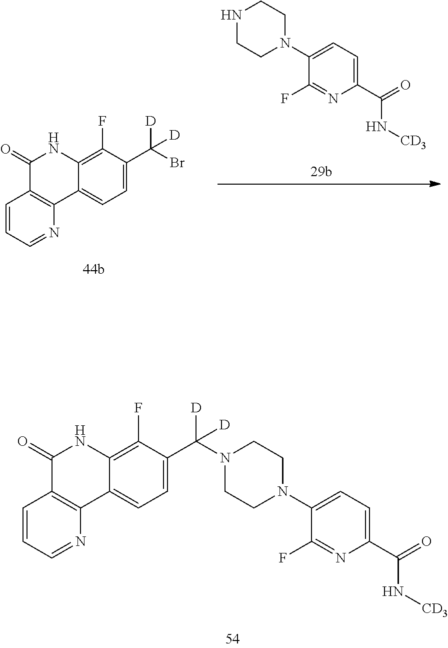 [SMILES: [2H]C([2H])(Br)c1ccc2c([nH]c(=O)c3cccnc32)c1F ; [2H]C([2H])([2H])NC(=O)c1ccc(N2CCNCC2)c(F)n1 ; [2H]C([2H])([2H])NC(=O)c1ccc(N2CCN(C([2H])([2H])c3ccc4c([nH]c(=O)c5cccnc54)c3F)CC2)c(F)n1]

### Step 1: Synthesis of Compound 54

Intermediate 29b (23.87 mg, hydrochloride) was dissolved in DMF (1 mL). Triethylamine (43.49 mg, 429.80 μmol) was added, and the reaction mixture was stirred at 25° C. for 0.5 hours. KI (8.92 mg, 53.73 μmol) and intermediate 44b (33.22 mg, 107.45 μmol) were then added, and the mixture was stirred at 50° C. for 1 hour. The reaction mixture was added with 1 mL of water, resulting in the precipitation of a solid. The mixture was filtered to obtain the filter cake, which was compound 54. MS m/z: 470.2 [M+1]⁺. ¹H NMR (400 MHz, DMSO-d₆) δ ppm 11.90 (s, 1H), 9.09 (dd, J=1.8, 4.5 Hz, 1H), 8.64 (dd, J=1.6, 7.9 Hz, 1H), 8.43 (d, J=8.3 Hz, 1H), 8.38 (s, 1H), 7.84 (d, J=8.5 Hz, 1H), 7.72 (dd, J=4.5, 8.0 Hz, 1H), 7.57 (dd, J=8.2, 10.7 Hz, 1H), 7.39-7.34 (m, 1H), 3.17-3.21 (m, 4H), 2.59-2.65 (m, 4H).

### Example 55

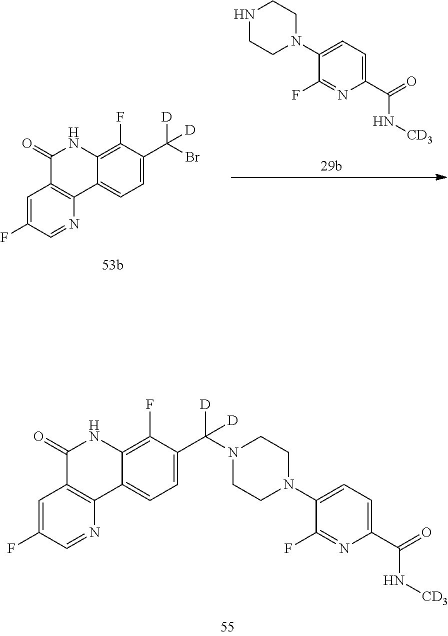 [SMILES: [2H]C([2H])(Br)c1ccc2c([nH]c(=O)c3cc(F)cnc32)c1F ; [2H]C([2H])([2H])NC(=O)c1ccc(N2CCNCC2)c(F)n1 ; [2H]C([2H])([2H])NC(=O)c1ccc(N2CCN(C([2H])([2H])c3ccc4c([nH]c(=O)c5cc(F)cnc54)c3F)CC2)c(F)n1]

### Step 1: Synthesis of Compound 55

Intermediate 29b (25 mg, hydrochloride) was dissolved in DMF (1 mL). Triethylamine (40.48 mg, 400.05 μmol) was added, and the reaction mixture was stirred at 25° C. for 0.5 hours. KI (8.30 mg, 50.01 μmol) and intermediate 53b (32.72 mg, 100.01 μmol) were then added, and the mixture was stirred at 50° C. for 1 hour. After cooling, the reaction mixture was added with 1 mL of water, resulting in the precipitation of a solid. The mixture was filtered to obtain the filter cake, which was compound 55. MS m/z: 488.2 [M+1]⁺. ¹H NMR (400 MHz, DMSO-d₆) δ ppm 12.06 (s, 1H), 9.14 (d, J=2.8 Hz, 1H), 8.42 (dd, J=3.0, 8.5 Hz, 1H), 8.39-8.34 (m, 2H), 7.84 (d, J=8.0 Hz, 1H), 7.56 (dd, J=8.0, 10.5 Hz, 1H), 7.38 (dd, J=6.5, 8.3 Hz, 1H), 3.15-3.19 (m, 4H), 2.56-2.64 (m, 4H).

### Bioassay Data:

### Experimental Example 1: Experiment on Binding Activity Between Compound and PARP1 Using Fluorescence Polarization Assay

### Experimental Methods:

The fluorescence polarization assay was performed in 96-well black-walled plates (Greiner). The reaction buffer consisted of 50 mM Tris (pH 8), 0.001% Triton X100, 10 mM MgCl₂, and 150 mM NaCl. Both the fluorescent probe and His-Avi-tagged PARP1 protein were manufactured by WuXi AppTec (Shanghai). In a 100 μL reaction system, 8 nM PARP1, 5 nM fluorescent probe, and the compounds were added respectively. The compound concentration was prepared following the starting concentration and dilution gradient in Table 1. For example, compound 1 started from 100 nM with a 3-fold dilution. The test sample was incubated at room temperature for 4 hours, protected from light. Fluorescence polarization values were measured using an En Vision plate reader. The obtained fluorescence polarization values and compound concentrations were subjected to nonlinear fitting using GraphPad 8.0 software to determine the IC₅₀ values.

### Experimental Results:

TABLE 1
PARP1 binding activity assay for the
compounds of the present disclosure

| | PARP1 | Starting concentration and dilution |
|---|---|---|
| Compound No. | IC₅₀ (nM) | gradient for test compounds |
| Trifluoroacetate | 11 | starting concentration at 100 nM, |
| of compound 1 | | 3-fold dilution gradient |
| Trifluoroacetate | 16 | starting concentration at 50 nM, |
| of compound 15 | | 2-fold dilution gradient |
| Trifluoroacetate | 13 | starting concentration at 50 nM, |
| of compound 16 | | 2-fold dilution gradient |
| Trifluoroacetate | 14 | starting concentration at 50 nM, |
| of compound 19 | | 2-fold dilution gradient |
| Compound 21 | 22 | starting concentration at 50 nM, |
| | | 2-fold dilution gradient |
| Trifluoroacetate | 14 | starting concentration at 50 nM, |
| of compound 22 | | 2-fold dilution gradient |
| Compound 29 | 9.7 | starting concentration at 1000 nM, |
| | | 3-fold dilution gradient |
| Compound 31 | 4.7 | starting concentration at 1000 nM, |
| | | 3-fold dilution gradient |
| Compound 33 | 4.8 | starting concentration at 1000 nM, |
| | | 3-fold dilution gradient |
| Compound 38 | 6.8 | starting concentration at 1000 nM, |
| | | 3-fold dilution gradient |
| Compound 44 | 10 | starting concentration at 1000 nM, |
| | | 3-fold dilution gradient |
| Compound 46 | 6.8 | starting concentration at 1000 nM, |
| | | 3-fold dilution gradient |
| Compound 50 | 7.9 | starting concentration at 1000 nM, |
| | | 3-fold dilution gradient |
| Compound 51 | 5.2 | starting concentration at 100 nM, |
| | | 3-fold dilution gradient |
| Compound 52 | 5.5 | starting concentration at 100 nM, |
| | | 3-fold dilution gradient |
| Compound 53 | 4.5 | starting concentration at 100 nM, |
| | | 3-fold dilution gradient |
| Compound 54 | 6.0 | starting concentration at 100 nM, |
| | | 3-fold dilution gradient |
| Compound 55 | 5.2 | starting concentration at 100 nM, |
| | | 3-fold dilution gradient |

Conclusion: The compounds of the present disclosure exhibit excellent binding activity to PARP1.

### Experimental Example 2: Experiment on Binding Activity Between Compound and PARP2 Using Fluorescence Polarization Assay

### Experimental Methods:

The fluorescence polarization assay was performed in 96-well black-walled plates (Greiner). The reaction buffer consisted of 50 mM Tris (pH 8), 0.001% Triton X100, 10 mM MgCl₂, and 150 mM NaCl. Both the fluorescent probe and His-Avi-tagged PARP2 protein were manufactured by WuXi AppTec (Shanghai). In a 100 μL reaction system, 3 nM PARP2, 5 nM fluorescent probe, and the compounds were added respectively. The compound concentration was prepared following the starting concentration and dilution gradient in Table 2. The test sample was incubated at room temperature for 4 hours, protected from light. Fluorescence polarization values were measured using an EnVision plate reader. The obtained fluorescence polarization values and compound concentrations were subjected to nonlinear fitting using GraphPad 8.0 software to determine the IC₅₀ values.

### Experimental Results:

TABLE 2
PARP2 binding activity assay for the
compounds of the present disclosure

| | PARP2 | Starting concentration and dilution |
|---|---|---|
| Compound No. | IC₅₀ (μM) | gradient for test compounds |
| Trifluoroacetate | >20 | starting concentration at 20 μM, |
| of compound 1 | | 3-fold dilution gradient |
| Trifluoroacetate | >50 | starting concentration at 50 μM, |
| of compound 15 | | 3-fold dilution gradient |
| Trifluoroacetate | >50 | starting concentration at 50 μM, |
| of compound 16 | | 3-fold dilution gradient |
| Trifluoroacetate | >50 | starting concentration at 50 μM, |
| of compound 19 | | 3-fold dilution gradient |
| Compound 21 | >50 | starting concentration at 50 μM, |
| | | 3-fold dilution gradient |
| Trifluoroacetate | >50 | starting concentration at 50 μM, |
| of compound 22 | | 3-fold dilution gradient |
| Compound 29 | >20 | starting concentration at 20 μM, |
| | | 3-fold dilution gradient |
| Compound 31 | >20 | starting concentration at 20 μM, |
| | | 3-fold dilution gradient |
| Compound 33 | >20 | starting concentration at 20 μM, |
| | | 3-fold dilution gradient |
| Compound 38 | >20 | starting concentration at 20 μM, |
| | | 3-fold dilution gradient |
| Compound 44 | >20 | starting concentration at 20 μM, |
| | | 3-fold dilution gradient |
| Compound 46 | >20 | starting concentration at 20 μM, |
| | | 3-fold dilution gradient |
| Compound 50 | >20 | starting concentration at 20 μM, |
| | | 3-fold dilution gradient |

Conclusion: The compounds of the present disclosure exhibit relatively weak binding activity to PARP2.

### Experimental Example 3: Anti-Proliferation Assay of Compounds on MDA-MB-436 Cells (BRCA1 Mutation)

### Experimental Methods:

MDA-MB-436 cells (BRCA1 mutation) were seeded in black 96-well plates with clear bottoms. Each well contained 135 μL of cell suspension with 3,500 MDA-MB-436 cells (BRCA1 mutation). The cell plate was incubated overnight in a CO₂ incubator. A 400× stock solution of the test compound was prepared. The test compounds were 5-fold diluted with a multi-channel pipette to the 9th concentration, i.e., diluted from 4 mM to 104 nM, and duplicate experiment was set. 78 μL of medium was added to an intermediate plate. Subsequently, 2 μL of the serially diluted compound per well was transferred to the corresponding wells of the intermediate plate. 2 μL of DMSO was added to the vehicle control and blank control. After mixing well, 15 μL of the mixture per well was then transferred to the cell plate. Concentrations of compounds transferred to the cell plate ranged from 10 μM to 0.26 nM, with a final DMSO concentration of 0.25%. The cell plate was incubated for 7 days in a CO₂ incubator. The cell plate was removed and equilibrated to room temperature for 30 minutes. 75 μL of chemiluminescent cell viability assay reagent was added to each well. The cell plate was shaken on an orbital shaker for 3 minutes to induce cell lysis, followed by 10 minutes of incubation at room temperature to stabilize the luminescent signal. Luminescent signals were detected using a 2104 EnVision plate reader. The inhibition rate (IR) of the test compounds was calculated using the following formula: IR (%)=(1-(RLU compound-RLU blank control)/(RLU vehicle control-RLU blank control))×100% The inhibition rates of compounds at different concentrations were calculated in Excel, and the inhibition curves were plotted and related parameters were calculated using GraphPad Prism software.

Experimental results: As shown in Table 3:

TABLE 3
Proliferation inhibition assay on
MDA-MB-436 cells (BRCA1 mutation)

| Compound No. | IC₅₀ (nM) |
|---|---|
| Trifluoroacetate | 1.2 |
| of compound 5 | |
| Trifluoroacetate | 1.58 |
| of compound 15 | |
| Trifluoroacetate | 5.73 |
| of compound 22 | |
| Compound 27 | 0.69 |
| Compound 28 | 0.37 |
| Compound 29 | 1.14 |
| Compound 31 | 0.75 |
| Compound 33 | 0.41 |
| Compound 37 | 4.54 |
| Compound 38 | 0.56 |
| Compound 41 | 2.92 |
| Compound 44 | 0.65 |
| Compound 45 | 0.27 |
| Compound 46 | 0.36 |
| Compound 50 | 0.41 |

Experimental conclusion: The compounds of the present disclosure exhibit excellent proliferation inhibitory activity against MDA-MB-436 cells (BRCA1 mutation).

### Experimental Example 4: Anti-Proliferation Assay of Compounds on DLD1 Cells (BRCA2 KO)

### Experimental Methods:

DLD1 (BRCA2 KO) cells were seeded in white a 96-well plate with 80 μL of cell suspension per well, containing 1,000 DLD1 (BRCA2 KO) cells per well. The cell plate was incubated overnight in a CO₂ incubator. The test compounds were 5-fold diluted with a multi-channel pipette to the 8th concentration, i.e., diluted from 2 mM to 0.0256 μM, and duplicate experiment was set. 78 μL of medium was added to an intermediate plate. Subsequently, 2 μL of the serially diluted compound per well was transferred to the corresponding wells of the intermediate plate and mixed well. 20 μL of the mixture per well was then transferred to the cell plate. The concentration of the compound transferred to the cell plate ranged from 10 μM to 0.128 nM. The cell plate was incubated for 7 days in a CO₂ incubator. An additional cell plate was prepared to read the signal value on the day of compound addition, which served as the maximum value (Max in the following equation) for data analysis. The cell plate was added with 25 μL of chemiluminescent cell viability assay reagent per well and incubated at room temperature for 10 minutes to stabilize the luminescent signal. A multimode microplate reader was used for reading. After the incubation of the cell plate with the compound, the cell plate was added with 25 μL of chemiluminescent cell viability assay reagent per well and incubated at room temperature for 10 minutes to stabilize the luminescent signal. A multimode microplate reader was used for reading. Using the equation (Sample−Min)/(Max−Min)×100% to convert the raw data into inhibition rate, the IC₅₀ value can be obtained by curve fitting with four parameters (obtained by “log (inhibitor) vs. response—Variable slope” mode in GraphPad Prism).

Experimental results: as shown in Table 4.

TABLE 4
Anti-proliferation assay on DLD1 (BRCA2 KO) cells

| Compound No. | IC₅₀ (nM) |
|---|---|
| Trifluoroacetate | 1.71 |
| of compound 15 | |
| Trifluoroacetate | 13.05 |
| of compound 16 | |
| Trifluoroacetate | 24.18 |
| of compound 19 | |
| Compound 21 | 10.01 |
| Trifluoroacetate | 4.43 |
| of compound 22 | |

Experimental conclusion: The compounds of the present disclosure exhibit good antiproliferative inhibitory effects on DLD1 (BRCA2 KO) cells.

### Experimental Example 5: In Vivo Pharmacokinetic Evaluation and Brain Penetration Evaluation of the Compounds of the Present Disclosure in Mice

### Experimental Methods:

The trifluoroacetate of compound 16 was evaluated using female BALB/C mice, and the trifluoroacetate of compound 15 was evaluated using male CD-1 mice. A clear solution of the test compound (0.4 mg/mL) in 10% 2-hydroxypropyl-β-cyclodextrin and 90% water was administered to the mice (fasted overnight, aged 7 to 9 weeks) via tail vein injection at a dose of 2 mg/kg. A clear solution of the test compound (1 mg/mL) in 0.5% methylcellulose was administered to the mice (fasted overnight, aged 7 to 9 weeks) via oral gavage at a dose of 10 mg/kg. Blood samples (about 30 μL) were collected from the two groups of animals at the following time points: from the jugular vein at 0.0833, 0.25, 0.5, 1.0, 2.0, 4.0, 8.0, and 24 hours post-administration, and from the tail vein at 0.25, 0.5, 1.0, 2.0, 4.0, 6.0, 8.0, and 24 hours post-administration. The samples were placed in anticoagulant tubes containing EDTA-K2, followed by centrifugation to separate the plasma. For the trifluoroacetate of compound 15, one group received intravenous administration (IV, 2 mg/kg) with blood samples collected at the above time points, and another group received oral administration (PO, 10 mg/kg) with blood samples collected at the above time points. Additionally, for the oral administration group (PO, 10 mg/kg), plasma and brain tissue homogenate samples were collected at the 2-hour time point. Plasma concentrations were determined by LC-MS/MS method, and relevant pharmacokinetic parameters were calculated using WinNonlin™ Version 6.3 (Pharsight, Mountain View, CA) pharmacokinetic software with non-compartmental model linear-log trapezoidal method.

Experimental results: The pharmacokinetic data of mice are shown in Table 5, and the brain penetration data are shown in Table 6.

TABLE 5
Pharmacokinetic data of mice

| | Trifluoroacetate of compound 16 | | Trifluoroacetate of compound 15 | |
|---|---|---|---|---|
| | IV 2 | PO 10 | IV 2 | PO 10 |
| | mg/kg | mg/kg | mg/kg | mg/kg |
| C₀ (nM) or | 14017 | 42673 | 4210 | 13700 |
| Cₘₐₓ (nM) | | | | |
| Tₘₐₓ (h) | — | 0.5 | — | 0.25 |
| T_{1/2} (h) | 0.25 | 2 | 3.7 | 4.1 |
| Vdₛₛ (L/kg) | 1.54 | — | 2.1 | — |
| Cl (mL/min/kg) | 4.0 | — | 7.7 | — |
| Tₗₐₛₜ (h) | 24.0 | 24.0 | 24.0 | 24.0 |
| AUC₀₋ₗₐₛₜ (nM · h) | 17915 | 173382 | 9230 | 61600 |

Note:
“—” indicates that the parameter could not be calculated. C₀ represents initial concentration; Cₘₐₓ represents peak concentration; Tₘₐₓ represents time to reach peak concentration; T_{1/2} represents elimination half-life; Vdₛₛ represents apparent volume of distribution at steady state; Cl represents total clearance; Tₗₐₛₜ represents time point of the last quantifiable drug concentration; AUC₀₋ₗₐₛₜ represents area under the plasma concentration-time curve from time 0 to the last quantifiable time point.

TABLE 6
Brain penetration data of the compound of the present disclosure in mice

| | PO 10 mg/kg Drug concentrations at the 2-hour time point | | |
|---|---|---|---|
| | C_{2 h}(nM) | C_{2 h}(nM) | Brain-to-plasma |
| | Plasma drug | Brain drug | drug concentration |
| | concentration | concentration | ratio |
| Trifluoroacetate | 8467 | 5814 | 0.7 |
| of compound 15 | | | |

Experimental conclusion: The compounds of the present disclosure exhibit excellent metabolic stability in vivo and excellent drug exposure following oral administration. Oral administration result in superior drug concentration in brain tissue, with a high brain-to-plasma concentration ratio.

### Experimental Example 6: Permeability Evaluation of the Compounds of the Present Disclosure

### Cell Line:

This experiment used the MDR1-MDCK II cell line, authorized by the Piet Borst Laboratory at the Netherlands Cancer Institute, as the in vitro model for permeability evaluation experiment. Cells were seeded at a density of 2.3×10⁵ cells/cm² in a Transwell-96 well plate. After being cultured for 4 to 7 days in a CO₂ incubator, the cells were used for transport experiments.

### Experimental Conditions:

Test compound concentration: 2.00 μM;
Testing directions and replicates: Bidirectional (A (apical)-B (basolateral) and B-A), with 2 replicates;
Transport buffer (TB): HBSS solution containing 10 mM HEPES (pH 7.40±0.05) Incubation conditions: 37±1° C., 5% CO₂, incubated for 150 minutes

Control compounds: Nadolol and Metoprolol were used as control compounds for low and high permeability, respectively. Digoxin was used as a substrate for P-glycoprotein. The administration concentration of nadolol and metoprolol was 2.00 μM, and that of digoxin was 10.0 μM.

Monolayer cell membrane integrity test:

After the transport experiment, the integrity of the MDR1-MDCK II cell layer was assessed using the Lucifer Yellow Rejection Assay. The remaining solution in the apical and basolateral wells was removed, followed by the addition of 75 μL of TB containing 100 μM Lucifer Yellow to the apical wells and 250 μL of TB to the basolateral wells. The cell plates were incubated in a cell incubator at 37±1° C., 5% CO₂, and saturated humidity for 30 minutes. After incubation, 20 μL of the sample was collected from the apical side and mixed with 60 μL of TB, while 80 μL of the sample was collected from the basolateral side. The relative fluorescence intensity (RFU) was measured using a microplate reader at 425/528 nm (excitation/emission).

### Sample Analysis:

In this experiment, sample analysis for the test compounds and the control compounds—nadolol, metoprolol, and digoxin—was conducted using liquid chromatography-tandem mass spectrometry (LC-MS/MS). The retention time, chromatogram acquisition, and chromatogram integration of the analytes and internal standards were processed using Analyst software (Sciex, Framingham, MA, USA). The semi-quantitative determination of the sample analysis was performed by calculating the ratio of the peak area of the analyte to that of the internal standard.

### Data Analysis:

$${P}_{app} = \frac{{V}_{R}}{Area \times Time} \times \frac{{[ drug ]}_{receiver}}{{[ drug ]}_{initial , donor}} = \frac{{V}_{R}}{Area \times Time} \times Efflux Ratio = \frac{{P}_{app} ( B to A )}{{P}_{app} ( A to B )} \\ \% Solution Recovery = \frac{{C}_{R} \times {V}_{R} + {C}_{D} \times {V}_{D}}{{C}_{0} \times {V}_{D}}$$

V_{R} represents the volume of the solution at the receiving side (0.075 mL for the apical side and 0.25 mL for the basolateral side). Area represents the relative surface area of the cell monolayer (0.0804 cm²). Time represents the incubation time (9000 s). C₀ represents the peak area ratio of the compound on the donor side. V_{D} is the volume on the donor side (0.075 mL for the apical side and 0.25 mL for the basolateral side). C_{D} and C_{R} represent the peak area ratios of the compound on the donor and receiving sides, respectively.

The permeability of Lucifer Yellow (% Lucifer Yellow) was calculated using the following formula:

$$\% Lucifer Yellow = \frac{{V}_{Basolateral} \times {RFU}_{Basolateral}}{{V}_{Apical} \times {RFU}_{Apical} + {V}_{Basolateral} \times {RFU}_{Basolateral}} \times 100$$

RFU_{Apical} and RFU_{Basolateral} are the relative fluorescence intensities of Lucifer Yellow on the apical and basolateral sides, respectively. V_{Apical} and V_{Basolateral} are the loading volumes on the apical and basolateral sides, which are 0.0750 mL and 0.250 mL, respectively.

### Experimental Results:

The permeability test results of the compounds of the present disclosure on the MDR1-MDCK II cell line are summarized in Table 7.

TABLE 7
Summary of permeability evaluation of
the compounds in the present disclosure

| | Permeability Pₐₚₚ | | | |
|---|---|---|---|---|
| | (10⁻⁶ cm/s) | | Efflux | Permeability |
| Compound | A to B | B to A | ratio | conclusion |
| Trifluoroacetate | 25.2 | 17.7 | 0.703 | High |
| of compound 15 | | | | permeability |
| Compound 33 | 10.6 | 7.2 | 0.679 | High |
| | | | | permeability |

Note:
low permeability: Pₐₚₚ ≤ 1.0 (×10⁻⁶ cm/s); moderate permeability: 1.0 < Pₐₚₚ < 5.5 (×10⁻⁶ cm/s); high permeability: Pₐₚₚ ≥ 5.5 (×10⁻⁶ cm/s).

Conclusion: The compounds of the present disclosure demonstrate excellent membrane permeability in the cell membrane permeability study.

### Experimental Example 7: Pharmacokinetic Evaluation and Brain Penetration Evaluation of the Compounds of the Present Disclosure in Rats

### Experimental Methods:

A 0.2 mg/mL clear solution of the test compound in 10% 2-hydroxypropyl-β-cyclodextrin and 90% water was administered to male SD rats (fasted overnight, 200 to 230 g) via tail vein injection at a dose of 1 mg/kg. A 0.5 mg/mL solution of the test compound in 10% 2-hydroxypropyl-β-cyclodextrin and 90% water was administered to male SD rats (fasted overnight, 200 to 230 g) via oral gavage at a dose of 5 mg/kg. Blood samples (about 30 μL) were collected from the two groups of animals at the following time points: from the jugular vein at 0.0833, 0.25, 0.5, 1.0, 2.0, 4.0, 8.0, and 24 hours post-administration, and from the tail vein at 0.25, 0.5, 1.0, 2.0, 4.0, 8.0, and 24 hours post-administration. The samples were placed in anticoagulant tubes containing EDTA-K2, followed by centrifugation to separate the plasma. For the trifluoroacetate of compound 15, one group received intravenous administration (IV, 1 mg/kg) with blood samples collected at the above time points, and another group received oral administration (PO, 5 mg/kg) with blood samples collected at the above time points. Additionally, for the oral administration group (PO, 5 mg/kg), plasma and brain tissue homogenate samples were collected at the 2-hour time point. Plasma concentrations were determined by LC-MS/MS method, and relevant pharmacokinetic parameters were calculated using WinNonlin™ Version 6.3 (Pharsight, Mountain View, CA) pharmacokinetic software with non-compartmental model linear-log trapezoidal method.

Experimental results: The pharmacokinetic data of rats are shown in Table 8, and the brain penetration data are shown in Table 9.

TABLE 8
Pharmacokinetic data of rats

Trifluoroacetate of compound 15

| | IV 1 mg/kg | PO 5 mg/kg |
|---|---|---|
| C₀ (nM) or | 2120 | 2830 |
| Cₘₐₓ (nM) | | |
| Tₘₐₓ (h) | — | 4 |
| T_{1/2} (h) | 5.2 | 3.3 |
| Vdₛₛ (L/kg) | 2.1 | — |
| Cl (mL/min/kg) | 6.0 | — |
| Tₗₐₛₜ (h) | 24.0 | 24.0 |
| AUC₀₋ₗₐₛₜ (nM · h) | 5820 | 22900 |
| F (%) | | 78% |

Note:
“—” indicates that the parameter could not be calculated; C₀ represents initial concentration; Cₘₐₓ represents peak concentration; Tₘₐₓ represents time to reach peak concentration; T_{1/2} represents elimination half-life; Vdₛₛ represents apparent volume of distribution at steady state; Cl represents total clearance; Tₗₐₛₜ represents time point of the last quantifiable drug concentration; AUC₀₋ₗₐₛₜ represents area under the plasma concentration-time curve from time 0 to the last quantifiable time point; F(%) represents bioavailability, calculated using AUCₒ₋ₗₐₛₜ.

TABLE 9
Brain penetration data of the compound of the present disclosure in rats

| | PO 5 mg/kg Drug concentrations at the 2-hour time point | | |
|---|---|---|---|
| | C_{2 h}(nM) | C_{2 h}(nM) | Brain-to-plasma |
| | Plasma drug | Brain drug | drug concentration |
| | concentration | concentration | ratio |
| Trifluoroacetate | 8467 | 5814 | 0.7 |
| of compound 15 | | | |

Experimental conclusion: The compounds of the present disclosure exhibit excellent metabolic stability in vivo and excellent drug exposure following oral administration. Oral administration result in superior drug concentration in brain tissue, with a high brain-to-plasma concentration ratio.

### Experimental Example 8: In Vitro Liver Microsomal Stability of the Compound of the Present Disclosure

Experimental objective: To investigate the metabolic stability of the compound of the present disclosure in humans through in vitro metabolic stability testing using human liver microsomes.

Experimental conditions: The compound (1 μM) was incubated with human liver microsomes supplemented with an NADPH regeneration system at 37° C. for a specified duration, lasting up to 60 minutes. The concentration of the compound in the samples was determined by LC-MS/MS.

Experimental process: The incubation was performed using 96-well incubation plates, with individual plates labeled as T0, T5, T15, T30, T45, T60, Blank60, and NCF60. The reaction time points corresponding to the incubation plates were set at 0, 5, 15, 30, 45, and 60 minutes. In the Blank60 plate, no test compound or control compound was added, and the sample was collected after 60 minutes of incubation. In the NCF60 plate, potassium phosphate buffer was used in place of the NADPH regeneration system and incubated for 60 minutes. All time point samples were taken from individual wells.

For the T0, T5, T15, T30, T45, T60, and NCF60 plates, 5 μL of either the test or control compound working solution and 100 μL of microsomal working solution (with a liver microsomal protein concentration of 0.5 mg/mL) were added. Only microsomal working solution was added to the Blank60 well. In the Blank60 plate, only the microsomal working solution was added. Then, the incubation plates, except for TO and NCF60, including Blank60, T5, T15, T30, T45, and T60, were pre-incubated in a 37° C. water bath for approximately 10 minutes.

For samples in the TO plate, 180 μL of stop solution (where the stop solution was a solution of acetonitrile and methanol (95:5, V/V) containing 100 ng/mL tolbutamide) was added before adding the NADPH regeneration system working solution.

50 μL of potassium phosphate buffer was added to each well of the NCF60 plate and incubated for 60 minutes.

After the pre-incubation of Blank60, T5, T15, T30, T45, and T60 incubation plates, 44 μL of NADPH regeneration system working solution was added to each sample well to initiate the reaction. Therefore, for sample wells containing either the test compound or the control compound working solutions, the final reaction concentration was 1 μM, with the concentration of liver microsomes at 0.5 mg/mL. The final concentrations of DMSO and acetonitrile in the reaction system were 0.01% (v/v) and 0.99% (v/v), respectively.

After incubation for an appropriate period (e.g., 5, 15, 30, 45, and 60 minutes), 180 μL of stop solution was added to each test compound and control compound sample well in the Blank60, T5, T15, T30, T45, T60, and NCF60 plates to stop the reaction.

All sample plates were shaken thoroughly and centrifuged for 10 minutes. A volume of 80 μL of the supernatant from each test compound was diluted to 240 L with a solution of acetonitrile (1:9, v/v) containing 0.1% formic acid for LC-MS/MS analysis.

The half-life (T_{1/2}) and clearance (CLᵢₙₜ₍ₗᵢᵥₑᵣ₎) of the compound in human liver microsomes were determined by calculating the percentage of the compound remaining at each corresponding time point.

$${C}_{t} = {C}_{0} \cdot {e}^{- {k}_{e} \cdot t} \\ when \\ {C}_{t} = \frac{1}{2} {C}_{0} , \\ {T}_{1 / 2} = \frac{Ln 2}{{k}_{e}} = \frac{0.693}{{k}_{e}} \\ {CL}_{i n t ( mic )} = \frac{0.693}{In vitro {T}_{1 / 2}} \cdot \frac{1}{mg / mL microsomal protein in reaction system} \\ {CL}_{i n t ( liver )} = {CL}_{i n t ( mic )} \cdot \frac{mg microsomes}{g liver} \cdot \frac{g liver}{kg body weight}$$

Experimental results: As shown in Table 10.

TABLE 10
In vitro metabolic stability study results of the compounds
of the present disclosure in human and mouse liver microsomes

| | Human liver microsomes | | Mouse liver microsomes | |
|---|---|---|---|---|
| | CLᵢₙₜ₍ₗᵢᵥₑᵣ₎ | T_{1/2} | CLᵢₙₜ₍ₗᵢᵥₑᵣ₎ | T_{1/2} |
| | (mL/min/kg) | (minutes) | (mL/min/kg) | (minutes) |
| Trifluoroacetate | <8.6 | >145 | <38 | >145 |
| of compound 1 | | | | |
| Compound 15 | <8.6 | >145 | 74 | 77 |
| Compound 27 | 9.5 | 132 | NA | NA |
| Compound 29 | NA | NA | <38 | >145 |
| Compound 31 | <8.6 | >145 | NA | NA |
| Compound 37 | NA | NA | 64 | 85 |

NA: Not tested

Conclusion: The compounds of the present disclosure demonstrate outstanding stability in both human and mouse liver microsomes in vitro.

### Experimental Example 9: In Vivo Pharmacodynamic Study of the Compounds of the Present Disclosure in a Human Breast Cancer MDA-MB-436 Cell Subcutaneous Xenograft Tumor Model Using BALB/c Nude Mice

### Experimental Objective:

To investigate the inhibitory effect of the compound of the present disclosure on the in vivo growth of tumors in a human breast cancer MDA-MB-436 xenograft model in nude mice.

### Experimental Methods:

### 1) Cell Culture:

Routine cell culture was performed under 5% CO₂ at 37° C. in MEM medium containing 10% fetal bovine serum. The cells were passaged using 0.25% trypsin digestion. Depending on the growth condition of the cells, the cells were passaged two to three times per week at a ratio of 1:3 to 1:6.

### 2) Preparation of Animal Model

Animals: Female BALB/c nude mice, 6 to 8 weeks of age.

MDA-MB-436 cells in the logarithmic growth phase were collected, counted, and then resuspended in a mixture of 50% serum-free MEM medium and 50% Matrigel to adjust the cell concentration to 5.0×10⁷ cells/mL. After dispersing the cells evenly with a pipette, the suspension was transferred into a 50 mL centrifuge tube, which was placed in an icebox. Using a 1 mL syringe, the cell suspension was injected subcutaneously into the right axilla of each nude mouse, with 200 μL (1.1×10⁷ cells/mouse) injected per animal, establishing the MDA-MB-436 nude mouse xenograft model. After inoculation, the animals were regularly observed for their condition and tumor growth. Tumor diameters were measured using an electronic caliper, and the data were directly input into an Excel spreadsheet to calculate tumor volume. When the tumor volume reached 100 to 200 mm³, healthy animals with similar tumor sizes were selected and grouped (n=6) based on a randomized block design, while ensuring that the average body weight of each group remained consistent. The day of grouping was considered as the first day of the experiment (D1). After the experiment commenced, the tumor diameter was measured twice weekly, and tumor volume was calculated, while the body weight of the animals was weighed and recorded.

The formula used for calculating tumor volume (TV) is as follows: TV (mm³)=a×w²/2; where a represents the long diameter of the tumor (mm) and w represents the short diameter of the tumor (mm).

3) Dose: 1.0 mg/Kg; Administration Route: Oral Gavage (p.o.), Once Daily (QD)

### 4) Data Recording

The formula for calculating Relative Tumor Volume (RTV) is as follows: RTV=TVt/TVinitial; where TVinitial is the tumor volume measured at the time of grouping and dosing, and TVt is the tumor volume measured at each time point during administration.

The formula for calculating the Relative Tumor Proliferation Rate (% T/C) is: % T/C=100%×(RTVT/RTVC); where RTVT represents the RTV of the treatment group, and RTVC represents the RTV of the vehicle control group.

The formula for calculating Tumor Growth Inhibition (TGI) is: TGI=100%×[1−(TVt(T)−TVinitial(T))/(TVt(C)−TVinitial(C))]; where TVt(T) is the tumor volume of the treatment group at each measurement, TVinitial(T) is the tumor volume of the treatment group at the time of grouping and administration, TVt(C) is the tumor volume of the vehicle group at each measurement, and TVinitial(C) is the tumor volume of the vehicle group at the time of grouping and administration.

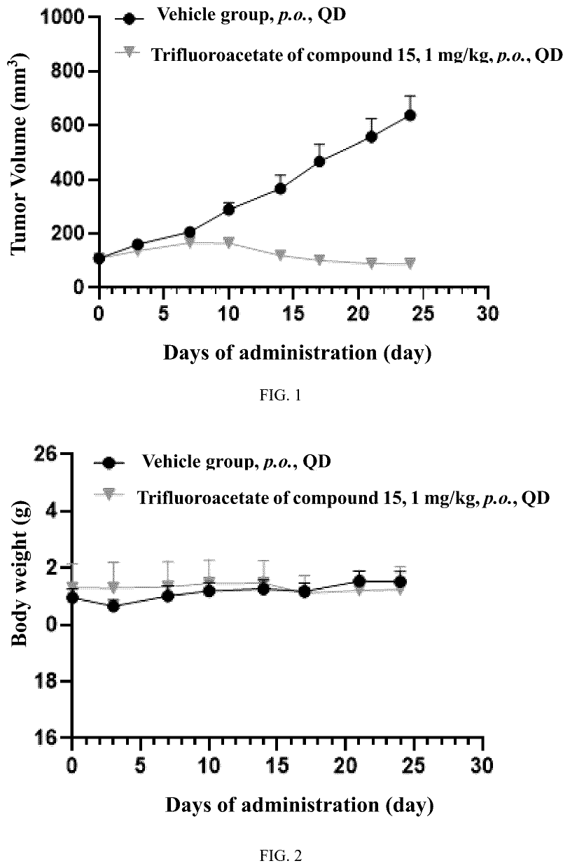

Experimental results: Tumor growth volumes are shown in FIG. 1. The body weight changes of mice during the administration are shown in FIG. 2. Tumor growth inhibition rates and relative tumor proliferation rates are shown in Table 11.

TABLE 11
Summary of tumor growth inhibition rate and relative
tumor proliferation rate in MDA-MB-436

| | | TGI (%) | T/C (%) | |
|---|---|---|---|---|
| | | (tumor growth | (relative tumor | |
| | Dose | inhibition | proliferation | |
| | (p.o.) | rate) D24 | rate) D24 | p-value¹ |
| Trifluoroacetate | 1.0 mg/kg | 104% | 14% | **** |
| of compound 15 | | | | |

¹Comparisons between the two groups were analyzed by T-test. A p-value < 0.05 was considered statistically significant: *p < 0.05, **p < 0.01, ***p < 0.001, ****p < 0.0001.

Experimental conclusion: The compounds of the present disclosure exhibit significant anti-tumor activity.

1. A compound of formula (XII) or a pharmaceutically acceptable salt thereof, 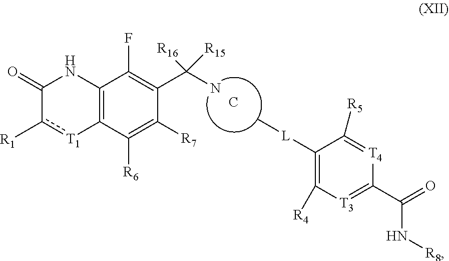
wherein
X is selected from O and S;
the structural moiety 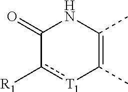
is selected from 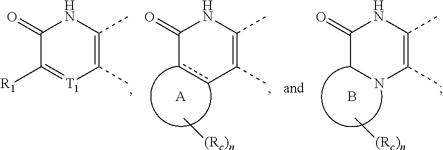
ring A is selected from phenyl and 6-membered heteroaryl;
ring B is selected from 6-membered heteroaryl;
L is selected from a single bond, and ring C is selected from 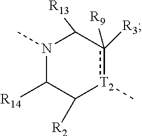 [symbol]
is selected from a single bond or a double bond;
T₂ is selected from C, N, and CH;
T₃ and T₄ are each independently selected from N and CR₁₀;
R₁ is selected from C₁₋₃ alkyl and C₃₋₅ cycloalkyl, and the C₁₋₃ alkyl and C₃₋₅ cycloalkyl are each independently and optionally substituted by 1, 2, or 3 halogens;
R₂ is selected from H and C₁₋₃ alkyl, and the C₁₋₃ alkyl is optionally substituted by 1, 2, or 3 halogens;
R₃ is selected from H and halogen;
R₉ is absent, or selected from H and halogen;
R₄ is selected from H, halogen, CN, C₁₋₃ alkyl, and C₁₋₃ alkoxy;
R₅ is selected from H, halogen, CN, C₁₋₃ alkyl, and C₁₋₃ alkoxy;
R₆ and R₇ are each independently selected from H, halogen, C₁₋₃ alkyl, and C₁₋₃ alkoxy, and the C₁₋₃ alkyl and C₁₋₃ alkoxy are each independently and optionally substituted by 1, 2, or 3 halogens;
R₈ is selected from C₁₋₃ alkyl and C₃₋₅ cycloalkyl, and the C₁₋₃ alkyl and C₃₋₅ cycloalkyl are each independently and optionally substituted by 1, 2, or 3 R;
R₁₀ is selected from H and halogen;
R₁₃ and R₁₄ are each independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are each independently and optionally substituted by 1, 2, or 3 halogens;
R₁₅ and R₁₆ are each independently selected from H, D, C₁₋₃ alkyl, and C₁₋₃ alkoxy, and the C₁₋₃ alkyl and C₁₋₃ alkoxy are each independently and optionally substituted by 1, 2, or 3 halogens;
or, R₂ and R₄ form a ring, so that the structural moiety 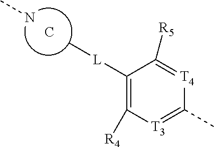
is selected from 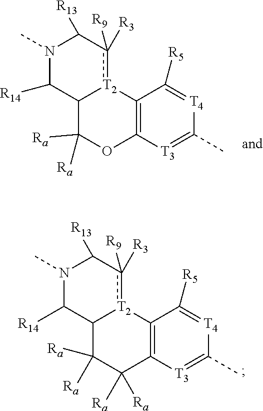
or, R₃ and R₅ form a ring, so that the structural moiety 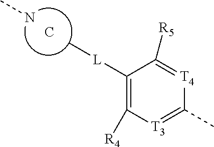
is selected from 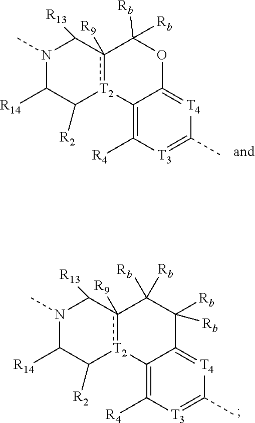
each Rₐ is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
or, two Rₐ on adjacent atoms, together with the atom to which they are attached, form a double bond or a cyclopropyl group;
each R_{b} is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
each R_{c} is independently selected from H, halogen, C₁₋₃ alkyl, C₁₋₃ alkoxy, and C₃₋₅ cycloalkyl, and the C₁₋₃ alkyl, C₁₋₃ alkoxy, and C₃₋₅ cycloalkyl are optionally substituted by 1, 2, or 3 halogens;
each R is independently selected from halogen and D;
n is selected from 0, 1, 2, 3, and 4;
given that when the structural moiety 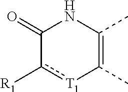
is selected from 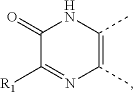
L is selected from a single bond, and ring C is selected from 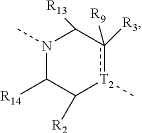
then R₂ and R₄ form a ring, or R₃ and R₅ form a ring;
the halogen represents F, Cl, Br, and I atoms;
“hetero” in the 5- to 6-membered heteroaryl represents 1, 2, 3, or 4 heteroatoms or heteroatom groups, each independently selected from —O—, —S—, and —N—.

2. The compound or the pharmaceutically acceptable salt thereof according to claim 1, wherein the compound has a structure of formula (XII-1), 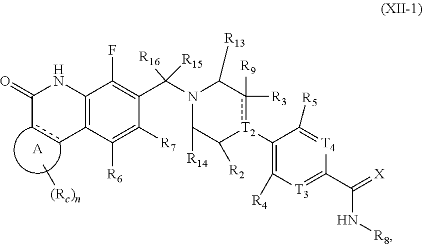
wherein
ring A is selected from 6-membered heteroaryl.

3. The compound or the pharmaceutically acceptable salt thereof according to claim 1, wherein it satisfies one or more of the following conditions:
(1) X is selected from O;
(2) each Rₐ, each R_{b}, and each R_{c} are independently selected from H, F, and CH₃;
(3) T₃ is selected from N, CH, and CF;
(4) T₄ is selected from N and CH;
(5) R₁ is selected from CH₃ and CH₂CH₃;
(6) R₂ is selected from H and CH₃;
(7) R₆ and R₇ are each independently selected from H, F, and CH₃;
(8) R₄ and R₅ are each independently selected from H, F, Cl, CN, and CH₃;
(9) R₈ is selected from CH₃, CH₂CH₃, CH₂CF₃, cyclopropyl, and CD₃;
(10) R₃ and R₉ are each independently selected from H and F;
(11) R₁₀ is selected from H and F;
(12) R₁₃ and R₁₄ are each independently selected from H and CH₃;
(13) R₁₅ and R₁₆ are each independently selected from H, D, and CH₃;
(14) ring A is selected from phenyl, pyridyl, pyrazinyl, and pyrimidinyl.

4. (canceled)

5. The compound or the pharmaceutically acceptable salt thereof according to claim 1, wherein T₃ is selected from N.

6. (canceled)

7. The compound or the pharmaceutically acceptable salt thereof according to claim 1, wherein R₁ is selected from CH₃.

8. (canceled)

9. The compound or the pharmaceutically acceptable salt thereof according to claim 1, wherein R₆ and R₇ are each independently selected from H and F.

10-16. (canceled)

17. The compound or the pharmaceutically acceptable salt thereof according to claim 1, wherein ring B is selected from pyridyl, pyrazinyl, and pyrimidinyl.

18. The compound or the pharmaceutically acceptable salt thereof according to claim 1, wherein the structural moiety 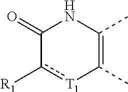
is selected from

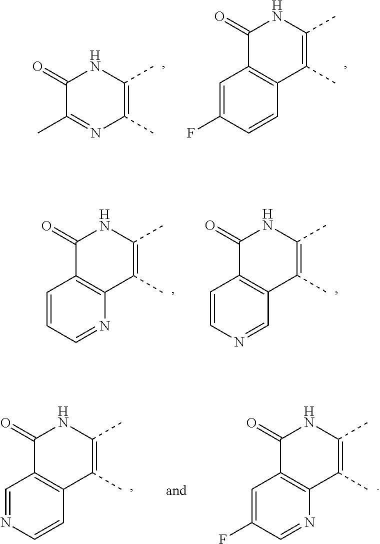

19. The compound or the pharmaceutically acceptable salt thereof according to claim 1, wherein the structural moiety 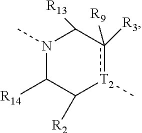
is selected from

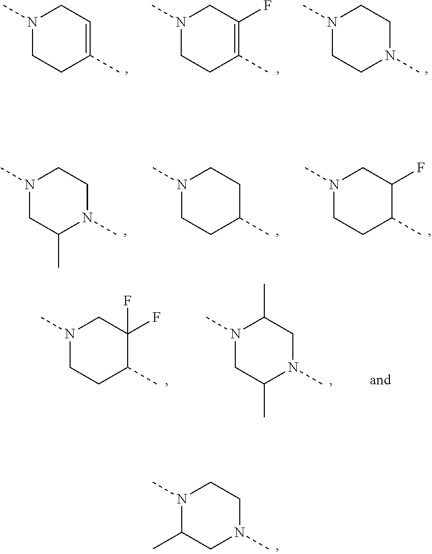

20. The compound or the pharmaceutically acceptable salt thereof according to claim 1, wherein the structural moiety 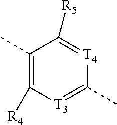
is selected from

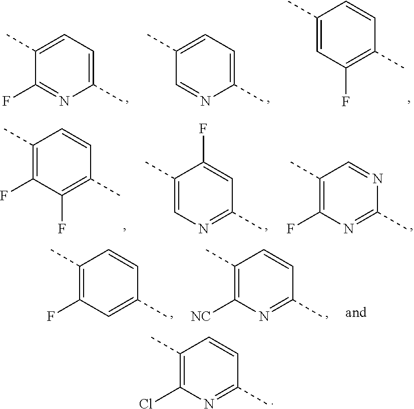

21. The compound or the pharmaceutically acceptable salt thereof according to claim 1, wherein L is selected from a single bond, and the structural moiety 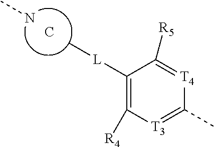
is selected from

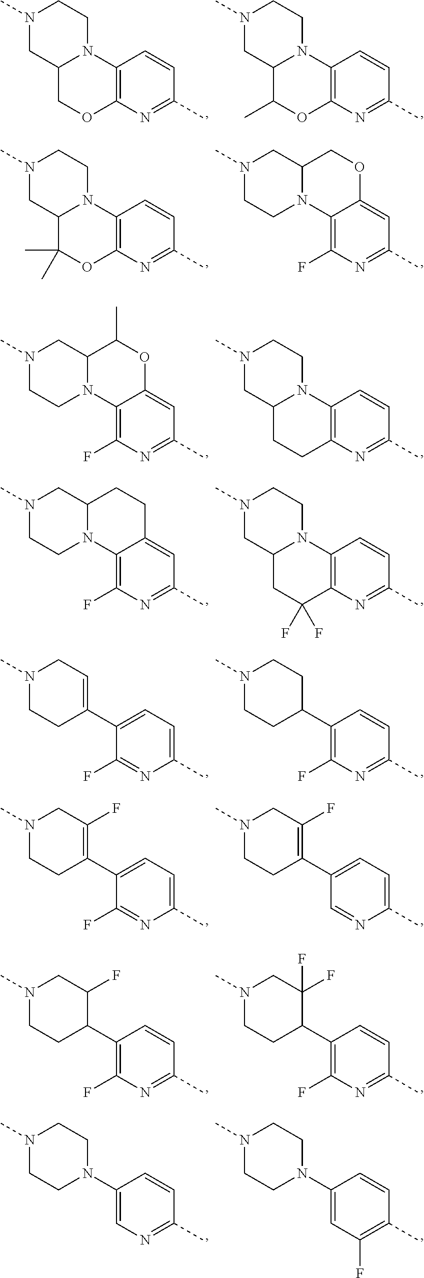

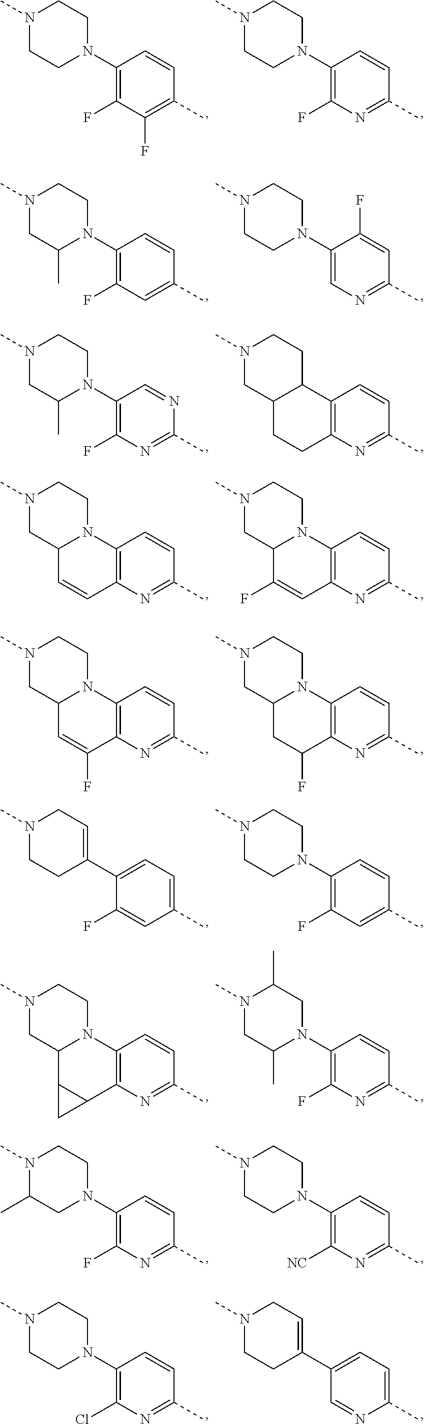

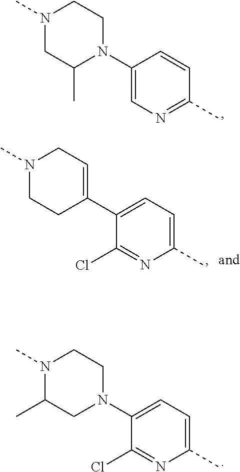

22. The compound or the pharmaceutically acceptable salt thereof according to claim 1, wherein the compound has a structure of formula (P-1), 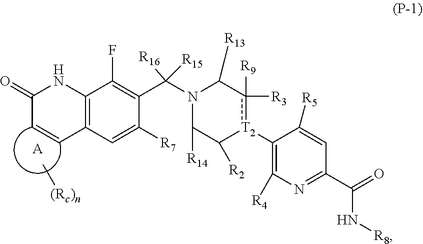
wherein ring A is selected from pyridyl.

23. The compound or the pharmaceutically acceptable salt thereof according to claim 1, wherein the compound has a structure of formula (P-2), 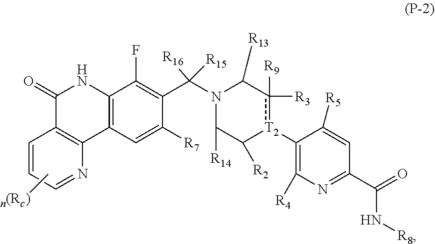
wherein [symbol] is selected from a single bond or a double bond;
T₂ is selected from C and N;
R₂ is selected from H and C₁₋₃ alkyl, and the C₁₋₃ alkyl is optionally substituted by 1, 2, or 3 halogens;
R₃ is selected from H and halogen;
R₄ is selected from H, halogen, CN, C₁₋₃ alkyl, and C₁₋₃ alkoxy;
R₅ is selected from H, halogen, CN, C₁₋₃ alkyl, and C₁₋₃ alkoxy;
R₉ is absent, or selected from H and halogen.

24. A compound as shown below or a pharmaceutically acceptable salt thereof,

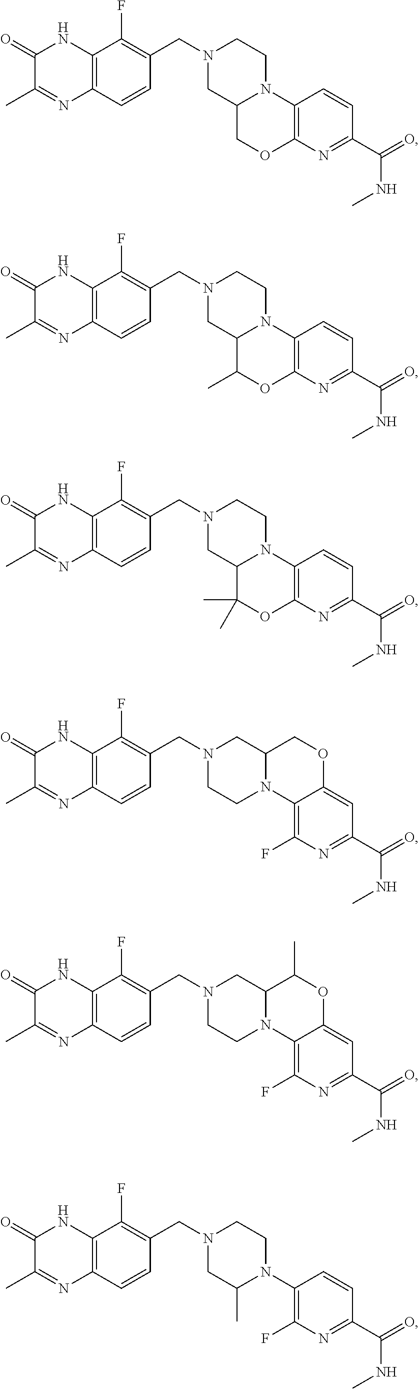 [SMILES: CNC(=O)c1ccc2c(n1)OCC1CN(Cc3ccc4nc(C)c(=O)[nH]c4c3F)CCN21 ; CNC(=O)c1ccc2c(n1)OC(C)C1CN(Cc3ccc4nc(C)c(=O)[nH]c4c3F)CCN21 ; CNC(=O)c1ccc2c(n1)OC(C)(C)C1CN(Cc3ccc4nc(C)c(=O)[nH]c4c3F)CCN21 ; CNC(=O)c1cc2c(c(F)n1)N1CCN(Cc3ccc4nc(C)c(=O)[nH]c4c3F)CC1CO2 ; CNC(=O)c1cc2c(c(F)n1)N1CCN(Cc3ccc4nc(C)c(=O)[nH]c4c3F)CC1C(C)O2 ; CNC(=O)c1ccc(N2CCN(Cc3ccc4nc(C)c(=O)[nH]c4c3F)CC2C)c(F)n1]

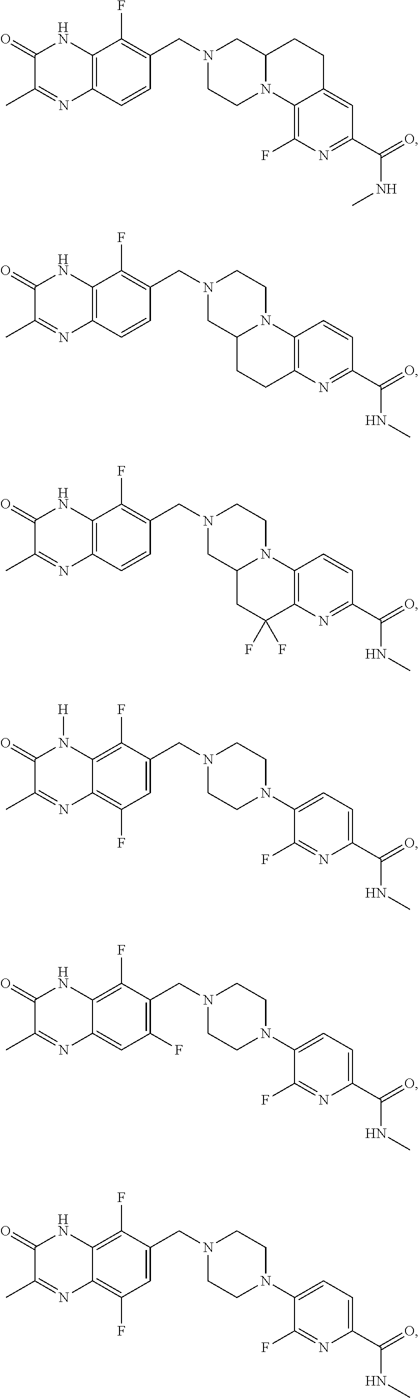 [SMILES: CNC(=O)c1cc2c(c(F)n1)N1CCN(Cc3ccc4nc(C)c(=O)[nH]c4c3F)CC1CC2 ; CNC(=O)c1ccc2c(n1)CCC1CN(Cc3ccc4nc(C)c(=O)[nH]c4c3F)CCN21 ; CNC(=O)c1ccc2c(n1)C(F)(F)CC1CN(Cc3ccc4nc(C)c(=O)[nH]c4c3F)CCN21 ; CNC(=O)c1ccc(N2CCN(Cc3cc(F)c4nc(C)c(=O)[nH]c4c3F)CC2)c(F)n1 ; CNC(=O)c1ccc(N2CCN(Cc3c(F)cc4nc(C)c(=O)[nH]c4c3F)CC2)c(F)n1 ; CNC(=O)c1ccc(N2CCN(Cc3cc(F)c4nc(C)c(=O)[nH]c4c3F)CC2)c(F)n1]

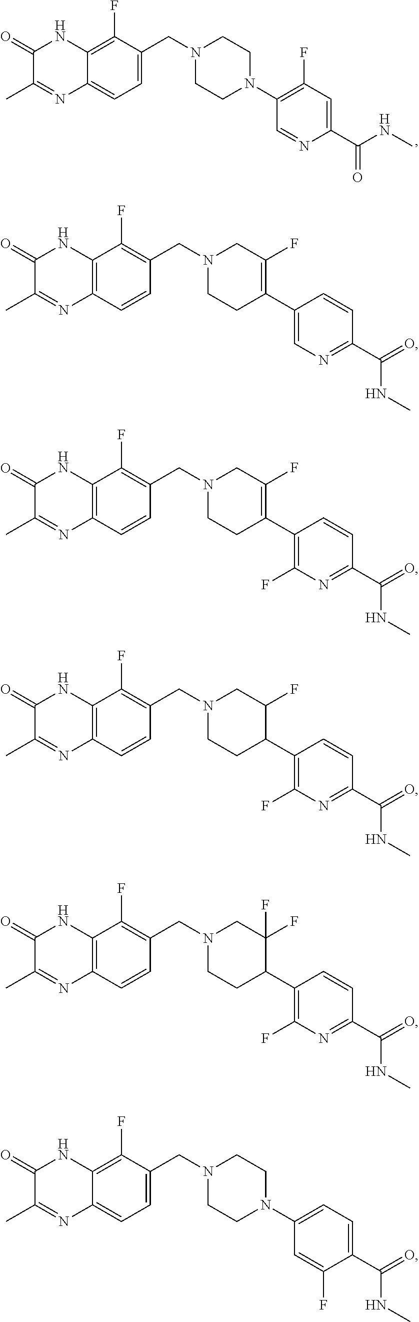 [SMILES: CNC(=O)c1cc(F)c(N2CCN(Cc3ccc4nc(C)c(=O)[nH]c4c3F)CC2)cn1 ; CNC(=O)c1ccc(C2=C(F)CN(Cc3ccc4nc(C)c(=O)[nH]c4c3F)CC2)cn1 ; CNC(=O)c1ccc(C2=C(F)CN(Cc3ccc4nc(C)c(=O)[nH]c4c3F)CC2)c(F)n1 ; CNC(=O)c1ccc(C2CCN(Cc3ccc4nc(C)c(=O)[nH]c4c3F)CC2F)c(F)n1 ; CNC(=O)c1ccc(C2CCN(Cc3ccc4nc(C)c(=O)[nH]c4c3F)CC2(F)F)c(F)n1 ; CNC(=O)c1ccc(N2CCN(Cc3ccc4nc(C)c(=O)[nH]c4c3F)CC2)cc1F]

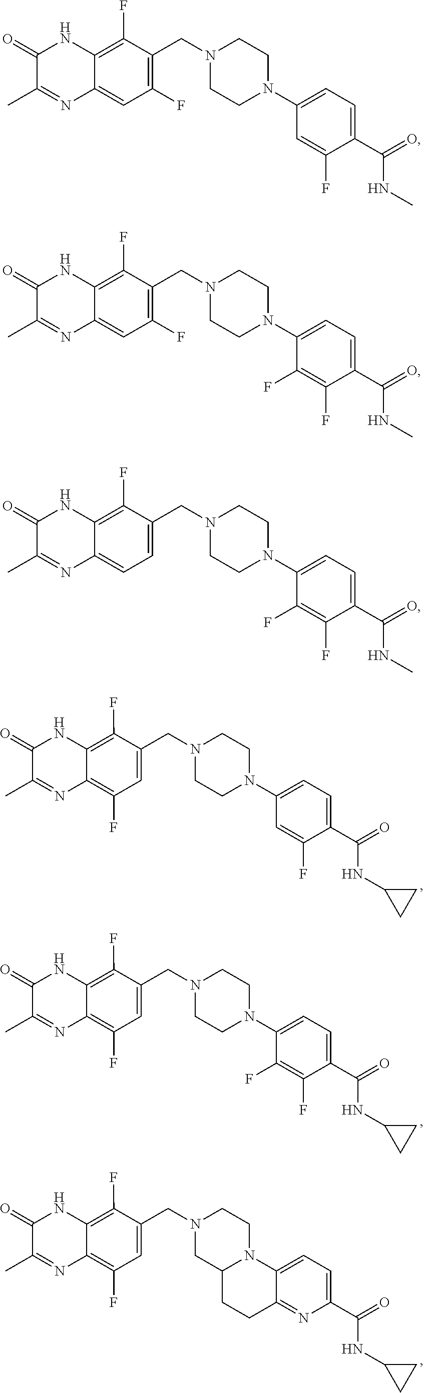 [SMILES: CNC(=O)c1ccc(N2CCN(Cc3c(F)cc4nc(C)c(=O)[nH]c4c3F)CC2)cc1F ; CNC(=O)c1ccc(N2CCN(Cc3c(F)cc4nc(C)c(=O)[nH]c4c3F)CC2)c(F)c1F ; CNC(=O)c1ccc(N2CCN(Cc3ccc4nc(C)c(=O)[nH]c4c3F)CC2)c(F)c1F ; Cc1nc2c(F)cc(CN3CCN(c4ccc(C(=O)NC5CC5)c(F)c4)CC3)c(F)c2[nH]c1=O ; Cc1nc2c(F)cc(CN3CCN(c4ccc(C(=O)NC5CC5)c(F)c4F)CC3)c(F)c2[nH]c1=O ; Cc1nc2c(F)cc(CN3CCN4c5ccc(C(=O)NC6CC6)nc5CCC4C3)c(F)c2[nH]c1=O]

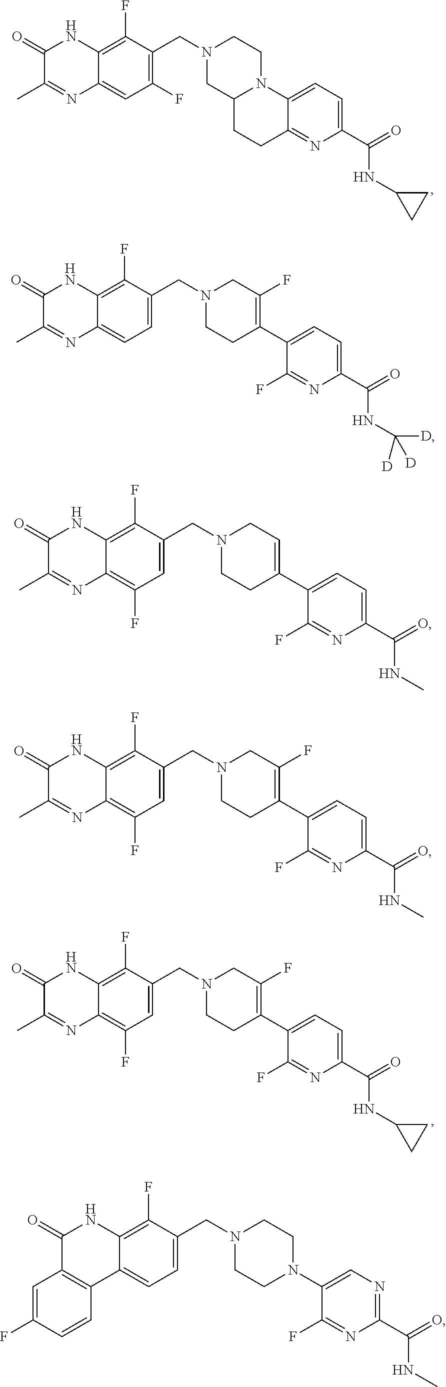

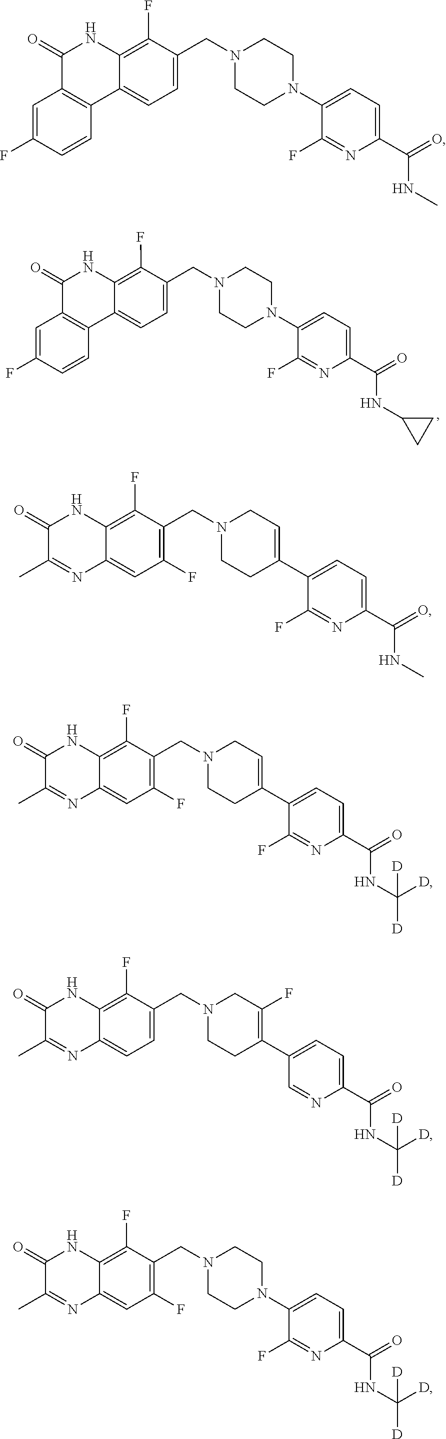

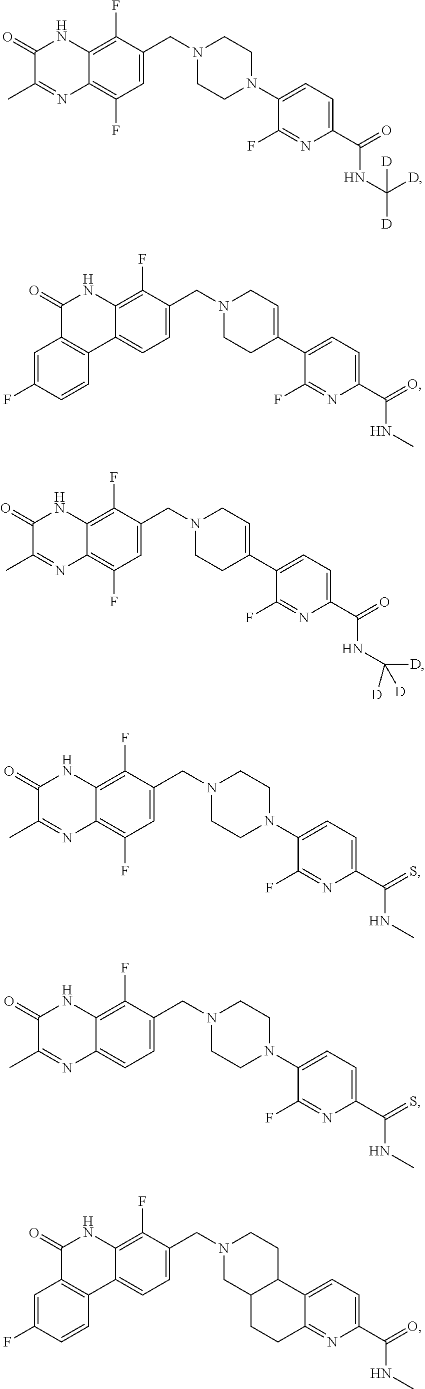

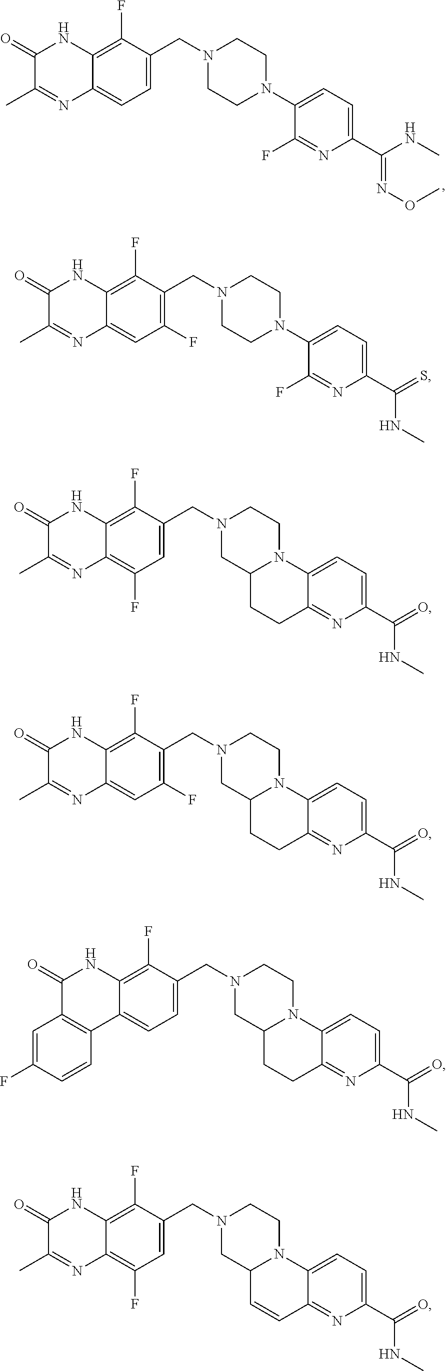 [SMILES: CN/C(=N\OC)c1ccc(N2CCN(Cc3ccc4nc(C)c(=O)[nH]c4c3F)CC2)c(F)n1 ; CNC(=S)c1ccc(N2CCN(Cc3c(F)cc4nc(C)c(=O)[nH]c4c3F)CC2)c(F)n1 ; CNC(=O)c1ccc2c(n1)CCC1CN(Cc3cc(F)c4nc(C)c(=O)[nH]c4c3F)CCN21 ; CNC(=O)c1ccc2c(n1)CCC1CN(Cc3c(F)cc4nc(C)c(=O)[nH]c4c3F)CCN21 ; CNC(=O)c1ccc2c(n1)CCC1CN(Cc3ccc4c([nH]c(=O)c5cc(F)ccc54)c3F)CCN21 ; CNC(=O)c1ccc2c(n1)C=CC1CN(Cc3cc(F)c4nc(C)c(=O)[nH]c4c3F)CCN21]

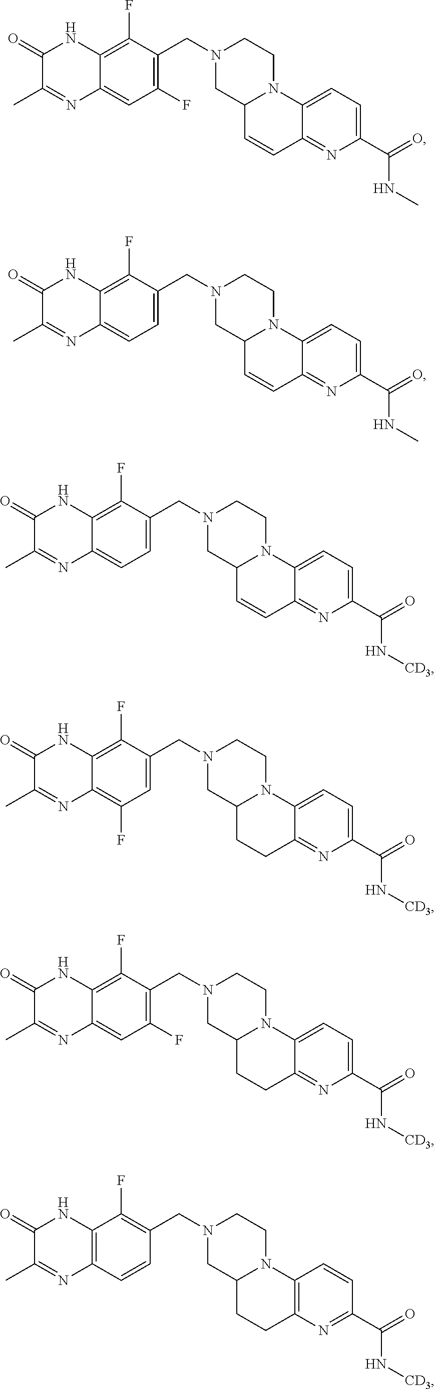 [SMILES: CNC(=O)c1ccc2c(n1)C=CC1CN(Cc3c(F)cc4nc(C)c(=O)[nH]c4c3F)CCN21 ; CNC(=O)c1ccc2c(n1)C=CC1CN(Cc3ccc4nc(C)c(=O)[nH]c4c3F)CCN21 ; [2H]C([2H])([2H])NC(=O)c1ccc2c(n1)C=CC1CN(Cc3ccc4nc(C)c(=O)[nH]c4c3F)CCN21 ; [2H]C([2H])([2H])NC(=O)c1ccc2c(n1)CCC1CN(Cc3cc(F)c4nc(C)c(=O)[nH]c4c3F)CCN21 ; [2H]C([2H])([2H])NC(=O)c1ccc2c(n1)CCC1CN(Cc3c(F)cc4nc(C)c(=O)[nH]c4c3F)CCN21 ; [2H]C([2H])([2H])NC(=O)c1ccc2c(n1)CCC1CN(Cc3ccc4nc(C)c(=O)[nH]c4c3F)CCN21]

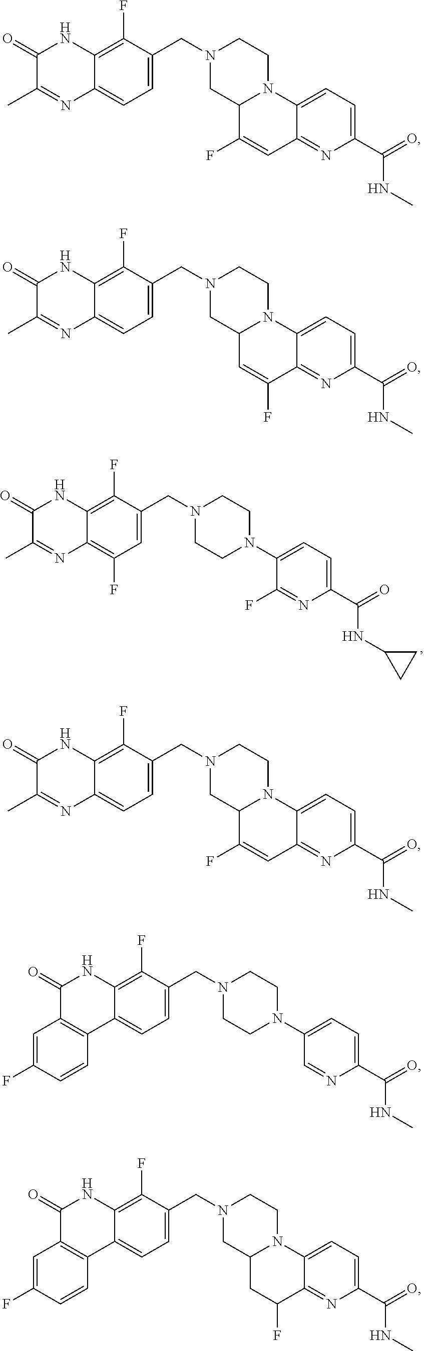 [SMILES: CNC(=O)c1ccc2c(n1)C=C(F)C1CN(Cc3ccc4nc(C)c(=O)[nH]c4c3F)CCN21 ; CNC(=O)c1ccc2c(n1)C(F)=CC1CN(Cc3ccc4nc(C)c(=O)[nH]c4c3F)CCN21 ; Cc1nc2c(F)cc(CN3CCN(c4ccc(C(=O)NC5CC5)nc4F)CC3)c(F)c2[nH]c1=O ; CNC(=O)c1ccc2c(n1)C=C(F)C1CN(Cc3ccc4nc(C)c(=O)[nH]c4c3F)CCN21 ; CNC(=O)c1ccc(N2CCN(Cc3ccc4c([nH]c(=O)c5cc(F)ccc54)c3F)CC2)cn1 ; CNC(=O)c1ccc2c(n1)C(F)CC1CN(Cc3ccc4c([nH]c(=O)c5cc(F)ccc54)c3F)CCN21]

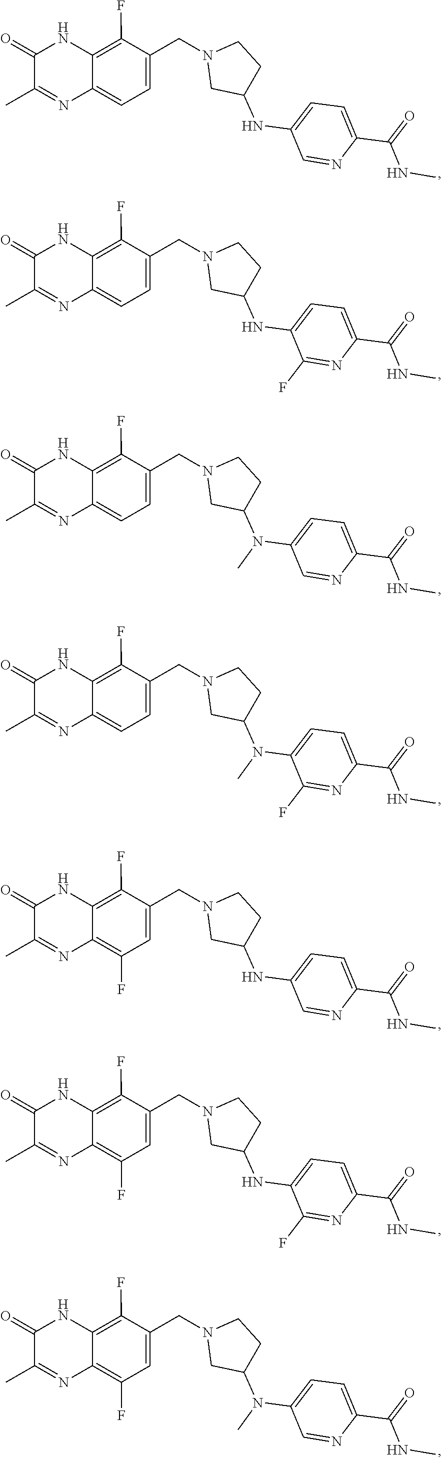

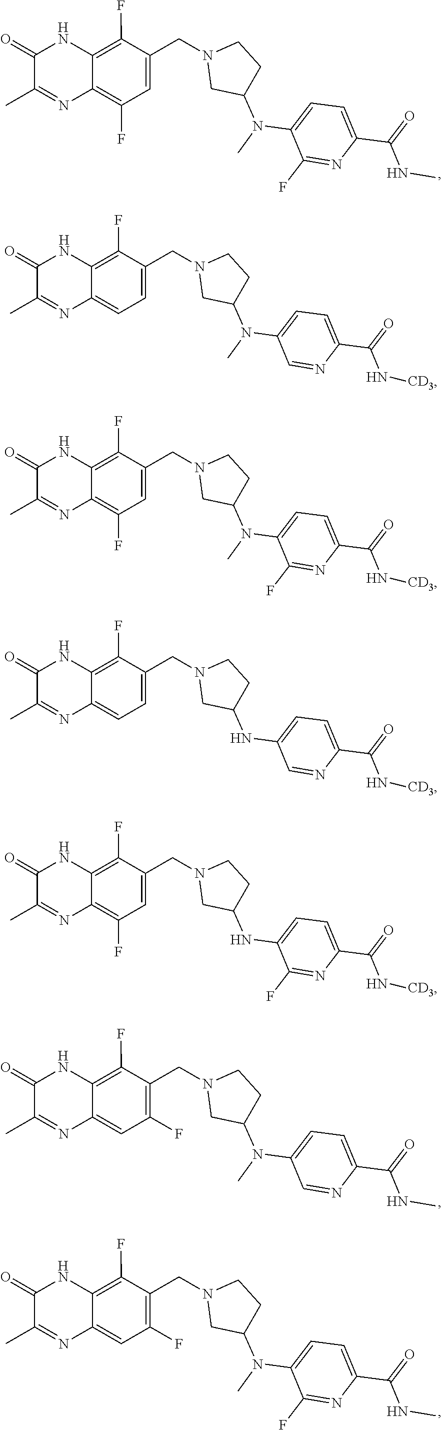

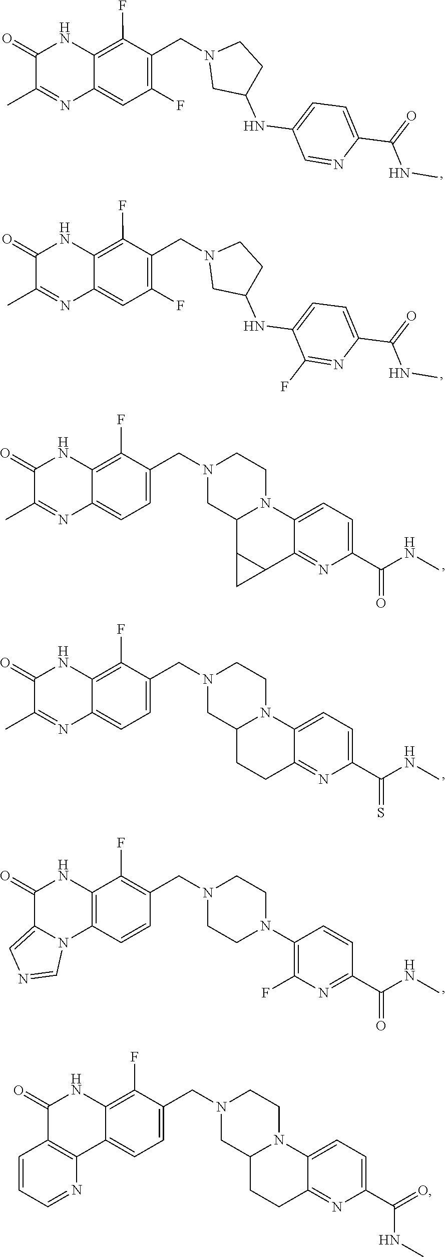 [SMILES: CNC(=O)c1ccc(NC2CCN(Cc3c(F)cc4nc(C)c(=O)[nH]c4c3F)C2)cn1 ; CNC(=O)c1ccc(NC2CCN(Cc3c(F)cc4nc(C)c(=O)[nH]c4c3F)C2)c(F)n1 ; CNC(=O)c1ccc2c(n1)C1CC1C1CN(Cc3ccc4nc(C)c(=O)[nH]c4c3F)CCN21 ; CNC(=S)c1ccc2c(n1)CCC1CN(Cc3ccc4nc(C)c(=O)[nH]c4c3F)CCN21 ; CNC(=O)c1ccc(N2CCN(Cc3ccc4c([nH]c(=O)c5cncn54)c3F)CC2)c(F)n1 ; CNC(=O)c1ccc2c(n1)CCC1CN(Cc3ccc4c([nH]c(=O)c5cccnc54)c3F)CCN21]

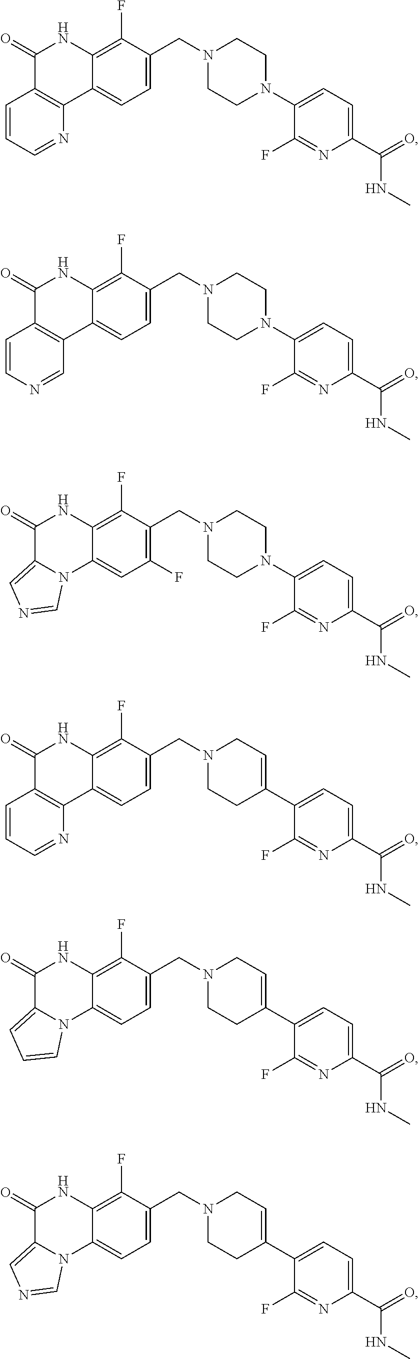 [SMILES: CNC(=O)c1ccc(N2CCN(Cc3ccc4c([nH]c(=O)c5cccnc54)c3F)CC2)c(F)n1 ; CNC(=O)c1ccc(N2CCN(Cc3ccc4c([nH]c(=O)c5ccncc54)c3F)CC2)c(F)n1 ; CNC(=O)c1ccc(N2CCN(Cc3c(F)cc4c([nH]c(=O)c5cncn54)c3F)CC2)c(F)n1 ; CNC(=O)c1ccc(C2=CCN(Cc3ccc4c([nH]c(=O)c5cccnc54)c3F)CC2)c(F)n1 ; CNC(=O)c1ccc(C2=CCN(Cc3ccc4c([nH]c(=O)c5cccn54)c3F)CC2)c(F)n1 ; CNC(=O)c1ccc(C2=CCN(Cc3ccc4c([nH]c(=O)c5cncn54)c3F)CC2)c(F)n1]

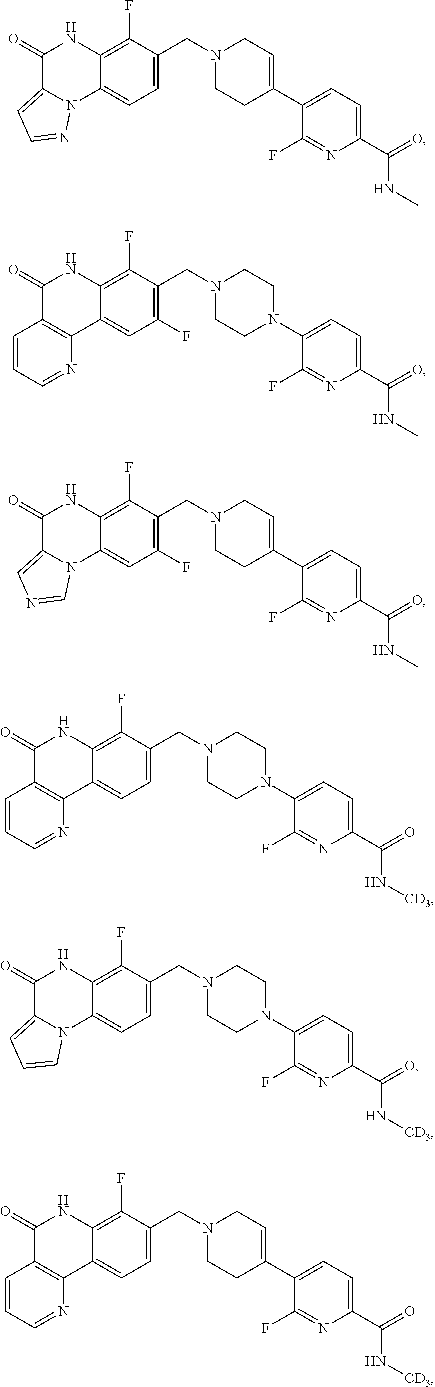 [SMILES: CNC(=O)c1ccc(C2=CCN(Cc3ccc4c([nH]c(=O)c5ccnn54)c3F)CC2)c(F)n1 ; CNC(=O)c1ccc(N2CCN(Cc3c(F)cc4c([nH]c(=O)c5cccnc54)c3F)CC2)c(F)n1 ; CNC(=O)c1ccc(C2=CCN(Cc3c(F)cc4c([nH]c(=O)c5cncn54)c3F)CC2)c(F)n1 ; [2H]C([2H])([2H])NC(=O)c1ccc(N2CCN(Cc3ccc4c([nH]c(=O)c5cccnc54)c3F)CC2)c(F)n1 ; [2H]C([2H])([2H])NC(=O)c1ccc(N2CCN(Cc3ccc4c([nH]c(=O)c5cccn54)c3F)CC2)c(F)n1 ; [2H]C([2H])([2H])NC(=O)c1ccc(C2=CCN(Cc3ccc4c([nH]c(=O)c5cccnc54)c3F)CC2)c(F)n1]

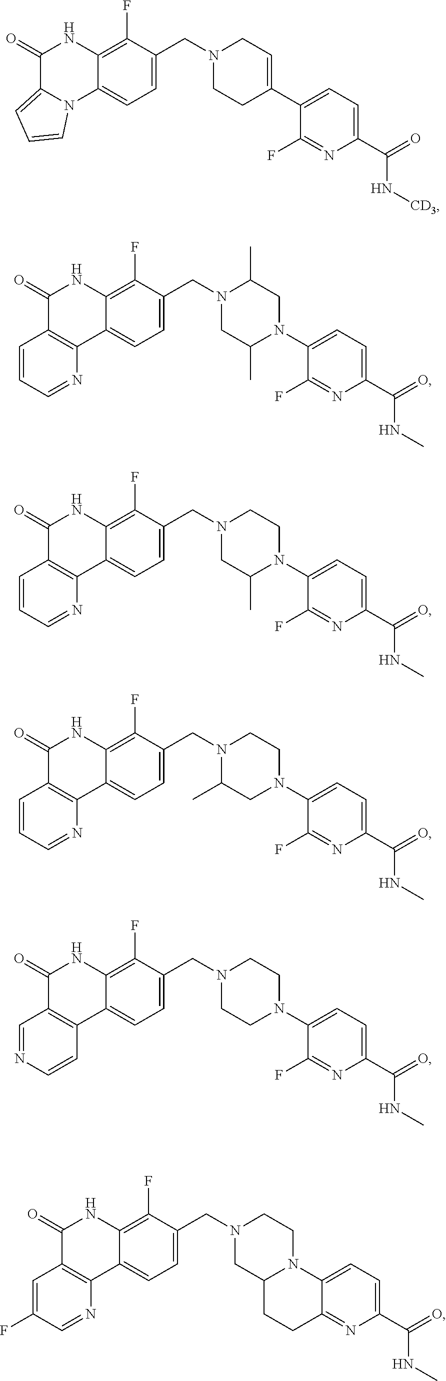 [SMILES: [2H]C([2H])([2H])NC(=O)c1ccc(C2=CCN(Cc3ccc4c([nH]c(=O)c5cccn54)c3F)CC2)c(F)n1 ; CNC(=O)c1ccc(N2CC(C)N(Cc3ccc4c([nH]c(=O)c5cccnc54)c3F)CC2C)c(F)n1 ; CNC(=O)c1ccc(N2CCN(Cc3ccc4c([nH]c(=O)c5cccnc54)c3F)CC2C)c(F)n1 ; CNC(=O)c1ccc(N2CCN(Cc3ccc4c([nH]c(=O)c5cccnc54)c3F)C(C)C2)c(F)n1 ; CNC(=O)c1ccc(N2CCN(Cc3ccc4c([nH]c(=O)c5cnccc54)c3F)CC2)c(F)n1 ; CNC(=O)c1ccc2c(n1)CCC1CN(Cc3ccc4c([nH]c(=O)c5cc(F)cnc54)c3F)CCN21]

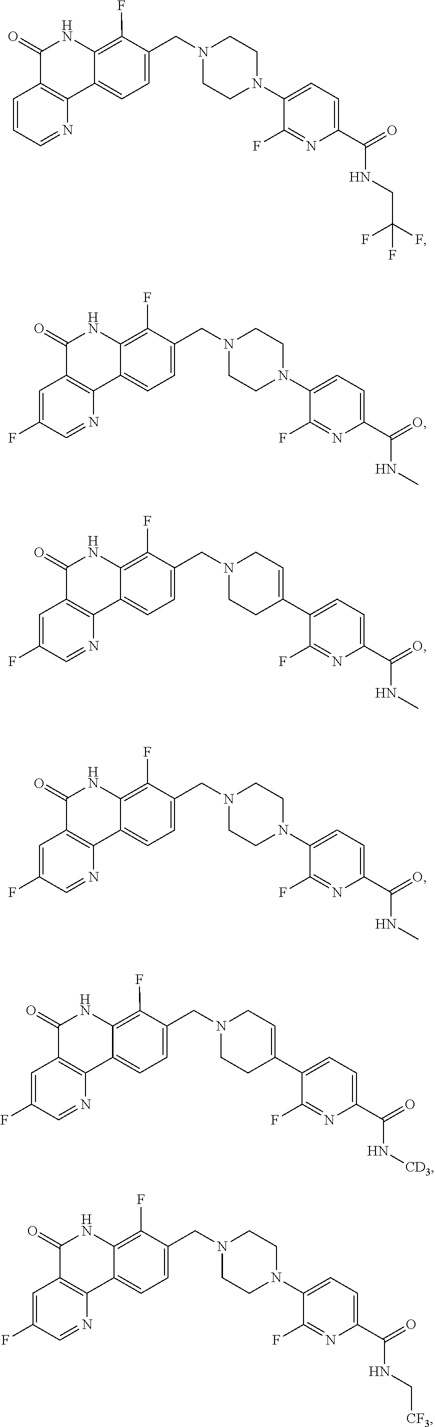 [SMILES: O=C(NCC(F)(F)F)c1ccc(N2CCN(Cc3ccc4c([nH]c(=O)c5cccnc54)c3F)CC2)c(F)n1 ; CNC(=O)c1ccc(N2CCN(Cc3ccc4c([nH]c(=O)c5cc(F)cnc54)c3F)CC2)c(F)n1 ; CNC(=O)c1ccc(C2=CCN(Cc3ccc4c([nH]c(=O)c5cc(F)cnc54)c3F)CC2)c(F)n1 ; CNC(=O)c1ccc(N2CCN(Cc3ccc4c([nH]c(=O)c5cc(F)cnc54)c3F)CC2)c(F)n1 ; [2H]C([2H])([2H])NC(=O)c1ccc(C2=CCN(Cc3ccc4c([nH]c(=O)c5cc(F)cnc54)c3F)CC2)c(F)n1 ; O=C(NCC(F)(F)F)c1ccc(N2CCN(Cc3ccc4c([nH]c(=O)c5cc(F)cnc54)c3F)CC2)c(F)n1]

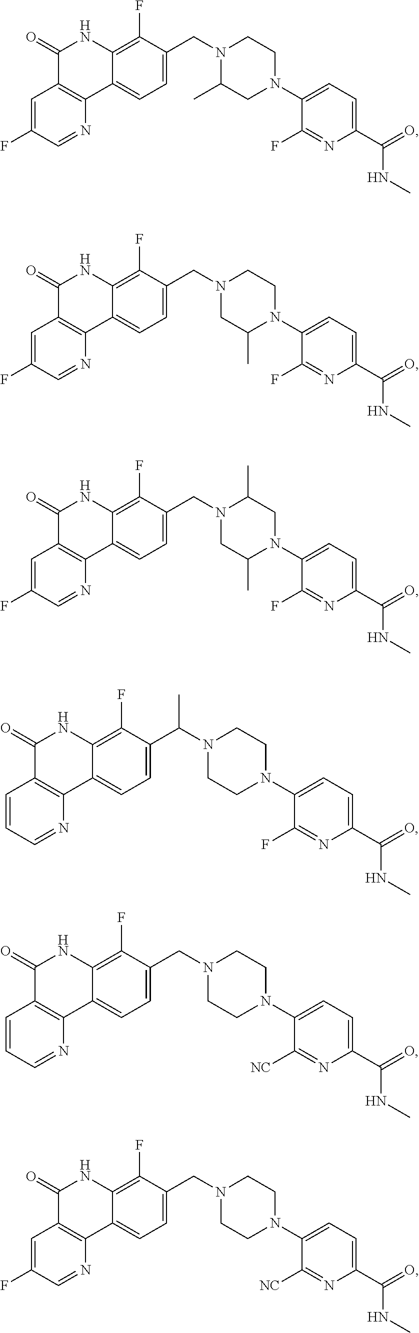 [SMILES: CNC(=O)c1ccc(N2CCN(Cc3ccc4c([nH]c(=O)c5cc(F)cnc54)c3F)C(C)C2)c(F)n1 ; CNC(=O)c1ccc(N2CCN(Cc3ccc4c([nH]c(=O)c5cc(F)cnc54)c3F)CC2C)c(F)n1 ; CNC(=O)c1ccc(N2CC(C)N(Cc3ccc4c([nH]c(=O)c5cc(F)cnc54)c3F)CC2C)c(F)n1 ; CNC(=O)c1ccc(N2CCN(C(C)c3ccc4c([nH]c(=O)c5cccnc54)c3F)CC2)c(F)n1 ; CNC(=O)c1ccc(N2CCN(Cc3ccc4c([nH]c(=O)c5cccnc54)c3F)CC2)c(C#N)n1 ; CNC(=O)c1ccc(N2CCN(Cc3ccc4c([nH]c(=O)c5cc(F)cnc54)c3F)CC2)c(C#N)n1]

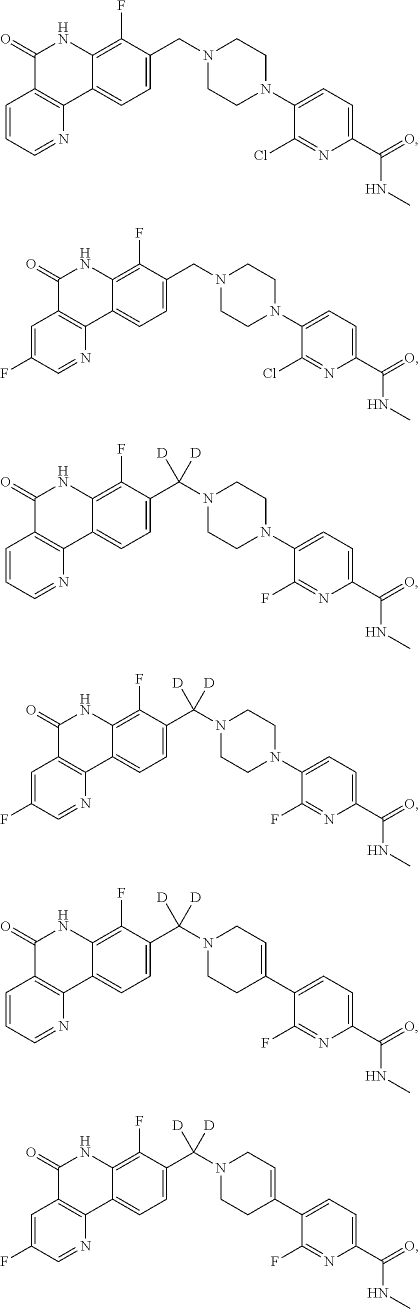 [SMILES: CNC(=O)c1ccc(N2CCN(Cc3ccc4c([nH]c(=O)c5cccnc54)c3F)CC2)c(Cl)n1 ; CNC(=O)c1ccc(N2CCN(Cc3ccc4c([nH]c(=O)c5cc(F)cnc54)c3F)CC2)c(Cl)n1 ; [2H]C([2H])(c1ccc2c([nH]c(=O)c3cccnc32)c1F)N1CCN(c2ccc(C(=O)NC)nc2F)CC1 ; [2H]C([2H])(c1ccc2c([nH]c(=O)c3cc(F)cnc32)c1F)N1CCN(c2ccc(C(=O)NC)nc2F)CC1 ; [2H]C([2H])(c1ccc2c([nH]c(=O)c3cccnc32)c1F)N1CC=C(c2ccc(C(=O)NC)nc2F)CC1 ; [2H]C([2H])(c1ccc2c([nH]c(=O)c3cc(F)cnc32)c1F)N1CC=C(c2ccc(C(=O)NC)nc2F)CC1]

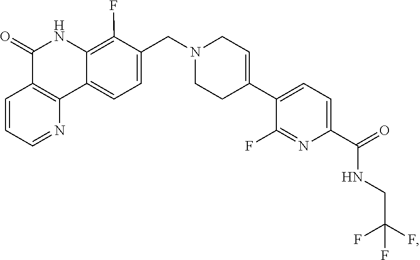 [SMILES: O=C(NCC(F)(F)F)c1ccc(C2=CCN(Cc3ccc4c([nH]c(=O)c5cccnc54)c3F)CC2)c(F)n1]

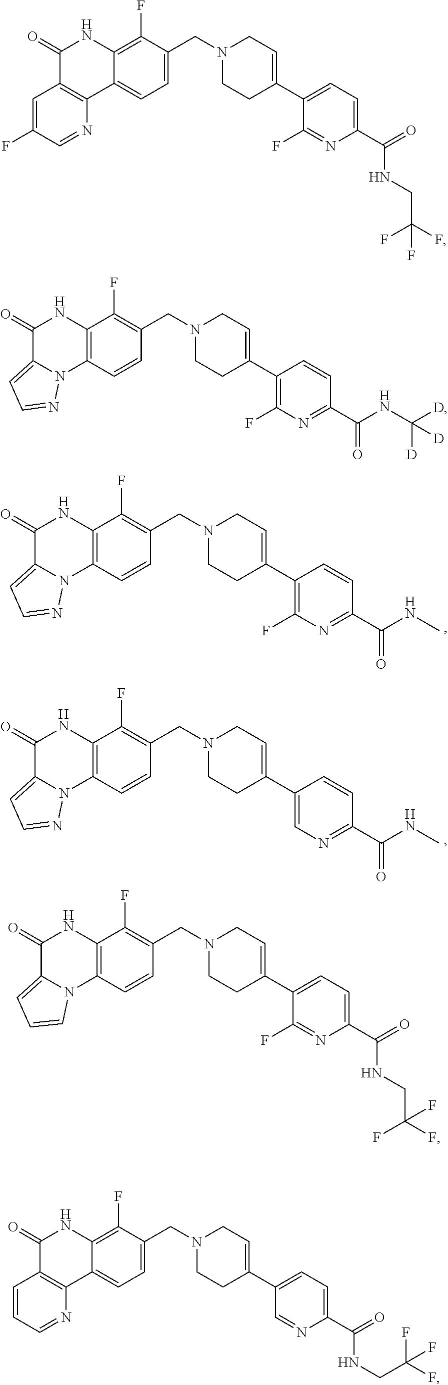

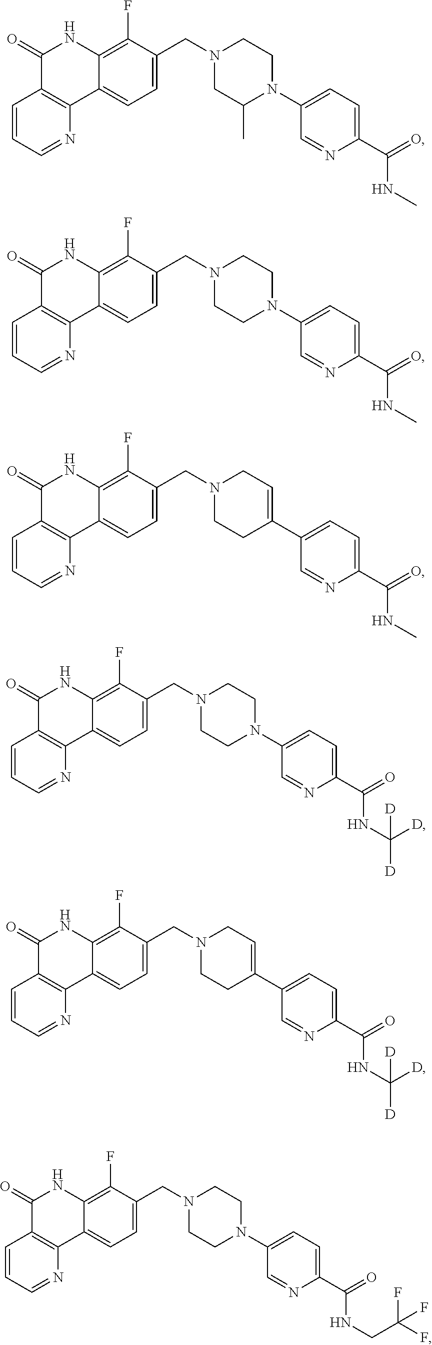

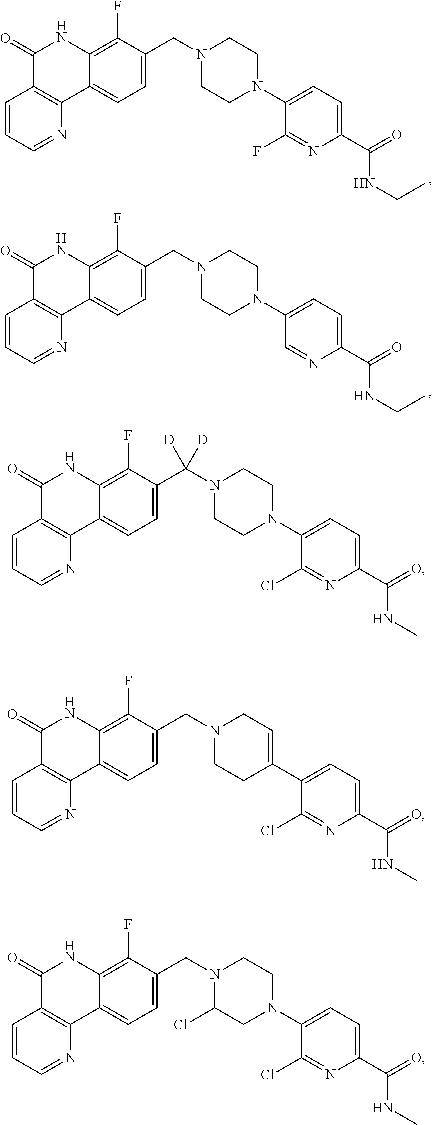 [SMILES: CCNC(=O)c1ccc(N2CCN(Cc3ccc4c([nH]c(=O)c5cccnc54)c3F)CC2)c(F)n1 ; CCNC(=O)c1ccc(N2CCN(Cc3ccc4c([nH]c(=O)c5cccnc54)c3F)CC2)cn1 ; [2H]C([2H])(c1ccc2c([nH]c(=O)c3cccnc32)c1F)N1CCN(c2ccc(C(=O)NC)nc2Cl)CC1 ; CNC(=O)c1ccc(C2=CCN(Cc3ccc4c([nH]c(=O)c5cccnc54)c3F)CC2)c(Cl)n1 ; CNC(=O)c1ccc(N2CCN(Cc3ccc4c([nH]c(=O)c5cccnc54)c3F)C(Cl)C2)c(Cl)n1]

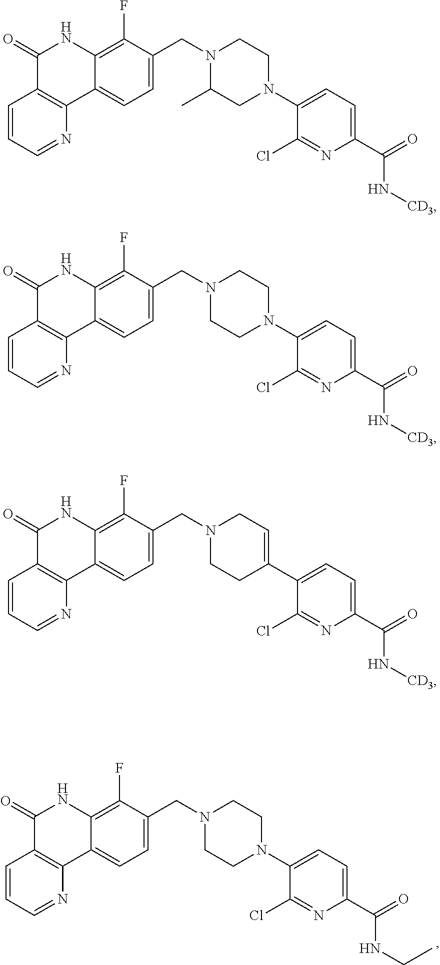 [SMILES: [2H]C([2H])([2H])NC(=O)c1ccc(N2CCN(Cc3ccc4c([nH]c(=O)c5cccnc54)c3F)C(C)C2)c(Cl)n1 ; [2H]C([2H])([2H])NC(=O)c1ccc(N2CCN(Cc3ccc4c([nH]c(=O)c5cccnc54)c3F)CC2)c(Cl)n1 ; [2H]C([2H])([2H])NC(=O)c1ccc(C2=CCN(Cc3ccc4c([nH]c(=O)c5cccnc54)c3F)CC2)c(Cl)n1 ; CCNC(=O)c1ccc(N2CCN(Cc3ccc4c([nH]c(=O)c5cccnc54)c3F)CC2)c(Cl)n1]

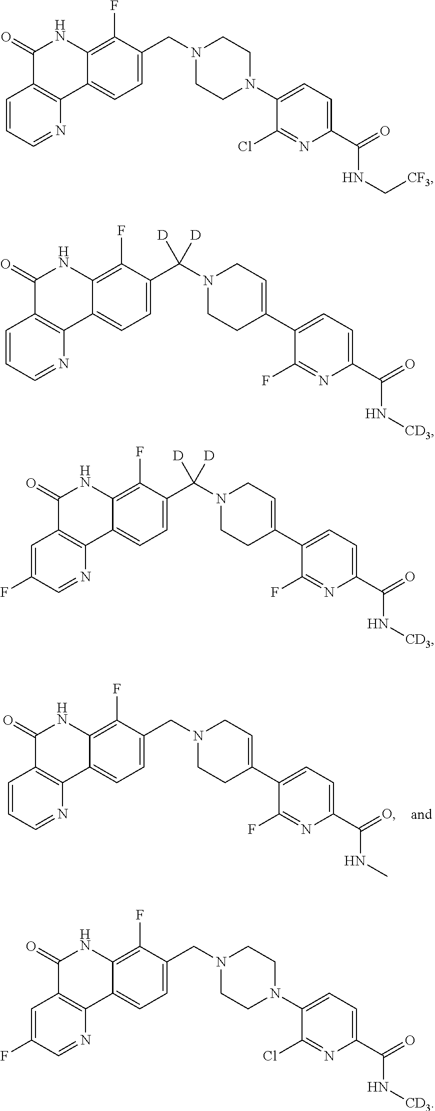 [SMILES: O=C(NCC(F)(F)F)c1ccc(N2CCN(Cc3ccc4c([nH]c(=O)c5cccnc54)c3F)CC2)c(Cl)n1 ; [2H]C([2H])([2H])NC(=O)c1ccc(C2=CCN(C([2H])([2H])c3ccc4c([nH]c(=O)c5cccnc54)c3F)CC2)c(F)n1 ; [2H]C([2H])([2H])NC(=O)c1ccc(C2=CCN(C([2H])([2H])c3ccc4c([nH]c(=O)c5cc(F)cnc54)c3F)CC2)c(F)n1 ; CNC(=O)c1ccc(C2=CCN(Cc3ccc4c([nH]c(=O)c5cccnc54)c3F)CC2)c(F)n1 ; "and" ; [2H]C([2H])([2H])NC(=O)c1ccc(N2CCN(Cc3ccc4c([nH]c(=O)c5cc(F)cnc54)c3F)CC2)c(Cl)n1]

25. The compound or the pharmaceutically acceptable salt thereof according to claim 24, wherein the compound is selected from:

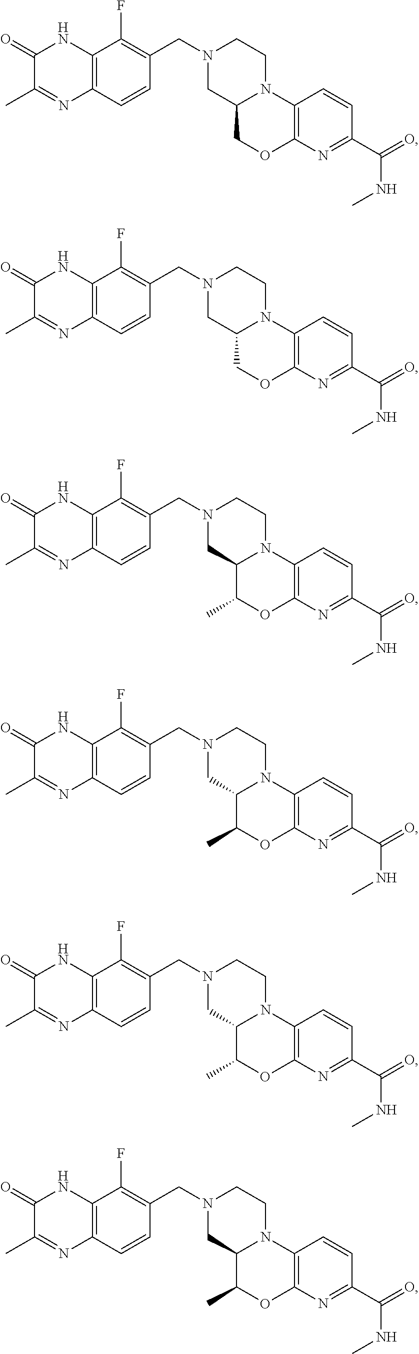 [SMILES: CNC(=O)c1ccc2c(n1)OC[C@H]1CN(Cc3ccc4nc(C)c(=O)[nH]c4c3F)CCN21 ; CNC(=O)c1ccc2c(n1)OC[C@@H]1CN(Cc3ccc4nc(C)c(=O)[nH]c4c3F)CCN21 ; CNC(=O)c1ccc2c(n1)O[C@H](C)[C@H]1CN(Cc3ccc4nc(C)c(=O)[nH]c4c3F)CCN21 ; CNC(=O)c1ccc2c(n1)O[C@@H](C)[C@@H]1CN(Cc3ccc4nc(C)c(=O)[nH]c4c3F)CCN21 ; CNC(=O)c1ccc2c(n1)O[C@H](C)[C@@H]1CN(Cc3ccc4nc(C)c(=O)[nH]c4c3F)CCN21 ; CNC(=O)c1ccc2c(n1)O[C@@H](C)[C@H]1CN(Cc3ccc4nc(C)c(=O)[nH]c4c3F)CCN21]

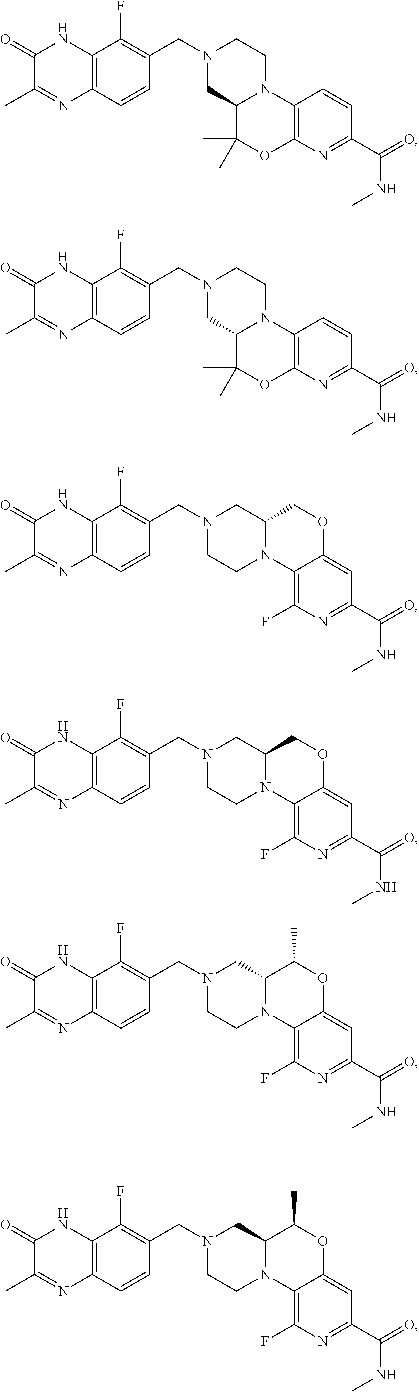 [SMILES: CNC(=O)c1ccc2c(n1)OC(C)(C)[C@H]1CN(Cc3ccc4nc(C)c(=O)[nH]c4c3F)CCN21 ; CNC(=O)c1ccc2c(n1)OC(C)(C)[C@@H]1CN(Cc3ccc4nc(C)c(=O)[nH]c4c3F)CCN21 ; CNC(=O)c1cc2c(c(F)n1)N1CCN(Cc3ccc4nc(C)c(=O)[nH]c4c3F)C[C@@H]1CO2 ; CNC(=O)c1cc2c(c(F)n1)N1CCN(Cc3ccc4nc(C)c(=O)[nH]c4c3F)C[C@H]1CO2 ; CNC(=O)c1cc2c(c(F)n1)N1CCN(Cc3ccc4nc(C)c(=O)[nH]c4c3F)C[C@@H]1[C@H](C)O2 ; CNC(=O)c1cc2c(c(F)n1)N1CCN(Cc3ccc4nc(C)c(=O)[nH]c4c3F)C[C@H]1[C@@H](C)O2]

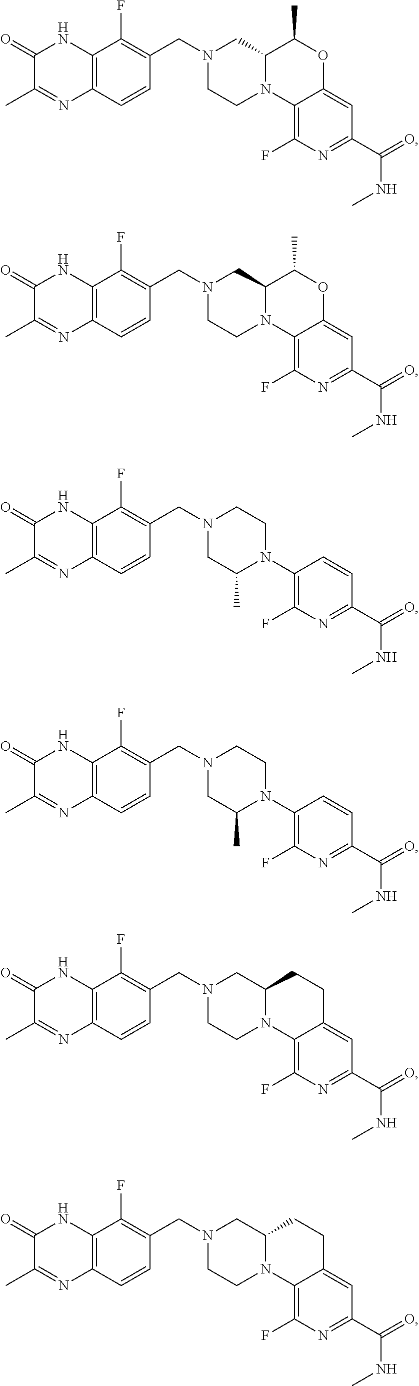 [SMILES: CNC(=O)c1cc2c(c(F)n1)N1CCN(Cc3ccc4nc(C)c(=O)[nH]c4c3F)C[C@@H]1[C@@H](C)O2 ; CNC(=O)c1cc2c(c(F)n1)N1CCN(Cc3ccc4nc(C)c(=O)[nH]c4c3F)C[C@H]1[C@H](C)O2 ; CNC(=O)c1ccc(N2CCN(Cc3ccc4nc(C)c(=O)[nH]c4c3F)C[C@H]2C)c(F)n1 ; CNC(=O)c1ccc(N2CCN(Cc3ccc4nc(C)c(=O)[nH]c4c3F)C[C@@H]2C)c(F)n1 ; CNC(=O)c1cc2c(c(F)n1)N1CCN(Cc3ccc4nc(C)c(=O)[nH]c4c3F)C[C@H]1CC2 ; CNC(=O)c1cc2c(c(F)n1)N1CCN(Cc3ccc4nc(C)c(=O)[nH]c4c3F)C[C@@H]1CC2]

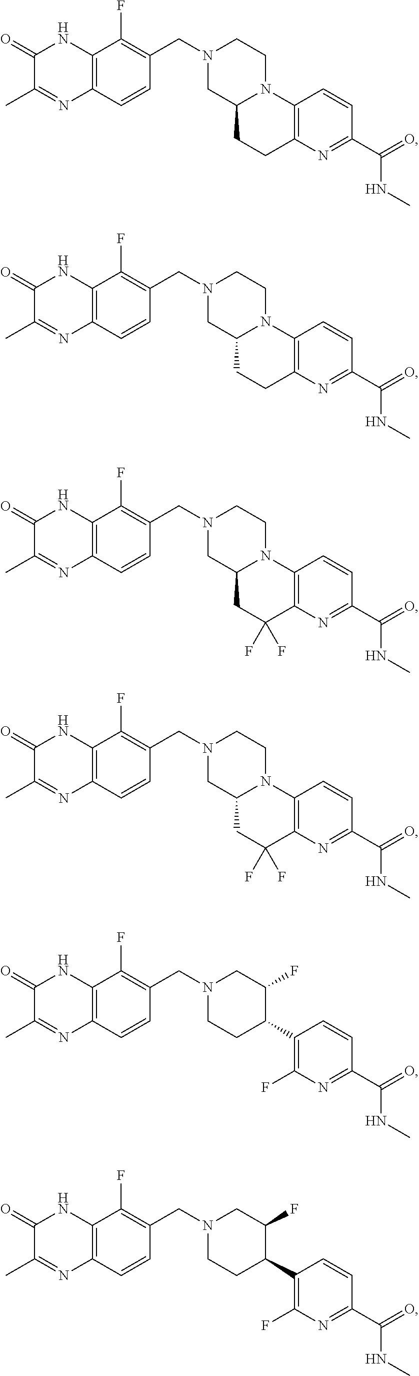 [SMILES: CNC(=O)c1ccc2c(n1)CC[C@H]1CN(Cc3ccc4nc(C)c(=O)[nH]c4c3F)CCN21 ; CNC(=O)c1ccc2c(n1)CC[C@@H]1CN(Cc3ccc4nc(C)c(=O)[nH]c4c3F)CCN21 ; CNC(=O)c1ccc2c(n1)C(F)(F)C[C@H]1CN(Cc3ccc4nc(C)c(=O)[nH]c4c3F)CCN21 ; CNC(=O)c1ccc2c(n1)C(F)(F)C[C@@H]1CN(Cc3ccc4nc(C)c(=O)[nH]c4c3F)CCN21 ; CNC(=O)c1ccc([C@@H]2CCN(Cc3ccc4nc(C)c(=O)[nH]c4c3F)C[C@@H]2F)c(F)n1 ; CNC(=O)c1ccc([C@H]2CCN(Cc3ccc4nc(C)c(=O)[nH]c4c3F)C[C@H]2F)c(F)n1]

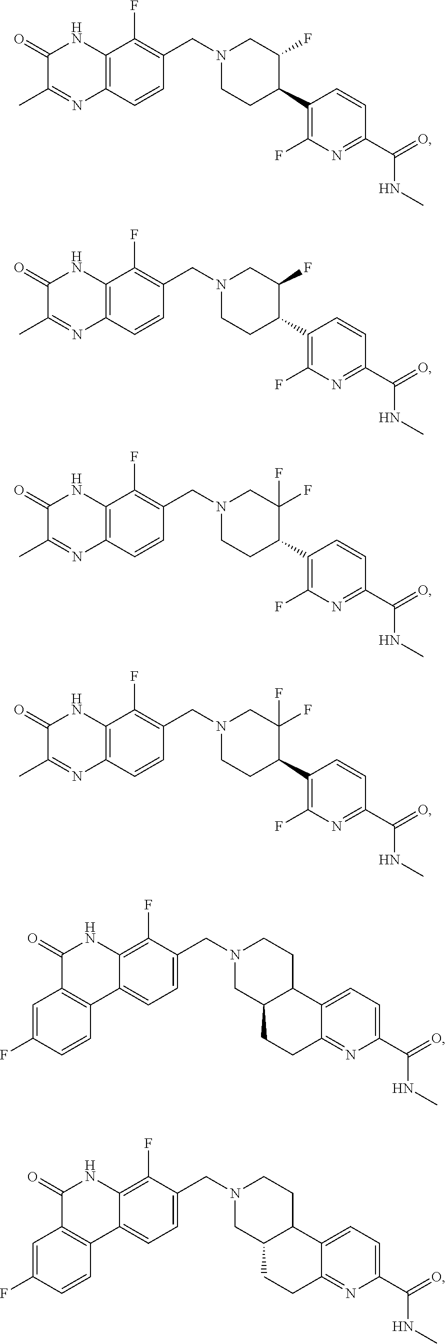

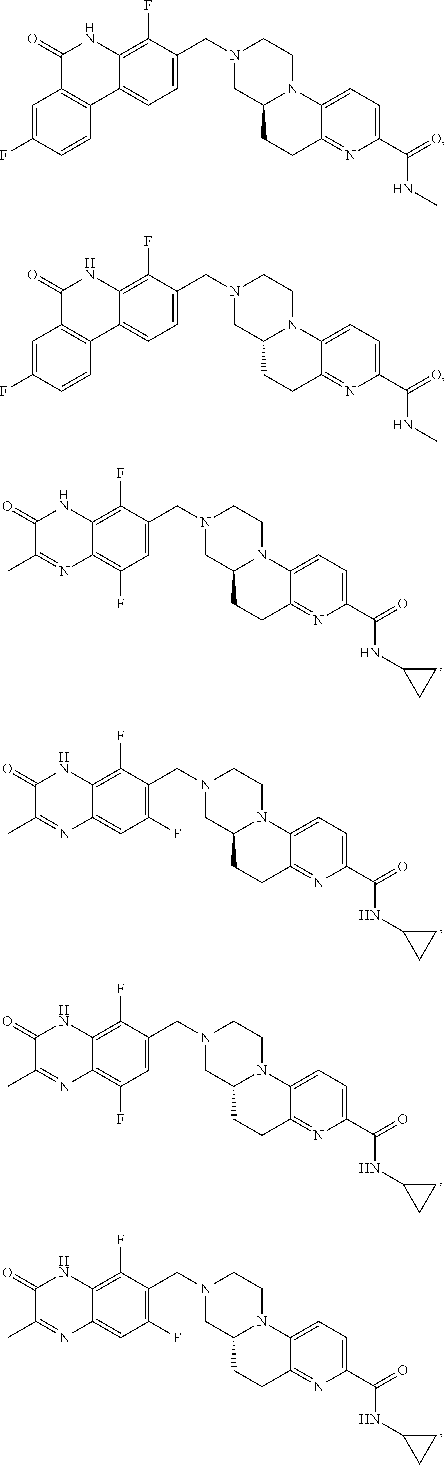

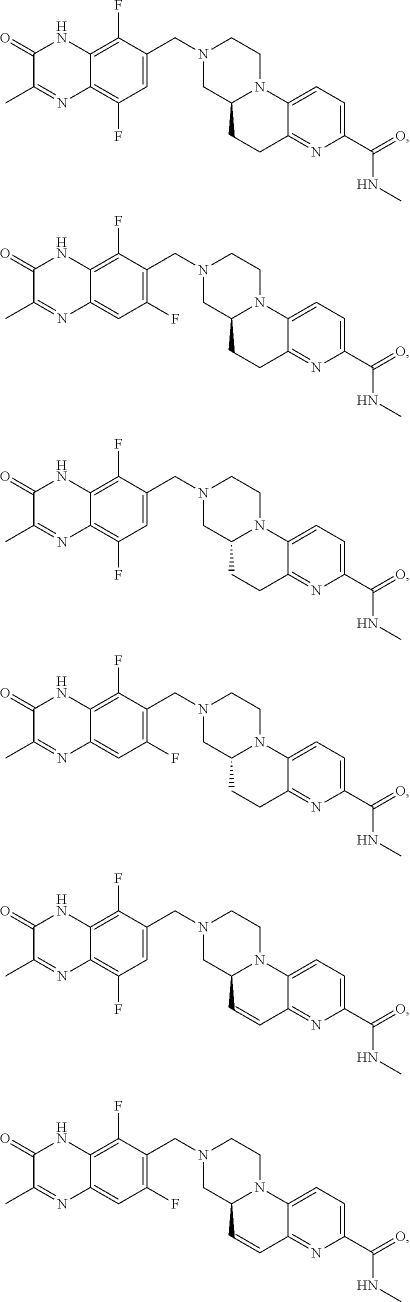

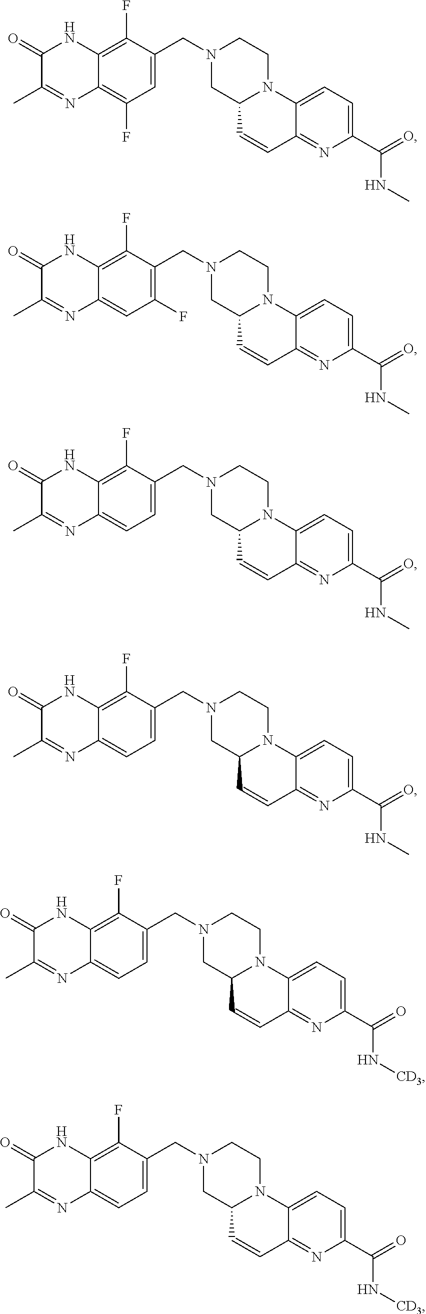

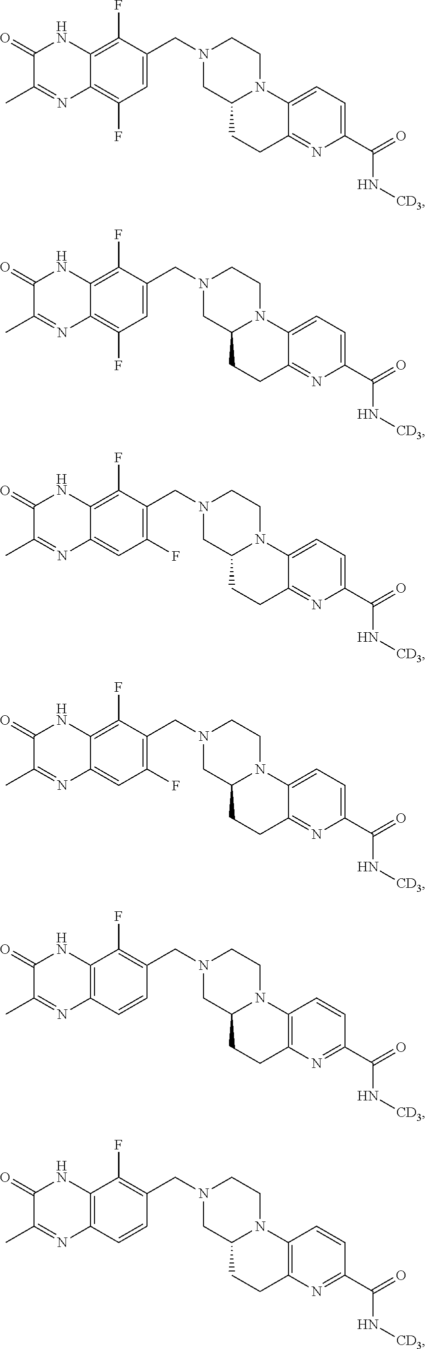 [SMILES: [2H]C([2H])([2H])NC(=O)c1ccc2c(n1)CC[C@@H]1CN(Cc3cc(F)c4nc(C)c(=O)[nH]c4c3F)CCN21 ; [2H]C([2H])([2H])NC(=O)c1ccc2c(n1)CC[C@H]1CN(Cc3cc(F)c4nc(C)c(=O)[nH]c4c3F)CCN21 ; [2H]C([2H])([2H])NC(=O)c1ccc2c(n1)CC[C@@H]1CN(Cc3c(F)cc4nc(C)c(=O)[nH]c4c3F)CCN21 ; [2H]C([2H])([2H])NC(=O)c1ccc2c(n1)CC[C@H]1CN(Cc3c(F)cc4nc(C)c(=O)[nH]c4c3F)CCN21 ; [2H]C([2H])([2H])NC(=O)c1ccc2c(n1)CC[C@H]1CN(Cc3ccc4nc(C)c(=O)[nH]c4c3F)CCN21 ; [2H]C([2H])([2H])NC(=O)c1ccc2c(n1)CC[C@@H]1CN(Cc3ccc4nc(C)c(=O)[nH]c4c3F)CCN21]

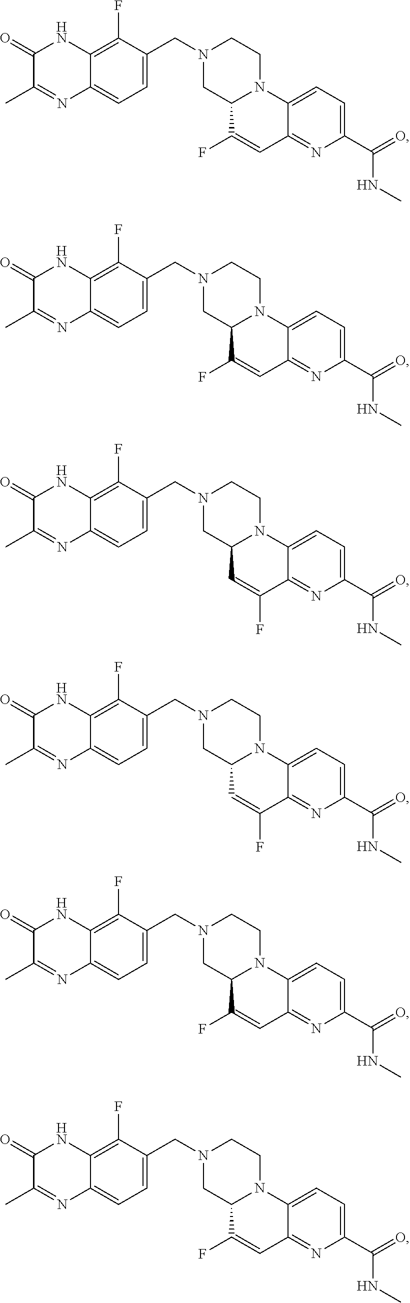

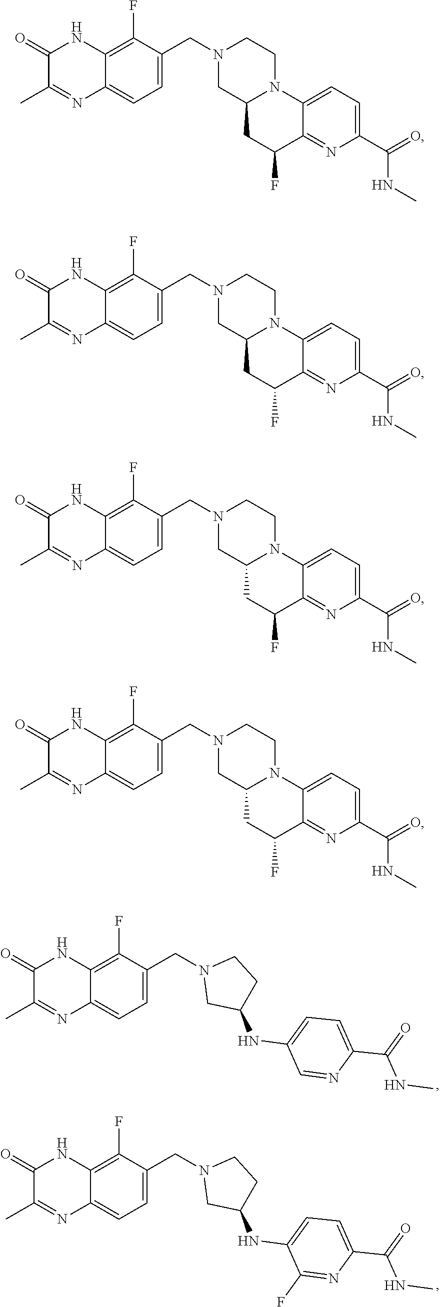

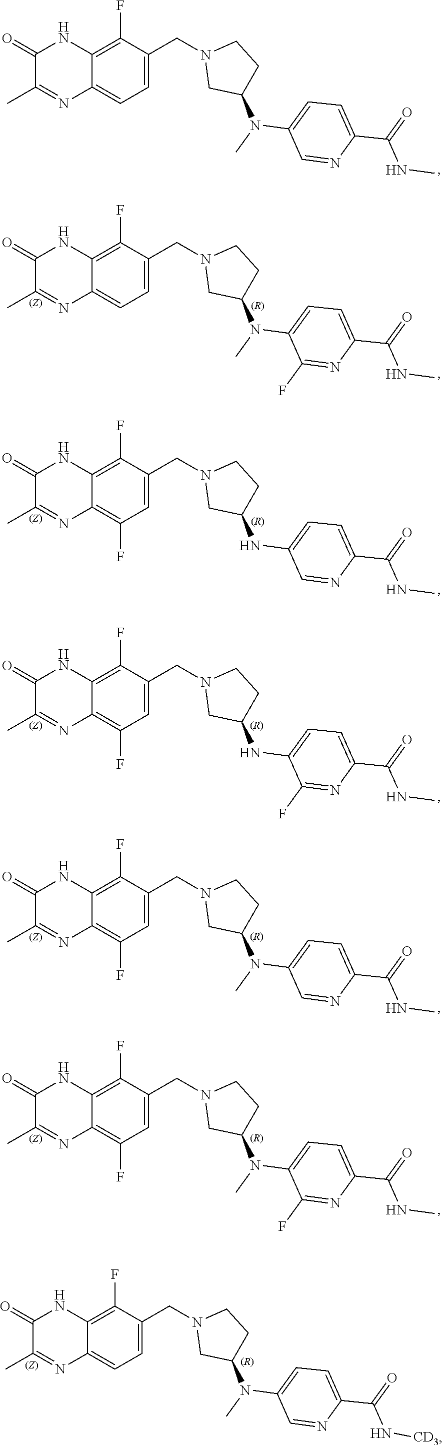

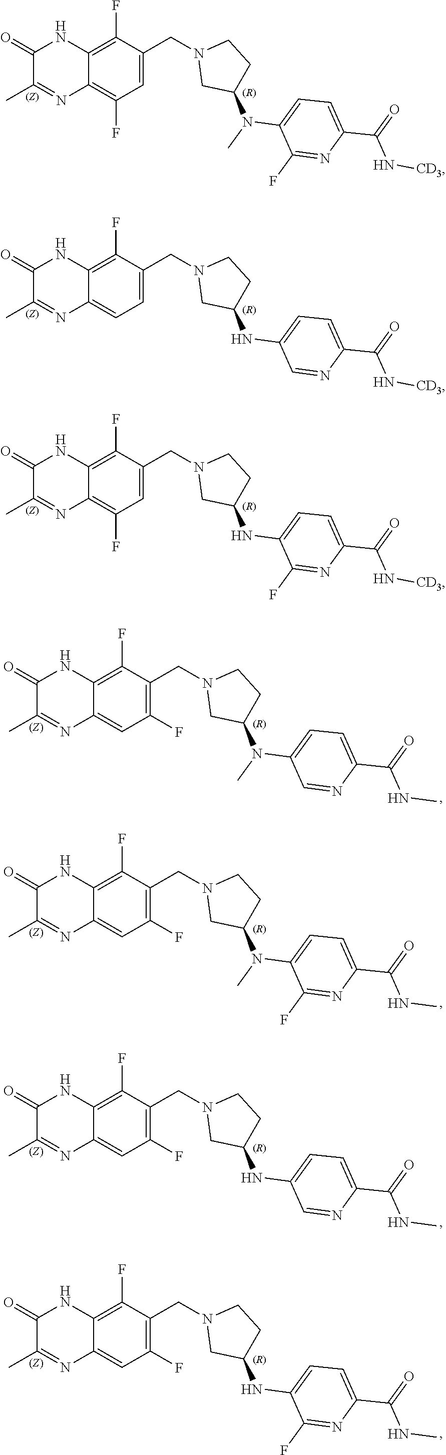

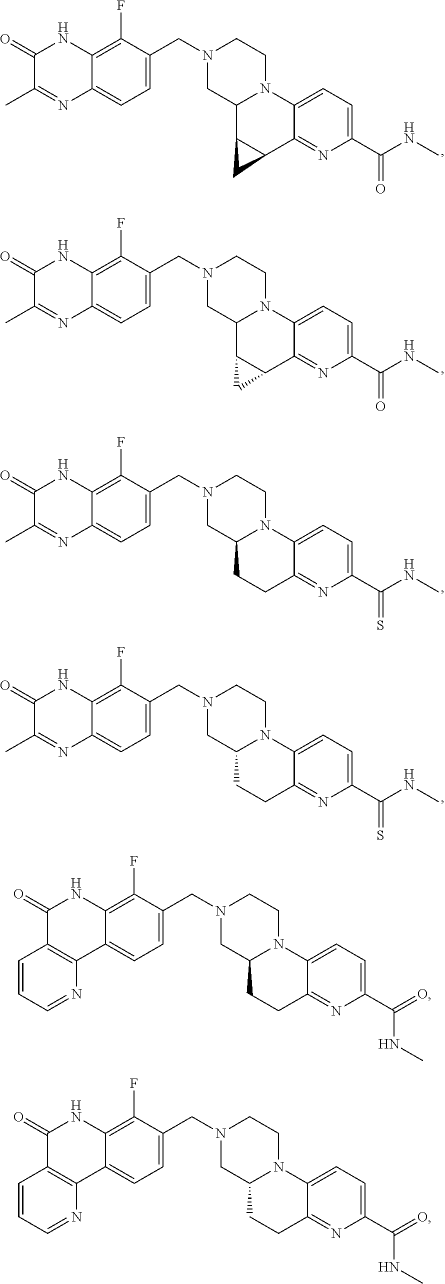 [SMILES: CNC(=O)c1ccc2c(n1)[C@H]1C[C@H]1C1CN(Cc3ccc4nc(C)c(=O)[nH]c4c3F)CCN21 ; CNC(=O)c1ccc2c(n1)[C@@H]1C[C@@H]1C1CN(Cc3ccc4nc(C)c(=O)[nH]c4c3F)CCN21 ; CNC(=S)c1ccc2c(n1)CC[C@H]1CN(Cc3ccc4nc(C)c(=O)[nH]c4c3F)CCN21 ; CNC(=S)c1ccc2c(n1)CC[C@@H]1CN(Cc3ccc4nc(C)c(=O)[nH]c4c3F)CCN21 ; CNC(=O)c1ccc2c(n1)CC[C@H]1CN(Cc3ccc4c([nH]c(=O)c5cccnc54)c3F)CCN21 ; CNC(=O)c1ccc2c(n1)CC[C@@H]1CN(Cc3ccc4c([nH]c(=O)c5cccnc54)c3F)CCN21]

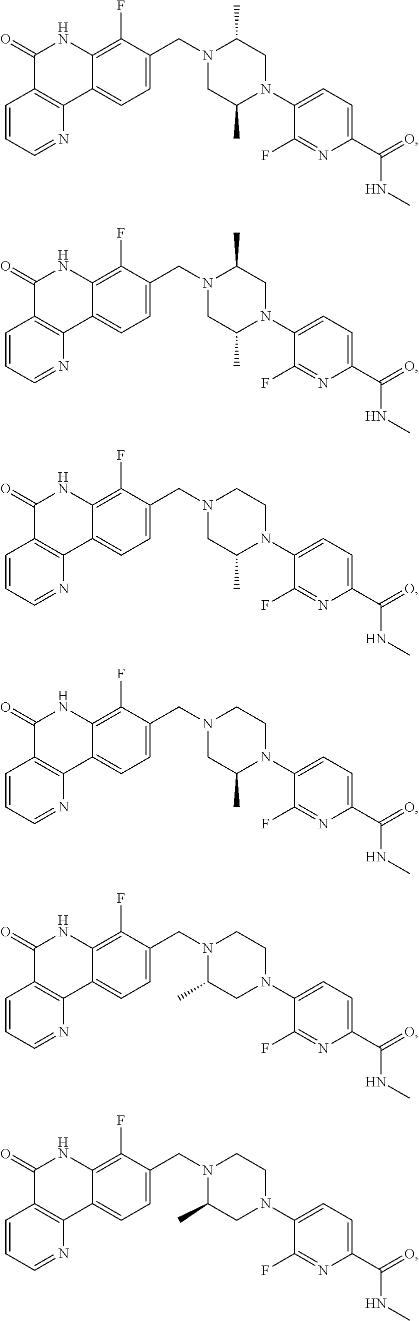 [SMILES: CNC(=O)c1ccc(N2C[C@@H](C)N(Cc3ccc4c([nH]c(=O)c5cccnc54)c3F)C[C@@H]2C)c(F)n1 ; CNC(=O)c1ccc(N2C[C@H](C)N(Cc3ccc4c([nH]c(=O)c5cccnc54)c3F)C[C@H]2C)c(F)n1 ; CNC(=O)c1ccc(N2CCN(Cc3ccc4c([nH]c(=O)c5cccnc54)c3F)C[C@H]2C)c(F)n1 ; CNC(=O)c1ccc(N2CCN(Cc3ccc4c([nH]c(=O)c5cccnc54)c3F)C[C@@H]2C)c(F)n1 ; CNC(=O)c1ccc(N2CCN(Cc3ccc4c([nH]c(=O)c5cccnc54)c3F)[C@@H](C)C2)c(F)n1 ; CNC(=O)c1ccc(N2CCN(Cc3ccc4c([nH]c(=O)c5cccnc54)c3F)[C@H](C)C2)c(F)n1]

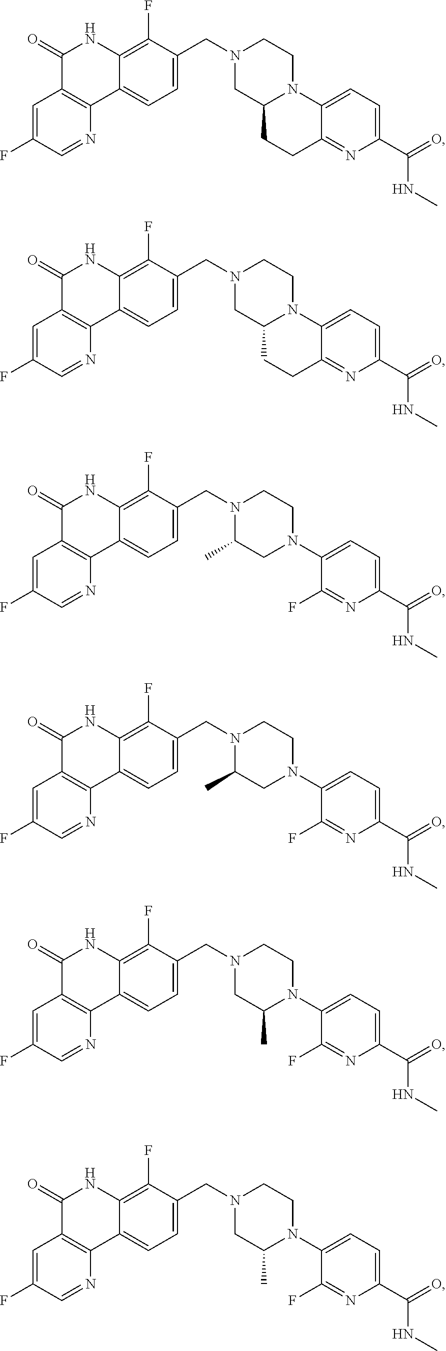 [SMILES: CNC(=O)c1ccc2c(n1)CC[C@H]1CN(Cc3ccc4c([nH]c(=O)c5cc(F)cnc54)c3F)CCN21 ; CNC(=O)c1ccc2c(n1)CC[C@@H]1CN(Cc3ccc4c([nH]c(=O)c5cc(F)cnc54)c3F)CCN21 ; CNC(=O)c1ccc(N2CCN(Cc3ccc4c([nH]c(=O)c5cc(F)cnc54)c3F)[C@@H](C)C2)c(F)n1 ; CNC(=O)c1ccc(N2CCN(Cc3ccc4c([nH]c(=O)c5cc(F)cnc54)c3F)[C@H](C)C2)c(F)n1 ; CNC(=O)c1ccc(N2CCN(Cc3ccc4c([nH]c(=O)c5cc(F)cnc54)c3F)C[C@@H]2C)c(F)n1 ; CNC(=O)c1ccc(N2CCN(Cc3ccc4c([nH]c(=O)c5cc(F)cnc54)c3F)C[C@H]2C)c(F)n1]

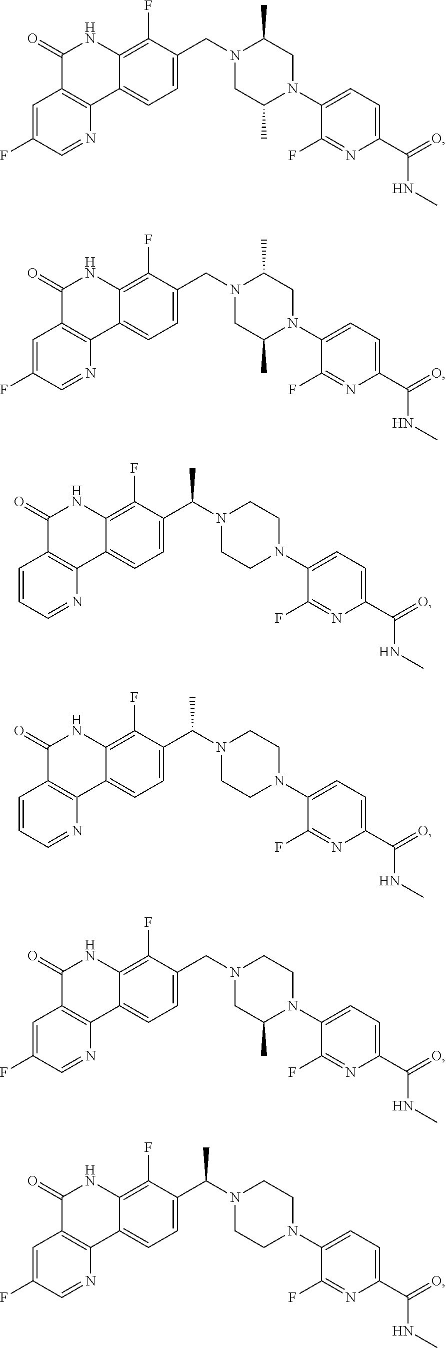 [SMILES: CNC(=O)c1ccc(N2C[C@H](C)N(Cc3ccc4c([nH]c(=O)c5cc(F)cnc54)c3F)C[C@H]2C)c(F)n1 ; CNC(=O)c1ccc(N2C[C@@H](C)N(Cc3ccc4c([nH]c(=O)c5cc(F)cnc54)c3F)C[C@@H]2C)c(F)n1 ; CNC(=O)c1ccc(N2CCN([C@H](C)c3ccc4c([nH]c(=O)c5cccnc54)c3F)CC2)c(F)n1 ; CNC(=O)c1ccc(N2CCN([C@@H](C)c3ccc4c([nH]c(=O)c5cccnc54)c3F)CC2)c(F)n1 ; CNC(=O)c1ccc(N2CCN(Cc3ccc4c([nH]c(=O)c5cc(F)cnc54)c3F)C[C@@H]2C)c(F)n1 ; CNC(=O)c1ccc(N2CCN([C@H](C)c3ccc4c([nH]c(=O)c5cc(F)cnc54)c3F)CC2)c(F)n1]

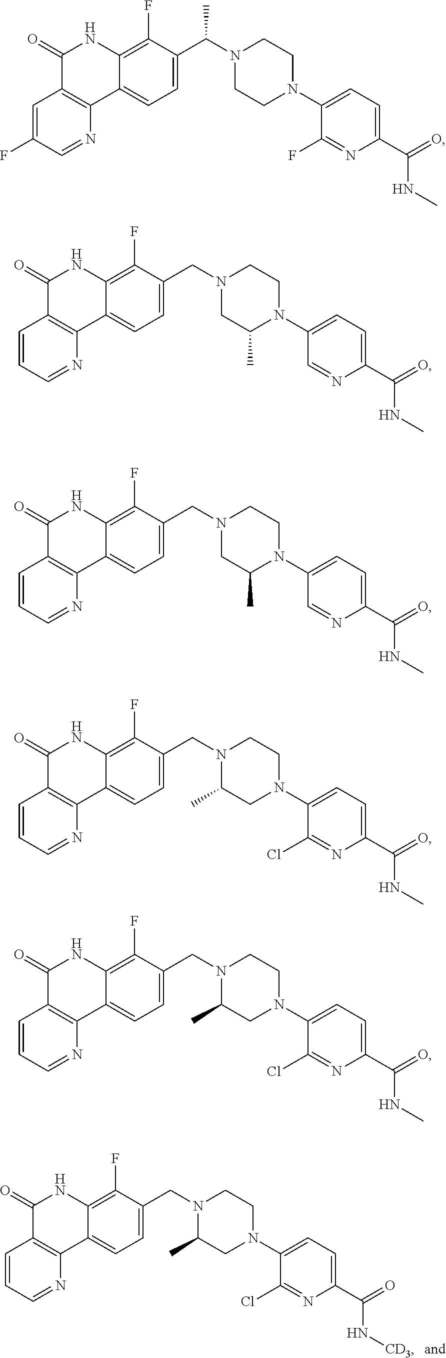 [SMILES: CNC(=O)c1ccc(N2CCN([C@@H](C)c3ccc4c([nH]c(=O)c5cc(F)cnc54)c3F)CC2)c(F)n1 ; CNC(=O)c1ccc(N2CCN(Cc3ccc4c([nH]c(=O)c5cccnc54)c3F)C[C@H]2C)cn1 ; CNC(=O)c1ccc(N2CCN(Cc3ccc4c([nH]c(=O)c5cccnc54)c3F)C[C@@H]2C)cn1 ; CNC(=O)c1ccc(N2CCN(Cc3ccc4c([nH]c(=O)c5cccnc54)c3F)[C@@H](C)C2)c(Cl)n1 ; CNC(=O)c1ccc(N2CCN(Cc3ccc4c([nH]c(=O)c5cccnc54)c3F)[C@H](C)C2)c(Cl)n1 ; [2H]C([2H])([2H])NC(=O)c1ccc(N2CCN(Cc3ccc4c([nH]c(=O)c5cccnc54)c3F)[C@H](C)C2)c(Cl)n1]

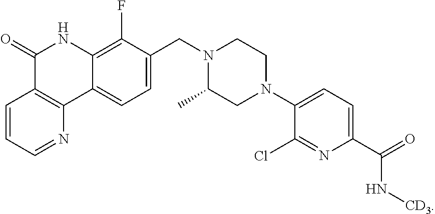 [SMILES: [2H]C([2H])([2H])NC(=O)c1ccc(N2CCN(Cc3ccc4c([nH]c(=O)c5cccnc54)c3F)[C@@H](C)C2)c(Cl)n1]

26. A pharmaceutical composition comprising a therapeutically effective amount of the compound or the pharmaceutically acceptable salt thereof according to claim 1 as an active ingredient, and a pharmaceutically acceptable carrier, diluent, or excipient.

27. A method for treating solid tumors in a subject in need thereof, comprising: administering a therapeutically effective amount of the compound or the pharmaceutically acceptable salt thereof according to claim 1 to the subject.

28. The method according to claim 27, wherein the solid tumors are selected from ovarian cancer, breast cancer, prostate cancer, and glioma.

29. A method for treating solid tumors in a subject in need thereof, comprising: administering a therapeutically effective amount of the composition according to claim 26 to the subject.

30. The method according to claim 29, wherein the solid tumors are selected from ovarian cancer, breast cancer, prostate cancer, and glioma.
